(19) United States
(12) Patent Application Publication
(10) Pub. No.: US 2018/0216090 A1
(43) Pub. Date: Aug. 2, 2018

(54) COMPOSITIONS AND METHODS COMPRISING LG12-CLADE PROTEASE VARIANTS

(71) Applicant: Danisco US Inc., Palo Alto, CA (US)

(72) Inventors: Harm Mulder, Palo Alto, CA (US); Jeffrey Veach Miller, Palo Alto, CA (US); Frits Goedegebuur, Palo Alto, CA (US); David A. Estell, Palo Alto, CA (US); Katherine Augustyn, Palo Alto, CA (US); Lilia Maria Babe, Palo Alto, CA (US); Richard R. Bott, Palo Alto, CA (US); Miles Christopher Scotcher, Palo Alto, CA (US); Jian Yao, Palo Alto, CA (US)

(21) Appl. No.: 15/557,158

(22) PCT Filed: Mar. 14, 2016
(86) PCT No.: PCT/US2016/022282

§ 371 Date: Sep. 11, 2017

Related U.S. Application Data
(60) Provisional application No. 62/131,834, filed on Mar. 12, 2015.

(51) Int. Cl. C12N 9/50; C11D 3/386

(52) U.S. Cl. CPC C12N 9/50; C11D 3/38663; C11D 3/386; C12Y 304/21062

(57) ABSTRACT

The present disclosure provides LG12-clade enzyme variants, compositions comprising these enzyme variants, and method of using these enzymes and compositions, as well as the polynucleotides encoding these enzymes, the vectors comprising these polynucleotides, and the host cells transformed with such vectors.

Serine proteases are a class of enzymes that cleave peptide bonds in proteins. The serine proteases contain a nucleophilic serine residue at the active site. Subtilisins are a large family of serine proteases, and although they have long been known in the art of industrial enzymes, there remains a need for engineered proteases that are suitable for particular conditions and uses.

The present disclosure provides, inter alia, LG12-clade protease enzymes, including variant LG12-clade protease enzymes, nucleic acids encoding the same, and compositions and methods related to the production and use thereof.

In some embodiments, the invention is a composition comprising at least one variant disclosed herein. In some embodiments, the invention is a method of cleaning using a cleaning composition comprising at least one variant disclosed herein.

Variants, compositions and methods disclosed herein relate to a protease variant, or a recombinant polypeptide or an active fragment thereof generated through conventional molecular biology techniques (see, e.g., Sambrook et al, Molecular Cloning: Cold Spring Harbor Laboratory Press). Some embodiments are directed to a protease variant, or a recombinant polypeptide or an active fragment thereof comprising an amino acid sequence comprising two or more substitutions at one or more positions selected from: (i) 1, 3, 7, 9, 19, 24, 27, 30, 38, 40, 43, 45, 48, 52, 53, 57, 72, 76, 78, 79, 86, 87, 88, 89, 90, 91, 93, 97, 99, 100, 101, 103, 104, 106, 107, 111, 114, 115, 116, 117, 118, 120, 122, 123, 124, 128, 129, 130, 131, 133, 134, 136, 139, 140, 141, 143, 144, 145, 147, 148, 149, 150, 153, 156, 158, 159, 160, 166, 181, 182, 185, 188, 194, 209, 212, 213, 215, 216, 217, 222, 224, 228, 234, 236, 238, 240, 242, 245, 246, 248, 251, 252, 256, 265, 267, 269, 270, 271, 272, 273, and 275; (ii) 1, 3, 9, 19, 24, 27, 30, 38, 40, 43, 45, 48, 52, 53, 57, 72, 76, 78, 79, 86, 87, 88, 89, 90, 91, 93, 97, 99, 100, 101, 103, 104, 106, 107, 111, 114, 115, 116, 117, 118, 120, 122, 123, 124, 128, 129, 130, 131, 133, 134, 136, 139, 140, 141, 143, 144, 145, 147, 148, 149, 150, 153, 156, 158, 159, 160, 166, 181, 182, 185, 188, 194, 209, 212, 213, 215, 216, 217, 222, 224, 228, 234, 236, 238, 240, 242, 245, 246, 248, 251, 252, 256, 265, 267, 271, 272, and 275; (iii) 1, 7, 9, 27, 38, 40, 45, 48, 52, 53, 76, 78, 91, 100, 114, 117, 118, 122, 128, 129, 130, 140, 147, 149, 156, 158, 159, 160, 166, 181, 182, 194, 209, 217, 222, 228, 234, 236, 238, 245, 248, 256, 269, 270, 271, 272, 273, and 275; (iv) 1, 24, 30, 38, 40, 48, 52, 53, 76, 78, 79, 86, 87, 88, 89, 91, 93, 97, 100, 103, 104, 106, 111, 114, 115, 116, 117, 118, 120, 122, 124, 128, 129, 130, 131, 133, 134, 136, 139, 141, 143, 144, 147, 148, 149, 150, 153, 156, 158, 159, 166, 182, 185, 194, 209, 212, 213, 216, 217, 222, 224, 236, 238, 248, 251, 252, 256, 272, and 275; (v) 1, 24, 30, 38, 40, 52, 53, 76, 78, 79, 86, 87, 88, 89, 93, 97, 100, 103, 104, 106, 111, 114, 115, 116, 117, 118, 120, 122, 124, 128, 129, 131, 133, 134, 136, 139, 141, 143, 144, 147, 148, 149, 150, 153, 156, 158, 159, 166, 182, 185, 194, 209, 212, 213, 216, 217, 222, 224, 236, 238, 248, 251, 252, 256, 272, and 275; or (vi) 1, 38, 40, 48, 52, 53, 76, 91, 114, 117, 118, 128, 129, 130, 147, 149, 156, 158, 159, 166, 182, 217, 238, 248, 256, and 272; wherein the amino acid positions of the variant or recombinant polypeptide or active fragment thereof are numbered by correspondence with the amino acid sequence of SEQ ID NO: 10. Another embodiment is directed to a protease variant, or a recombinant polypeptide or an active fragment thereof comprising an amino acid sequence comprising two or more substitutions at one or more position selected from: (i) 52, 53, 76, 86, 97, 103, 104, 106, 111, 114, 115, 116, 118, 120, 122, 124, 128, 129, 130, 131, 134, 136, 139, 141, 143, 144, 147, 148, 150, 156, 158, 159, 166, 217, 248, and 272; (ii) 53, 76, 106, 128, 131, 134, 139, 158, 159, 166, 217, 248, and 272: (iii) 52, 53, 76, 106, 118, 128, 129, 130, 131, 134, 139, 156, 158, 166, 217, 248, and 272; (iv) 52, 53, 76, 128, 118, 156, 158, 166, 217, and 248; (v) 53, 76, 128, 158, 166, and 248; (vi) 52, 53, 76, 118, 128, 156, 158, 166, 217, and 248; or (vii) 52-129, 53-166, 76-166, 166-159, 166-272, 53-76-166, 53-118-166, 53-166-272, 76-118-166, 76-166-248, 76-166-256, and 76-166-272; wherein the amino acid positions of the variant or recombinant polypeptide or active fragment thereof are numbered by correspondence with the amino acid sequence of SEQ ID NO: 10.

A still further embodiment is directed to a protease variant, or a recombinant polypeptide or an active fragment thereof comprising an amino acid sequence comprising two or more substitutions selected from: (i) A1X, T3X, G7X, P9X, A19X, S24X, K27X, I30X, N38X, A40X, N43X, K45X, A48X, S52X, G53X, A57X, V72X, N76X, T78X, T79X, Y86X, N87X, A88X, D89X, L90X, Y91X, V93X, S97X, S99X, G100X, S110X, T103X, L104X, G106X, I107X, I111X, S114X, I115X, S116X, N117X, G118X, N120X, I122X, N123X, M124X, G128X, S129X, S130X, G131X, T133X, A134X, Q136X, C139X, N140X, N141X, Y143X, N144X, R145X, I147X, V148X, V149X, I150X, A153X, S156X, S158X, S159X, G160X, G166X, S181X, S182X, T185X, S188X, S194X, T209X, N212X, N213X, A215X, S216X, F217X, M222X, A224X, A228X, I234X, A236X, Y238X, S240X, T242X, Q245X, I246X, E248X, K251X, N252X, N256X, K265X, V267X, N269X, V270X, E271X, S272X, A273X, and Q275X; (ii) A1X, T3X, P9X, A19X, S24X, K27X, I30X, N38X, A40X, N43X, K45X, A48X, S52X, G53X, A57X, V72X, N76X, T78X, T79X, Y86X, N87X, A88X, D89X, L90X, Y91X, V93X, S97X, S99X, G100X, S10X, T103X, L04X, G106X, I107X, I111X, S114X, I115X, S116X, N117X, G118X, N120X, I122X, N123X, M124X, G128X, S129X, S130X, G131X, T133X, A134X, Q136X, C139X, N140X, N141X, Y143X, N144X, R145X, I147X, V148X, V149X, I150X, A153X, S156X, S158X, S159X, G160X, G166X, S181X, S182X, T185X, S188X, S194X, T209X, N212X, N213X, A215X, S216X, F217X, M222X, A224X, A228X, I234X, A236X, Y238X, S240X, T242X, Q245X, I246X, E248X, K251X, N252X, N256X, K265X, V267X, E271X, S272X, and Q275X; (iii) A1X, G7X, P9X, K27X, N38X, A40X, K45X, A48X, S52X, G53X, N76X, T78X, Y91X, G160X, S14X, N117X, G118X, I122X, G128X, S129X, S130X, N140X, I147X, V149X, S156X, S158X, S159X, G160X, G166X, S181X, S182X, S194X, T209X, F217X, M222X, A228X, I234X, A236X, Y238X, Q245X, E248X, N256X, N269X, V270X, E271X, S272X, A273X, and Q275X; (iv) A1X, S24X, I30X, N38X, A40X, A48X, S52X, G53X, N76X, T78X, T79X, Y86X, N87X, A88X, D89X, Y91X, V93X, S97X, G100X, T103X, L104X, G106X, I111X, S114X, I115X, S116X, N117X, G118X, N120X, I122X, M124X, G128X, S129X, S130X, G131X, T133X, A134X, Q136X, C139X, N141X, Y143X, N144X, I147X, V148X, V149X, I150X, A153X, S156X, S158X, S159X, G166X, S182X, T185X, S194X, T209X, N212X, N213X, S216X, F217X, M222X, A224X, A236X, Y238X, E248X, K251X, N252X, N256X, S272X, and Q275X; (v) A1X, S24X, I30X, N38X, A40X, S52X, G53X, N76X, T78X, T79X, Y86X, N87X, A88X, D89X, V93X, S97X, G100X, T103X, L104X, G106X, I111X, S114X, I115X, S116X, N117X, G118X, N120X, I122X, M124X, G128X, S129X, G131X, T133X, A134X, Q136X, C139X, N141X, Y143X, N144X, I147X, V148X, V149X, I150X, A153X, S156X, S158X, S159X, G166X, S182X, T185X, S194X, T209X, N212X, N213X, S216X, F217X, M222X, A224X, A236X, Y238X, E248X, K251X, N252X, N256X, S272X, and Q275X; or (vi) A1X, N38X, A40X, A48X, S52X, G53X, N76X, Y91X, S114X, N117X, G118X, G128X, S129X, S130X, I147X, V149X, S156X, S158X, S159X, G166X, S182X, F217X, Y238X, E248X, N256X, and S272X; wherein X is any amino acid and the amino acid positions of the variant or recombinant polypeptide or active fragment thereof are numbered by correspondence with the amino acid sequence of SEQ ID NO: 10. Yet still another embodiment is directed to a protease variant, or a recombinant polypeptide or an active fragment thereof comprising an amino acid sequence comprising two or more substitutions selected from: (i) S52X, G53X, N76X, Y86X, S97X, T103X, L104X, G106X, I111X, S114X, I115X, S116X, G118X, N120X, I122X, M124X, G128X, S129X, S130X, G131X, A134X, Q136X, C139X, N141X, Y143X, N144X, I147X, V148X, I150X, S156X, S158X, S159X, G166X, F217X, E248X, and S272X; (ii) G53X, N76X, G106X, G128X, G131X, A134X, C139X, S158X, S159X, G166X, F217X, E248X, and S272X; (iii) S52X, G53X, N76X, G106X, G118X, G128X, S129X, S130X, G131X, A134X, C139X, S156X, G166X, F217X, E248X, and S272X; (iv) S52X, G53X, N76X, G118X, G128X, S156X, S158X, G166X, F217X, and E248X; (v) G53X, N76X, G128X, S158X, G166X, and E248X; (vi) S52X, G53X, N76X, G118X, G128X, S156X, S158X, G166X, F217X, and E248X; or (vii) S52X-S129X, G53X-G166X, N76X-G166X, G166X-S159X, G166X-S272X, G53X-N76X-G166X, G53X-G118X-G166X, G53X-G166X-S272X, N76X-G118X-G166X, N76X-G166X-E248X, N76X-G166X-N256X, and N76X-G166X-S272X; wherein X is any amino acid and the amino acid positions of the variant or recombinant polypeptide or active fragment thereof are numbered by correspondence with the amino acid sequence of SEQ ID NO: 10.

An even still further embodiment is directed to a protease variant, or a recombinant polypeptide or an active fragment thereof comprising an amino acid sequence comprising two or more substitutions selected from: (i) X1G, X1T, X3E, X3V, X7C, X9I, X9T, X9V, X19R, X24R, X24T, X27S, X27T, X30V, X38A, X38Q, X38T, X40D, X40E, X40L, X40V, X43D, X43H, X43L, X43S, X45Q, X45R, X48E, X48Q, X52A, X52N, X52Q, X52T, X53D, X53E, X53N, X57P, X72A, X76D, X78A, X78D, X78G, X78S, X79I, X86P, X87S, X88V, X89E, X89S, X90P, X91H, X93T, X97G, X99L, X100A, X100K, X101T, X103E, X103S, X104V, X106S, X107T, X107V, X111L, X114A, X14T, X115G, X116E, X116N, X117E, X117S, X118R, X120H, X122A, X123D, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130A, X130E, X130F, X130G, X130H, X130I, X130L, X130M, X130N, X130Q, X130R, X130T, X130V, X130W, X130Y, X131P, X133A, X133D, X133V, X134T, X136E, X139V, X140D, X140Y, X141S, X143T, X144S, X145N, X145W, X147A, X147V, X148L, X149G, X149L, X149N, X149S, X150V, X153S, X156A, X156H, X156N, X158T, X158V, X159D, X159E, X159N, X159Q, X160D, X166A, X166D, X166H, X166N, X166S, X166Y, X181A, X181N, X182E, X185Q, X188Y, X194P, X209F, X209V, X209Y, X212S, X213T, X215D, X215E, X216T, X217L, X217R, X222L, X224T, X228S, X234V, X236N, X236Q, X238F, X240F, X242S, X245A, X245N, X246V, X248K, X248N, X251R, X251S, X252S, X256E, X256K, X256P, X256R, X256Y, X265S, X267N, X269K, X270C, X271F, X271W, X272K, X272N, X272Q, X273V, X275H, and X275R; (ii) X1G, X1T, X3E, X3V, X9I, X9T, X9V, X19R, X24R, X24T, X27S, X27T, X30V, X38A, X38Q, X38T, X40D, X40E, X40L, X40V, X43D, X43H, X43L, X43S, X45Q, X45R, X48E, X48Q, X52A, X52N, X52Q, X52T, X53D, X53E, X53N, X57P, X72A, X76D, X78A, X78D, X78G, X78S, X79I, X86P, X87S, X88V, X89E, X89S, X90P, X91H, X93T, X97G, X99L, X100A, X100K, X111T, X103E, X103S, X104V, X106S, X107T, X107V, X111L, X114A, X114T, X115G, X116E, X116N, X117E, X117S, X118R, X120H, X122A, X123D, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130A, X130E, X130F, X130G, X130H, X130I, X130L, X130M, X130N, X130Q, X130R, X130T, X130V, X130W, X130Y, X131P, X133A, X133D, X133V, X134T, X136E, X139V, X140D, X140Y, X141S, X143T, X144S, X145N, X145W, X147A, X147V, X148L, X149G, X149L, X149N, X149S, X150V, X153S, X156A, X156H, X156N, X158T, X158V, X159D, X159E, X159N, X159Q, X160D, X166A, X166D, X166H, X166N, X166S, X166Y, X181A, X181N, X182E, X185Q, X188Y, X194P, X209F, X209V, X209Y, X212S, X213T, X215D, X215E, X216T, X217L, X217R, X222L, X224T, X228S, X234V, X236N, X236Q, X238F, X240F, X242S, X245A, X245N, X246V, X248K, X248N, X251R, X251S, X252S, X256E, X256K, X256P, X256R, X256Y, X265S, X267N, X271F, X271W, X272K, X272N, X272Q, X275H, and X275R; (iii) X1G, X7C, X9T, X27M, X38Q, X38T, X40D, X45Q, X45R, X48E, X48Q, X52F, X52Q, X52T, X53D, X53E, X53Y, X76D, X78A, X91H, X100A, X114T, X17S, X118R, X122V, X128A, X128D, X128E, X128H, X128K, X128L, X128M, X128N, X128Q, X128R, X128S, X128T, X128V, X128W, X128Y, X129D, X129E, X129F, X129G, X129H, X129I, X129K, X129L, X129M, X129N, X129P, X129Q, X129R, X129T, X129V, X129Y, X130A, X130D, X130E, X130F, X130G, X130H, X130I, X130L, X130M, X130N, X130Q, X130R, X130T, X130V, X130W, X130Y, X140D, X147A, X149G, X156D, X156H, X156N, X156T, X158T, X158V, X159D, X159N, X160D, X166D, X166H, X166Y, X181N, X182E, X194P, X209V, X217R, X222L, X228S, X234V, X236N, X236Q, X238F, X245A, X248D, X248K, X248N, X256E, X256P, X256R, X269K, X270C, X271G, X272K, X272Q, X273V, X275R, and X275Y, (iv) X1G, X1T, X24R, X24T, X30V, X38A, X38Q, X38T, X40D, X40E, X40L, X40V, X48Q, X52A, X52N, X52Q, X52T, X53D, X53E, X53N, X76D, X78A, X78D, X78G, X78S, X79I, X86P, X87S, X88V, X89E, X91H, X93T, X97G, X100A, X100K, X103E, X103S, X104V, X106S, X111L, X114A, X114T, X115G, X116E, X116N, X117E, X117S, X18R, X120H, X122A, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130F, X130H, X130I, X130Y, X131P, X133A, X133D, X133V, X134T, X136E, X139V, X141S, X143T, X144S, X147A, X147V, X148L, X149G, X149L, X149N, X149S, X150V, X153S, X156A, X156H, X156N, X158T, X158V, X159D, X159E, X159N, X159Q, X166A, X166D, X166H, X166N, X166S, X166Y, X182E, X185Q, X194P, X209F, X209V, X209Y, X212S, X213T, X216T, X217L, X217R, X222L, X224T, X236N, X236Q, X238F, X248K, X248N, X251R, X251S, X252S, X256E, X256K, X256P, X256R, X256Y, X272K, X272N, X272Q, X275H, and X275R; (v) X1G, X1T, X24R, X24T, X30V, X38A, X38Q, X38T, X400D, X40E, X40L, X40V, X52A, X52N, X52Q, X52T, X53D, X53E, X53N, X76D, X78A, X78D, X78G, X78S, X79I, X86P, X87S, X88V, X89E, X93T, X97G, X100A, X100K, X103E, X103S, X104V, X106S, X111L, X14A, X114T, X115G, X116E, X116N, X117E, X117S, X18R, X120H, X122A, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X131P, X133A, X133D, X133V, X134T, X136E, X139V, X141S, X143T, X144S, X147A, X147V, X148L, X149G, X149L, X149N, X149S, X150V, X153S, X156A, X156H, X156N, X158T, X158V, X159D, X159E, X159N, X159Q, X166A, X166D, X166H, X166N, X166S, X166Y, X182E, X185Q, X194P, X209F, X209V, X209Y, X212S, X213T, X216T, X217L, X217R, X222L, X224T, X236N, X236Q, X238F, X248K, X248N, X251R, X251S, X252S, X256E, X256K, X256P, X256R, X256Y, X272K, X272N, X272Q, X275H, and X275R; or (vi) X1G, X38Q, X40D, X48Q, X52F, X52T, X53E, X53Y, X76D, X91H, X114T, X117S, X118R, X128D, X128M, X128V, X129F, X129M, X129R, X129V, X130F, X130H, X130I, X130Y, X147A, X149G, X156N, X158T, X159N, X166D, X166H, X182E, X217R, X238F, X248N, X256E, X256P, X256R, and X272K; wherein X is any amino acid and the amino acid positions of the variant or recombinant polypeptide or active fragment thereof are numbered by correspondence with the amino acid sequence of SEQ ID NO:10. Another embodiment is directed to a protease variant, or a recombinant polypeptide or an active fragment thereof comprising an amino acid sequence comprising two or more substitutions selected from: (i) X52T, X53D, X76D, X86P, X97G, X103S, X104V, X106S, X111L, X114A, X115G, X116N, X118R, X120H, X122A, X124L, X128D, X128S, X129F, X130Y, X131P, X134T, X136E, X139V, X141S, X143T, X144S, X147V, X148L, X150V, X156N, X158T, X159D, X166D, X217R, X248N, and X272K; (ii) X53D, X76D, X106S, X128S, X131P, X134T, X139V, X158T, X159D, X166D, X217R, X248N, and X272K: (iii) X52T, X53D, X76D, X106S, X118R, X128D, X129F, X130Y, X131P, X134T, X139V, X156N, X166D, X217R, X248N, and X272K; (iv) X52T, X53D, X76D, X118R, X128S, X156N, X158T, X166D, X217R, and X248N; (v) X53D, X76D, X128S, X158T, X166D, and X248N; (vi) X52T, X53D, X76D, X118R, X128S, X156N, X158T, X166D, X217R, and X248N; or (vii) X52T-X129F, X53D-X66D, X76D-X166D, X166H-X159D, X166D-X272K, X53D-X76D-X66D, X53D-X18R-X166D, X53D-X166D-X272K, X76D-X118R-X166D, X76D-X166D-X248K, X76D-X166D-X256R, and X76D-X166D-X272K; wherein X is any amino acid and the amino acid positions of the variant or recombinant polypeptide or active fragment thereof are numbered by correspondence with the amino acid sequence of SEQ ID NO: 10.

An even further embodiment is directed to a protease variant, or a recombinant poly peptide or an active fragment thereof comprising an amino acid sequence comprising two or more substitutions selected from: (i) A001G, A001T, T003E, T003V, G007C, P009I, P009T, P009V, A019R, S024R, S024T, K027S, K027T, I030V, N038A, N038Q, N038T, A040D, A040E, A040L, A040V, N043D, N043H, N043L, N043S, K045Q, K045R, A048E, A048Q, S052A, S052N, S052Q, S052T, G053D, G053E, G053N, A057P, V072A, N076D, T078A, T078D, T078G, T078S, T079I, Y086P, N087S, A088V, D089E, D089S, L090P, Y091H, V093T, S097G, S099L, G100A, G100K, S101T, T103E, T103S, L104V, G106S, I007T, I107V, I111L, S114A, S114T, I115G, S116E, S116N, N117E, N117S, G118R, N120H, I122A, N123D, M124L, G128A, G128D, G128E, G128H, G128K, G128M, G128N, G128Q, G128S, G128Y, S129D, S129E, S129G, S129H, S129I, S129K, S129L, S129M, S129N, S129P, S129Q, S129R, S129T, S129V, S129Y, S130A, S130E, S130F, S130G, S130H, S130I, S130L, S130M, S130N, S130Q, S130R, S130T, S130V, S130W, S130Y, G131P, T133A, T133D, T133V, A134T, Q136E, C139V, N140D, N140Y, N14I, Y143T, N144S, R145N, R145W, I147A, I147V, V148L, V149G, V149L, V149N, V149S, I150V, A153S, S156A, S156H, S156N, S158T, S158V, S159D, S159E, S159N, S159Q, G160D, G166A, G166D, G166H, G166N, G166S, G166Y, S181A, S181N, S182E, T185Q, S188Y, S194P, T209F, T209V, T209Y, N212S, N213T, A215D, A215E, S216T, F217L, F217R, M222L, A224T, A228S, I234V, A236N, A236Q, Y238F, S240F, T242S, Q245A, Q245N, I246V, E248K, E248N, K251R, K251S, N252S, N256E, N256K, N256P, N256R, N256Y, K265S, V267N, N269K, V270C, E271F, E271W, S272K, S272N, S272Q, A273V, Q275H, and Q275R; (ii) A001G, A001T, T003E, T003V, P009I, P009T, P009V, A019R, S024R, S024T, K027S, K027T, I030V, N038A, N038Q, N038T, A040D, A040E, A040L, A040V, N043D, N043H, N043L, N043S, K045Q, K045R, A048E, A048Q, S052A, S052N, S052Q, S052T, G053D, G053E, G053N, A057P, V072A, N076D, T078A, T078D, T078G, T078S, T079I, Y086P, N087S, A088V, D089E, D089S, L090P, Y091H, V093T, S097G, S099L, G100A, G100K, S101T, T103E, T103S, L104V, G106S, I107T, I107V, I111L, S114A, S114T, I115G, S116E, S116N, N117E, N117S, G118R, N120H, I122A, N123D, M124L, G128A, G128D, G128E, G128H, G128K, G128M, G128N, G128Q, G128S, G128Y, S129D, S129E, S129G, S129H, S129I, S129K, S129L, S129M, S129N, S129P, S129Q, S129R, S129T, S129V, S129Y, S130A, S130E, S130F, S130G, S130H, S130I, S130L, S130M, S130N, S130Q, S130R, S130T, S130V, S130W, S130Y, G131P, T133A, T133D, T133V, A134T, Q136E, C139V, N40D, N4Y, N14I, Y143T, N144S, R145N, R145W, I147A, I147V, V148L, V149N, V149L, V149N, V149S, I150V, A153S, S156A, S156H, S156N, S158T, S158V, S159D, S159E, S159N, S159Q, G160D, G166A, 0166D, G166H, G166N, G166S, G166Y, S181A, S181N, S182E, T185Q, S188Y, S194P, T209F, T209V, T209Y, N212S, N213T, A215D, A215E, S216T, F217L, F217R, M222L, A224T, A228S, I234V, A236N, A236Q, Y238F, S240F, T242S, Q245A, Q245N, I246V, E248K, E248N, K251R, K251S, N252S, N256E, N256K, N256P, N256R, N256Y, K26S, V267N, E271F, E271W, S272K, S272N, S272Q, Q275H, and Q275R: (iii) A001G, G007C, P009T, K027M, N038Q, N038T, A040D, K045Q, K045R, A048E, A048Q, S052F, S052Q, S052T, G053D, G053E, G053Y, N076D, T078A, Y091H, G100A, S114T, N17S, G118R, I122V, G128A, G128D, G128E, G128H, G128K, G128L, G128M, G128N, G128Q, G128R, G128S, G128T, G128V, G128W, G128Y, S129D, S129E, S129F, S129G, S129H, S129I, S129K, S129L, S129M, S129N, S129P, S129Q, S129R, S129T, S129V, S129Y, S130A, S130D, S130E, S130F, S130G, S130H, S130I, S130L, S130M, S130N, S130G, S130R, S130T, S130V, S130W, S130Y, N140D, I147A, V149G, S156D, S156H, S156N, S156T, S158T, S158V, S159D, S159N, G160D, G166D, G166H, G166Y, S181N, S182E, S194P, T209V, F217R, M222L, Y238F, Q245A, E248D, E248K, E248N, N256E, N256P, N256R, N269K, V270C, E271G, S272K, S272Q, A273V, Q275R, and Q275Y; (iv) A001G, A001T, S024T, I030V, N038Q, A040D, A048Q, S052A, S052Q, S052T, G053D, G053E, N076D, T078D, T078S, T079I, Y086P, N087S, A088V, D089E, D089S, Y091H, V093T, S097G, G100A, T103E, T103S, L104V, G106S, I111L, S114A, S114T, I115G, S116N, N117S, G118R, N120H, I122A, M124L, G128S, S129P, S130I, S130H, S130I, S130Y, G131P, T133A, T133V, A134T, Q136E, C139V, N141S, Y143T, N144S, I147A, I147V, V148L, V149G, V149L, I150V, A153S, S156H, S156N, S158T, S159D, S159N, G166D, G166H, G166S, S182E, T185Q, S194P, T209Y, N212S, N213T, S216T, F217L, F217R, M222L, A224T, A236N, Y238F, E248N, K251S, N252S, N256P, N256R, N256Y, S272K, S272Q, and Q275H; (v) A001G, A001T, S024T, I030V, N038Q, A040D, S052A, S052Q, S052T, G053D, G053E, N076D, T078D, T078S, T079I, Y086P, N087S, A088V, D089E, D089S, V093T, S097G, G100A, T103E, T103S, L104V, G106S, I111L, S114A, S114T, I115G, S116N, N117S, G118R, N120H, I122A, M124L, G128S, S129P, G131P, T133A, T133V, A134T, Q136E, C139V, N141S, Y143T, N144, I147A, I147V, V148L, V149G, V149L, I150V, A153S, S156H, S156N, S158T, S159D, S159N, G166D, G166H, G166S, S182E, T185Q, S194P, T209Y, N212S, N213T, S216T, F217L, F217R, M222L, A224T, A236N, Y238F, E248N, K251S, N252S, N256P, N256R, N256Y, S272K, S272Q, and Q275H; or (vi) A001G, N038Q, A040D, A048Q, S052F, S052T, G053E, G053Y, N076D, Y091H, S114T, N117S, G118R, G128D, G128M, G128V, S129F, S129M, S129R, S129V, S130F, S130H, S130I, S130Y, I147A, V149G, S156N, S158T, S159N, G166D, G166H, S182E, F217R, Y238F, E248N, N256E, N256P, N256R, and S272K; wherein the amino acid positions of the variant or recombinant polypeptide or active fragment thereof are numbered by correspondence with the amino acid sequence of SEQ ID NO: 10. A still further embodiment is directed to a protease variant, or a recombinant polypeptide or an active fragment thereof comprising an amino acid sequence comprising two or more substitutions selected from: (i) S52T, G53D, N76D, Y086P, S097G, T103S, L104V, G106S, I111L, S114A, I115G, S116N, G118R, N120H, I122A, M124L, G128D, G128S, S129F, S129P, S130Y, G131P, A134T, Q136E, C139V, N141S, Y143T, N144S, I147V, V148L, I150V, S156N, S158T, S159D, G166D, F217R, E248N, and S272K; (ii) G53D, N76D, G106S, G128S, G131P, A134T, C139V, S158T, S159D, G166D, F217R, E248N, and S272K; (iii) S52T, G53D, N76D, G118R, G128D, S129F, S130Y, G131P, A134T, C139V, S156N, G166D, F217R, E248N, and S272K; (iv) S52T, G53D, N76D, G118R, G128S, S156N, S158T, G166D, F217R, and E248N; (v) G53D, N76D, G128S, S158T, G166D, and E248N; (vi) S52T, G53D, N76D, G118R, S156N, S158T, G166D, F217R, and E248N; or (vii) S52T-S129F, G53D-G166D, N76D-G166D, G166H-S159D, G166D-S272K, G53D-N76D-G166D, G53D-G118R-G166D, G53D-G166D-S272K, N76D-G118R-G166D, N76D-G166D-E248K, N76D-G166D-N256R, and N76D-G166D-S272K; wherein the amino acid positions of the variant or recombinant polypeptide or active fragment thereof are numbered by correspondence with the amino acid sequence of SEQ ID NO: 10.

An even still further embodiment is directed to a protease variant, or a recombinant polypeptide or an active fragment thereof comprising an amino acid sequence comprising one or more deletions at one or more positions selected from 53, 55, 56, 57, and 58 or G053, P055. N056. A057 and L058; wherein the amino acid positions of the variant, or recombinant polypeptide or active fragment thereof are numbered by correspondence with the amino acid sequence of SEQ ID NO: 10.

Another embodiment is directed to an LG12-Clade of subtilisins comprising one or more protease variants described herein, or recombinant polypeptide or an active fragment thereof, wherein said variant, or recombinant polypeptide or active fragment thereof comprises an EPNALQDGNGHGTH (SEQ ID NO:44) motif. In a still further embodiment, one or more protease variant described herein, or recombinant polypeptide or an active fragment thereof comprises an amino acid sequence having at least 75% amino acid sequence identity to SEQ ID NO:10. In yet still other embodiments, one or more protease variant described herein, or recombinant polypeptide or an active fragment thereof has protease activity in the presence of a surfactant, cleaning activity in a detergent composition, or a combination thereof. In an even further embodiment, one or more protease variant described herein, or recombinant polypeptide or an active fragment thereof has one or more improved property selected from improved cleaning performance, improved stability, and improved residual protease activity when compared to SEQ ID NO: 10 and/or 48. A still further embodiment is directed to a polynucleotide comprising a nucleic acid sequence that encodes a protease variant, or recombinant polypeptide or active fragment thereof described herein.

An even further embodiment is directed to a composition containing one or more protease variants described herein, or recombinant polypeptide or an active fragment thereof. In yet still an even further embodiment, the composition described herein contains phosphate, is phosphate-free, contains borate, is boron-free, or combinations thereof. In other embodiments, the composition is a boron-free composition. In some embodiments, a boron-free composition is a composition to which a borate stabilizer has not been added. In another embodiment, a boron-free composition is a composition that contains less than 5.5% boron. In a still further embodiment, a boron-free composition is a composition that contains less than 4.5% boron. In yet still another embodiment, a boron-free composition is a composition that contains less than 3.5% boron. In yet still further embodiments, a boron-free composition is a composition that contains less than 2.5% boron. In even further embodiments, a boron-free composition is a composition that contains less than 1.5% boron. In another embodiment, a boron-free composition is a composition that contains less than 1.0% boron. In still further embodiments, a boron-free composition is a composition that contains less than 0.5% boron. In other embodiments, the composition is a cleaning composition. In yet further embodiments, the cleaning composition is a detergent composition. In still further embodiments, a boron-free composition is a composition substantially-free of boron.

In some embodiments, the serine protease enzymes disclosed herein find use in soil removal, grain ethanol production, animal feed, food processing and digestive aids, leather processing, and/or personal care. In still further embodiments, the serine protease enzyme variants disclosed herein can be combined with other enzymes useful in detergent compositions. Still further embodiments provide methods for using the serine protease enzyme variants disclosed herein.

Serine protease enzymes can be improved by engineering the enzyme to have better cleaning performance, stability in the presence of chemical agents, and/or thermostability over currently available serine protease enzymes. In some embodiments, the serine protease enzyme variants disclosed herein have a minimum performing index (PI) for wash performance, stability of the enzyme in detergent compositions and thermostability of the enzyme, while having at least one of these characteristics improved from a parent serine protease enzyme. In further embodiments, the serine protease enzymes disclosed herein are particularly well suited to and useful in a variety of cleaning applications. In some embodiments, the serine protease enzymes are LG12-clade serine protease variants. In other embodiments, the serine protease enzyme variants disclosed herein have one or more modifications, such as a substitution, insertion or deletion, as compared to a parent serine protease enzyme. In some embodiments, the protease variant, or recombinant polypeptide or active fragment thereof is derived from a reference polypeptide selected from SEQ ID NOs:10, 18, 19, 45, 46, 57, 58, and 66. In other embodiments the reference polypeptide is LG12 wild-type protease (SEQ ID NO:10).

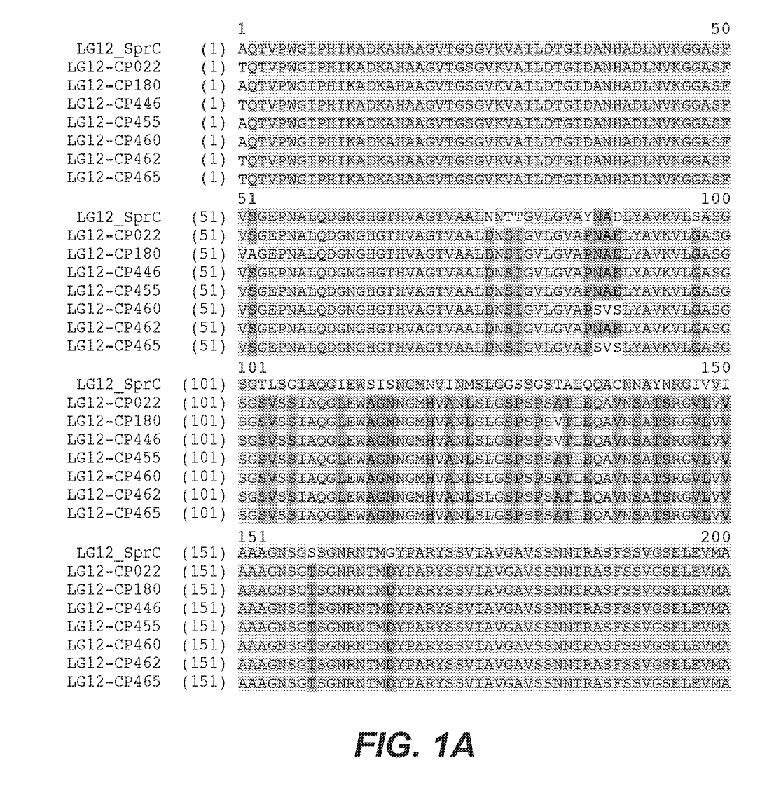

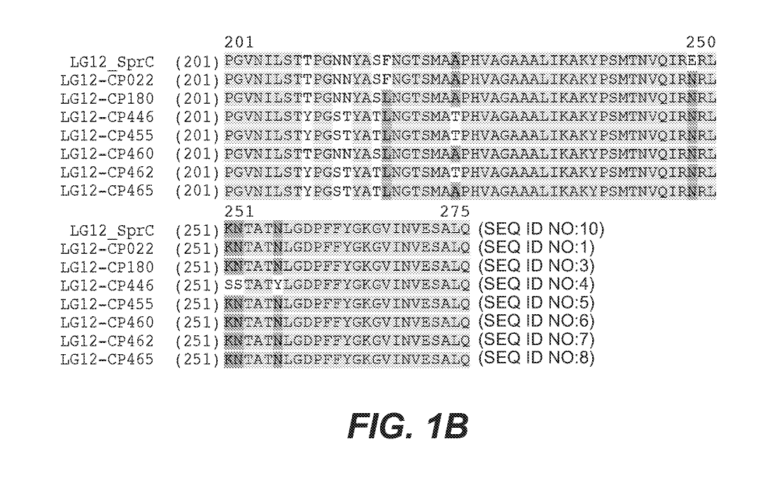

FIG. 1A-1B provides a Clustal W protein sequence alignment of LG12-Clade proteases (LG12-CP022, LG12-CP180, LG12-CP446, LG12-CP455, LG12-CP460, LG12-CP462, and LG12-CP465) and parent LG12 wild-type protease.

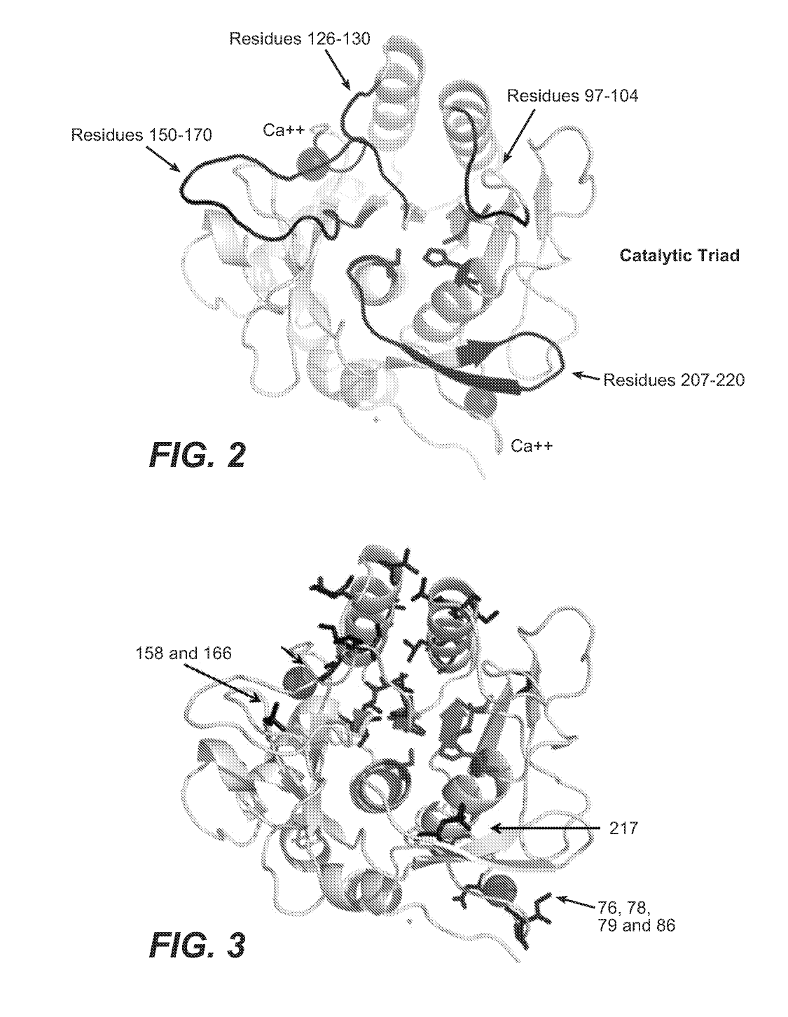

FIG. 2 provides a structure of the LG12 wild-type protease depicting the key loops comprising residues 97-104, 126-130, 150-170 and 207-220 that form the substrate binding region in which the overall folding is represented as a schematic in light gray; the catalytic triad formed by Aspartic acid 32. Histidine 64 and Serine 221 is shown as black stick figures; the two calcium sites common to all commercial bacterial subtilisins are represented as dark gray spheres; and the key loops forming the substrate binding region are set forth as black lines.

FIG. 3 provides a 3D structure of the LG12 wild-type protease depicting (i) side chains of substitutions common to the best LG2-Clade protease variants, which substitutions are primarily localized in two segments that form the walls of the substrate binding site and the following helical segments that serve to anchor the residue 97-148 segment, (ii) substitutions 158, 166 and 217, which line the substrate binding cavity, and (iii) substitutions at positions 76, 78, 79 and 86, which are near one of the critical calcium binding sites.

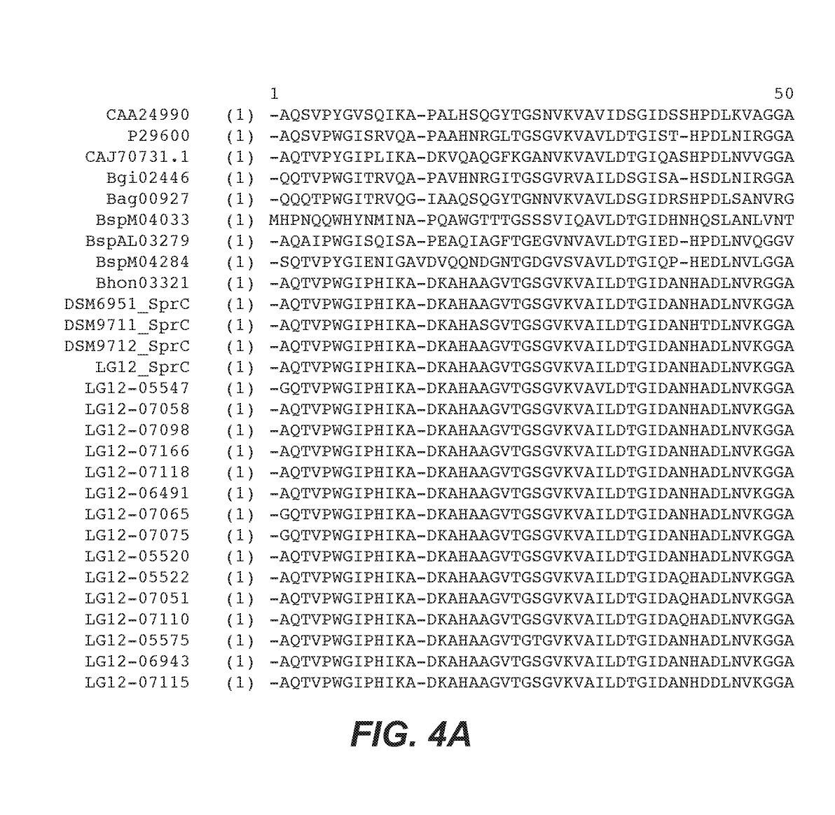

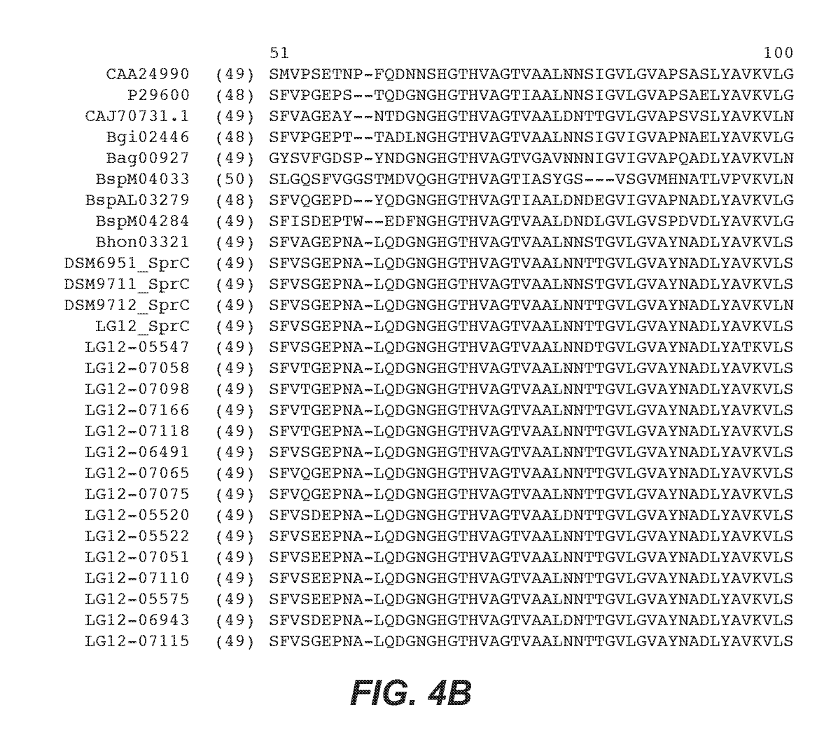

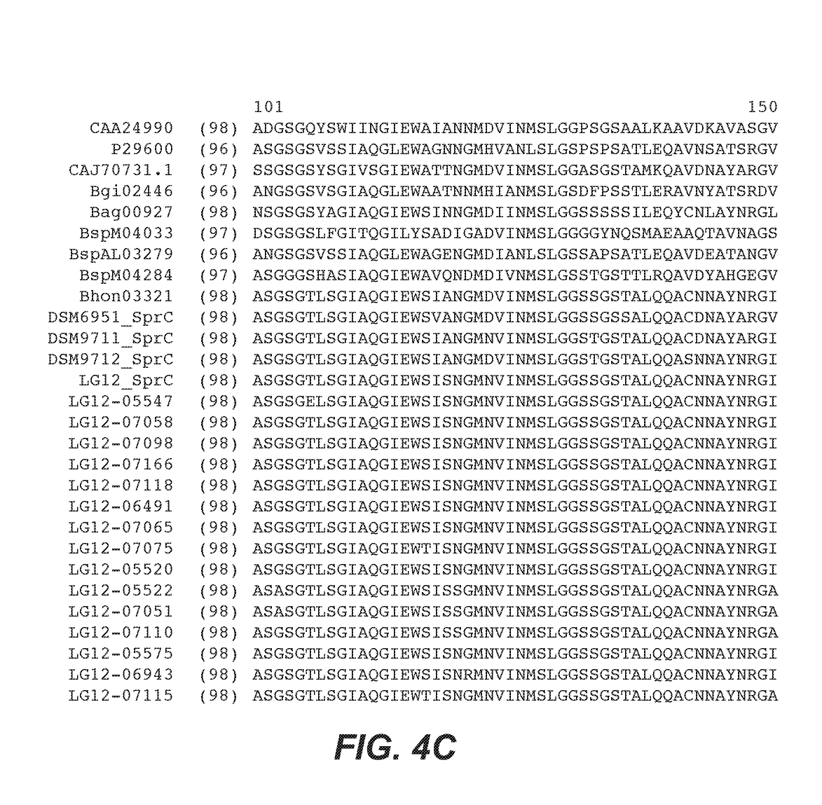

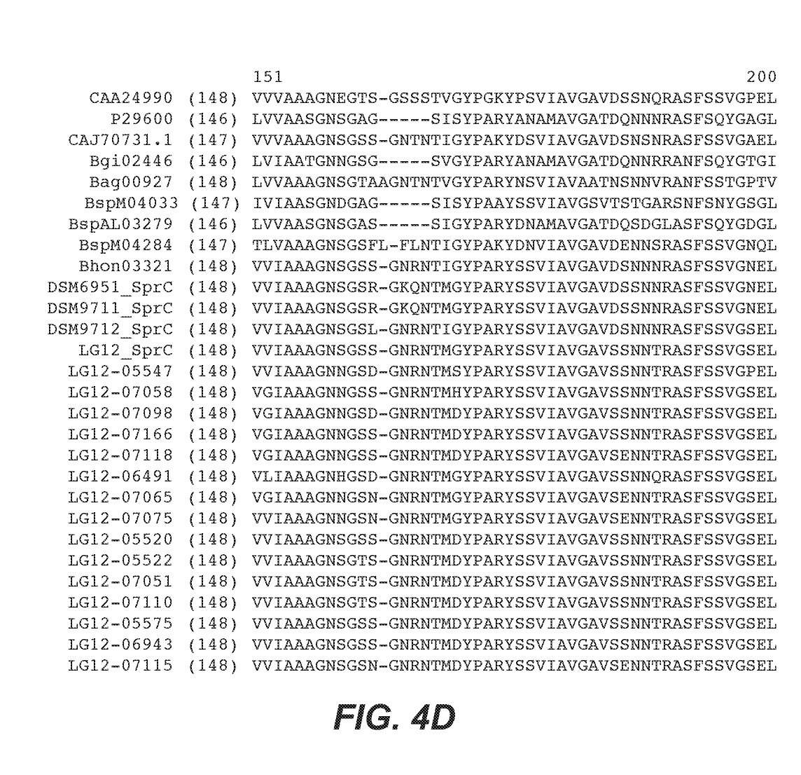

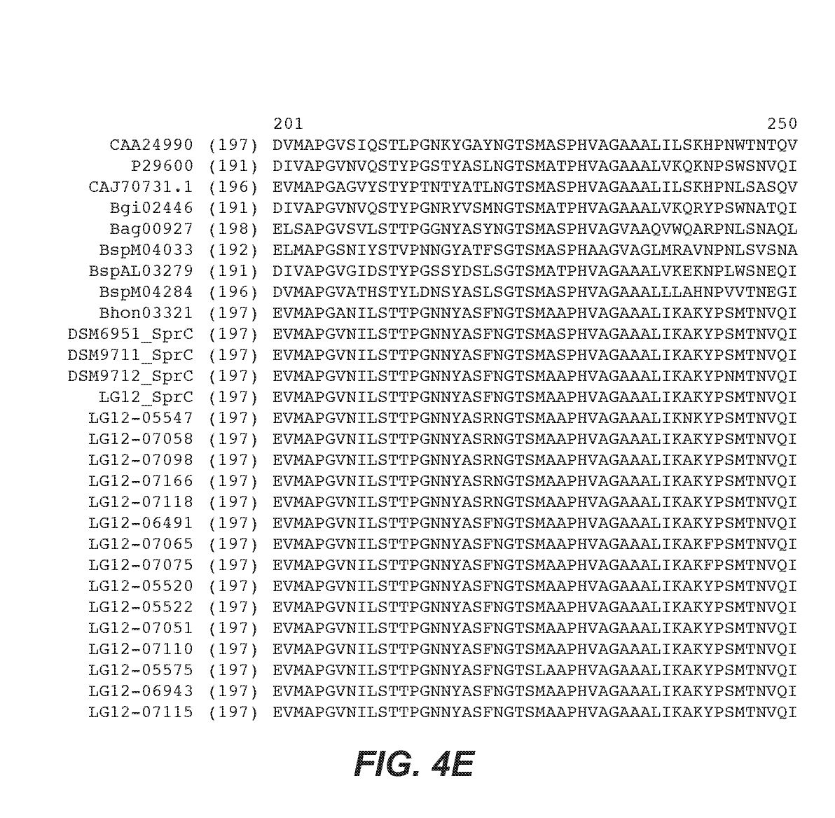

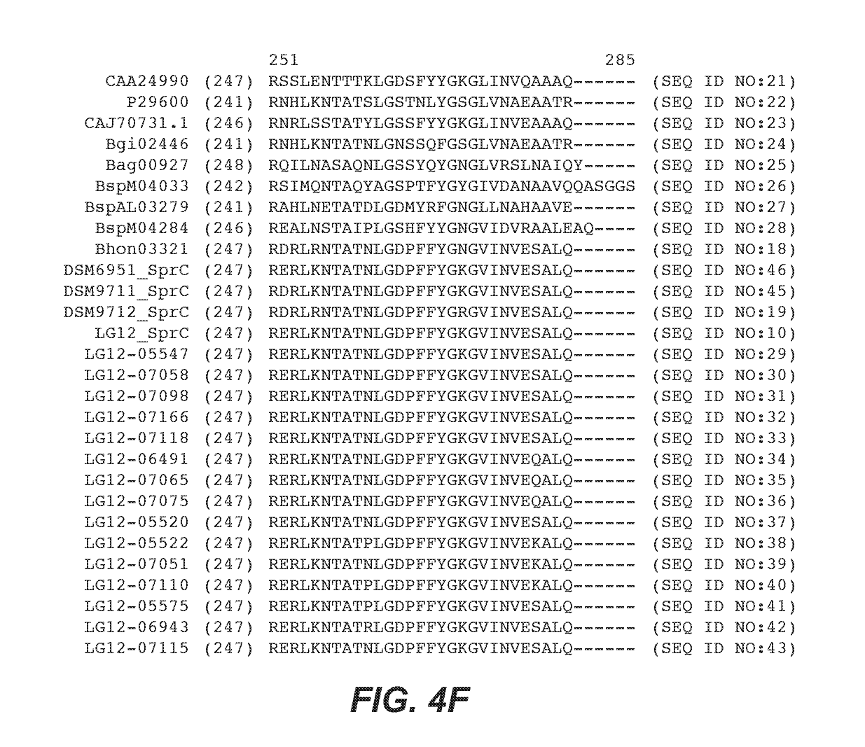

FIG. 4A-4F provides the Clustal W alignment of various Bacillus subtilisin mature protease sequences including members of the LG2-clade.

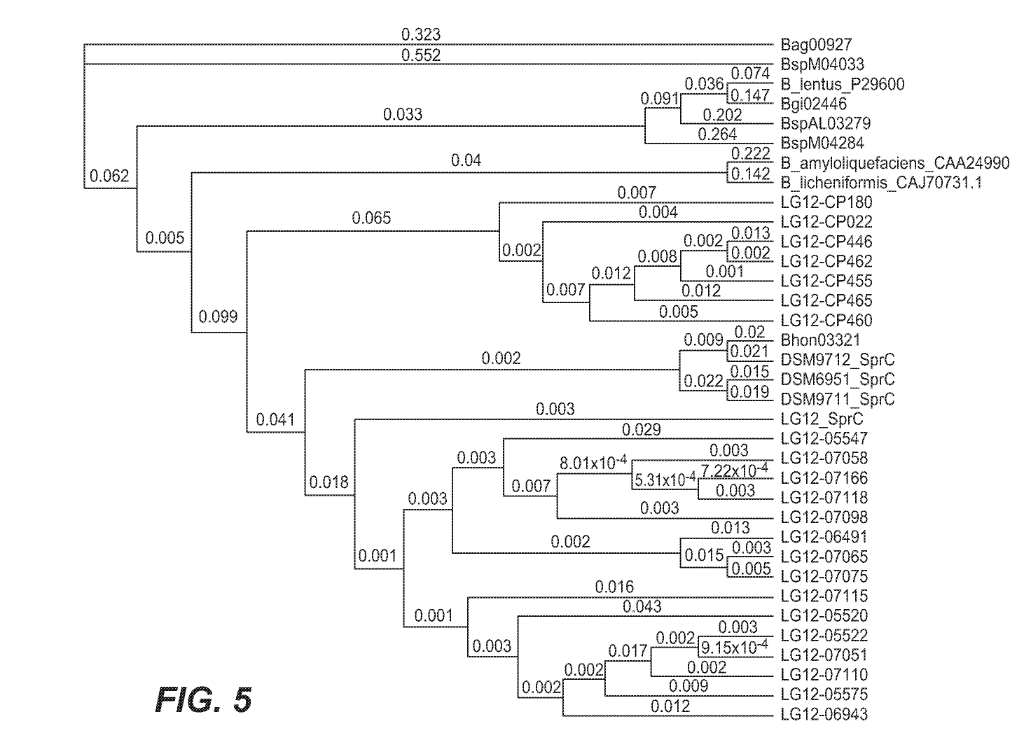

FIG. 5 provides the phylogenetic tree of LG12-clade proteases and various other Bacillus subtilisins.

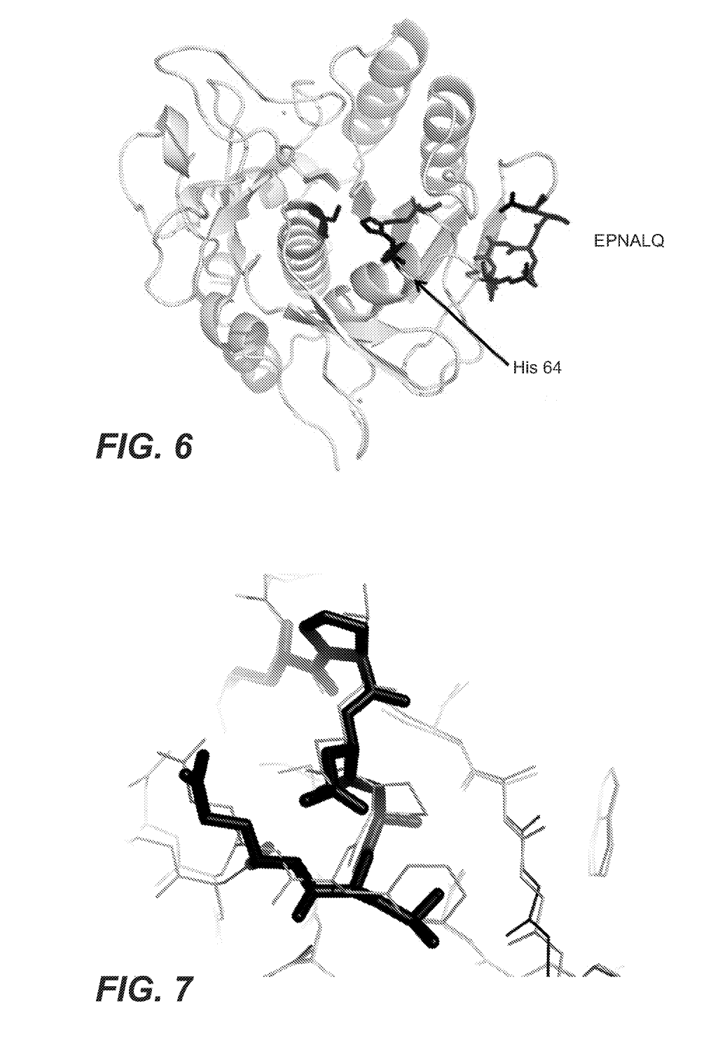

FIG. 6 provides a structure of LG12 wild-type protease depicting the catalytic triad formed by Aspartic acid 32. Histidine 64 and Serine 221 and the region including residues 54-59 leading into His 64 of the triad in which the overall fold of the main chain is shown in light gray; the side chains of the catalytic triad formed by Aspartic acid 32, Histidine 64 and Serine 221 are shown as black sticks; Histidine 64 is indicated by the arrow: the region including residues 54-59 leading into Histidine 64 of the triad are also shown as black sticks.

FIG. 7 provides a crystallographic structure of LG12 wild-type protease in which the region 54-59 in the sequence EPNALQ of LG12, shown as black sticks, is compared with the equivalent loop segment in subtilisin BPN′ (pdb entry 2ST1), shown as gray lines.

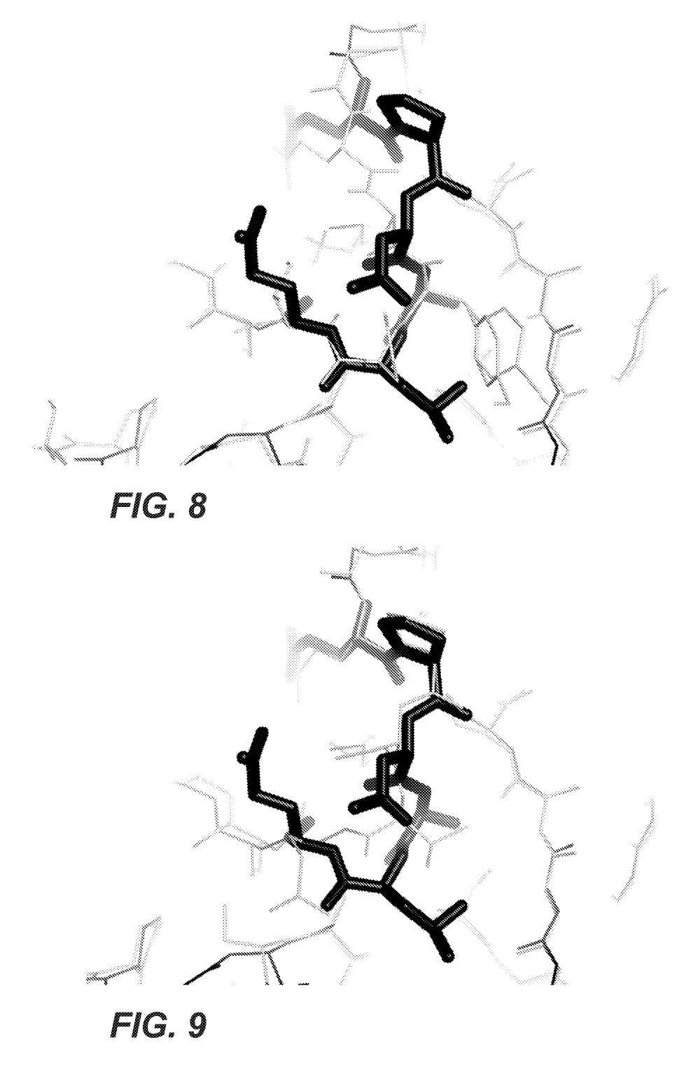

FIG. 8 provides a crystallographic structure of LG2 wild-type protease in which the region 54-59 in the sequence EPNALQ of LG12, shown as black sticks, is compared with the equivalent loop segment in subtilisin Carlsberg (pdb entry 3UNX), shown as gray lines.

FIG. 9 provides a crystallographic structure of LG12 wild-type protease in which the region 54-59 in the sequence EPNALQ of LG12, shown as black sticks, is compared with the equivalent loop segment in subtilisin from B. Lentus, shown as gray lines.

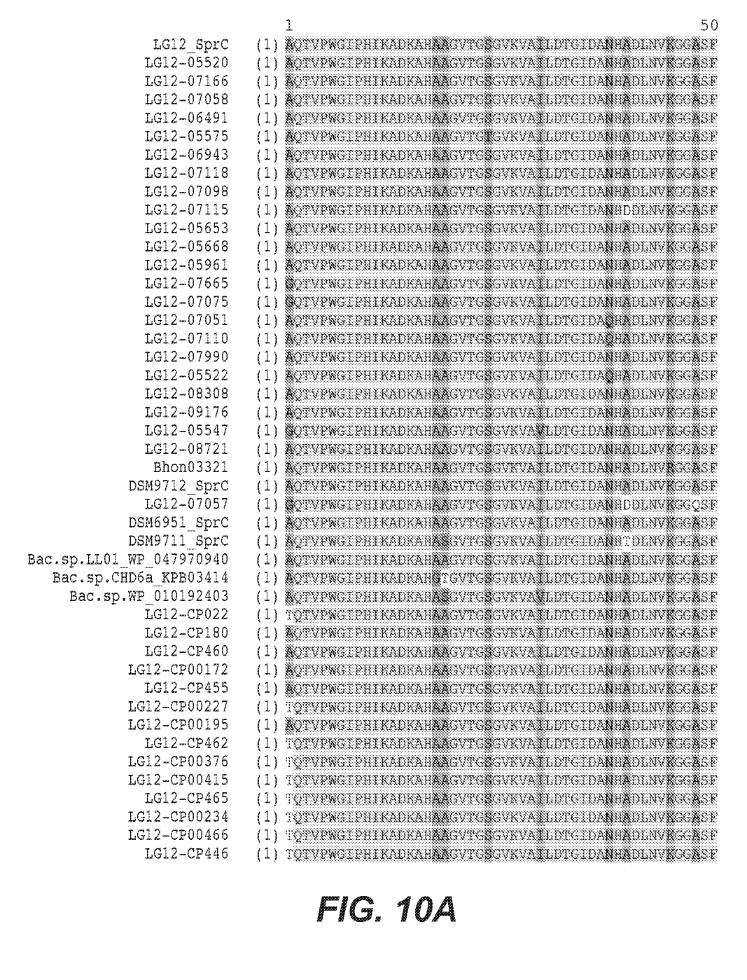

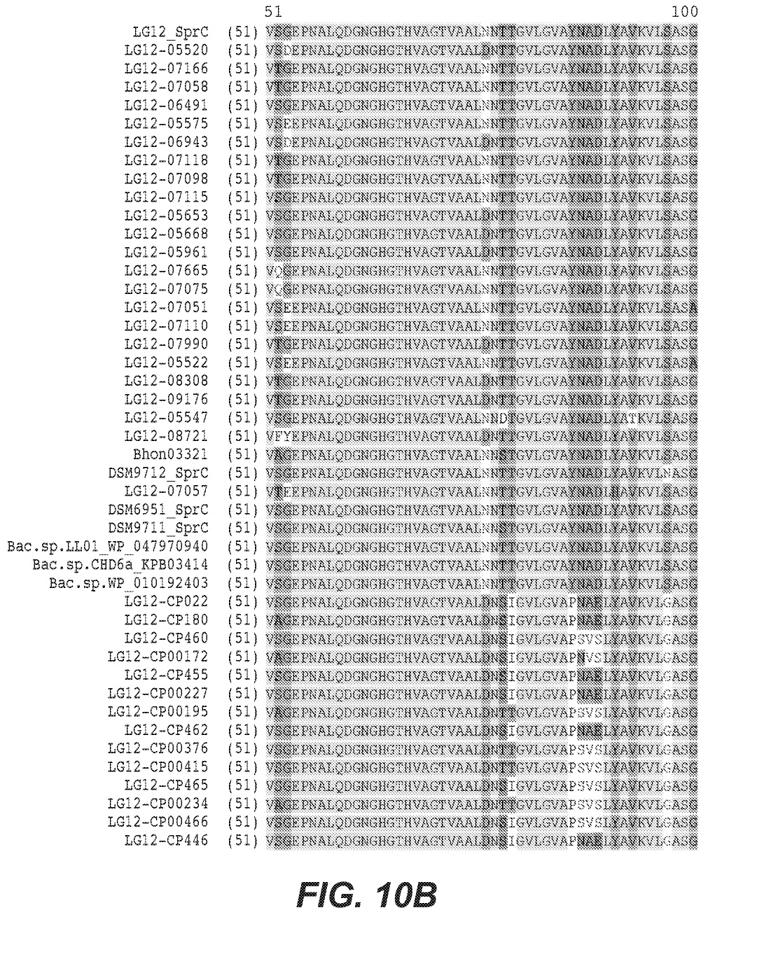

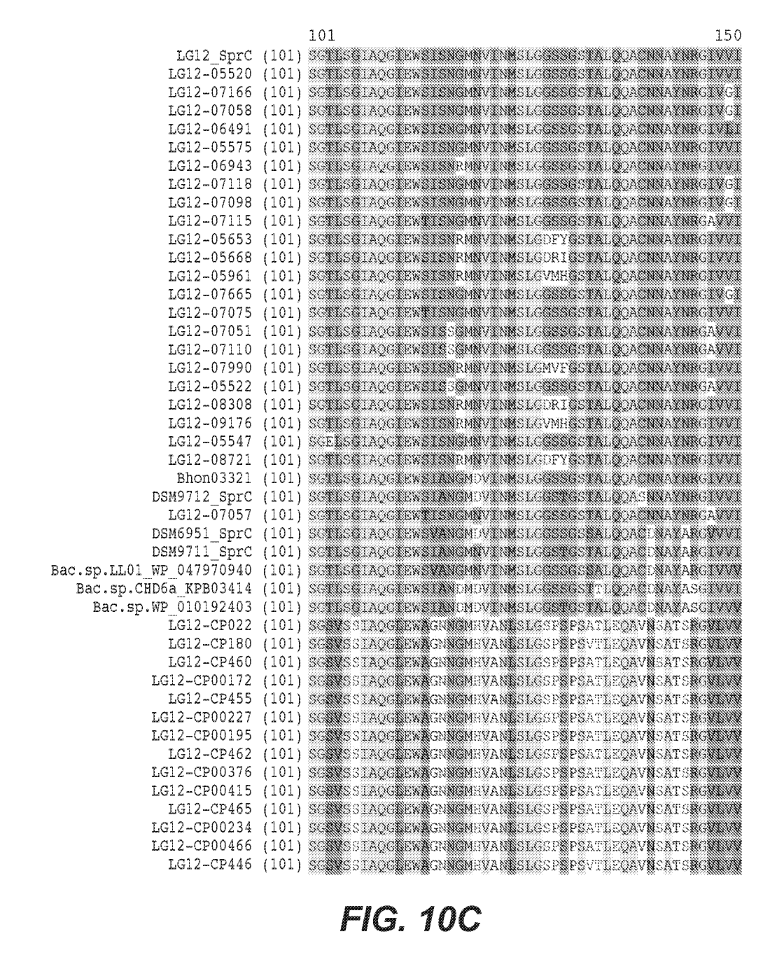

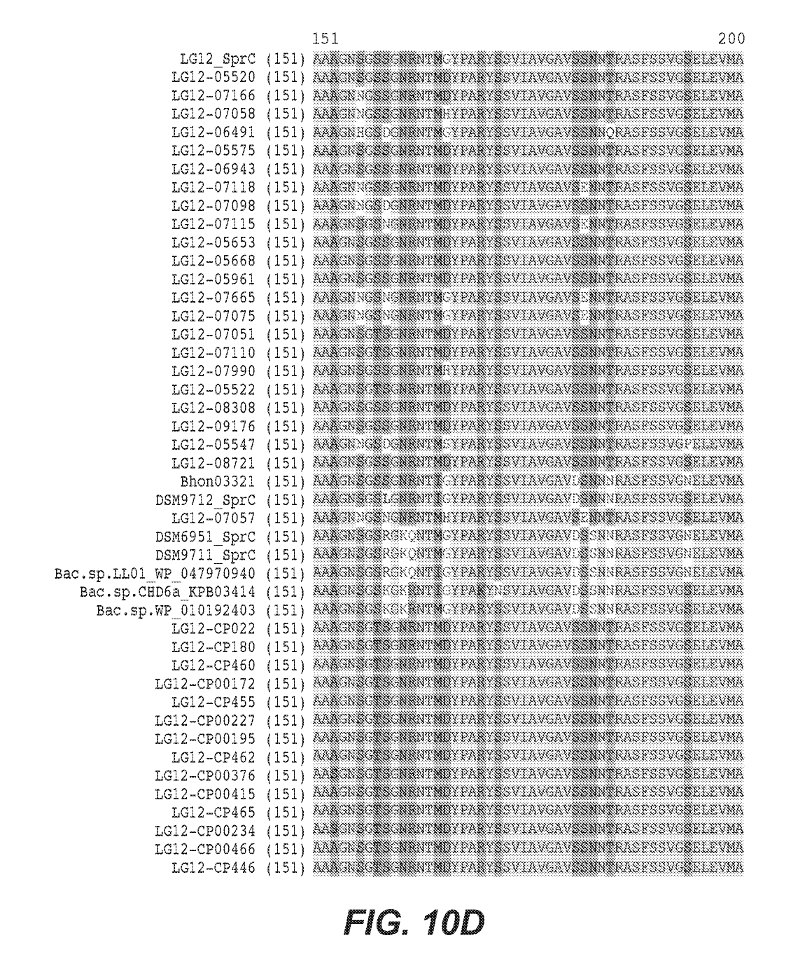

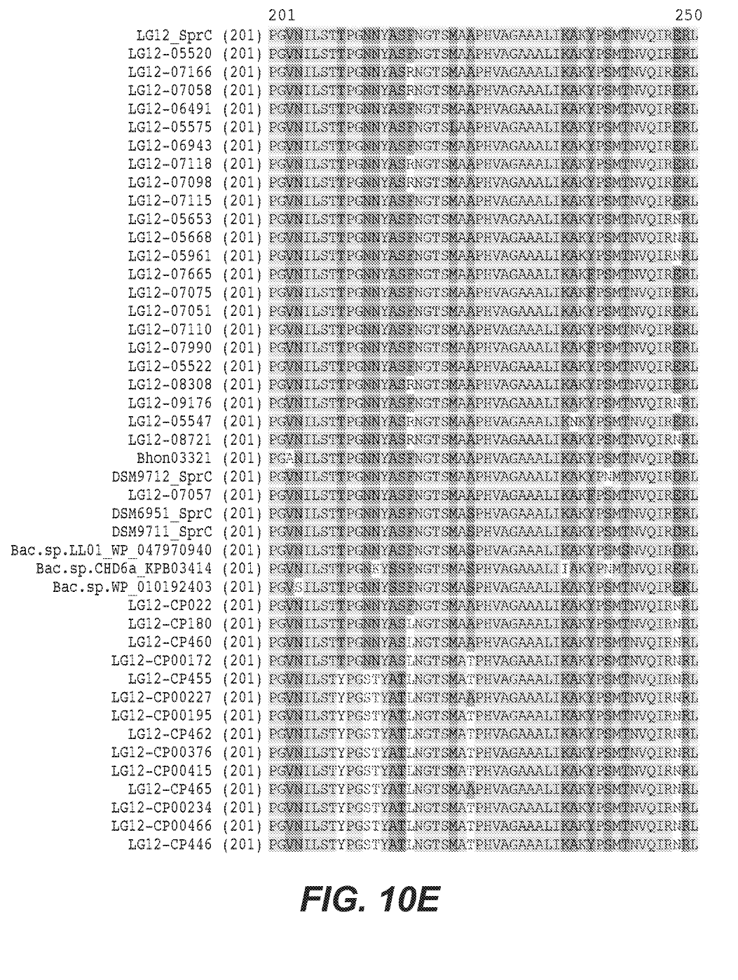

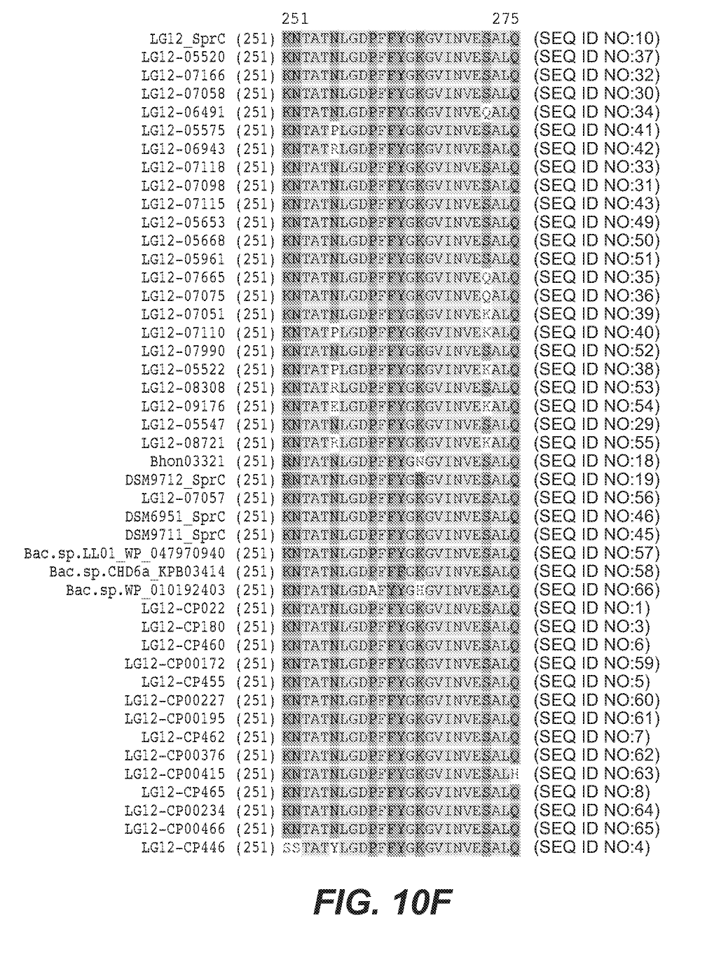

FIG. 10 A-10F provides the Clustal W alignment of various LG12-clade mature protease sequences.

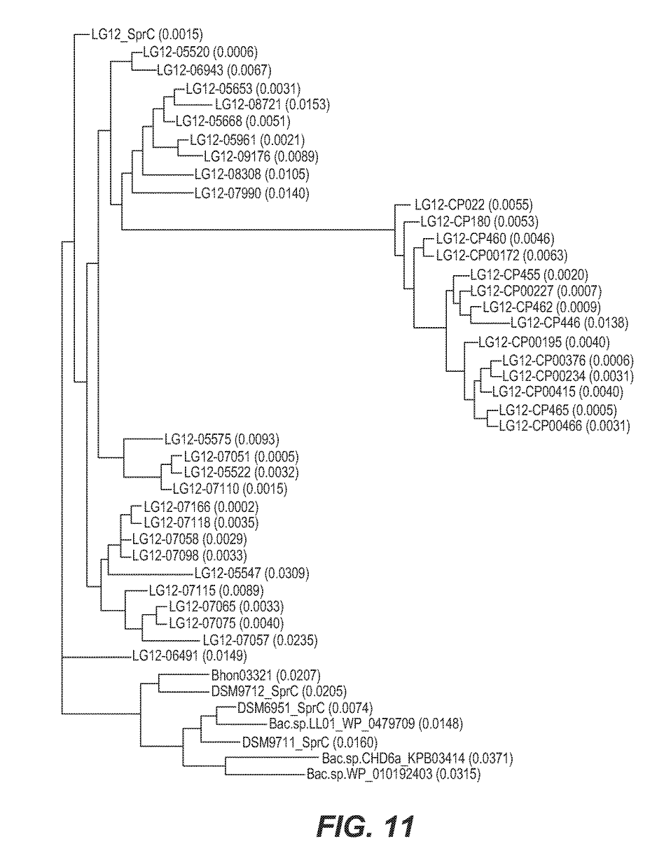

FIG. 11 provides a phylogenetic tree of LG12-clade proteases.

It is to be appreciated those certain feature of the invention, which are, for clarity, described above and below in the context of separate embodiments, may also be provided in combination in a single embodiment. Conversely, various features of the invention that are, for brevity, described in the context of a single embodiment, may also be provided separately or in any sub-combination.

Unless otherwise indicated, the practice of the present invention involves conventional techniques commonly used in molecular biology, protein engineering, microbiology, and recombinant DNA, which are within the skill of the art. Such techniques are known to those of skill in the art and are described in numerous texts and reference works well known to those of skill in the art. All patents, patent applications, articles and publications mentioned herein, both supra and infra, are hereby expressly incorporated herein by reference.

Unless defined otherwise herein, all technical and scientific terms used herein have the same meaning as commonly understood by one of ordinary skill in the art to which this invention pertains. Many technical dictionaries are known to those of skill in the art. Although any methods and materials similar or equivalent to those described herein find use in the practice of the present invention, some suitable methods and materials are described herein. Accordingly, the terms defined immediately below are more fully described by reference to the Specification as a whole. Also, as used herein, the singular “a”, “an” and “the” includes the plural reference unless the context clearly indicates otherwise. Numeric ranges are inclusive of the numbers defining the range. Unless otherwise indicated, nucleic acids are written left to right in 5′ to 3′ orientation: amino acid sequences are written left to right in amino to carboxy orientation, respectively. It is to be understood that this invention is not limited to the particular methodology, protocols, and reagents described, as these may vary, depending upon the context they are used by those of skill in the art.

The practice of the present invention employs, unless otherwise indicated, conventional techniques of protein purification, molecular biology, microbiology, recombinant DNA techniques and protein sequencing, all of which are within the skill of those in the art.

Furthermore, the headings provided herein are not limitations of the various aspects or embodiments of the invention which can be had by reference to the specification as a whole. Accordingly, the terms defined immediately below are more fully defined by reference to the specification as a whole. Nonetheless, in order to facilitate understanding of the invention, a number of terms are defined below.

It is intended that every maximum numerical limitation given throughout this specification include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The terms “protease” and “proteinase” refer to an enzyme protein that has the ability to break down other proteins. A protease has the ability to conduct “proteolysis,” which begins protein catabolism by hydrolysis of peptide bonds that link amino acids together in a peptide or polypeptide chain forming the protein. This activity of a protease as a protein-digesting enzyme is referred to as “proteolytic activity.” Many well known procedures exist for measuring proteolytic activity (See e.g., Kalisz. “Microbial Proteinases.” In: Fiechter (ed.), Advances in Biochemical Engineering/Biotechnology, (1988)). For example, proteolytic activity may be ascertained by comparative assays which analyze the respective protease's ability to hydrolyze a commercial substrate. Exemplary substrates useful in the analysis of protease or proteolytic activity, include, but are not limited to, di-methyl casein (Sigma C-9801), bovine collagen (Sigma C-9879), bovine elastin (Sigma E-1625), and bovine keratin (ICN Biomedical 902111). Colorimetric assays utilizing these substrates are well known in the art (See e.g., WO 99/34011 and U.S. Pat. No. 6,376,450, both of which are incorporated herein by reference). The pNA assay (See e.g., Del Mar et al., Anal. Biochem. 99:316-320 [1979]) also finds use in determining the active enzyme concentration for fractions collected during gradient elution. This assay measures the rate at which p-nitroaniline is released as the enzyme hydrolyzes a soluble synthetic peptide substrate, such as succinyl-alanine-alanine-proline-phenylalanine-p-nitroanilide (suc-AAPF-pNA), and cleavage occurs between the C-terminal amino acid (phenylalanine) and the p-NA, causing the production of yellow color from the hydrolysis reaction, which is measured at 410 nm on a spectrophotometer and is proportional to the active enzyme concentration. Measurement of the color change allows calculation of the rate of the reaction. In addition, absorbance measurements at 280 nanometers (nm) can be used to determine the total protein concentration. The active enzyme/total protein ratio gives the enzyme purity when a reference standard is used.

The phrase “composition(s) substantially-free of boron” or “detergent(s) substantially-free of boron” refers to composition(s) or detergent(s), respectively, that contain trace amounts of boron, for example, about 1000 ppm (1 mg/kg or litre equals 1 ppm), about 100 ppm, about 50 ppm, about 10 ppm, or about 5 ppm, or about 1 ppm, perhaps from other composition or detergent constituents.

The phrase “variant polypeptide” refers to a polypeptide comprising an amino acid sequence that differs in at least one amino acid residue from the amino acid sequence of a parent or reference polypeptide (including but not limited to wild-type polypeptides).

The phrase “protease variant” refers to a polypeptide that is derived from a reference polypeptide by the substitution, addition, or deletion, of one or more amino acids, typically by recombinant DNA techniques.

The phrase “the genus Bacillus” includes all species within the genus “Bacillus,” as known to those of skill in the art, including but not limited to B. subtilis, B. lichenformis, B. lentis, B. brevis, B, stearothermophilus, B. alkolophilus, B. amyloliquefaciens, B. clausii, B. halodurans, B. pumilus, B, gibsonii. B. pseudofirmus, B. lehensis, B. megaterium, B. coagulans. B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacilluts, Aneurinibacillus. Anoxybacillus, Brevibacillus, Filobacillus, Gracilibacillus, Halobacillus, Paenibacillus, Salibacillus, Thermobacillus, Ureibacillus, and Virgibacillus.

The terms “polynucleotide” and “nucleic acid,” which are used interchangeably herein, refer to a polymer of any length of nucleotide monomers covalently bonded in a chain. DNA (deoxyribonucleic acid), a polynucleotide comprising deoxyribonucleotides, and RNA (ribonucleic acid), a polymer of ribonucleotides, are examples of polynucleotides or nucleic acids having distinct biological function. Polynucleotides or nucleic acids include, but are not limited to, a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The following are non-limiting examples of polynucleotides: genes, gene fragments, chromosomal fragments, expressed sequence tag(s) (EST(s)), exons, introns, messenger RNA (mRNA), transfer RNA (tRNA), ribosomal RNA (rRNA), ribozymes, complementary DNA (cDNA), recombinant polynucleotides, branched polynucleotides, plasmids, vectors, isolated DNA of any sequence, isolated RNA of any sequence, nucleic acid probes, and primers. In some embodiments, polynucleotides comprise modified nucleotides, such as methylated nucleotides and nucleotide analogs, uracyl, other sugars and linking groups such as fluororibose and thioate, and nucleotide branches. In a particular embodiment, a sequence of nucleotides is interrupted by non-nucleotide components.

The term “mutation” refers to changes made in a starting amino acid or nucleic acid sequence. It is intended that the term encompass substitutions, insertions and deletions.

The term “vector” refers to a nucleic acid construct or polynucleotide construct used to introduce or transfer nucleic acid(s) or polynucleotide(s) into a target cell or tissue. A vector is typically used to introduce foreign DNA into another cell or tissue. A vector generally comprises a DNA sequence that is a transgene and a larger polynucleotide sequence that serves as the “backbone” of the vector. The vector typically serves to transfers genetic information, such as the inserted transgene, to a target cell or tissue so as to isolate, multiply, or express the insert in the target cell or tissue. Vectors include plasmids, cloning vectors, bacteriophages, viruses (e.g., viral vector), cosmids, expression vectors, shuttle vectors, cassettes, and the like. A vector typically includes an origin of replication, a multicloning site, and a selectable marker. The process of inserting a vector into a target cell is typically referred to as transfection. The transfection of a cell with a viral vector is typically referred to as transduction. The present invention includes, in some embodiments, a vector that comprises a DNA sequence encoding a variant protease (e.g., precursor or mature variant protease) that is operably linked to a suitable pro sequence (e.g., secretory, signal peptide sequence, etc.) capable of effecting the expression of the DNA sequence in a suitable host.

The phrase “expression cassette,” “expression plasmid” or “expression vector” refers to a nucleic acid construct or vector generated recombinantly or synthetically for the expression of a nucleic acid of interest (e.g., a foreign nucleic acid or transgene) in a target cell. The nucleic acid of interest typically expresses a protein of interest. An expression vector or expression cassette typically comprises a promoter nucleotide sequence that drives or promotes expression of the foreign nucleic acid. The expression vector or cassette also typically includes any other specified nucleic acid elements that permit transcription of a particular nucleic acid in a target cell. A recombinant expression cassette can be incorporated into a plasmid, chromosome, mitochondrial DNA, plastid DNA, virus, or nucleic acid fragment. Some expression vectors have the ability to incorporate and express heterologous DNA fragments in a host cell. Many prokaryotic and eukaryotic expression vectors are commercially available. Selection of appropriate expression vectors is within the knowledge of those of skill in the art. Selection of appropriate expression vectors for expression of a protein from a nucleic acid sequence incorporated into the expression vector is within the knowledge of those of skill in the art.

A DNA construct is an artificially constructed segment of nucleic acid that may be introduced into a target cell or tissue. A DNA construct typically comprises a DNA insert comprising a nucleotide sequence encoding a protein of interest that has been subcloned into a vector. The vector may contain bacterial resistance genes for growth in bacteria and a promoter for expression of the protein of interest in an organism. The DNA may be generated in vitro by PCR or any other suitable technique(s) known to those in the art. In some embodiments, the DNA construct comprises a nucleic acid sequence of interest. In some embodiments, the sequence is operably linked to additional elements such as control elements (e.g., promoters, etc.). The DNA construct may further comprise a selectable marker and may further comprise an incoming sequence flanked by homology boxes. The construct may comprise other non-homologous sequences, added to the ends (e.g., stuffer sequences or flanks). In some embodiments, the ends of the sequence are closed such that the DNA construct forms a closed circle. The nucleic acid sequence of interest, which is incorporated into the DNA construct, using techniques well known in the art, may be a wild-type, mutant, or modified nucleic acid. In some embodiments, the DNA construct comprises one or more nucleic acid sequences homologous to the host cell chromosome. In other embodiments, the DNA construct comprises one or more non-homologous nucleotide sequences. Once the DNA construct is assembled in vitro, it may be used, for example, to: 1) insert heterologous sequences into a desired target sequence of a host cell; and/or 2) mutagenize a region of the host cell chromosome (i.e., replace an endogenous sequence with a heterologous sequence); 3) delete target genes; and/or 4) introduce a replicating plasmid into the host. “DNA construct” is used interchangeably herein with “expression cassette.”

The term “plasmid” refers to an extrachromosomal DNA molecule which is capable of replicating independently from the chromosomal DNA. A plasmid is double stranded (ds) and may be circular and is typically used as a cloning vector.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, electroporation, conjugation, and transduction (See e.g., Ferrari et al., “Genetics.” in Hardwood et al. (eds.). Bacillus Plenum Publishing Corp., pp. 57-72 [1989]).

The term “transformation refers to the genetic alteration of a cell which results from the uptake, genomic incorporation, and expression of genetic material (e.g., DNA).

As used herein, a nucleic acid is “operably linked” with another nucleic acid sequence when it is placed into a functional relationship with another nucleic acid sequence. For example, a promoter or enhancer is operably linked to a nucleotide coding sequence if the promoter affects the transcription of the coding sequence. A ribosome binding site may be operably linked to a coding sequence if it is positioned so as to facilitate translation of the coding sequence. Typically, “operably linked” DNA sequences are contiguous. However, enhancers do not have to be contiguous. Linking is accomplished by ligation at convenient restriction sites. If such sites do not exist, synthetic oligonucleotide adaptors or linkers may be used in accordance with conventional practice.

The term “gene” refers to a polynucleotide (e.g., a DNA segment), that encodes a polypeptide and includes regions preceding and following the coding regions as well as intervening sequences (introns) between individual coding segments (exons).

The term “recombinant” when used with reference to a cell typically indicates that the cell has been modified by the introduction of a heterologous nucleic acid sequence or that the cell is derived from a cell so modified. For example, a recombinant cell may comprise a gene not found in identical form within the native (non-recombinant) form of the cell, or a recombinant cell may comprise a native gene (found in the native form of the cell) but which has been modified and re-introduced into the cell. A recombinant cell may comprise a nucleic acid endogenous to the cell that has been modified without removing the nucleic acid from the cell; such modifications include those obtained by gene replacement, site-specific mutation, and related techniques known to those of ordinary skill in the art. Recombinant DNA technology includes techniques for the production of recombinant DNA in vitro and transfer of the recombinant DNA into cells where it may be expressed or propagated, thereby producing a recombinant polypeptide. “Recombination,” “recombining,” and “recombined” of polynucleotides or nucleic acids refer generally to the assembly or combining of two or more nucleic acid or polynucleotide strands or fragments to generate a new polynucleotide or nucleic acid. The recombinant polynucleotide or nucleic acid is sometimes referred to as a chimera. A nucleic acid or polypeptide is “recombinant” when it is artificial or engineered, or derived from an artificial or engineered protein or nucleic acid.

A nucleic acid or polynucleotide is said to “encode” a polypeptide if, in its native state or when manipulated by methods known to those of skill in the art, it can be transcribed and/or translated to produce a protease variant described herein, or a recombinant polypeptide or a fragment thereof. The anti-sense strand of such a nucleic acid is also said to encode the sequence.

The phrase “host strain” or “host cell” refers to a suitable host for an expression vector comprising a polynucleotide sequence that expresses a protease variant described herein, or recombinant polypeptide or active fragment thereof in the host strain or host cell.

A “protein” or “polypeptide” comprises a polymeric sequence of amino acid residues. The terms “protein” and “polypeptide” are used interchangeably herein. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Mutations are named by the one letter code for the parent amino acid, followed by a three or two digit position number and then the one letter code for the variant amino acid. For example, mutating glycine (G) at position 87 to serine (S) is represented as “G087S” or “G87S”. Multiple mutations are indicated by inserting a “-” between the mutations. Mutations at positions 87 and 90 are represented as either “G087S-A090Y” or “G87S-A90Y” or “G87S+A90Y” or “G087S+A090Y”.

A “prosequence” or “propeptide sequence” refers to an amino acid sequence between the signal peptide sequence and mature protease sequence that is necessary for proper folding and the secretion of the protease. Cleavage of the prosequence or propeptide sequence results in a mature active protease. The prosequence may be endogenous or the sequence may occur is a highly related protein such as another subtilisin. The prosequence may be a naturally occurring sequence or a variant sequence derived by recombinant technology, such as, for example, is described in U.S. provisional patent application No. 62/181,192, filed Jun. 17, 2015

The phrase “signal sequence” or “signal peptide” refers to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The phrase “hybrid signal sequence” refers to signal sequences in which part of sequence is obtained from the expression host fused to the signal sequence of the gene to be expressed. In some embodiments, synthetic sequences are utilized.

The term “mature” form of a protein, polypeptide, or peptide refers to the functional form of the protein, polypeptide, or peptide without the signal peptide sequence and propeptide sequence.

The term “precursor” form of a protein or peptide refers to a mature form of the protein having a prosequence operably linked to the amino or carbonyl terminus of the protein. The precursor may also have a “signal” sequence operably linked to the amino terminus of the prosequence. The precursor may also have additional polynucleotides that are involved in post-translational activity (e.g., polynucleotides cleaved therefrom to leave the mature form of a protein or peptide).

The term “wild-type” in reference to an amino acid sequence or nucleic acid sequence indicates that the amino acid sequence or nucleic acid sequence is native or naturally occurring sequence. As used herein, the term “naturally-occurring” refers to anything (e.g., proteins, amino acids, or nucleic acid sequences) that are found in nature (i.e., have not been manipulated by means of recombinant methods).

The phrase “non-naturally occurring” refers to anything that is not found in nature (e.g., recombinant polynucleotides or polypeptides produced in the laboratory).

As used herein with regard to amino acid residue positions, “corresponding to” or “corresponds to” or “corresponds” refers to an amino acid residue at the enumerated position in a protein or peptide, or an amino acid residue that is analogous, homologous, or equivalent to an enumerated residue in a protein or peptide. As used herein. “corresponding region” generally refers to an analogous position in a related proteins or a reference protein.

The phrases “derived from” and “obtained from” refer to not only a protein produced or producible by a strain of the organism in question, but also a protease encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protease in question. To exemplify, “proteases derived from Bacillus” refers to those enzymes having proteolytic activity which are naturally produced by Bacillus, as well as to serine proteases like those produced by Bacillus sources but which through the use of genetic engineering techniques are produced by a Bacillus strain or non-Bacillus organisms transformed with a nucleic acid encoding the serine proteases.

The phrase “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

The term “homology” refers to sequence similarity or identity, with identity being preferred. Homology, may be determined using standard techniques known in the art (&e e.g., Smith and Waterman. Adv. Appl. Math. 2:482 [1981]; Needleman and Wunsch. J. Mol. Biol. 48:443 [1970]; Pearson and Lipman. Proc. Natl. Acad. Sci. USA 85:2444 [1988]; software programs such as GAP, BESTFIT, FASTA, and TFASTA in the Wisconsin Genetics Software Package (Genetics Computer Group, Madison, Wis.); and Devereux et al., Nucl. Acid Res. 12:387-395 [1984]). One example of a useful algorithun is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (&e. Feng and Doolittle, J. Mol. Evol. 35:351-360 [1987]). The method is similar to that described by Higgins and Sharp (See, Higgins and Sharp, CABIOS 5:151-153 [1989]). Useful PILEUP parameters including a default gap weight of 3.00, a default gap length weight of 0.10, and weighted end gaps.

Percent “identical” or “identity” in the context of two or more nucleic acid or polypeptide sequences refers to two or more sequences that are the same or have a specified percentage of nucleic acid residues or amino acid residues, respectively, that are the same, when compared and aligned for maximum similarity, as determined using a sequence comparison algorithm or by visual inspection. “Percent sequence identity” or “% identity” or “% sequence identity or “% amino acid sequence identity” of a subject amino acid sequence to a reference (i.e., query) amino acid sequence means that the subject amino acid sequence is identical (i.e., on an amino acid-by-amino acid basis) by a specified percentage to the query amino acid sequence over a comparison length when the sequences are optimally aligned. Thus. 80% amino acid sequence identity or 80% identity with respect to two amino acid sequences means that 80% of the amino acid residues in two optimally aligned amino acid sequences are identical.

In some embodiments, the “percent sequence identity” or “% sequence identity” or “% identity” of a subject sequence to a query sequence can be calculated by optimally aligning the two sequences and comparing the two optimally aligned sequences over the comparison length. The number of positions in the optimal alignment at which identical residues occur in both sequences is determined, thereby providing the number of matched positions, and the number of matched positions is then divided by the total number of positions of the comparison length (which, unless otherwise specified, is the length of the query sequence). The resulting number is multiplied by 100 to yield the percent sequence identity of the subject sequence to the query sequence. The percent sequence identity between a reference sequence and a protease variant described herein may be readily determined by one skilled in the art. The percent identity shared by polynucleotide or polypeptide sequences is determined by direct comparison of the sequence information between the molecules by aligning the sequences and determining the identity by methods known in the art.

Sequence identity may be determined using known programs such as BLAST, ALIGN, and CLUSTAL using standard parameters. (See, e.g., Altschul et al. [1990] J. Mol. Biol. 215:403-410; Henikoff et al. [1989] Proc. Natl. Acad. Sci. USA 89:10915; Karin et al. [1993] Proc. Natl. Acad. Sci USA 90:5873; and Higgins et al. [1988] Gene 73:237-244). Software for performing BLAST analyses is publicly available through the National Center for Biotechnology Information. Databases may also be searched using FASTA (Pearson et al. [1988] Proc. Natl. Acad. Sci. USA 85:2444-2448). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another useful algorithm for comparison of multiple protein sequences is the MUSCLE program from Geneious software (Biomatters Ltd.) (Robert C. Edgar. MUSCLE: multiple sequence alignment with high accuracy and high throughput Nucl. Acids Res. (2004) 32 (5): 1792-1797).

The phrase “optimal alignment” or “optimally aligned” refers to the alignment of two (or more) sequences giving the highest percent identity score. For example, optimal alignment of two protein sequences can be achieved by manually aligning the sequences such that the maximum number of identical amino acid residues in each sequence are aligned together or by using software programs or procedures described herein or known in the art. Optimal alignment of two nucleic acid sequences can be achieved by manually aligning the sequences so that the maximum number of identical nucleotides in each sequence are aligned together or by using software programs or procedures described herein or known in the art. In some embodiments, two polypeptide sequences are deemed “optimally aligned” when they are aligned using defined parameters, such as a defined amino acid substitution matrix, gap existence penalty (also termed gap open penalty), and gap extension penalty, so as to achieve the highest similarity score possible for that pair of sequences. The BLOSUM62 scoring matrix (See, Henikoff and Henikoff, supra) is often used as a default scoring substitution matrix in polypeptide sequence alignment algorithms (e.g., BLASTP). The gap existence penalty is imposed for the introduction of a single amino acid gap in one of the aligned sequences, and the gap extension penalty is imposed for each residue position in the gap. Exemplary alignment parameters employed are: BLOSUM62 scoring matrix, gap existence penalty=11, and gap extension penalty=1. The alignment score is defined by the amino acid positions of each sequence at which the alignment begins and ends (e.g., the alignment window), and optionally by the insertion of a gap or multiple gaps into one or both sequences, so as to achieve the highest possible similarity score.

A nucleic acid or polynucleotide is “isolated” when it is partially or completely separated from other components, including but not limited to for example, other proteins, nucleic acids, cells, etc. Similarly, a polypeptide, protein or peptide is “isolated” when it is partially or completely separated from other components, including but not limited to for example, other proteins, nucleic acids, cells, etc. On a molar basis, an isolated species is more abundant than are other species in a composition. For example, an isolated species may comprise at least about 50%, about 70%, about 80%, about 85%, about 90%, about 91%, about 92%, about 93%, about 94%, about 95%, about 96%, about 97%, about 98%, about 99%, or about 100% (on a molar basis) of all macromolecular species present. Preferably, the species of interest is purified to essential homogeneity (i.e., contaminant species cannot be detected in the composition by conventional detection methods). Purity and homogeneity can be determined using a number of techniques well known in the art, such as agarose or polyacrylamide gel electrophoresis of a protein or nucleic acid sample, followed by visualization upon staining. If desired, a high-resolution technique, such as high performance liquid chromatography (HPLC) or a similar means can be utilized for purification of the material.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 75%, about 80%, about 85%, about 90%, about 91%, about 92%, about 93%, about 94%, about 95%, about 96/%, about 97%, about 98%, about 99%, about 99.5%, about 99.6%, about 99.7%, about 99.8% or more pure (e.g., percent by weight on a molar basis). In a related sense, the invention provides methods of enriching compositions for one or more molecules of the invention, such as one or more polypeptides or polynucleotides of the invention. A composition is enriched for a molecule when there is a substantial increase in the concentration of the molecule after application of a purification or enrichment technique. A substantially pure polypeptide or polynucleotide of the invention (e.g., substantially pure variant protease or polynucleotide encoding a variant protease of the invention, respectively) will typically comprise at least about 55%, about 60%, about 70%, about 80%, about 85%, about 90%, about 91%, about 92%, about 93%, about 94%, about 95%, about 96%, about 97%, about 98, about 99%, about 99.5% or more by weight (on a molar basis) of all macromolecular species in a particular composition.

In a related sense, the invention provides methods of enriching compositions for one or more molecules of the invention, such as one or more polypeptides of the invention (e.g., one or more variant proteases of the invention) or one or more nucleic acids of the invention (e.g., one or more nucleic acids encoding one or more variant proteases of the invention). A composition is enriched for a molecule when there is a substantial increase in the concentration of the molecule after application of a purification or enrichment technique.

The phrase “improved property” when used in connection with a protease variant described herein refers to a protease variant with improved residual protease activity, improved cleaning performance and/or improved stability optionally with retained cleaning performance relative to the corresponding reference polypeptide.

The phrase “functional assay” refers to an assay that provides an indication of a protein's activity. In some embodiments, the term refers to assay systems in which a protein is analyzed for its ability to function in its usual capacity. For example, in the case of enzymes, a functional assay involves determining the effectiveness of the enzyme in catalyzing a reaction.

The phrase “thermally stable” and “thermostable” and “thermostability” refer to proteases that retain a specified amount of enzymatic activity after exposure to identified temperatures over a given period of time under conditions prevailing during the proteolytic, hydrolyzing, cleaning or other process of the invention, while being exposed to altered temperatures. “Altered temperatures” encompass increased or decreased temperatures. In some embodiments, the proteases retain at least about 50%, about 60%, about 70%, about 75%, about 80%, about 85%, about 90%, about 92%, about 95%, about 96%, about 97%, about 98%, or about 99% proteolytic activity after exposure to altered temperatures over a given time period, for example, at least about 60 minutes, about 120 minutes, about 180 minutes, about 240 minutes, about 300 minutes, etc.

The phrase “improved stability” in the context of an oxidation, chelator, thermal and/or pH stable protease refers to a higher retained proteolytic activity over time as compared to other proteases (e.g., LG12 and BPN′-Y217L proteases) and/or wild-type enzymes.

The phrase “diminished stability” in the context of an oxidation, chelator, thermal and/or pH stable protease refers to a lower retained proteolytic activity over time as compared to other proteases (e.g., LG12 proteases) and/or wild-type enzymes.

The phrase “cleaning activity” refers to a cleaning performance achieved by a variant protease or reference protease under conditions prevailing during the proteolytic, hydrolyzing, cleaning, soil/stain removal, or other process of the invention. In some embodiments, cleaning performance of a protease variant described herein or reference protease/polypeptide may be determined by using various assays for cleaning one or more various enzyme sensitive stains on an item or surface (e.g., a stain resulting from food, grass, blood, ink, milk, oil, and/or egg protein). Cleaning performance of a protease variant or reference protease/polypeptide can be determined by subjecting a protein stain on the item or surface to standard wash condition(s) and assessing the degree to which the stain is removed by using various chromatographic, spectrophotometric, or other quantitative methodologies. A protein stain includes, for example, egg yolk, blood, milk, ink, grass, pigment, oil, creme brulee, pasta, cheese, and combinations thereof. Exemplary cleaning assays and methods are known in the art and include, but are not limited to those described in WO 99/34011 and U.S. Pat. No. 6,605,458, both of which are herein incorporated by reference, as well as those cleaning assays and methods included in the Examples provided below.

The phrase “cleaning effective amount” of a variant protease or reference protease refers to the amount of protease that achieves a desired level of enzymatic activity in a specific cleaning composition. Such effective amounts are readily ascertained by one of ordinary skill in the art and are based on many factors, such as the particular protease used, the cleaning application, the specific composition of the cleaning composition, and whether a liquid or dry (e.g., granular, tablet, bar) composition is required, etc. The term “soil removal index” (SRI) of a variant protease or reference protease refers to the degree of soil removal achieved by a certain quantity of enzyme or enzymatic activity.

The phrase “cleaning adjunct material” refers to any liquid, solid, or gaseous material included in cleaning composition other than a variant protease of the invention. In some embodiments, the cleaning compositions of the present invention include one of more cleaning adjunct materials. Each cleaning adjunct material is typically selected depending on the particular type and form of cleaning composition (e.g., liquid, granule, powder, bar, paste, spray, tablet, gel, foam, or other composition). Preferably, each cleaning adjunct material is compatible with the protease enzyme used in the composition.

The phrase “improved cleaning performance” in the context of cleaning activity refers to an increased or greater cleaning activity by an enzyme on certain enzyme sensitive stains such as, for example, egg, milk, grass, ink, oil, pigment, and/or blood, as determined by usual evaluation after a standard wash cycle and/or multiple wash cycles. The phrase “diminished cleaning performance” in the context of cleaning activity refers to a decreased or lesser cleaning activity by an enzyme on certain enzyme sensitive stains such as, for example, egg, milk, grass, ink, oil pigment and/or blood, as determined by usual evaluation after a standard wash cycle. Cleaning performance can be determined by comparing the protease variants of the present invention with reference proteases in various cleaning assays concerning enzyme sensitive stains such as, for example, egg, grass, blood, ink, oil, pigment and/or milk as determined by usual spectrophotometric or analytical methodologies after standard wash cycle conditions.

The phrase “consumer product” means fabric and home care product.

The phrase “fabric and home care product” or “fabric and household care product” includes products generally intended to be used or consumed in the form in which they are sold and that are for treating fabrics, hard surfaces and any other surfaces, and cleaning systems all for the care and cleaning of inanimate surfaces, as well as fabric conditioner products and other products designed specifically for the care and maintenance of fabrics, and air care products, including: air care including air fresheners and scent delivery systems, car care, pet care, livestock care, personal care, jewelry care, dishwashing, fabric conditioning (including softening and/or freshening), laundry detergency, laundry and rinse additive and/or care, pre-treatment cleaning compositions, hard surface cleaning and/or treatment including floor and toilet bowl cleaners, glass cleaners and/or treatments, tile cleaners and/or treatments, ceramic cleaners and/or treatments, and other cleaning for consumer or institutional use. In some embodiments, the fabric and home care products are suitable for use on wounds and/or skin. “Fabric and home care product” includes consumer and institutional products.

The phrase “institutional cleaning composition” refers to products suitable for use in institutions including but not limited to schools, hospitals, factories, stores, corporations, buildings, restaurants, office complexes and buildings, processing and/or manufacturing plants, veterinary hospitals, factory farms, factory ranches, etc.

The phrase “cleaning and/or treatment composition” is a subset of fabric and home care products that includes, unless otherwise indicated, compositions suitable for cleaning and/or treating items. Such products include, but are not limited to, products for treating fabrics, hard surfaces and any other surfaces in the area of fabric and home care, including: air care including air fresheners and scent delivery systems, car care, dishwashing, fabric conditioning (including softening and/or freshening), laundry detergency, laundry and rinse additive and/or care, hard surface cleaning and/or treatment including floor and toilet bowl cleaners, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use: car or carpet shampoos, bathroom cleaners including toilet bowl cleaners; as well as cleaning auxiliaries such as bleach additives and “stain-stick” or pre-treat types, substrate-laden products such as dryer added sheets.

The phrase “cleaning composition” or “cleaning formulation” of the invention refers to any composition of the invention useful for removing or eliminating a compound (e.g., undesired compound) from an object, item or surface to be cleaned, including, but not limited to for example, a fabric, fabric item, dishware item, tableware item, glassware item, contact lens, other solid substrate, hair (shampoo) (including human or animal hair), skin (soap or and cream), teeth (mouthwashes, toothpastes), surface of an item or object (e.g., hard surfaces, such as the hard surface of a table, table top, wall furniture item, floor, ceiling, non-dishware item, non-tableware item, etc.), filters, membranes (e.g., filtration membranes, including but not limited to ultrafiltration membranes), etc. The term encompasses any material and/or added compound selected for the particular type of cleaning composition desired and the form of the product (e.g., liquid, gel, granule, spray, or other composition), as long as the composition is compatible with the protease and other enzyme(s) used in the composition. The specific selection of cleaning composition materials are readily made by considering the surface, object, item, or fabric to be cleaned, and the desired form of the composition for the cleaning conditions during use.

Cleaning compositions and cleaning formulations include any composition that is suited for cleaning, bleaching, disinfecting, and/or sterilizing any object, item, and/or surface. Such compositions and formulations include, but are not limited to for example, liquid and/or solid compositions, including cleaning or detergent compositions, such as, for example, liquid, tablet, gel, bar, granule, and/or solid laundry cleaning or detergent compositions and fine fabric detergent compositions: hard surface cleaning compositions and formulations, such as, for example, glass, wood, ceramic and metal counter tops and windows; carpet cleaners; oven cleaners; fabric fresheners; fabric softeners: and textile, laundry booster cleaning or detergent compositions, laundry additive cleaning compositions, and laundry pre-spotter cleaning compositions; dishwashing compositions, including hand or manual dishwash compositions (e.g., “hand” or “manual” dishwashing detergents) and automatic dishwashing (ADW) compositions (e.g., “automatic dishwashing detergents”).

Cleaning composition or cleaning formulations, as used herein, include, unless otherwise indicated, granular or powder-form all-purpose or heavy-duty washing agents, especially cleaning detergents: liquid, granular, gel, solid, tablet, or paste-form all-purpose washing agents, especially the so-called heavy-duty liquid (HDL) detergent or heavy-duty powder detergent (HDD) types; liquid fine-fabric detergents; hand or manual dishwashing agents, including those of the high-foaming type; hand or manual dishwashing, automatic dishwashing, or dishware or tableware washing agents, including the various tablet, powder, solid, granular, liquid, gel, and rinse-aid types for household and institutional use; liquid cleaning and disinfecting agents, including antibacterial hand-wash types, cleaning bars, mouthwashes, denture cleaners, car shampoos, carpet shampoos, bathroom cleaners; hair shampoos and/or hair-rinses for humans and other animals; shower gels and foam baths and metal cleaners; as well as cleaning auxiliaries, such as bleach additives and “stain-stick” or pre-treat types. In some embodiments, granular compositions are in “compact” form; in some embodiments, liquid compositions are in a “concentrated” form.

As used herein. “fabric cleaning compositions” include hand and machine laundry detergent compositions including laundry additive compositions and compositions suitable for use in the soaking and/or pretreatment of stained fabrics (e.g., clothes, linens, and other textile materials).

The phrase “non-fabric cleaning compositions” include non-textile (i.e., non-fabric) surface cleaning compositions, including, but not limited to for example, hand or manual or automatic dishwashing detergent compositions, oral cleaning compositions, denture cleaning compositions, and personal cleansing compositions.

The phrase “fabric and/or hard surface cleaning and/or treatment composition” is a subset of cleaning and treatment compositions that includes, unless otherwise indicated, granular or powder-form all-purpose or “heavy-duty” washing agents, especially cleaning detergents; liquid, gel or paste-form all-purpose washing agents, especially the so-called heavy-duty liquid types liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, car or carpet shampoos, bathroom cleaners including toilet bowl cleaners; fabric conditioning products including softening and/or freshening that may be in liquid, solid and/or dryer sheet form: as well as cleaning auxiliaries such as bleach additives and “stain-stick” or pre-treat types, substrate-laden products such as dryer added sheets. All of such products which are applicable may be in standard, concentrated or even highly concentrated form even to the extent that such products may in certain aspect be non-aqueous.

The phrase “detergent composition” or “detergent formulation” is used in reference to a composition intended for use in a wash medium for the cleaning of soiled or dirty objects, including particular fabric and/or non-fabric objects or items. Such compositions of the present invention are not limited to any particular detergent composition or formulation. Indeed, in some embodiments, the detergents of the invention comprise at least one variant protease of the invention and, in addition, one or more surfactants, transferase(s), hydrolytic enzymes, oxido reductases, builders (e.g., a builder salt), bleaching agents, bleach activators, bluing agents, fluorescent dyes, caking inhibitors, masking agents, enzyme activators, antioxidants, and/or solubilizers. In some instances, a builder salt is a mixture of a silicate salt and a phosphate salt, preferably with more silicate (e.g., sodium metasilicate) than phosphate (e.g., sodium tripolyphosphate). Some compositions of the invention, such as, but not limited to, cleaning compositions or detergent compositions, do not contain any phosphate (e.g., phosphate salt or phosphate builder).

The term “bleaching” refers to the treatment of a material (e.g., fabric, laundry, pulp, etc.) or surface for a sufficient length of time and/or under appropriate pH and/or temperature conditions to effect a brightening (i.e., whitening) and/or cleaning of the material. Examples of chemicals suitable for bleaching include, but are not limited to, for example, ClO₂, H₂O₂, peracids, NO₂, etc.

The term “disinfecting” refers to the removal of contaminants from the surfaces, as well as the inhibition or killing of microbes on the surfaces of items. It is not intended that the present invention be limited to any particular surface, item, or contaminant(s) or microbes to be removed.

The term “compact” form of the cleaning compositions herein is best reflected by density and, in terms of composition, by the amount of inorganic filler salt. Inorganic filler salts are conventional ingredients of detergent compositions in powder form. In conventional detergent compositions, the filler salts are present in substantial amounts, typically about 17 to about 35% by weight of the total composition. In contrast, in compact compositions, the filler salt is present in amounts not exceeding about 15% of the total composition. In some embodiments, the filler salt is present in amounts that do not exceed about 10%, or more preferably, about 5%, by weight of the composition. In some embodiments, the inorganic filler salts are selected from the alkali and alkaline-earth-metal salts of sulfates and chlorides. In some embodiments, the filler salt is sodium sulfate.

The position of an amino acid residue in a given amino acid sequence is typically numbered herein using the numbering of the position of the corresponding amino acid residue of the LG2 SprC (described as LG-12 SprC in Example 2, page 86, line 16 to page 87, line 14 of PCT Published Application WO2015/038792, filed Sep. 11, 2014, which is hereby incorporated herein by reference) amino acid sequence shown in SEQ ID NO: 10, which is also referred to interchangeably throughout as LG12 wild-type. A given amino acid sequence, such as a variant protease amino acid sequence described herein, can be aligned with the LG2 SprC sequence using an alignment algorithm as described herein, and an amino acid residue in the given amino acid sequence that aligns (preferably optimally aligns) with an amino acid residue in the LG12 SprC sequence can be conveniently numbered by reference to the corresponding amino acid residue in the LG2 SprC sequence.

Variants, compositions and methods disclosed herein relate to a protease variant, or a recombinant polypeptide or an active fragment thereof generated through conventional molecular biology techniques (see, e.g., Sambrook et al, Molecular Cloning: Cold Spring Harbor Laboratory Press). Some embodiments are directed to a protease variant, or a recombinant polypeptide or an active fragment thereof comprising an amino acid sequence comprising two or more substitutions at one or more positions selected from: (i) 1, 3, 7, 9, 19, 24, 27, 30, 38, 40, 43, 45, 48, 52, 53, 57, 72, 76, 78, 79, 86, 87, 88, 89, 90, 91, 93, 97, 99, 100, 101, 103, 104, 106, 107, 111, 114, 115, 116, 117, 118, 120, 122, 123, 124, 128, 129, 130, 131, 133, 134, 136, 139, 140, 141, 143, 144, 145, 147, 148, 149, 150, 153, 156, 158, 159, 160, 166, 181, 182, 185, 188, 194, 209, 212, 213, 215, 216, 217, 222, 224, 228, 234, 236, 238, 240, 242, 245, 246, 248, 251, 252, 256, 265, 267, 269, 270, 271, 272, 273, and 275; (ii) 1, 3, 9, 19, 24, 27, 30, 38, 40, 43, 45, 48, 52, 53, 57, 72, 76, 78, 79, 86, 87, 88, 89, 90, 91, 93, 97, 99, 100, 101, 103, 104, 106, 107, 111, 114, 115, 116, 117, 118, 120, 122, 123, 124, 128, 129, 130, 131, 133, 134, 136, 139, 140, 141, 143, 144, 145, 147, 148, 149, 150, 153, 156, 158, 159, 160, 166, 181, 182, 185, 188, 194, 209, 212, 213, 215, 216, 217, 222, 224, 228, 234, 236, 238, 240, 242, 245, 246, 248, 251, 252, 256, 265, 267, 271, 272, and 275; (iii) 1, 7, 9, 27, 38, 40, 45, 48, 52, 53, 76, 78, 91, 100, 114, 117, 118, 122, 128, 129, 130, 140, 147, 149, 156, 158, 159, 160, 166, 181, 182, 194, 209, 217, 222, 228, 234, 236, 238, 245, 248, 256, 269, 270, 271, 272, 273, and 275; (iv) 1, 24, 30, 38, 40, 48, 52, 53, 76, 78, 79, 86, 87, 88, 89, 91, 93, 97, 100, 103, 104, 106, 111, 114, 115, 116, 117, 118, 120, 122, 124, 128, 129, 130, 131, 133, 134, 136, 139, 141, 143, 144, 147, 148, 149, 150, 153, 156, 158, 159, 166, 182, 185, 194, 209, 212, 213, 216, 217, 222, 224, 236, 238, 248, 251, 252, 256, 272, and 275; (v) 1, 24, 30, 38, 40, 52, 53, 76, 78, 79, 86, 87, 88, 89, 93.97, 100, 103, 104, 106, 111, 114, 115, 116, 117.118, 120, 122, 124, 128, 129, 131, 133, 134, 136, 139, 141, 143, 144, 147, 148, 149, 150, 153, 156, 158, 159, 166, 182, 185, 194, 209, 212, 213, 216, 217, 222, 224, 236, 238, 248, 251, 252, 256, 272, and 275; or (vi) 1, 38, 40, 48, 52, 53, 76, 91, 114, 117, 118, 128, 129, 130, 147, 149, 156, 158, 159, 166, 182, 217, 238, 248, 256, and 272; wherein the amino acid positions of the variant or recombinant polypeptide or active fragment thereof are numbered by correspondence with the amino acid sequence of SEQ ID NO: 10. A further embodiment is directed to a protease variant, or a recombinant polypeptide or an active fragment thereof comprising an amino acid sequence comprising two or more substitutions at one or more positions selected from: (i) 1, 3, 9, 19, 24, 27, 30, 38, 40, 43, 45, 48, 52, 53, 57, 72, 76, 78, 79, 86, 87, 88, 89, 90, 91, 93, 97, 99, 100, 101, 103, 104, 106, 107, 111, 114, 115, 116, 117, 118, 120, 122, 123, 124, 128, 129, 130, 131, 133, 134, 136, 139, 140, 141, 143, 144, 145, 147, 148, 149, 150, 153, 156, 158, 159, 160, 166, 181, 182, 185, 188, 194, 209, 212, 213, 215, 216, 217, 222, 224, 228, 234, 236, 238, 240, 242, 245, 246, 248, 251, 252, 256, 265, 267, 271, 272, and 275; (ii) 1, 3, 9, 19, 24, 27, 30, 38, 40, 43, 45, 48, 52, 53, 57, 72, 76, 78, 79, 86, 87, 88, 89, 90, 91, 93, 97, 99, 100, 101, 103, 104, 106, 107, 111, 114, 115, 116, 117, 118, 120, 122, 123, 124, 128, 129, 130, 131, 133, 134, 136, 139, 140, 141, 143, 144, 145, 147, 148, 149, 150, 156, 158, 159, 160, 166, 181, 182, 185, 188, 194, 209, 212, 213, 215, 216, 217, 222, 224, 228, 234, 236, 238, 240, 242, 245, 246, 248, 251, 252, 256, 265. 267, 271, 272, and 275; (iii) 9, 38, 40, 45, 48, 52, 53, 76, 78, 100, 114, 117, 118, 128, 129, 130, 140, 147, 149, 156, 158, 159, 160, 166, 181, 182, 194, 209, 217, 234, 236, 238, 245, 248, 256, 272, and 275; (iv) 1, 24, 30, 38, 40, 48, 52, 53, 76, 78, 79, 86, 87, 88, 89, 91, 93, 97, 100, 103, 104, 106, 111, 114, 115, 116, 117, 118, 120, 122, 124, 128, 129, 130, 131, 133, 134, 136, 139, 141, 143, 144, 147, 148, 149, 150, 156, 158, 159, 166, 182, 185, 194, 209, 212, 213, 216, 217, 222, 224, 236, 238, 248, 251, 252, 256, 272, and 275; (v) 1, 24, 30, 38, 40, 52, 53, 76, 78, 79, 86, 87, 88, 89, 93, 97, 100, 103, 104, 106, 111, 114, 115, 116, 117, 118, 120, 122, 124, 128, 129, 131, 133, 134, 136, 139, 141, 143, 144, 147, 148, 149, 150, 156, 158, 159, 166, 182, 185, 194, 209, 212, 213, 216, 217, 222, 224, 236, 238, 248, 251, 252, 256, 272, and 275; or (vi) 38, 40, 48, 52, 53, 76, 114, 117, 118, 128, 129, 130, 147, 149, 156, 158, 159, 166, 182, 217, 238, 248, 256, and 272; wherein the amino acid positions of the variant or recombinant polypeptide or active fragment thereof are numbered by correspondence with the amino acid sequence of SEQ ID NO: 10. Another embodiment is directed to a protease variant, or a recombinant polypeptide or an active fragment thereof comprising an amino acid sequence comprising two or more substitutions at one or more position selected from: (i) 52, 53, 76, 86, 97, 103, 104, 106, 111, 114, 115, 116, 118, 120, 122, 124, 128, 129, 130, 131, 134, 136, 139, 141, 143, 144, 147, 148, 150, 156, 158, 159, 166, 217, 248, and 272; (ii) 53, 76, 106, 128, 131, 134, 139, 158, 159, 166, 217, 248, and 272; (iii) 52, 53, 76, 106, 118, 128, 129, 130, 131, 134, 139, 156, 158, 166, 217, 248, and 272; (iv) 52, 53, 76, 128, 118, 156, 158, 166, 217, and 248; (v) 53, 76, 128, 158, 166, and 248; (vi) 52, 53, 76, 118, 128, 156, 158, 166, 217, and 248; or (vii) 52-129, 53-166, 76-166, 166-159, 166-272, 53-76-166, 53-118-166, 53-166-272, 76-118-166, 76-166-248, 76-166-256, and 76-166-272; wherein the amino acid positions of the variant or recombinant polypeptide or active fragment thereof are numbered by correspondence with the amino acid sequence of SEQ ID NO: 10.

A still further embodiment is directed to a protease variant, or a recombinant polypeptide or an active fragment thereof comprising an amino acid sequence comprising two or more substitutions selected from: (i) A1X, T3X, G7X, P9X, A19X, S24X, K27X, I30X, N38X, A40X, N43X, K45X, A48X, S52X, G53X, A57X, V72X, N76X, T78X, T79X, Y86X, N87X, A88X, D89X, L90X, Y91X, V93X, S97X, S99X, G100X, S101X, T103X, L104X, G106X, I107X, I111X, S114X, I115X, S116X, N117X, G118X, N120X, I122X, N123X, M124X, G128X, S129X, S130X, G131X, T133X, A134X, Q136X, C139X, N140X, N141X, Y143X, N144X, R145X, I147X, V148X, V149X, I150X, A153X, S156X, S158X, S159X, G160X, G166X, S181X, S182X, T185X, S188X, S194X, T209X, N212X, N213X, A215X, S216X, F217X, M222X, A224X, A228X, I234X, A236X, Y238X, S240X, T242X, Q245X, I246X, E248X, K251X, N252X, N256X, K265X, V267X, N269X, V270X, E271X, S272X, A273X, and Q275X; (ii) A1X, T3X, P9X, A19X, S24X, K27X, I30X, N38X, A40X, N43X, K45X, A48X, S52X, G53X, A57X, V72X, N76X, T78X, T79X, Y86X, N87X, A88X, D89X, L90X, Y91X, V93X, S97X, S99X, G100X, S101X, T103X, L104X, G106X, I107X, I111X, S114X, I115X, S116X, N117X, G118X, N120X, I122X, N123X, M124X, G128X, S129X, S130X, G131X, T133X, A134X, Q136X, C139X, N140X, N141X, Y143X, N144X, R145X, I147X, V148X, V149X, I150X, A153X, S156X, S158X, S159X, G160X, G166X, S181X, S182X, T185X, S188X, S194X, T209X, N212X, N213X, A215X, S216X, F217X, M222X, A224X, A228X, I234X, A236X, Y238X, S240X, T242X, Q245X, I246X, E248X, K251X, N252X, N256X, K265X, V267X, E271X, S272X, and Q275X; (iii) A1X, G7X, P9X, K27X, N38X, A40X, K45X, A48X, S52X, G53X, N76X, T78X, Y91X, G160X, S114X, N117X, G118X, I122X, G128X, S129X, S130X, N140X, I147X, V149X, S156X, S158X, S159X, G160X, G166X, S181X, S182X, S194X, T209X, F217X, M222X, A228X, I234X, A236X, Y238X, Q245X, E248X, N256X, N269X, V270X, E271X, S272X, A273X, and Q275X; (iv) A1X, S24X, I30X, N38X, A40X, A48X, S52X, G53X, N76X, T78X, T79X, Y86X, N87X, A88X, D89X, Y91X, V93X, S97X, G100X, T003X, L104X, G106X, I111X, S114X, I115X, S116X, N117X, G118X, N120X, I122X, M124X, G128X, S129X, S130X, G113X, T133X, A134X, Q136X, C139X, N114X, Y143X, N144X, I147X, V148X, V149X, I150X, A153X, S156X, S158X, S159X, G166X, S182X, T185X, S194X, T209X, N212X, N213X, S216X, F217X, M222X, A224X, A236X, Y238X, E248X, K251X, N252X, N256X, S272X, and Q275X; (v) A1X, S24X, I30X, N38X, A40X, S52X, G53X, N76X, T78X, T79X, Y86X, N87X, A88X, D89X, V93X, S97X, G160X, T103X, L104X, G106X, I111X, S114X, I115X, S116X, N117X, G118X, N120X, I122X, M124X, G128X, S129X, G113l X, T133X, A134X, Q136X, C139X, N141X, Y143X, N144X, I147X, V148X, V149X, I150X, A153X, S156X, S158X, S159X, G166X, S182X, T185X, S194X, T209X, N212X, N213X, S216X, F217X, M222X, A224X, A236X, Y238X, E248X, K251X, N252X, N256X, S272X, and Q275X; or (vi) A1X, N38X, A40X, A48X, S52X, G53X, N76X, Y91X, S114X, N117X, G118X, G128X, S129X, S130X, I147X, V149X, S156X, S158X, S159X, G166X, S182X, F217X, Y238X, E248X, N256X, and S272X; wherein X is any amino acid and the amino acid positions of the variant or recombinant polypeptide or active fragment thereof are numbered by correspondence with the amino acid sequence of SEQ ID NO: 10. An an even still further embodiment is directed to a protease variant, or a recombinant polypeptide or an active fragment thereof comprising an amino acid sequence comprising two or more substitutions selected from: (i) A1X, T3X, P9X, A19X, S24X, K27X, I30X, N38X, A40X, N43X, K45X, A48X, S52X, G53X, A57X, V72X, N76X, T78X, T79X, Y86X, N87X, A88X, D89X, L90X, Y91X, V93X, S97X, S99X, G100X, S101X, T103X, L104X, G106X, I107X, I111X, S114X, I115X, S116X, N117X, G118X, N112X, I122X, N123X, M124X, G128X, S129X, S130X, G131X, T133X, A134X, Q136X, C139X, N140X, N141X, Y143X, N144X, R145X, I147X, V148X, V149X, I150X, A153X, S156X, S158X, S159X, G160X, G166X, S181X, S182X, T185X, S188X, S194X, T209X, N212X, N213X, A215X, S216X, F217X, M222X, A224X, A228X, I234X, A236X, Y238X, S240X, T242X, Q245X, I246X, E248X, K251X, N252X, N256X, K265X, V267X, E271X, S272X, and Q275X; (ii) A1X, T3X, P9X, A19X, S24X, K27X, I30X, N38X, A40X, N43X, K45X, A48X, S52X, G53X, A57X, V72X, N76X, T78X, T79X, Y86X, N87X, A88X, D89X, L90X, Y91X, V93X, S97X, S99X, G160X, S101X, T103X, L104X, G100X, I107X, I111X, S114X, I115X, S116X, N117X, G118X, N120X, I122X, N123X, M124X, G128X, S129X, S130X, G131X, T133X, A134X, Q136X, C139X, N140X, N141X, Y143X, N144X, R145X, I147X, V148X, V149X, I150X, S156X, S158X, S159X, G160X, G166X, S181X, S182X, T185X, S188X, S194X, T209X, N212X, N213X, A215X, S216X, F217X, M222X, A224X, A228X, 1234X, A236X, Y238X, S240X, T242X, Q245X, I246X, E248X, K251X, N252X, N256X, K265X, V267X, E271X, S272X, and Q275X; (iii) P9X, N38X, A40X, K45X, A48X, S52X, G53X, N76X, T78X, G100X, S114X, N117X, G118X, G128X, S129X, S30X, N140X, I147X, V149X, S156X, S158X, S159X, G160X, G166X, S181X, S182X, S194X, T209X, F217X, I234X, A236X, Y238X, Q245X, E248X, N256X, S272X, and Q275X; (iv) A1X, S24X, I30X, N38X, A40X, A48X, S52X, G53X, N76X, T78X, T79X, Y86X, N87X, A88X, D89X, Y91X, V93X, S97X, G100X, T103X, L104X, G106X, I111X, S114X, I115X, S116X, N117X, G118X, N120X, I122X, M124X, G128X, S129X, S130X, G131X, T133X, A134X, Q136X, C139X, N141X, Y143X, N144X, I147X, V148X, V149X, I150X, S156X, S158X, S159X, G166X, S182X, T185X, S194X, T209X, N212X, N213X, S216X, F217X, M222X, A224X, A236X, Y238X, E248X, K251X, N252X, N256X, S272X, and Q275X; (v) A1X, S24X, I30X, N38X, A40X, S52X, G53X, N76X, T78X, T79X, Y86X, N87X, A88X, D89X, V93X, S97X, G100X, T103X, L104X, G106X, I111X, S114X, I115X, S116X, N117X, G118X, N120X, I122X, M124X, G128X, S129X, G131X, T133X, A134X, Q136X, C139X, N141X, Y143X, N144X, I147X, V148X, V149X, I150X, S156X, S158X, S159X, G166X, S182X, T185X, S194X, T209X, N212X, N213X, S216X, F217X, M222X, A224X, A236X, Y238X, E248X, K251X, N252X, N256X, S272X, and Q275X; or (vi) N38X, A40X, A48X, S52X, G53X, N76X, S114X, N117X, G118X, G128X, S129X, S130X, I147X, V149X, S156X, S158X, S159X, G166X, S182X, F217X, Y238X, E248X, N256X, and S272X; wherein X is any amino acid and the amino acid positions of the variant or recombinant polypeptide or active fragment thereof are numbered by correspondence with the amino acid sequence of SEQ ID NO: 10. Yet still another embodiment is directed to a protease variant, or a recombinant polypeptide or an active fragment thereof comprising an amino acid sequence comprising two or more substitutions selected from: (i) S52X, G53X, N76X, Y86X, S97X, T103X, L04X, G106X, I111X, S114X, I115X, S116X, G118X, N120X, I122X, M124X, G128X, S129X, S130X, G131X, A134X, Q136X, C139X, N141X, Y143X, N144X, I147X, V148X, I150X, S156X, S158X, S159X, G166X, F217X, E248X, and S272X; (ii) G53X, N76X, G106X, G128X, G131X, A134X, C139X, S158X, S159X, G166X, F217X, E248X, and S272X; (iii) S52X, G53X, N76X, G106X, G118X, G128X, S129X, S130X, G131X, A134X, C139X, S156X, G166X, F217X, E248X, and S272X; (iv) S52X, G53X, N76X, G118X, G128X, S156X, S158X, G166X, F217X, and E248X; (v) G53X, N76X, G128X, S158X, G166X, and E248X; (vi) S52X, G53X, N76X, G118X, G128X, S156X, S158X, G166X, F217X, and E248X, or (vii) S52X-S129X, G53X-G166X, N76X-G166X, G166X-S159X, G166X-S272X, G53X-N76X-G166X, G53X-G118X-G166X, G53X-G166X-S272X, N76X-G166X, N76X-G166X-E248X, N76X-G166X-N256X, and N76X-G166X-S272X; wherein X is any amino acid and the amino acid positions of the variant or recombinant polypeptide or active fragment thereof are numbered by correspondence with the amino acid sequence of SEQ ID NO:10.

An even still further embodiment is directed to a protease variant, or a recombinant polypeptide or an active fragment thereof comprising an amino acid sequence comprising two or more substitutions selected from: (i) X1G, X1T, X3E, X3V, X7C, X9I, X9T, X9V, X19R, X24R, X24T, X27S, X27T, X30V, X38A, X38Q, X38T, X40D, X40E, X40L, X40V, X43D, X43H, X43L, X43S, X45Q, X45R, X48E, X48Q, X52A, X52N, X52Q, X52T, X53D, X53E, X53N, X57P, X72A, X76D, X78A, X78D, X78G, X78S, X79I, X86P, X87S, X88V, X89E, X89S, X90P, X91H, X93T, X97G, X99L, X100A, X100K, X101T, X103E, X103S, X104V, X106S, X107T, X107V, X111L, X114A, X114T, X115G, X116E, X116N, X117E, X117S, X118R, X120H, X122A X123D, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130A, X130E, X130F, X130G, X130H, X130I, X130L, X130M, X130N, X130Q, X130R, X130T, X130V, X130W, X130Y, X131P, X133A, X133D, X133V, X134T, X136E, X139V, X140D, X140Y, X141S, X143T, X144S, X145N, X145W, X147A, X147V, X148L, X149G, X149L, X149N, X149S, X150V, X153S, X156A, X156H, X156N, X158T, X158V, X159D, X159E, X159N, X159Q, X160D, X166A, X166D, X166H, X166N, X166S, X166Y, X181A, X181N, X182E, X185Q, X188Y, X194P, X209F, X209V, X209Y, X212S, X213T, X215D, X215E, X216T, X217L, X217R, X222L, X224T, X228S, X234V, X236N, X236Q, X238F, X240F, X242S, X245A, X245N, X246V, X248K, X248N, X251R, X251S, X252S, X256E, X256K, X256P, X256R, X256Y, X265S, X267N, X269K, X270C, X271F, X271W, X272K, X272N, X272Q, X273V, X275H, and X275R; (ii) X1G, X1T, X3E, X3V, X9I, X9T, X9V, X19R, X24R, X24T, X27S, X27T, X30V, X38A, X38Q, X38T, X40D, X40E, X40L, X40V, X43D, X43H, X43L, X43S, X45Q, X45R, X48E, X48Q, X52A, X52N, X52Q, X52T, X53D, X53E, X53N, X57P, X72A, X76D, X78A, X78D, X78G, X78S, X79I, X86P, X87S, X88V, X89E, X89S, X90P, X91H, X93T, X97G, X99L, X100A, X100K, X101T, X103E, X103S, X104V, X106S, X107T, X107V, X111L, X114A, X114T, X115G, X116E, X116N, X117E, X117S, X118R, X120H, X122A, X123D, X124L, X128A, X128D, X128E, X128H, X128K, X128M, X128N, X128Q, X128S, X128Y, X129D, X129E, X129O, X129H, X129I, X129K, X129L, X129M, X129N, X129P, X129Q, X129R, X129T, X129V, X129Y, X130A, X130E, X130F, X130G, X130H, X301, X130L, X130M, X130N, X130Q, X130R, X130T, X130V, X130W, X130Y, X131P, X133A, X133D, X133V, X134T, X136E, X139V, X140D, X140Y, X141S, X143T, X144S, X145N, X145W, X147A, X147V, X148L, X149G, X149L, X149N, X149S, X150V, X153S, X156A, X156H, X156N, X158T, X158V, X159D, X159E, X159N, X159Q, X160D, X166A, X166D, X166H, X166N, X166S, X166Y, X181A, X181N, X182E, X185Q, X188Y, X194P, X209F, X209V, X209Y, X212S, X213T, X215D, X215E, X216T, X217L, X217R, X222L, X224T, X228S, X234V, X236N, X236Q, X238F, X240F, X242S, X245A, X245N, X246V, X248K, X248N, X251R, X251S, X252S, X256E, X256K, X256P, X256R, X256Y, X265S, X267N, X271F, X271W, X272K, X272N, X272Q, X275H, and X275R; (iii) X1G, X7C, X9T, X27M, X38Q, X38T, X40D, X45Q, X45R, X48E, X48Q, X52F, X52Q, X52T, X53D, X53E, X53Y, X76D, X78A, X91H, X100A, X114T, X117S, X118R, X122V, X128A, X128D, X128E, X128H, X128K, X128L, X128M, X128N, X128Q, X128R, X128S, X128T, X128V, X128W, X128Y, X129D, X129E, X129F, X129G, X129H, X129I, X129K, X129L, X129M, X129N, X129P, X129Q, X129R, X129T, X129V, X129Y, X130A, X130D, X130E, X130F, X130G, X130H, X130I, X130L, X130M, X130N, X130Q, X130R, X130T, X130V, X130W, X130Y, X140D, X147A, X149G, X156D, X156H, X156N, X156T, X158T, X158V, X159D, X159N, X160D, X166D, X166H, X166Y, X181N, X182E, X194P, X209V, X217R, X222L, X228S, X234V, X236N, X236Q, X238F, X245A, X248D, X248K, X248N, X256E, X256P, X256R, X269K, X270C, X271G, X272K, X272Q, X273V, X275R, and X275Y, (iv) X1G, X1T, X24R, X24T, X30V, X38A, X38Q, X38T, X40D, X40E, X40L, X40V, X48Q, X52A, X52N, X52Q, X52T, X53D, X53E, X53N, X76D, X78A, X78D, X78G, X78S, X79I, X86P, X87S, X88V, X89E, X91H, X93T, X97G, X100A, X100K, X103E, X103S, X104V, X106S, X111L, X114A, X114T, X115G, X116E, X116N, X17E, X17S, X18R, X120H, X122A, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130F, X130H, X130I, X130Y, X131P, X133A, X133D, X133V, X134T, X136E, X139V, X141S, X143T, X144S, X147A, X147V, X148L, X149G, X149L, X149N, X149S, X150V, X153S, X156A, X156H, X156N, X158T, X158V, X159D, X159E, X159N, X159Q, X166A, X166D, X166H, X166N, X166S, X166Y, X182E, X185Q, X194P, X209F, X209V, X209Y, X212S, X213T, X216T, X217L, X217R, X222L, X224T, X236N, X236Q, X238F, X248K, X248N, X251R, X251S, X252S, X256E, X256K, X256P, X256R, X256Y, X272K, X272N, X272Q, X275H, and X275R; (v) X1G, X1T, X24R, X24T, X30V, X38A, X38Q, X38T, X40D, X40E, X40L, X40V, X52A, X52N, X52Q, X52T, X53D, X53E, X53N, X76D, X78A, X78D, X78G, X78S, X79I, X86P, X87S, X88V, X89E, X93T, X97G, X100A, X100K, X103E, X103S, X104V, X106S, X111L, X114A, X114T, X115G, X116E, X116N, X117E, X117S, X118R, X120H, X122A, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X131P, X133A, X133D, X133V, X134T, X136E, X139V, X141S, X143T, X144S, X147A, X147V, X148L, X149G, X149L, X149N, X149S, X150V, X153S, X156A, X156H, X156N, X158T, X158V, X159D, X159E, X159N, X159Q, X166A, X166D, X166H, X166N, X166S, X166Y, X182E, X185Q, X194P, X209F, X209V, X209Y, X212S, X213T, X216T, X217L, X217R, X222L, X224T, X236N, X236Q, X238F, X248K, X248N, X251R, X251S, X252S, X256E, X256K, X256P, X256R, X256Y, X272K, X272N, X272Q, X275H, and X275R; or (vi) X1G, X38Q, X40D, X48Q, X52F, X52T, X53E, X53Y, X76D, X91H, X114T, X117S, X118R, X128D, X128M, X128V, X129F, X129M, X129R, X129V, X130F, X130H, X130I, X130Y, X147A, X149G, X156N, X158T, X159N, X166D, X166H, X182E, X217R, X238F, X248N, X256E, X256P, X256R, and X272K; wherein X is any amino acid and the amino acid positions of the variant or recombinant polypeptide or active fragment thereof are numbered by correspondence with the amino acid sequence of SEQ ID NO: 10. An even yet still further embodiment is directed to a protease variant, or a recombinant polypeptide or an active fragment thereof comprising an amino acid sequence comprising two or more substitutions selected from: (i) X1G, X11T, X3E, X3V, X9I, X9T, X9V, X119R, X24R, X24T, X27S, X27T, X30V, X38A, X38Q, X38T, X40D, X40E, X40L, X40V, X43D, X43H, X43L, X43S, X45Q, X45R, X48E, X48Q, X52A, X52N, X52Q, X52T, X53D, X53E, X53N, X57P, X72A, X76D, X78A, X78D, X78G, X78S, X79I, X86P, X87S, X88V, X89E, X89S, X90P, X91H, X93T, X97G, X99L, X100A, X100K, X101T, X103E, X103S, X104V, X100S, X107T, X107V, X111L, X114A, X114T, X115G, X116E, X116N, X117E, X117S, X18R, X120H, X122A, X123D, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130A, X130E, X130F, X130G, X130H, X130I, X130L, X130M, X130N, X130Q, X130R, X130T, X130V, X130W, X130Y, X131P, X133A, X133D, X133V, X134T, X136E, X139V, X140D, X140Y, X141S, X143T, X144S, X145N, X145W, X147A, X147V, X148L, X149G, X149L, X149N, X149S, X150V, X153S, X156A, X156H, X156N, X158T, X158V, X159D, X159E, X159N, X159Q, X160D, X166A, X166D, X166H, X166N, X166S, X166Y, X181A, X181N, X182E, X185Q, X188Y, X194P, X209F, X209V, X209Y, X212S, X213T, X215D, X215E, X216T, X217L, X217R, X222L, X224T, X228S, X234V, X236N, X236Q, X238F, X240F, X242S, X245A, X245N, X246V, X248K, X248N, X251R, X251S, X252S, X256E, X256K, X256P, X256R, X256Y, X265S, X267N, X271F, X271W, X272K, X272N, X272Q, and X275R; (ii) X1G, X1T, X3E, X3V, X9I, X9T, X9V, X19R, X24R, X24T, X27S, X27T, X30V, X38A, X38Q, X38T, X40D, X40E, X40L, X40V, X43D, X43H, X43L, X43S, X45Q, X45R, X48E, X48Q, X52A, X52N, X52Q, X52T, X53D, X53E, X53N, X57P, X72A, X76D, X78A, X78D, X78G, X78S, X79I, X86P, X87S, X88V, X89E, X89S, X90P, X91H, X93T, X97G, X99L, X100A, X100K, X101T, X103E, X103S, X104V, X106S, X107T, X107V, X111L, X114A, X114T, X115G, X116E, X116N, X117E, X17S, X118R, X120H, X122A, X123D, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130A, X130E, X130F, X130G, X130H, X130I, X130L, X130M, X130N, X130Q, X130R, X130T, X130V, X130W, X130Y, X131P, X133A, X133D, X133V, X134T, X136E, X139V, X140D, X140Y, X141S, X143T, X144S, X145N, X145W, X147A, X147V, X148L, X149G, X149L, X149N, X149S, X150V, X156A, X156H, X156N, X158T, X158V, X159D, X159E, X159N, X159Q, X160D, X166A, X166D, X166H, X166N, X166S, X166Y, X181A, X181N, X182E, X185Q, X188Y, X194P, X209F, X209V, X209Y, X212S, X213T, X215D, X215E, X216T, X217L, X217R, X222L, X224T, X228S, X234V, X236N, X236Q, X238F, X240F, X242S, X245A, X245N, X246V, X248K, X248N, X251R, X251S, X252S, X256E, X256K, X256P, X256R, X256Y, X265S, X267N, X271F, X271W, X272K, X272N, X272Q, and X275R; (iii) X9T, X38Q, X38T, X40D, X45Q, X45R, X48E, X52T, X53D, X53E, X76D, X78A X100A, X114T, X117S, X118R, X128A X128D, X128E, X128H, X128K, X128M, X128N, X128Q, X128S, X128Y, X129D, X129E, X129G, X129H, X129I, X129K, X129L, X129M, X129N, X129Q, X129R, X129T, X129V, X129Y, X130A, X130E, X130F, X130G, X130H, X130I, X130L, X130M, X130N, X130Q, X130R, X130T, X130V, X130W, X130Y, X140D, X147A, X149G, X156N, X158T, X158V, X159D, X159N, X160D, X166D, X166H, X181N, X182E, X194P, X209V, X217R, X234V X236N, X236Q, X245A, X248K, X248N, X256P, X256R, X272K, X272Q, and X275R; (iv) X1G, X1T, X24R, X24T, X30V, X38A, X38Q, X38T, X40D, X40E, X40L, X40V, X48Q, X52A, X52N, X52Q, X52T, X53D, X53E, X53N, X76D, X78A, X78D, X78G, X78S, X79I, X86P, X87S, X88V, X89E, X91H, X93T, X97G, X100A, X100K, X103E, X103S, X104V, X106S, X111L, X114A, X114T, X115G, X116E, X16N X117E X117S, X118R, X120H, X122A X124L X128A, X128D X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130F, X130H, X130I, X130Y, X131P, X133A, X133D, X133V, X134T, X136E, X139V, X141S, X143T, X144S, X1147A, X147V, X148L, X149G, X149L, X149N, X149S, X150V, X156A, X1156H, X156N, X158T, X158V, X159D, X159E, X159N, X159Q, X166A, X166D, X166H, X166N, X166S, X166Y, X182E, X185Q, X194P, X209F, X209V, X209Y, X212S, X213T, X216T, X217L, X217R, X222L, X224T, X236N, X236Q, X238F, X248K, X248N, X251R, X251S, X252S, X256E, X256K, X256P, X256R, X256Y, X272K, X272N, X272Q, and X275R: (v) X1G, X1T, X24R, X24T, X30V, X38A, X38Q, X38T, X40D, X40E, X40L, X40V, X52A, X52N, X52Q, X52T, X53D, X53E, X53N, X76D, X78A, X78D, X78G, X78S, X79I, X86P, X87S, X88V, X89E, X93T, X97G, X100A, X100K, X103E, X103S, X104V, X106S, X111L, X114A, X114T, X115G, X116E, X116N, X117E, X117S, X118R, X120H, X122A, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X131P, X133A, X133D, X133V, X134T, X136E, X139V, X141S, X143T, X144S, X147A, X147V, X148L, X149G, X149L, X149N, X149S, X150V, X156A, X156H, X156N, X158T, X158V, X1159D, X159E, X159N, X1159Q, X160A, X166D, X166H, X160N, X166S, X166Y, X182E, X185Q, X194P, X209F, X209V, X209Y, X212S, X213T, X216T, X217L, X217R, X222L, X224T, X236N, X236Q, X238F, X248K, X248N, X251R, X251S, X252S, X256E, X256K, X256P, X256R, X256Y, X272K, X272N, X272Q, and X275R; or (vi) X38Q, X40D, X52T, X53E, X76D, X114T, X117S, X118R, X128D, X128M, X129M, X129R, X129V, X130F, X130H, X130I, X130Y, X1147A, X149G, X156N, X158T, X159N, X166D, X166H, X182E, X217R, X248N, X256P, X256R, and X272K; wherein X is any amino acid and the amino acid positions of the variant or recombinant polypeptide or active fragment thereof are numbered by correspondence with the amino acid sequence of SEQ ID NO: 10. Another embodiment is directed to a protease variant, or a recombinant polypeptide or an active fragment thereof comprising an amino acid sequence comprising two or more substitutions selected from: (i) X52T, X53D, X76D, X86P, X97G, X103S, X104V, X106S, X111L, X114A, X115G, X116N, X18R, X120H, X122A-X124L, X128D, X128S, X129F, X130Y, X131P, X134T, X136E, X139V, X141S, X143T, X144S, X147V, X148L, X150V, X156N, X158T, X159D, X166D, X217R, X248N, and X272K; (ii) X53D, X76D, X106S, X128S, X131P, X134T, X139V, X158T, X159D, X166D, X217R, X248N, and X272K; (iii) X52T, X53D, X76D, X106S, X118R X128D, X129F, X130Y, X1131P, X134T, X139V, X156N, X1166D, X217R, X248N, and X272K; (iv) X52T, X53D, X76D, X118R, X128S, X156N, X158T, X166D, X217R, and X248N; (v) X53D, X76D, X128S, X158T, X166D, and X248N; (vi) X52T, X53D, X76D, X118R, X128S, X156N, X158T, X160D, X217R, and X248N; or (vii) X52T-X129F, X53D-X166D, X76D-X166D, X166H-X159D, X166D-X272K, X53D-X76D-X166D, X53D-X118R-X166D, X53D-X166D-X272K, X76D-X118R-X166D, X76D-X166D-X248K, X76D-X166D-X256R, and X76D-X166D-X272K; wherein X is any amino acid and the amino acid positions of the variant or recombinant polypeptide or active fragment thereof are numbered by correspondence with the amino acid sequence of SEQ ID NO: 10.

An even further embodiment is directed to a protease variant, or a recombinant polypeptide or an active fragment thereof comprising an amino acid sequence comprising two or more substitutions selected from: (i) A001G, A001T, T003E, T003V, G007C, P009I, P009T, P09V, A019R, S024R, S024T, K027S, K027T, I030V, N038A, N038Q, N038T, A040D, A040E, A040L, A040V, N043D, N043H, N043L, N043S, K045Q, K045R, A048E, A048Q, S052A, S052N, S052Q, S052T, G053D, G053E, G053N, A057P, V072A, N076D, T078A, T078D, T0780, T078S, T079I, Y086P, N087S, A088V, D089E, D089S, L090P, Y091H, V093T, S097G, S099L, G100A, G100K, S101T, T103E, T103S, L104V, G106S, I107T, I107V, I111L, S114A, S114T, I115G, S116E, S116N, N17E, N117S, G118R, N120H, I122A, N123D, M124L, G128A, G128D, G128E, G128H, G128K, G128M, G128N, G128Q, G128S, G128Y, S129D, S129E, S129G, S129H, S129I, S129K, S129L, S129M, S129N, S129P, S129Q, S129R, S129T, S129V, S129Y, S130A, S130E, S130F, S300, S130H, S130I, S130L, S130M, S130N, S130Q, S130R, S130T, S130V, S130W, S130Y, G131P, T133A, T133D, T133V, A134T, Q136E, C139V, N140D, N140Y, N141S, Y143T, N144S, R145N, R145W, I147A, I147V, V148L, V149G, V149L, V149N, V149S, I150V, A153S, S156A, S156H, S156N, S158T, S158V, S159D, S159E, S159N, S159Q, G160D, G166A, G166D, G166H, G166N, G166S, G166Y, S181A, S181N, S182E, T185Q, S188Y, S194P, T209F, T209V, T209Y, N212S, N213T, A215D, A215E, S216T, F217L, F217R, M222L, A224T, A228S, I234V, A236N, A236Q, Y238F, S240F, T242S, Q245A, Q245N, I246V, E248K, E248N, K251R, K251S, N252S, N256E, N256K, N256P, N256R, N256Y, K265S, V267N, N269K, V270C, E271F, E271W, S272K, S272N, S272Q, A273V, Q275H, and Q275R; (ii) A001G, A001T, T003E, T003V, P009I, P009T, P009V, A019R, S024R, S024T, K027S, K027T, I030V, N038A, N038Q, N038T, A040D, A040E, A040L, A040V, N043D, N043H, N043L, N043S, K045Q, K045R, A048E, A048Q, S052A, S052N, S052Q, S052T, G053D, G053E, G053N, A057P, V072A, N076D, T078A, T078D, T078G, T078S, T079I, Y086P, N087S, A088V, D089E, D089S, L090P, Y091H, V093T, S097D, S099L, G100A, G100K, S101T, T103E, T103S, L104V, G106S, I107T, I107V, I111L, S114A, S114T, I115G, S116E, S116N, N117E, N117S, G118R, N120H, I122A, N123D, M124L, G128A, G281D, G128E, G128H, G128K, G128M, G128N, G128Q, G128S, G128Y, S129D, S129E, S129G, S129H, S129I, S129K, S129L, S129M, S129N, S129P, S129Q, S129R, S129T, S129V, S129Y, S130A, S130E, S130F, S130G, S130H, S130I, S130L, S130M, S130N, S130Q, S130R, S130T, S130V, S130W, S130Y, G131P, T133A, T133D, T133V, A134T, Q136E, C139V, N140D, N140Y, N141S, Y143T, N144S, R145N, R145W, I147A, I147V, V148L, V149G, V149L, V149N, V149S, I150V, A153S, S156A, S156H, S156N, S158T, S158V, S159D, S159E, S159N, S159Q, G160D, G166A, G166D, G166H, G166N, G166S, G166Y, S181A, S181N, S182E, T185Q, S188Y, S194P, T209F, T209V, T209Y, N212S, N213T, A215D, A215E, S216T, F217L, F217R, M222L, A224T, A228S, I234V, A236N, A236Q, Y238F, S240F, T242S, Q245A, Q245N, I246V, E248K, E248N, K251R, K251S, N252S, N256E, N256K, N256P, N256R, N256Y, K265S, V267N, E271F, E271W, S272K, S272N, S272Q, Q275H, and Q275R; (iii) A001G, G007C, P009T, K027M, N038Q, N038T, A040D, K045Q, K045R, A048E, A048Q, S052F, S052Q, S052T, G053D, G053E, G053Y, N076D, T078A, Y091H, G100A, S114T, N17S, G118R, I122V, G128A, G281D, G128E, G128H, G128K, G128L, G128M, G128N, G128Q, G128R, G128S, G128T, G128V, G128W, G128Y, S129D, S129E, S129F, S129G, S129H, S129I, S129K, S129L, S129M, S129N, S129P, S129Q, S129R, S129T, S129V, S129Y, S130A, S130D, S130E, S130F, S130G, S130H, S130I, S130L, S130M, S130N, S130Q, S130R, S130T, S130V, S130W, S130Y, N140D, I147A, V149G, S156D, S156H, S156N, S156T, S158T, S158V, S159D, S159N, G160D, G166D, G166H, G166Y, S181N, S182E, S194P, T209V, F217R, M222L, Y238F, Q245A, E248D, E248K, E248N, N256E, N256P, N256R, N269K, V270C, E271G, S272K, S272Q, A273V, Q275R, and Q275Y; (iv) A001G, A001T, S024T, I030V, N038Q, A040D, A048Q, S052A, S052Q, S052T, G053D, G053E, N076D, T078D, T078S, T079I, Y086P, N087S, A088V, D089E, D089S, Y091H, V093T, S097G, G100A, T103E, T103S, L104V, G106S, I111L, S114A, S114T, I115G, S116N, N117S, G118R, N120H, I122A, M124L, G128S, S129P, S130F, S130H, S130I, S130Y, G131P, T133A, T133V, A134T, Q136E, C139V, N141S, Y143T, N144S, I147A, I147V, V148L, V149G, V149L, I150V, A153S, S156H, S156N, S158T, S159D, S159N, G166D, G166H, G166S, S182E, T185Q, S194P, T209Y, N212S, N213T, S216T, F217L, F217R, M222L, A224T, A236N, Y238F, E248N, K251S, N252S, N256P, N256R, N256Y, S272K, S272Q, and Q275H; (v) A001G, A001T, S024T, I030V, N038Q, A040D, S052A, S052Q, S052T, G053D, G053E, N076D, T078D, T078S, T079I, Y086P, N087S, A088V, D089E, D089S, V093T, S097G, G100A, T103E, T103S, L104V, G106S, T111L, S114A, S114T, I115G, S116N, N117S, G118R, N120H, I122A, M124L, G128S, S129P, G131P, T133A, T133V, A134T, Q136E, C139V, N141S, Y143T, N144S, I147A, I147V, V148L, V149G, V149L, I150V, A153S, S156H, S156N, S158T, S159D, S159N, G166D, G166H, G166S, S182E, T185Q, S194P, T209Y, N212S, N213T, S216T, F217L, F217R, M222L, A224T, A236N, Y238F, E248N, K251S, N252S, N256P, N256R, N256Y, S272K, S272Q, and Q275H; or (vi) A001G, N038Q, A040D, A048Q, S052F, S052T, G053E, G053Y, N076D, Y091H, S114T, N117S, G118R, G128D, G128M, G128V, S129F, S129M, S129R, S129V, S130F, S130H, S130I, S130Y, I147A, V149G, S156N, S158T, S159N, G166D, G166H, S182E, F217R, Y238F, E248N, N256E, N256P, N256R, and S272K; wherein the amino acid positions of the variant or recombinant polypeptide or active fragment thereof are numbered by correspondence with the amino acid sequence of SEQ ID NO: 10, A still even further embodiment is directed to a protease variant, or a recombinant polypeptide or an active fragment thereof comprising an amino acid sequence comprising two or more substitutions selected from: (i) A001G, A001T, T003E, T003V, P0091, P009T, P009V, A019R, S024R, S024T, K027S, K027T, I030V, N038A, N038Q, N038T, A040D, A040E, A040L, A040V, N043D, N043H, N043L, N043S, K045Q, K045R, A048E, A048Q, S052A, S052N, S052Q, S052T, G053D, G053E, G053N, A057P, V072A, N076D, T078A, T078D, T0780, T078S, T079I, Y086P, N087S, A088V, D089E, D089S, L090P, Y091H, V093T, S097G, S099L, G100A, G100K, S101T, T103E, T103S, L104V, G106S, I107T, I107V, I111L, S114A, S114T, I115G, S116E, S116N, N117E, N117S, G118R, N120H, I122A, N123D, M124L, G128A, G128D, G128E, G128H, G128K, G128M, G128N, G128Q, G128S, G128Y, S129D, S129E, S129G, S129H, S129I, S129K, S129L, S129M, S129N, S129P, S129Q, S129R, S129T, S129V, S129Y, S130A, S130E, S130F, S130G, S130H, S130I, S130L, S130M, S130N, S130Q, S130R, S130T, S130V, S130W, S130Y, G131P, T133A, T133D, T133V, A134T, Q136E, C139V, N140D, N140Y, N141S, Y143T, N144S, R45N, R145W, I147A, I147V, V148L, V149G, V149L, V149N, V149S, I150V, S156A, S156H, S156N, S158T, S158V, S159D, S159E, S159N, S159Q, G160D, G166A, G166D, G166H, G166N, G166S, G166Y, S181A, S181N, S182E, T185Q, S188Y, S194P, T209F, T209V, T209Y, N212S, N213T, A215D, A215E, S216T, F217L, F217R, M222L, A224T, A228S, I234V, A236N, A236Q, Y238F, S240F, T242S, Q245A, Q245N, I246V, E248K, E248N, K251R, K251S, N252S, N256E, N256K, N256P, N256R, N256Y, K265S, V267N, E271F, E271W, S272K, S272N, S272Q, and Q275R; (ii) A001G, A001T, T003E, T003V, P009I, P009T, P009V, A019R, S024R, S024T, K027S, K027T, I030V, N038A, N038Q, N038T, A040D, A040E, A040L, A040V, N043D, N043H, N043L, N043S, K045Q, K045R, A048E, A048Q, S052A, S052N, S052Q, S052T, G053D, G053E, G053N, A057P, V072A, N076D, T078A, T078D, T078G, T078S, T079I, Y086P, N087S, A088V, D089E, D089S, L090P, Y091H, V093T, S097G, S099L, G100A, G100K, S101T, T103E, T103S, L104V, G106S, I071T, I107V, I111L, S114A, S114T, I115G, S116E, S116N, N117E, N117S, G118R, N120H, I122A, N123D, M124L, G128A, G128D, G128E, G128H, G128K, G128M, G128N, G128Q, G128S, G128Y, S129D, S129E, S129G, S129H, S129I, S129K, S129L, S129M, S129N, S129P, S129Q, S129R, S129T, S129V, S129Y, S130A, S130E, S130F, S130G, S130H, S130I, S130L, S130M, S130N, S130Q, S130R, S130T, S130V, S130W, S130Y, G131P, T133A, T133D, T133V, A134T, Q136E, C139V, N140D, N140Y, N141S, Y143T, N144S, R145N, R145W, I147A, I147V, V148L, V149G, V149L, V149N, V149S, I150V, S156A, S156H, S156N, S158T, S158V, S159D, S159E, S159N, S159Q, G160D, G166A, G166D, G166H, G166N, G166S, G166Y, S181A, S181N, S182E, T185Q, S188Y, S194P, T209F, T209V, T209Y, N212S, N213T, A215D, A215E, S216T, F217L, F217R, M222L, A224T, A228S, I234V, A236N, A236Q, Y238F, S240F, T242S, Q245A, Q245N, I246V, E248K, E248N, K251R, K251S, N252S, N256E, N256K, N256P, N256R, N256Y, K265S, V267N, E271F, E271W, S272K, S272N, S272Q, and Q275R; (iii) P009T, N038Q, A040D, K045Q, K045R, A048E S052T, G053D, G053E, N076D, T078A, G100A, S114T, N117S, G118R, G128A, G128D, G128E, G128H, G128K, G128M, G128N, G128Q, G128S, G128Y, S129D, S129E, S129G, S129H, S129I, S129K, S129L, S129M, S129N, S129Q, S129R, S129T, S129V, S129Y, S130A, S130E, S130F, S130G, S130H, S130I, S130L, S130M, S130N, S130Q, S130R, S130T, S130V, S130W, S130Y, N140D, I147A, V149G, S156N, S158T, S158V, S159D, S159N, G160D, G166D, G166H, S181N, S182E, S194P, T209V, F217R, Q245A, E248K, E248N, N256P, N256R, S272K, S272Q, and Q275R; (iv) A001G, A001T, S024T, I030V, N038Q, A040D, A048Q, S052A, S052Q, S052T, G053D, G053E, N076D, T078D, T078S, T079I, Y086P, N087S, A088V, D089E, D089S, Y091H, V093T, S097G, G100A, T103E, T103S, L104V, G106S, I111L, S114A, S114T, I115G, S116N, N117S, G118R, N120H, I122A, M124L, G128S, S129P, S130F, S130H, S130I, S130Y, G131P, T133A, T133V, A134T, Q136E, C139V, N141S, Y143T, N144S, I147A, I147V, V148L, V149G, V149L, I150V, S156H, S156N, S158T, S159D, S159N, G166D, G166H, G166S, S182E, T185Q, S194P, T209Y, N212S, N213T, S216T, F217L, F217R, M222L, A224T, A236N, Y238F, E248N, K251S, N252S, N256P, N256R, N256Y, S272K, S272Q, and Q275H; (v) A001G, A001T, S024T, I030V, N038Q, A040D, S052A, S052Q, S052T, G053D, G053E, N076D, T078D, T078S, T079I, Y086P, N087S, A088V, D089E, D089S, V093T, S097G, G100A, T103E, T003S, L104V, G106S, I111L, S114A, S114T, I115G, S116N, N117S, G118R, N120H, I122A, M124L, G128S, S129P, G131P, T133A, T133V, A134T, Q136E, C139V, N141S, Y143T, N144S, I147A, I147V, V148L, V149G, V149L, I150V, S156H, S156N, S158T, S159D, S159N, G166D, G166H, G166S, S182E, T185Q, S194P, T209Y, N212S, N213T, S216T, F217L, F217R, M222L, A224T, A236N, Y238F, E248N, K251S, N252S, N256P, N256R, N256Y, S272K, S272Q, and Q275H; or (vi) N038Q, A040D, S052T, G053E, N076D, S114T, N117S, G118R, G128D, G128M, S129M, S129R, S129V, S130F, S130H, S130I, S130Y, I147A, V149G, S156N, S158T, S159N, G166D, G166H, S182E, F217R, E248N, N256P, N256R, and S272K; wherein the amino acid positions of the variant or recombinant polypeptide or active fragment thereof are numbered by correspondence with the amino acid sequence of SEQ ID NO: 10, A still further embodiment is directed to a protease variant, or a recombinant polypeptide or an active fragment thereof comprising an amino acid sequence comprising two or more substitutions selected from: (i) S52T, G53D, N76D, Y086P, S097G, T103S, L104V, G106S, I111L, S114A, I115G, S116N, G118R, N120H, I122A, M124L, G128D, G128S, S129F, S129P, S130Y, G131P, A134T, Q136E, C139V, N141S, Y143T, N144S, I147V, V148L, I150V, S156N, S158T, S159D, G166D, F217R, E248N, and S272K; (ii) G53D, N76D, G106S, G128S, G131P, A134T, C139V, S158T, S159D, G166D, F217R, E248N, and S272K; (iii) S52T, G53D, N76D, G106S, G118R G128D, S129F, S130Y, G131P, A134T, C139V, S156N, G166D, F217R, E248N, and S272K: (iv) S52T, G53D, N76D, G118R, G128S, S156N, S158T, G166D, F217R, and E248N; (v) G53D, N76D, G128S, S158T, G166D, and E248N; (vi) S52T, G53D, N76D, G118R, S156N, S158T, G166D, F217R, and E248N; or (vii) S52T-S29F, G53D-G166D, N76D-G166D, G66H-S159D, G166D-S272K, G53D-N76D-G166D, G53D-G118R-G166D, G53D-G166D-S272K, N76D-G118R-G166D, N76D-G166D-E248K, N76D-G166D-N256R, and N76D-G166D-S272K: wherein the amino acid positions of the variant or recombinant polypeptide or active fragment thereof are numbered by correspondence with the amino acid sequence of SEQ ID NO: 10.

As used herein, a LG12-clade protease enzyme includes an enzyme, polypeptide, or protein, or an active fragment thereof, exhibiting a proteolytic activity. This includes members of the LG12-clade, as described in the Examples, alignments shown in FIGS. 1A-1B, 4A-4F, and FIGS. 10A-10F and/or the phylogenetic tree of FIGS. 5 and/or 11. Members of the LG12-clade were identified by selecting subtilisin molecules with close homology to LG12 SprC, generating a structure-based alignment of these sequences to other subtilisins, and identifying regions of unique sequences conserved for all the LG12-clade enzymes. One embodiment is directed to an LG12-Clade of subtilisins comprising one or more protease variants described herein, or recombinant polypeptide or an active fragment thereof. In some embodiments, the LG12 enzyme clade comprises subtilisin proteases where the parent, wild-type and/or protease variant sequence contains one or more of the following amino acid motifs: a catalytic triad comprising a Ser221, His64, and Asp32 motif and an EPNALQDGNGHGTH (SEQ ID NO:44) motif. In a further embodiment, the protease variant, or recombinant polypeptide or active fragment thereof comprises an EPNALQDGNGHGTH (SEQ ID NO:44) motif. In a still further embodiment, the protease variant, or recombinant polypeptide or active fragment thereof comprises an EPNALQDGNGHGTH (SEQ ID NO:44) motif, with the proviso that the variant or recombinant polypeptide or active fragment thereof does not comprise WP_047970940, WP_010192403, KPB_03414, BAD11988, BAD21128. Bhon03321, DSM9712_SprC. DSM6951_SprC, or DSM9711_SprC. In another embodiment, the motif extends from residue E54 to H67, wherein the amino acid positions of the motif are numbered by correspondence with the amino acid sequence of LG12 wild-type set forth in SEQ ID NO:10. Any LG12-clade protease enzyme sequence can be used as a parent sequence for generation of variant subtilisin enzymes of the present invention. In one embodiment, the protease variant, or recombinant polypeptide or active fragment thereof is derived from a reference polypeptide selected from SEQ ID NOs:10, 18, 19, 45, 46, 57, 58, and 66. In another embodiment, the protease variant, or recombinant polypeptide or active fragment thereof is derived from a reference polypeptide selected from SEQ ID NO: 10. In another embodiment, the protease variant, or recombinant polypeptide or active fragment thereof is derived from a reference polypeptide selected from SEQ ID NOs:10 and said protease variant, or recombinant polypeptide or active fragment thereof has one or more substitutions in one or more molecular segments selected from Segment 1 comprising positions 76-79 and 86. Segment 2 comprising positions 97-148, Segment 3 comprising positions 150-170, and Segment 4 comprising positions 207-220. Members of the LG12-clade were confirmed to cluster to the same branching points in a phylogentic tree created using the Neighbor Joining method (Saitou, N, and Nei, M. (1987) Mol. Biol. Evol. 4:406-425).

In some embodiments, the sequence identity for the LG12-clade includes enzymes that are at least 60%, 65%, 70%, 75%, 80%, 85%, 90%, 91%, 92%, 93%, 94%, 95%, 96%, 97%, 98%, 99% or 100% identical to the LG12 SprC sequence (SEQ ID NO:10). In other embodiments, the protease variant, or recombinant polypeptide or active fragment thereof comprises an amino acid sequence having at least 75, 80, 85, 90, 95, 96, 97, 98, or 99% amino acid sequence identity to SEQ ID NO:10. In yet other embodiments, the protease variant, or recombinant polypeptide or active fragment thereof comprises an amino acid sequence having at least 75% amino acid sequence identity to SEQ ID NO: 10, with the proviso that the variant or recombinant polypeptide or active fragment thereof does not comprise WP_047970940. WP_010192403. KPB_03414, BAD11988, BAD21128, Bhon03321, DSM9712_SprC, DSM6951_SprC, or DSM9711_SprC. In a still further embodiment, the protease variant, or recombinant polypeptide or active fragment thereof comprises an amino acid sequence having at least 80% amino acid sequence identity to SEQ ID NO: 10, with the proviso that the variant or recombinant polypeptide or active fragment thereof does not comprise WP_047970940, WP_010192403, KPB_03414, Bhon03321, DSM9712_SprC. DSM6951_SprC, or DSM9711_SprC. In an even still further embodiment, the protease variant, or recombinant polypeptide or active fragment thereof comprises an amino acid sequence having at least 95% amino acid sequence identity to SEQ ID NO: 10, with the proviso that the variant or recombinant polypeptide or active fragment thereof does not comprise Bhon03321, DSM9712_SprC, or DSM6951_SprC.

The serine protease polypeptides of the present disclosure include isolated, recombinant, substantially pure, or non-naturally occurring polypeptides. In some embodiments, the polypeptides are useful in cleaning applications and can be incorporated into cleaning compositions that are useful in methods of cleaning an item or a surface in need thereof.

In some embodiments, the polypeptide has a specified degree of amino acid sequence homology to an LG12-clade protease variant disclosed herein. In some embodiments, an LG12-clade protease variant has 70%, 71%, 72%, 73%, 74%, 75%, 76%, 77%, 78%, 79%, 80%, 81%, 82%, 83%, 84%, 85%. 86%, 87%, 88%, 89%, 90%, 91%, 92%, 93%, 94%, 95%, 96%, 97%, 98%, 99% or 100% sequence identity to an amino acid sequence selected from SEQ ID NO:1, 3, 4, 5, 6, 7, 8, 29, 30, 31, 32, 33, 34, 35, 36, 37, 38, 39, 40, 41, 42, and 43. In further embodiments, an LG12-clade protease variant has 70%. 75%, 80%, 85%, 90%, 95%, 96%, 97%, 98%, 99% or 100% sequence identity to an amino acid sequence selected from SEQ ID NO:1, 3, 4, 5, 6, 7, 8, 29, 30, 31, 32, 33, 34, 35, 36, 37, 38, 39, 40, 41, 42, and 43. In another embodiment, an isolated, recombinant, substantially pure, or non-naturally occurring LG12-clade protease variant has protease activity and comprises a polypeptide sequence having at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or 100% sequence identity to an amino acid sequence selected from SEQ ID NO:1, 3, 4, 5, 6, 7, 8, 29, 30, 31, 32, 33, 34, 35, 36, 37, 38, 39, 40, 41, 42, and 43. In a still further embodiment, an LG12-clade protease variant is selected from SEQ ID NO:1, 3, 4, 5, 6, 7, 8, 29, 30, 31, 32, 33, 34, 35, 36, 37, 38, 39, 40, 41, 42, and 43.

Homology can be determined by amino acid sequence alignment, e.g., using a program such as BLAST. ALIGN, or CLUSTAL, as described herein. In some embodiments, the polypeptide is an isolated, recombinant, substantially pure, or non-naturally occurring enzyme having protease activity, such as subtilisin activity, or casein hydrolysis activity (for example, dimethylcasein hydrolysis activity).

Also provided is an LG12-clade protease variant having protease activity, such as alkaline protease activity, said variant comprising an amino acid sequence which differs from the amino acid sequence of SEQ ID NOs: 1, 3, 4, 5, 6, 7, 8, 29, 30, 31, 32, 33, 34, 35, 36, 37, 38, 39, 40, 41, 42, or 43 by no more than 50, no more than 45, no more than 40, no more than 35, no more than 30, no more than 25, no more than 20, no more than 19, no more than 18, no more than 17, no more than 16, no more than 15, no more than 14, no more than 13, no more than 12, no more than 11, no more than 10, no more than 9, no more than 6, no more than 5, no more than 4, no more than 3, no more than 2 amino acid residues, or no more than 1 amino acid residue(s), as determined by alignment of the variant protease amino acid sequence with the LG12-clade wild-type amino acid sequence.

In another embodiment, a protease variant described herein, or recombinant polypeptide or active fragment thereof has one or more improved property selected from improved cleaning performance, improved stability, and improved residual protease activity when compared to SEQ ID NO: 10 and/or 48. In another embodiment, the improved property is (i) improved residual protease activity and said protease variant, or recombinant polypeptide or active fragment thereof has 15-100%, 20-100%, 25-100%, 30-100%, 35-100%, or 40-100% residual protease activity when measured in accordance with the Stability Assay of Example 1; (ii) improved cleaning performance and said protease variant, or recombinant polypeptide or active fragment thereof has a BMI cleaning PI of ≥0.7 in comparison to SEQ ID NO:48 when measured in accordance with the Cleaning Performance in Detergent assay of Example 1: (iii) improved stability and said protease variant, or recombinant polypeptide or active fragment thereof has a stability PI≥1.01 or 1.1 or 1.2 in comparison to SEQ ID NO:10 when measured in accordance with the Stability Assay of Example 1; and/or (iv) improved cleaning performance and said protease variant, or recombinant polypeptide or active fragment thereof has a cleaning PI≥1.01 or 1.1 or 1.2 in comparison to SEQ ID NO:10 when measured in accordance with the Cleaning Performance in Detergent Assay of Example 1.

In some embodiments, one or more LG12-clade protease variants has at least one beneficial mutation compared to the parent and/or BPN′-Y217L, wherein the benefit can be any one or more of the following: % residual protease activity in 10% HDL non-boron detergent (e.g., Persil non-bio HDL and Test HDL) that is greater than BPN′-Y217L; % residual protease activity in 10% HDL non-boron detergent (e.g., Persil non-bio HDL and Test HDL) that is 5-10 fold greater than BPN′-Y217L, laundry stability PI>1.01 over LG12 wild-type protease in 10% OMO HDL pH 8.2 at 51° C. and/or LAS/EDTA at 53.5° C.; laundry stability PI>0.75 over LG12 wild-type in 10% Kirkland HDL pH 8.2 at 56° C.; HDD cleaning PI>1.01 over LG12 wild-type protease in Kirkland Ultra HDD pH 10.6, OMO Color HDD pH 10.6, and/or Surf Excel HDD pH 10.6; or HDL cleaning PI>1.01 over LG12 wild-type in OMO HDL pH 8.2 and/or Kirkland ultraclean pH 8.2. In still other embodiments, one or more LG12-clade protease variants has at least one beneficial mutation compared to the parent and/or BPN′-Y217L, wherein the benefit can be any one or more of the following: laundry stability PI and laundry cleaning PI>1.01 over LG12 wild-type protease in all Example 1 stability and cleaning performance assays; ADW cleaning PI>1.01 over LG12 wild-type protease in GSM-B detergent pH10.5; ADW stability PI>1.01 in buffer and ADW cleaning PI>1.01 over LG12 wild-type protease in GSM-B detergent pH 10.5

In yet a further embodiment, the protease variant described herein, or recombinant polypeptide or active fragment thereof has protease activity in the presence of a surfactant, cleaning activity in a detergent composition, or a combination thereof. In still yet a further embodiment, the protease variant described herein, or recombinant polypeptide or active fragment thereof has protease activity in the presence of a surfactant, cleaning activity in a detergent composition, or a combination thereof, wherein the detergent composition is an automatic dish washing detergent or a laundry detergent. In an even still yet a further embodiment, the protease variant described herein, or recombinant polypeptide or active fragment thereof has protease activity in the presence of a surfactant, cleaning activity in a detergent composition, or a combination thereof, wherein the cleaning activity comprises hydrolysis of a protein stain. In another embodiment the protein stains is selected from egg yolk, blood, milk, ink, oil, and pigment.

In yet still an even further embodiment, the composition described herein contains phosphate, is phosphate-free, contains borate, is boron-free, or combinations thereof. In other embodiments, the composition is a boron-free composition. In some embodiments, a boron-free composition is a composition to which a borate stabilizer has not been added. In another embodiment, a boron-free composition is a composition that contains less than 5.5% boron. In a still further embodiment, a boron-free composition is a composition that contains less than 4.5% boron. In yet still another embodiment, a boron-free composition is a composition that contains less than 3.5% boron. In yet still further embodiments, a boron-free composition is a composition that contains less than 2.5% boron. In even further embodiments, a boron-free composition is a composition that contains less than 1.5% boron. In another embodiment, a boron-free composition is a composition that contains less than 1.0% boron. In still further embodiments, a boron-free composition is a composition that contains less than 0.5% boron. In other embodiments, the composition is a cleaning composition. In yet further embodiments, the cleaning composition is a detergent composition. In still further embodiments, a boron-free composition is a composition substantially-free of boron.

In another embodiment, an LG12-clade enzyme variant or an active fragment thereof contains at least two, three, four, five, six, seven, eight, nine or ten or more amino acid modifications to a parent LG12-clade enzyme, wherein a first amino acid modification is at a position of the enzyme variant selected from 52, 53, 76, 78, 79, 86, 97, 103, 104, 106, 111, 114, 115, 116, 118, 120, 122, 124, 128, 129, 130, 131, 134, 136, 139, 141, 143, 144, 147, 148, 149, 150, 156, 158, 159, 166, 217, 248, 256, and 272, wherein the amino acid positions of the variant are numbered by correspondence with the amino acid sequence of LG12 wild-type set forth in SEQ ID NO: 10.

In another embodiment, a LG2-clade enzyme variant or an active fragment thereof contains at least two, three, four, five, six, seven, eight, nine or ten or more amino acid modifications to a parent LG12-clade enzyme, wherein a first amino acid modification is at a position of the enzyme variant selected from 76, 78, 79, 86, 97, 103, 104, 106, 111, 114, 115, 116, 120, 122, 124, 128, 129, 131, 134, 136, 139, 141, 143, 144, 147, 148, 150, 158, 166, 217 and 248, wherein the amino acid positions of the variant are numbered by correspondence with the amino acid sequence of LG12 wild-type set forth in SEQ ID NO: 10.

In another embodiment, a LG12-clade enzyme variant or an active fragment thereof contains at least two, three, four, five, six, seven, eight, nine or ten or more amino acid modifications to a parent LG12-clade enzyme, wherein a first amino acid modification is at a position of the enzyme variant selected from 52, 53, 118, 129, 130, 149, 156, 159, 166, 248, 256, and 272, wherein the amino acid positions of the variant are numbered by correspondence with the amino acid sequence of LG12 wild-type set forth in SEQ ID NO:10.

In another embodiment, a LG2-clade enzyme variant or an active fragment thereof contains amino acid modifications to a parent LG12-clade enzyme, wherein said enzyme variant comprises amino acid modifications at positions 76 and 217 in combination with at least one amino acid modification selected from 52, 53, 118, 129, 130, 149, 156, 159, 166, 248, 256, and 272, wherein the amino acid positions of the variant are numbered by correspondence with the amino acid sequence of LG12 wild-type set forth in SEQ ID NO:10.

In another embodiment, a LG2-clade enzyme variant or an active fragment thereof contains amino acid modifications to a parent LG12-clade enzyme, wherein said enzyme variant comprises amino acid modifications at positions 76 and 166 in combination with at least one amino acid modification selected from 52, 53, 118, 129, 130, 149, 156, 159, 217, 248, 256, and 272, wherein the amino acid positions of the variant are numbered by correspondence with the amino acid sequence of LG12 wild-type set forth in SEQ ID NO: 10.

In another embodiment, a LG12-clade enzyme variant or an active fragment thereof contains at least two, three, four, five, six, seven, eight, nine or ten or more amino acid modifications to a parent LG12-clade enzyme, wherein a first amino acid modification is at a position of the enzyme variant selected from N76D, T78S, T79I, Y86P, S97G, T003S, L104V, G106S, I111L, S114A. I115G, S116N, N120H, I122A. M124L. G128S, G129P, G131P. A134T, Q136E, C139V. N141S, Y143T, N144S, I147V, V148L, I150V, S158T, G166D. F217L and E248N, wherein the amino acid positions of the variant are numbered by correspondence with the amino acid sequence of LG12 wild-type set forth in SEQ ID NO: 10.

An even still further embodiment is directed to a protease variant, or a recombinant polypeptide or an active fragment thereof comprising an amino acid sequence comprising one or more deletions at one or more positions selected from 53, 55, 56, 57, and 58 or G053, P055, N056, A057 and L058; wherein the amino acid positions of the variant, or recombinant polypeptide or active fragment thereof are numbered by correspondence with the amino acid sequence of SEQ ID NO: 10.

In another embodiment, a LG12-clade enzyme variant or an active fragment thereof contains at least one, two, or three or more amino acid modifications to a parent LG12-clade enzyme, wherein a first amino acid modification is a deletion at a position of the enzyme variant selected from 53, 54, 55, 56, 57, and 58, wherein the amino acid positions of the variant are numbered by correspondence with the amino acid sequence of LG12 wild-type set forth in SEQ ID NO:10.

In another embodiment, a LG12-clade enzyme variant or an active fragment thereof contains at least two, three or more amino acid modifications to a parent LG12-clade enzyme, wherein an amino acid modification is a deletion of two or more positions of the enzyme variant selected from 53, 54, 55, 56, 57, and 58, wherein the amino acid positions of the variant are numbered by correspondence with the amino acid sequence of LG12 wild-type set forth in SEQ ID NO:10.

In another embodiment, a LG12-clade enzyme variant or an active fragment thereof contains at least three or more amino acid modifications to a parent LG12-clade enzyme, wherein an amino acid modification is a deletion of three or more positions of the enzyme variant selected from 53, 54, 55, 56, 57, and 58, wherein the amino acid positions of the variant are numbered by correspondence with the amino acid sequence of LG12 wild-type set forth in SEQ ID NO:10.

In a still further embodiment, the protease variant, or recombinant polypeptide or active fragment thereof comprises an amino acid sequence comprising two or more substitutions selected from S052T-S129F: G053D-G166D; N076D-G166D; G166H-S159D; G166D-S272K; G053D-N076D-G166D: G053D-G118R-G166D; G053D-G166D-S272K; N076D-G118R-G166D; N076D-G166D-E248K; N076D-G166D-N256R; N076D-G166D-S272K; [S024T-G053E-G166D-M222L-N256P: V149L-S156H-T185Q-S272Q-S-S159D: G053D-N076D-G118R-G166D-N256R: S052T-V149G-S156N-G166H-F217R; S052T-V149G-S156N-G166D-F217R: S052T-V49G-S156N-S159D-G166D-F217R; S052T-V149G-S156N-G166D-S182E-F217R; A040D-S114T-I147A-S159N-G166D-S182E; N038Q-G053E-G100A-N117S-I147A-S158T-G166D-S272K; N038Q-G053E-N117S-I147A-S158T-G166D-N256P-S272K; A001G-S052Q-V149G-S156N-S159N-S182E-Y238F-S272Q: A001G-S052Q-S114T-S156N-S159N-S182E-Y238F-S272Q; N038Q-G053E-G100A-N117S-I147A-S158T-G166D-N256P-S272K; A001G-I030V-T078D-V093T-T103E-S156N-S159D-G166S-S194P-F217R-A236N; A001T-N076D-T078S-T079I-Y086P-D089E-S097G-T103S-L104V-G06S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-E248N; S052A-N076D-T078S-T079I-Y086P-D089E-S097G-T103S-L104V-G106S-I111L-S114A-I115G-S116N-N120H-I122A-M124L-G128S-S129P-G131P-T133V-A134T-Q136E-C139V-N141S-Y143T-N144S-I147V-V148L-I150V-S158T-G166D-F217L-E248N; A001T-N076D-T078S-T079I-Y086P-D089E-S097G-T103S-L104V-G106S-I111L-S114A-I115G-S116N-N120H-I122A-M124L-G128S-S129P-G131P-T133V-A134T-Q136E-C139V-N141S-Y143T-N144S-I147V-V148L-I150V-S158T-G166D-T209Y-N212S-N213T-S216T-F217L-A224T-E248N-K251S-N252S-N256Y; N076D-T078S-T079I-Y086P-D089E-S097G-T103S-L104V-G106S-I111L-S114A-I115G-S116N-N120H-I122A-M124L-G128S-S129P-G131P-T33A-A134T-Q136E-C139V-N141S-Y143T-N144S-I147V-V148L-I150V-S158T-G166D-T209Y-N212S-N213T-S216T-F217L-A224T-E248N; N076D-T078S-T079I-Y086P-N087S-A088V-D089S-S097G-T103S-L104V-G106S-I111L-S114A-I115G-S116N-N120H-I122A-M24L-G128S-S129P-G131P-T133A-A134T-Q136E-C139V-N141S-Y143T-N144S-I147V-V148L-I150V-S158T-G166D-F217L-E248N: A001T-N076D-T078S-T079I-Y086P-D089E-S097G-T103S-L104V-G106S-I111L-S114A-I115G-S116N-N120H-I122A-M124L-G128S-S129P-31P-T133A-A134T-Q136E-C39V-N141S-Y143T-N144S-S147V-V148L-I50V-S158T-G166D-T209Y-N212S-N213T-S216T-F217L-A224T-E248N: A001T-N076D-T078S-T079I-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-E248N; S052A-N076D-T078S-T079I-Y086P-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-F217L-A224T-E248N; S052A-N076D-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-A224T-E248N: A001T-N076D-T078S-T079I-Y086P-D089E-S097G-T103S-L104V-G106S-I111L-S114A-I115G-S16N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-E248N: A001T-S052A-N076D-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-A153S-S158T-G166D-T209Y-N212S-N213T-S216T-F217L-A224T-E248N; A001T-N076D-Y086P-N087S-A088V-D089S-S097G-T103S-L104V-G106S-I111L-S114A-I115G-S16N-N120H-I122A-M124L-G128S-S129P-G131P-T133A-A134T-Q136E-C139V-N141S-Y143T-N144S-I147V-V148L-I150V-A153S-S158T-G166D-T209Y-N212S-N213T-S216T-F217L-A224T-E248N; A001T-N076D-Y086P-N087S-A088V-D089S-S097G-T103S-L104V-G06S-I111L-S114A-I115G-S116N-N120H-I22A-M124L-G128S-S129P-G131P-T133A-A134T-Q136E-C139V-N141S-Y143T-N144S-I147V-V148L-I150V-S158T-G166D-T209Y-N212S-N213T-S216T-F217L-A224T-E248N-Q275H; A001T-N076D-T078S-T079I-Y086P-N087S-A088V-D089S-S097G-T103S-L104V-G106S-I111L-S114A-I115G-S116N-N120H-I122A-M124L-G128S-S129P-G131P-T33A-A134T-Q136E-C139V-N141S-Y143T-N144S-I147V-V148L-I150V-S158T-G166D-T209Y-N212S-N213T-S216T-F217L-A224T-E248N; N076D-G118R-G28D-S129R-S130I-G166D-E248N; N076D-G118R-G128D-S129F-S130Y-G166D-E248N; N076D-G118R-G128V-S129M-S130H-G166D-E248N; S052T-N076D-G118R-G128M-S129V-S130F-G166H-Y238F; S052T-N076D-G118R-G128D-S129R-S130I-G166D-F217R-N256R; S052T-N076D-G118R-G128V-S129M-S130H-G166D-E248N-N256E-S272K; S052F-G053Y-N076D-G118R-G128D-S129F-S130Y-G166D-F217R-E248N-N256R-S272K; and A001G-A040D-A048Q-S052T-G053E-Y091H-S14T-I147A-S156N-S159N-G166H-S182E-Y238F; wherein the amino acid positions of the variant or recombinant polypeptide or active fragment thereof are numbered by correspondence with the amino acid sequence of SEQ ID NO: 10.

In an even still further embodiment, the protease variant, or recombinant polypeptide or active fragment thereof comprises an amino acid sequence comprising two or more substitutions selected from S052T-S129F; G053D-G166D; N076D-G166D: G166H-S159D; G166D-S272K; G053D-N076D-G166D: G053D-G118R-G166D; G053D-G166D-S272K; N076D-G118R-G166D; N076D-G166D-E248K; N076D-G166D-N256R; N076D-G166D-S272K; S024T-G053E-G166D-M222L-N256P; V149L-S156H-T185Q-S272Q-S159D; G053D-N076D-G118R-G166D-N256R; S052T-V149G-S156N-G166H-F217R; S052T-V149G-S156N-G166D-F217R: S052T-V149G-S156N-S159D-G166D-F217R; S052T-V149G-S156N-G166D-S182E-F217R; A040D-S114T-I147A-S159N-G166D-S182E; N038Q-G053E-G100A-N117S-I147A-S158T-G166D-S272K; N038Q-G053E-N17S-I147A-S158T-G166D-N256P-S272K; A001G-S052Q-V149G-S156N-S159N-S182E-Y238F-S272Q: A01G-S052Q-S114T-S156N-S159N-S182E-Y238F-S272Q; N038Q-G053E-G100A-N117S-I147A-S158T-G166D-N256P-S272K; A001G-I030V-T078D-V093T-T103E-S156N-S159D-G166S-S194P-F217R-A236N; A001T-N076D-T078S-T079I-Y086P-D089E-S097G-T103S-L104V-G06S-I111L-S114A-I115G-S16N-N120H-I122A-M124L-G128S-S129P-G131P-T133A-A134T-Q136E-C139V-N141S-Y143T-N144S-I147V-V148L-I150V-S158T-G166D-E248N; S052A-N076D-T078S-T079I-Y086P-D089E-S197G-T103S-L104V-G106S-I111L-S114A-I115G-S16N-N120H-I122A-M124L-G128S-S129P-G131P-T133V-A134T-Q136E-C139V-N141S-Y143T-N144S-I147V-V148L-I150V-S158T-G166D-F217L-E248N; A001T-N076D-T078S-T079I-Y086P-D089E-S097G-T103S-L104V-G106S-I111L-S114A-I115G-S116N-N120H-I122A-M24L-G28S-S129P-G131P-T33V-A134T-Q136E-C139V-N141S-Y143T-N144S-I147V-V148L-I150V-S158T-G166D-T209Y-N212S-N213T-S216T-F217L-A224T-E248N-K251S-N252S-N256Y; N076D-T078S-T079I-Y086P-D089E-S097G-T103S-L104V-G106S-I111L-S114A-I115G-S16N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-A224T-E248N; N076D-T078S-T079I-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-F217L-E248N: A001T-N076D-T078S-T079I-Y086P-D089E-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-A224T-E248N; A001T-N076D-T078S-T079I-Y086P-N087S-A088V-D089S-S097G-T103S-L104V-G106S-I11l L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-E248N; and N076D-G118R-G128D-S129R-S130I-G166D-E248N; wherein the amino acid positions of the variant or recombinant polypeptide or active fragment thereof are numbered by correspondence with the amino acid sequence of SEQ ID NO:10.

In yet another embodiment, the protease variant, or recombinant polypeptide or active fragment thereof comprises an amino acid sequence comprising two or more substitutions selected from S052A-N076D-T078S-T079I-Y086P-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-F217L-A224T-E248N; S052A-N076D-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-A224T-E248N; A001T-N076D-T078S-T079I-Y086P-D089E-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-E248N; A001T-S052A-N076D-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-A153S-S158T-G166D-T209Y-N212S-N213T-S216T-F217L-A224T-E248N; A001T-N076D-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-A153S-S158T-G166D-T209Y-N212S-N213T-S216T-F217L-A224T-E248N: A001T-N076D-Y086P-N087S-A088V-D089S-S097G-T103S-L104V-G06S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-A224T-E248N-Q275H: A001T-N076D-T078S-T079I-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-A224T-E248N; N076D-G118R-G128D-S129F-S130Y-G166D-E248N; N076D-G118R-G128V-S129M-S130H-G166D-E248N; S052T-N076D-G118R-G128M-S129V-S130F-G166H-Y238F; S02T-N076D-G118R-G128D-S129R-S130I-G166D-F217R-N256R; S052T-N076D-G118R-G128V-S129M-S130H-G166D-E248N-N256E-S272K; S052F-G053Y-N076D-G118R-G128D-S129F-S130Y-G166D-F217R-E248N-N256R-S272K and A001G-A040D-A048Q-S052T-G053E-Y091H-S114T-I147A-S156N-S159N-G166H-S182E-Y238F; wherein the amino acid positions of the variant or recombinant polypeptide or active fragment thereof are numbered by correspondence with the amino acid sequence of SEQ ID NO:10.

In some embodiments of the invention, LG12-clade enzymes of the present invention comprise substitutions selected from: G053D-N076D-G166D: N038Q-G053E-G100A-N117S-I147A-S158T-G166D-N256P-S272K; A001G-I030V-T078D-V093T-T103E-S156N-S159D-G166S-S94P-F217R-A236N; S024T-G053E-G166D-M222L-N256P; V149L-S156H-T185Q-S272Q-S159D; G053D-N076D-G118R-G166D-N256R; N038Q-G053E-G100A-N117S-I147A-S158T-G166D-S272K; S052T-V149G-S156N-G166H-F217R; A001G-S052Q-V149G-S156N-S159N-S182E-Y238F-S272Q; A001G-S052Q-S114T-S156N-S159N-S182E-Y238F-S272Q; S052T-V149G-S156N-S159D-G166D-F217R: N038Q-G053E-N117S-I147A-S158T-G166D-N256P-S272K; A040D-S114T-I147A-S159N-G I66D-S182E: S052T-V149G-S156N-G166D-S182E-F217R; and S052T-V149G-S156N-G166D-F217R, wherein the amino acid positions of the variant are numbered by correspondence with the amino acid sequence of LG12 wild-type set forth in SEQ ID NO:10.

In some embodiments of the invention, LG12-clade enzymes of the present invention having a beneficial mutation in long-term stability in at least one boron-free HDL comprise substitutions selected from: G053D-N076D-G166D; V149L-S156H-T185Q-S272Q-S159D; G053D-N076D-G118R-G166D-N256R; N038Q-G053E-G100A-N117S-I147A-S158T-G166D-S272K; S052T-V149G-S156N-G166H-F217R: and S052T-V149G-S156N-G166D-F217R, wherein the amino acid positions of the variant are numbered by correspondence with the amino acid sequence of LG12 wild-type set forth in SEQ ID NO:10.

Also provided are LG12-clade protease variants useful in a detergent composition where favorable modifications result in a minimum performing index for cleaning performance, stability of the enzyme in detergent compositions and/or thermostability of the enzyme, while having at least one of these characteristics improved from its parent LG12-clade enzyme. In one embodiment, an LG12-clade protease variant is useful in a detergent composition and has a minimum performing index for cleaning performance, stability of the enzyme in detergent compositions and/or thermostability of the enzyme, while having at least one of these characteristics improved over the LG12-clade wild-type parent protease (SEQ ID NO:10).

The terms “thermal stability” and “thermostability” refer to LG12-clade protease variants of the present invention that retain a specified amount of enzymatic activity after exposure to an identified temperature, often over a given period of time under conditions prevailing during the proteolytic, hydrolyzing, cleaning or other process disclosed herein, for example while exposed to altered temperatures. Altered temperatures include increased or decreased temperatures. In some embodiments, the LG12-clade protease retains at least about 50%, about 60%, about 70%, about 75%, about 80%, about 85%, about 90%, about 92%, about 95%, about 96%, about 97%, about 98%, or about 99% protease activity after exposure to altered temperatures over a given time period, for example, at least about 60 minutes, about 120 minutes, about 180 minutes, about 240 minutes, about 300 minutes, etc.

As used herein, improved properties of an LG12-clade protease variant has improved residual protease activity, improved cleaning performance, and/or improved stability optionally with retained wash or cleaning performance, relative to the corresponding parent LG2-clade enzyme. In some embodiments, an LG12-clade protease variant has improved stability optionally with retained cleaning performance relative to the corresponding LG2-clade wild-type parent protease (SEQ ID NO:10). In some further embodiments, an LG12-clade protease variant has improved stability with retained cleaning performance relative to the corresponding LG12-clade wild-type parent protease (SEQ ID NO:10). The improved properties of an LG12-clade protease variant may comprise improved cleaning performance and/or improved stability. In some embodiments, the invention provides an LG12-clade protease variant that exhibits one of more of the following properties: improved hand wash performance, improved hand or manual dishwashing performance, improved automatic dishwashing performance, improved laundry performance, and/or improved stability relative to a reference parent LG12-clade enzyme. In some embodiments, the invention provides an LG12-clade protease variant that exhibits one of more of the following properties: improved hand wash performance, improved hand or manual dishwashing performance, improved automatic dishwashing performance, improved laundry performance, and/or improved stability relative to the corresponding LG12-clade wild-type parent protease (SEQ ID NO: 10).

As noted above, the variant enzyme polypeptides of the invention have enzymatic activities (e.g., protease activities) and thus are useful in cleaning applications, including but not limited to, methods for cleaning dishware items, tableware items, fabrics, and items having hard surfaces (e.g., the hard surface of a table, table top, wall, furniture item, floor, ceiling, etc.). Exemplary cleaning compositions comprising one or more variant serine protease enzyme polypeptides of the invention described infra. The enzymatic activity (e.g., protease enzyme activity) of an enzyme polypeptide of the invention can be determined readily using procedures well known to those of ordinary skill in the art. The Examples presented infra describe methods for evaluating the enzymatic activity and cleaning performance. The performance of polypeptide enzymes of the invention in removing stains (e.g., a protein stain such as blood/milk/ink or egg yolk), cleaning hard surfaces, or cleaning laundry, dishware or tableware item(s) can be readily determined using procedures well known in the art and/or by using procedures set forth in the Examples.

The serine protease polypeptides of the present invention can have protease activity over a broad range of pH conditions. In some embodiments, the serine protease polypeptides have protease activity on dimethylcasein as a substrate, as demonstrated in Examples below. In some embodiments, the serine protease polypeptides have protease activity at a pH of from about 4.0 to about 12.0. In some embodiments, the serine protease polypeptides have protease activity at a pH of from about 6.0 to about 12.0. In some embodiments, the serine protease polypeptides have at least 50%, 60%, 70%, 80% or 90% of maximal protease activity at a pH of from about 6.0 to about 12.0, or from about 7.0 to about 12.0. In some embodiments, the serine protease polypeptides have protease activity at a pH above 6.0, 6.5, 7.0, 7.5, 8.0, 8.5, 9.0, 9.5, 10.0, 10.5, 11.0 or 11.5. In some embodiments, the serine protease polypeptides have protease activity at a pH below 12.0, 11.5, 11.0, 10.5, 10.0, 9.5, 9.0, 8.5, 8.0, 7.5, 7.0, or 6.5.

In some embodiments, the serine protease polypeptides of the present invention have protease activity at a temperature range from about 10° C. to about 900C, or from about 30° C. to about 80° C. In some embodiments, the serine protease polypeptides of the present invention have protease activity at a temperature range of from about 55° C. to about 75° C. In some embodiments, the serine protease polypeptides have at least 50%, 60%, 70%, 80% or 90% of maximal protease activity at a temperature of from about 55° C. to about 75° C. In some embodiments, the serine proteases have activity at a temperature above 50° C., 50C, 60° C., 65° C. or 70° C. In some embodiments, the serine proteases have activity at a temperature below 80° C., 75° C., 70° C. 65C, 60° C., or 55° C.

In some embodiments, the serine protease polypeptides of the present invention demonstrate cleaning performance in a cleaning composition. Cleaning compositions often include ingredients harmful to the stability and performance of enzymes, making cleaning compositions a harsh environment for enzymes, e.g. serine proteases, to retain function. Thus, it is not trivial for an enzyme to be put in a cleaning composition and expect enzymatic function (e.g. serine protease activity, such as demonstrated by cleaning performance). In some embodiments, the serine protease polypeptides of the present invention demonstrate cleaning performance in automatic dishwashing (ADW) detergent compositions. In some embodiments, the cleaning performance in automatic dishwashing (ADW) detergent compositions includes cleaning of egg yolk stains. In some embodiments, the serine protease polypeptides of the present invention demonstrate cleaning performance in laundry detergent compositions. In some embodiments, the cleaning performance in laundry detergent compositions includes cleaning of blood/milk/ink stains. In each of the cleaning compositions, the serine protease polypeptides of the present invention demonstrate cleaning performance with or without a bleach component.

In some embodiments, the serine protease polypeptides of the present invention have stability in detergent compositions. In some embodiments, the serine protease polypeptides of the present invention have a thermostability T_{50%}, value of at least 60° C.

A polypeptide of the invention can be subject to various changes, such as one or more amino acid insertions, deletions, and/or substitutions, either conservative or non-conservative, including where such changes do not substantially alter the enzymatic activity of the polypeptide. Similarly, a nucleic acid of the invention can also be subject to various changes, such as one or more substitutions of one or more nucleotides in one or more codons such that a particular codon encodes the same or a different amino acid, resulting in either a silent variation (e.g., when the encoded amino acid is not altered by the nucleotide mutation) or non-silent variation, one or more deletions of one or more nucleic acids (or codons) in the sequence, one or more additions or insertions of one or more nucleic acids (or codons) in the sequence, and/or cleavage of or one or more truncations of one or more nucleic acids (or codons) in the sequence. Many such changes in the nucleic acid sequence may not substantially alter the enzymatic activity of the resulting encoded polypeptide enzyme compared to the polypeptide enzyme encoded by the original nucleic acid sequence. A nucleic acid sequence of the invention can also be modified to include one or more codons that provide for optimum expression in an expression system (e.g., bacterial expression system), while, if desired, said one or more codons still encode the same amino acid(s).

In some embodiments, the LG12-clade protease variant can be a variant of a parent LG12-clade enzyme from the Genus Bacillus. In other embodiments, the LG12-clade protease variant of the present invention is a variant of a parent LG12-clade enzyme from a genus selected from Bacillus, Geobacillus, Alicyclobacillus, Lactobacillus, Exiguobacterium, Brevbacillus, Paenibacillus, Herpetosiphon, Oceanobacillus, Shewanella, Clostridium, Slaphylococcus, Flavobacterium, Stigmatella, Myxococcus, Vibrio, Methanosarcina, Chryseobacterium, Streptomyces, Kribbella, Janibacter, Nocardioides, Xanthamonas, Micromonospora, Burkholderia, Dehalococcoides, Croceibacter, Kordia, Microscilla, Thermoacinomycves, Chloroflexus, Listeria, Plesioevstis, Haliscomenobacter, Cytophaga, Hahella, Arthrobacter, Brachybacterium, Clavibacter, Microbacterium, Intrasporangium, Frankia, Meiothermus, Pseudomonas, Ricinus, Catenulispora, Anabaena, Nostoc, Halomonas, Chromohalobacter, Bordetella, Variovorax, Dickeya, Pectobacterium, Citrobacter, Enterobacter, Salmonella, Erwinia, Pantoea, Rahnella, Serratia, Geodermatophilus, Gemmata, Xenorhabdus, Photorhabdus, Aspergillus, Neosartorya Pyrenophora, Saccharopolyspora, Nectria, Gibberella, Metarhizium, Waddlia, Cyanothece, Cellulphaga, Providencia, Bradvrhizobium, Agrobacterium, Mucilaginibacter, Serratia, Sorangium, Streptosporangium, Renibacterium, Aeromonas, Reinekea, Chromobacieriumn, Moritella, Halimangium, Kangiella, Marinomonas, Vibrionales, Listonella, Salinivibrio, Photobacterium, Alteromonadales, Legionella, Teredinibacter, Reinekea, Hyvdrogenivirga, and Pseudoalteromonas.

A polypeptide of the invention can be subject to various changes, such as one or more amino acid insertions, deletions, and/or substitutions, either conservative or non-conservative, including where such changes do not substantially alter the enzymatic activity of the polypeptide. In some embodiments, the present invention provides a genus of polypeptides comprising LG12-clade protease variants having the desired enzymatic activity (e.g., LG12-clade enzyme activity or cleaning performance activity) which comprise sequences having the amino acid substitutions described herein and also which comprise one or more additional amino acid substitutions, such as conservative and non-conservative substitutions, wherein the polypeptide exhibits, maintains, or approximately maintains the desired enzymatic activity (e.g., LG12-clade enzyme activity or proteolytic activity, as reflected in the cleaning activity or performance of the variant LG12-clade enzyme). Amino acid substitutions in accordance with the invention may include, but are not limited to, one or more non-conservative substitutions and/or one or more conservative amino acid substitutions. A conservative amino acid residue substitution typically involves exchanging a member within one functional class of amino acid residues for a residue that belongs to the same functional class (identical amino acid residues are considered functionally homologous or conserved in calculating percent functional homology). A conservative amino acid substitution typically involves the substitution of an amino acid in an amino acid sequence with a functionally similar amino acid. For example, alanine, glycine, serine, and threonine are functionally similar and thus may serve as conservative amino acid substitutions for one another. Aspartic acid and glutamic acid may serve as conservative substitutions for one another. Asparagine and glutamine may serve as conservative substitutions for one another. Arginine, lysine, and histidine may serve as conservative substitutions for one another. Isoleucine, leucine, methionine, and valine may serve as conservative substitutions for one another. Phenylalanine, tyrosine, and tryptophan may serve as conservative substitutions for one another.

Other conservative amino acid substitution groups can be envisioned. For example, amino acids can be grouped by similar function or chemical structure or composition (e.g., acidic, basic, aliphatic, aromatic, sulfur-containing). For instance, an aliphatic grouping may comprise: Glycine (G), Alanine (A), Valine (V). Leucine (L), Isoleucine (1). Other groups containing amino acids that are considered conservative substitutions for one another include: aromatic: Phenylalanine (F), Tyrosine (Y), Tryptophan (W); sulfur-containing: Methionine (M), Cysteine (C): Basic: Arginine (R), Lysine (K), Histidine (H); Acidic: Aspartic acid (D). Glutamic acid (E); non-polar uncharged residues, Cysteine (C), Methionine (M), and Proline (P); hydrophilic uncharged residues: Serine (S), Threonine (T), Asparagine (N), and Glutamine (Q). Additional groupings of amino acids are well-known to those of skill in the art and described in various standard textbooks. Listing of a polypeptide sequence herein, in conjunction with the above substitution groups, provides an express listing of all conservatively substituted polypeptide sequences.

More conservative substitutions exist within the amino acid residue classes described above, which also or alternatively can be suitable. Conservation groups for substitutions that are more conservative include: valine-leucine-isoleucine, phenylalanine-tyrosine, lysine-arginine, alanine-valine, and asparagine-glutamine.

Conservatively substituted variations of a polypeptide sequence of the invention (e.g., variant proteases of the invention) include substitutions of a small percentage, sometimes less than 250%, 20%, 15%, 14%, 13%, 12%, 11%, 10%, 9%, 8%, 7%, or 6% of the amino acids of the polypeptide sequence, or less than 5%, 4%, 3%, 2%, or 1%, or less than 10, 9, 8, 7, 6, 5, 4, 3, 2, or 1 amino acid substitution of the amino acids of the polypeptide sequence, with a conservatively selected amino acid of the same conservative substitution group.

In some embodiments, the protease variant comprises one or more mutations, and having a total net charge of −5, −4, −3, −2, −1, 0, 1, 2, 3, 4, or 5 relative to LG12-clade wild-type parent (SEQ ID NO:10).

### Nucleic Acids of the Invention

The invention provides isolated, non-naturally occurring, or recombinant nucleic acids (also referred to herein as “polynucleotides”), which may be collectively referred to as “nucleic acids of the invention” or “polynucleotides of the invention”, which encode polypeptides of the invention. Nucleic acids of the invention, including all described below, are useful in recombinant production (e.g., expression) of polypeptides of the invention, typically through expression of a plasmid expression vector comprising a sequence encoding the polypeptide of interest or fragment thereof. As discussed above, polypeptides include variant protease polypeptides, including LG12-clade protease variant polypeptides having enzymatic activity (e.g., proteolytic activity) which are useful in cleaning applications and cleaning compositions for cleaning an item or a surface (e.g., surface of an item) in need of cleaning.

In some embodiments, the invention provides an isolated, recombinant, substantially pure, or non-naturally occurring nucleic acid comprising a nucleotide sequence encoding any polypeptide (including any fusion protein, etc.) of the invention described above in the section entitled “Polypeptides of the Invention” and elsewhere herein. The present invention provides nucleic acids encoding a serine protease polypeptide of the present invention, wherein the serine protease polypeptide is a mature form having proteolytic activity. In some embodiments, the serine protease is expressed recombinantly with a homologous pro-peptide sequence. In other embodiments, the serine protease is expressed recombinantly with a heterologous pro-peptide sequence (e.g., GG36 pro-peptide sequence).

Some embodiments are directed to a polynucleotide comprising a nucleic acid sequence that encodes a protease variant described herein, or recombinant polypeptide or active fragment thereof. In some embodiments, a polynucleotide encodes an isolated, recombinant, substantially pure, or non-naturally occurring LG12-clade protease variant having protease activity, which polypeptide has at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or 100% sequence to an amino acid sequence selected from SEQ ID NO: 1, 2, 3, 4, 5, 6, 7, 8, 29, 30, 31, 32, 33, 34, 35, 36, 37, 38, 39, 40, 41, 42, and 43. In some embodiments, a polynucleotide encodes an isolated, recombinant, substantially pure, or non-naturally occurring LG12-clade protease variant having protease activity, which polypeptide has 70%, 71%, 72%, 73%, 74%, 75%, 76%, 77%, 78%, 79%, 80%, 81%, 82%, 83%, 84%, 85%, 86%, 87%, 88%, 89%, 90%. 91%, 92%, 93%, 94%, 95%, 96%, 97%, 98%, 99% or 100% sequence identity to an amino acid sequence selected from SEQ ID NO: 1, 2, 3, 4, 5, 6, 7, 8, 29, 30, 31, 32, 33, 34, 35, 36, 37, 38, 39, 40, 41, 42, and 43. In a still further embodiment, a polynucleotide encodes an isolated, recombinant, substantially pure, or non-naturally occurring LG12-clade protease variant having protease activity, which polypeptide has 70%, 75%, 80%, 85%, 90%, 95%, 96%, 97%, 98%, 990/% or 100% sequence identity to an amino acid sequence selected from SEQ ID NO: 1, 2, 3, 4, 5, 6, 7, 8, 29, 30, 31, 32, 33, 34, 35, 36, 37, 38, 39, 40, 41, 42, and 43. In an even still further embodiment, a polynucleotide encodes an isolated, recombinant, substantially pure, or non-naturally occurring LG12-clade protease variant having protease activity, wherein said polypeptide is selected from SEQ ID NO:1, 2, 3, 4, 5, 6, 7, 8, 29, 30, 31, 32, 33, 34, 35, 36, 37, 38, 39, 40, 41, 42, and 43.

Also provided is an isolated, recombinant, substantially pure, or non-naturally occurring nucleic acid comprising a polynucleotide sequence which encodes a LG12-clade protease variant having proteolytic activity, said variant comprising an amino acid sequence which differs from the amino acid sequence of SEQ ID NO:1, 2, 3, 4, 5, 6, 7, 8, 29, 30, 31, 32, 33, 34, 35, 36, 37, 38, 39, 40, 41, 42, or 43 by no more than 50, no more than 45, no more than 40, no more than 35, no more than 30, no more than 25, no more than 20, no more than 19, no more than 18, no more than 17, no more than 16, no more than 15, no more than 14, no more than 13, no more than 12, no more than 11, no more than 10, no more than 9, no more than 6, no more than 5, no more than 4, no more than 3, no more than 2 amino acid residues, or no more than 1 amino acid residue(s), as determined by alignment of the variant protease amino acid sequence with the LG12-clade wild-type amino acid sequence.

Nucleic acids of the invention can be generated by using any suitable synthesis, manipulation, and/or isolation techniques, or combinations thereof. For example, a polynucleotide of the invention may be produced using standard nucleic acid synthesis techniques, such as solid-phase synthesis techniques that are well-known to those skilled in the art. In such techniques, fragments of up to 50 or more nucleotide bases are typically synthesized, then joined (e.g., by enzymatic or chemical ligation methods, or polymerase mediated recombination methods) to form essentially any desired continuous nucleic acid sequence. The synthesis of the nucleic acids of the invention can be also facilitated (or alternatively accomplished) by any suitable method known in the art, including but not limited to chemical synthesis using the classical phosphoramidite method (See e.g., Beaucage et al. Tetrahedron Letters 22:1859-69 [1981]); or the method described by Matthes el al. (See, Matthes et al., EMBO J. 3:801-805 [1984], as is typically practiced in automated synthetic methods. Nucleic acids of the invention also can be produced by using an automatic DNA synthesizer. Customized nucleic acids can be ordered from a variety of commercial sources (e.g., The Midland Certified Reagent Company, the Great American Gene Company, Operon Technologies Inc., and DNA2.0). Other techniques for synthesizing nucleic acids and related principles are known in the art (See e.g., Itakura et al., Ann. Rev. Biochem. 53:323 [1984]; and Itakura et al., Science 198:1056 [1984]).

As indicated above, recombinant DNA techniques useful in modification of nucleic acids are well known in the art. For example, techniques such as restriction endonuclease digestion, ligation, reverse transcription and cDNA production, and polymerase chain reaction (e.g., PCR) are known and readily employed by those of skill in the art. Nucleotides of the invention may also be obtained by screening cDNA libraries (e.g., cDNA libraries generated using mutagenesis techniques commonly used in the art, including those described herein) using one or more oligonucleotide probes that can hybridize to or PCR-amplify polynucleotides which encode a variant protease polypeptide(s) of the invention. Procedures for screening and isolating cDNA clones and PCR amplification procedures are well known to those of skill in the art and described in standard references known to those skilled in the art. Some nucleic acids of the invention can be obtained by altering a naturally occurring polynucleotide backbone (e.g., that encodes an enzyme or parent protease) by, for example, a known mutagenesis procedure (e.g., site-directed mutagenesis, site saturation mutagenesis, and in vitro recombination).

A variety of methods are known in the art that are suitable for generating modified polynucleotides of the invention that encode variant proteases of the invention, including, but not limited to, for example, site-saturation mutagenesis, scanning mutagenesis, insertional mutagenesis, deletion mutagenesis, random mutagenesis, site-directed mutagenesis, synthetic gene construction by oligonucleotide synthesis and ligation, and directed-evolution, as well as various other recombinatorial approaches. Methods for making modified polynucleotides and proteins (e.g., variant proteases) include DNA shuffling methodologies, methods based on non-homologous recombination of genes, such as ITCHY (See. Ostermeier et al., 7:2139-44 [1999]), SCRACHY (See, Lutz et al. 98:11248-53 [2001]). SHIPREC (See. Sieber et al., 19:456-60 [200]), and NRR (See, Bittker et al., 20:1024-9 [20011]; Bittker et al., 101:7011-6 [2004]), and methods that rely on the use of oligonucleotides to insert random and targeted mutations, deletions and/or insertions (See. Ness et al., 20:1251-5 [2002]: Coco et al., 20:1246-50 [2002]; Zha et al., 4:34-9 [12003]. Glaser et al., 149:3903-13 [1992]).

The present invention provides isolated or recombinant vectors comprising at least one polynucleotide of the invention described herein (e.g., a polynucleotide encoding a variant protease of the invention described herein), isolated or recombinant expression vectors or expression cassettes comprising at least one nucleic acid or polynucleotide of the invention, isolated, substantially pure, or recombinant DNA constructs comprising at least one nucleic acid or polynucleotide of the invention, isolated or recombinant cells comprising at least one polynucleotide of the invention, cell cultures comprising cells comprising at least one polynucleotide of the invention, cell cultures comprising at least one nucleic acid or polynucleotide of the invention, and compositions comprising one or more such vectors, nucleic acids, expression vectors, expression cassettes, DNA constructs, cells, cell cultures, or any combination or mixtures thereof.

In some embodiments, the invention provides recombinant cells comprising at least one vector (e.g., expression vector or DNA construct) of the invention which comprises at least one nucleic acid or polynucleotide of the invention. Some such recombinant cells are transformed or transfected with such at least one vector. Such cells are typically referred to as host cells. Some such cells comprise bacterial cells, including, but are not limited to Bacillus sp. cells, such as B. subtilis cells. The invention also provides recombinant cells (e.g., recombinant host cells) comprising at least one variant protease of the invention.

In some embodiments, the invention provides a vector comprising a nucleic acid or polynucleotide of the invention. In some embodiments, the vector is an expression vector or expression cassette in which a polynucleotide sequence of the invention which encodes a variant protease of the invention is operably linked to one or additional nucleic acid segments required for efficient gene expression (e.g., a promoter operably linked to the polynucleotide of the invention which encodes a variant protease of the invention). A vector may include a transcription terminator and/or a selection gene, such as an antibiotic resistance gene that enables continuous cultural maintenance of plasmid-infected host cells by growth in antimicrobial-containing media.

An expression vector may be derived from plasmid or viral DNA, or in alternative embodiments, contains elements of both. Exemplary vectors include, but are not limited to pHYT, pC194, pJH101, pE194, pHP3 (See, Harwood and Cutting [eds.]. Chapter 3, Molecular Biological Methods for Bacillus, John Wiley & Sons [1990]; suitable replicating plasmids for B. subtilis include those listed on p. 92; See also, Perego. Integrational Vectors for Genetic Manipulations in Bacillus subtilis, in Sonenshein et al., [eds.] Bacillus subtilis and Other Gram-Positive Bacteria: Biochemistry., Physiology and Molecular Genetics, American Society for Microbiology, Washington, D.C. [1993]. pp. 615-624).

For expression and production of a protein of interest (e.g., variant protease) in a cell, at least one expression vector comprising at least one copy of a polynucleotide encoding the modified protease, and preferably comprising multiple copies, is transformed into the cell under conditions suitable for expression of the protease. In some embodiments of the present invention, a polynucleotide sequence encoding the variant protease (as well as other sequences included in the vector) is integrated into the genome of the host cell, while in other embodiments, a plasmid vector comprising a polynucleotide sequence encoding the variant protease remains as autonomous extra-chromosomal element within the cell. The invention provides both extrachromosomal nucleic acid elements as well as incoming nucleotide sequences that are integrated into the host cell genome. The vectors described herein are useful for production of the variant proteases of the invention. In some embodiments, a polynucleotide construct encoding the variant protease is present on an integrating vector that enables the integration and optionally the amplification of the polynucleotide encoding the variant protease into the bacterial chromosome. Examples of sites for integration are well known to those skilled in the art. In some embodiments, transcription of a polynucleotide encoding a variant protease of the invention is effectuated by a promoter that is the wild-type promoter for the selected precursor protease. In some other embodiments, the promoter is heterologous to the precursor protease, but is functional in the host cell. Specifically, examples of suitable promoters for use in bacterial host cells include, but are not limited to, for example, the AprE amyE, amyL, pstS, sacB, pSPAC, pVeg, pHpall promoters, the promoter of the B. stearothermophilus maltogenic amylase gene, the B. amyloliquefaciens (BAN) amylase gene, the B. subtilis alkaline protease gene, the B. clausii alkaline protease gene the B. pumilis xylosidase gene, the B. thuringiensis crylIIA, and the B. licheniformis alpha-amylase gene. Additional promoters include, but are not limited to the A4 promoter, as well as phage Lambda P_{R} or P_{L} promoters, and the E. coli lac, trp or tac promoters.

Variant proteases of the present invention can be produced in host cells of any suitable microorganism, including bacteria and fungi. For example, in some embodiments, the variant protease is produced in host cells of fungal and/or bacterial origin. In some embodiments, the host cells are Bacillus sp., Streptomyces sp., Escherichia sp. or Aspergillus sp. In some embodiments, the variant proteases are produced by Bacillus sp. host cells. Examples of Bacillus sp. host cells that find use in the production of the variant proteases of the invention include, but are not limited to B. licheniformis, B. lentus, B. subtilis. B. amyloliquefaciens, B. lentus, B. brevis, B. stearothermophilus. B, alkalophilus. B, coagulans. B, circulans, B. pumilis, B. thuringiensis, B. clausii, and B. megaterium, as well as other organisms within the genus Bacillus. In some embodiments, B. subtilis host cells are used for production of variant proteases. U.S. Pat. Nos. 5,264,366 and 4,760.025 (RE 34.606) describe various Bacillus host strains that can be used for producing variant proteases of the invention, although other suitable strains can be used.

Several industrial bacterial strains that can be used to produce variant proteases of the invention include non-recombinant (i.e., wild-type) Bacillus sp. strains, as well as variants of naturally-occurring strains and/or recombinant strains. In some embodiments, the host strain is a recombinant strain, wherein a polynucleotide encoding a polypeptide of interest has been introduced into the host. In some embodiments, the host strain is a B. subtilis host strain and particularly a recombinant Bacillus subtilis host strain. Numerous B. subtilis strains are known, including, but not limited to for example, 1A6 (ATCC 39085). 168 (1A01), SB19, W23, Ts85, B637, PB1753 through PB1758, PB3360, JH642, 1A243 (ATCC 39,087), ATCC 21332, ATCC 6051, MI113, DE100 (ATCC 39,094), GX4931, PBT 110, and PEP 211 strain (See e.g., Hoch et al., Genetics 73:215-228 [1973]; See also, U.S. Pat. Nos. 4,450,235 and 4,302,544, and EP 0134048, each of which is incorporated by reference in its entirety). The use of B. subtilis as an expression host cells is well known in the art (See e.g., Palva et al., Gene 19:81-87 [1982]; Fahnestock and Fischer, J. Bacteriol., 165:796-804 [1986] and Wang et al., Gene 69:39-47 [1988]). In some embodiments, the Bacillus host cell is a Bacillus sp. that includes a mutation or deletion in at least one of the following genes, degU, degS, degR and degQ. Preferably the mutation is in a degU gene, and more preferably the mutation is degU(Hv)32 (See e.g., Msadek et al., J. Bacteriol. 172:824-834 [1990]; and Olmos et al., Mol. Gen. Genet. 253:562-567 [1997]). One suitable host strain is a Bacillus subtilis carrying a degU32(Hy) mutation. In some embodiments, the Bacillus host comprises a mutation or deletion in scoC4 (See e.g., Caldwell et al., J. Bacteriol. 183:7329-7340 [2001]); spollE (See e.g., Arigoni et al., Mol. Microbiol. 31:1407-1415 [19991]) and/or oppA or other genes of the opp operon (See e.g., Perego et al., Mol. Microbiol. 5:173-185 [1991]). Indeed, it is contemplated that any mutation in the opp operon that causes the same phenotype as a mutation in the oppA gene will find use in some embodiments of the altered Bacillus strain of the invention. In some embodiments, these mutations occur alone, while in other embodiments, combinations of mutations are present. In some embodiments, an altered Bacillus host cell strain that can be used to produce a variant protease of the invention is a Bacillus host strain that already includes a mutation in one or more of the above-mentioned genes. In addition, Bacillus sp. host cells that comprise mutation(s) and/or deletions of endogenous protease genes find use. In some embodiments, the Bacillus host cell comprises a deletion of the aprE and the nprE genes. In other embodiments, the Bacillus sp. host cell comprises a deletion of 5 protease genes, while in other embodiments, the Bacillus sp. host cell comprises a deletion of 9 protease genes (See e.g., U.S. Pat. Appln. Pub. No. 2005/0202535, incorporated herein by reference).

Host cells are transformed with at least one nucleic acid encoding at least one variant protease of the invention using any suitable method known in the art. Whether the nucleic acid is incorporated into a vector or is used without the presence of plasmid DNA, it is typically introduced into a microorganism, in some embodiments, preferably an E. coli cell or a competent Bacillus cell. Methods for introducing a nucleic acid (e.g., DNA) into Bacillus cells or E. coli cells utilizing plasmid DNA constructs or vectors and transforming such plasmid DNA constructs or vectors into such cells are well known. In some embodiments, the plasmids are subsequently isolated from E. coli cells and transformed into Bacillus cells. However, it is not essential to use intervening microorganisms such as E. coli, and in some embodiments, a DNA construct or vector is directly introduced into a Bacillus host.

Those of skill in the art are well aware of suitable methods for introducing nucleic acid or polynucleotide sequences of the invention into Bacillus cells (See e.g., Ferrari et al., “Genetics,” in Harwood et al. [eds.], Bacillus. Plenum Publishing Corp. [1989], pp. 57-72; Saunders et al., J. Bacteriol. 157:718-726 [1984]; Hoch et al., J. Bacteriol. 93:1925-1937 [1967]: Mann et al., Current Microbiol. 13:131-135 [1986]; Holubova, Folia Microbiol. 30:97 [1985]; Chang et al., Mol. Gen. Genet. 168:11-115 [1979]; Vorobjeva et al., FEMS Microbiol. Lett. 7:261-263 [1980]; Smith et al., Appl. Env. Microbiol. 51:634 [1981]; Fisher et al., Arch. Microbiol. 139:213-217 [1981]; and McDonald, J. Gen. Microbiol. 130:203 [1984]). Indeed, such methods as transformation, including protoplast transformation and congression, transduction, and protoplast fusion are well known and suited for use in the present invention. Methods of transformation are used to introduce a DNA construct or vector comprising a nucleic acid encoding a variant protease of the present invention into a host cell. Methods known in the art to transform Bacillus cells include such methods as plasmid marker rescue transformation, which involves the uptake of a donor plasmid by competent cells carrying a partially homologous resident plasmid (See, Contente et al., Plasmid 2:555-571 [1979]; Haima et al., Mol. Gen. Genet. 223:185-191 [1990]; Weinrauch et al., J. Bacteriol. 154:1077-1087 [1983]: and Weinrauch et al., J. Bacteriol. 169:1205-1211 [1987]). In this method, the incoming donor plasmid recombines with the homologous region of the resident “helper” plasmid in a process that mimics chromosomal transformation.

In addition to commonly used methods, in some embodiments, host cells are directly transformed with a DNA construct or vector comprising a nucleic acid encoding a variant protease of the invention (i.e., an intermediate cell is not used to amplify, or otherwise process, the DNA construct or vector prior to introduction into the host cell). Introduction of the DNA construct or vector of the invention into the host cell includes those physical and chemical methods known in the art to introduce a nucleic acid sequence (e.g., DNA sequence) into a host cell without insertion into a plasmid or vector. Such methods include, but are not limited to calcium chloride precipitation, electroporation, naked DNA, liposomes and the like. In additional embodiments, DNA constructs or vector are co-transformed with a plasmid, without being inserted into the plasmid. In further embodiments, a selective marker is deleted from the altered Bacillus strain by methods known in the art (See, Stahl et al., J. Bacteriol. 158:411-418 [1984]; and Pahneros et al., Gene 247:255-264 [2000]).

In some embodiments, the transformed cells of the present invention are cultured in conventional nutrient media. The suitable specific culture conditions, such as temperature, pH and the like are known to those skilled in the art and are well described in the scientific literature. In some embodiments, the invention provides a culture (e.g., cell culture) comprising at least one variant protease or at least one nucleic acid of the invention. Also provided are compositions comprising at least one nucleic acid, vector, or DNA construct of the invention.

In some embodiments, host cells transformed with at least one polynucleotide sequence encoding at least one variant protease of the invention are cultured in a suitable nutrient medium under conditions permitting the expression of the present protease, after which the resulting protease is recovered from the culture. The medium used to culture the cells comprises any conventional medium suitable for growing the host cells, such as minimal or complex media containing appropriate supplements. Suitable media are available from commercial suppliers or may be prepared according to published recipes (See e.g., the catalogues of the American Type Culture Collection). In some embodiments, the protease produced by the cells is recovered from the culture medium by conventional procedures, including, but not limited to for example, separating the host cells from the medium by centrifugation or filtration, precipitating the proteinaceous components of the supernatant or filtrate by means of a salt (e.g., ammonium sulfate), chromatographic purification (e.g., ion exchange, gel filtration, affinity, etc.). Any method suitable for recovering or purifyiing a variant protease finds use in the present invention.

In some embodiments, a variant protease produced by a recombinant host cell is secreted into the culture medium. A nucleic acid sequence that encodes a purification facilitating domain may be used to facilitate purification of soluble proteins. A vector or DNA construct comprising a polynucleotide sequence encoding a variant protease may further comprise a nucleic acid sequence encoding a purification facilitating domain to facilitate purification of the variant protease (See e.g., Kroll et al., DNA Cell Biol. 12:441-53 [1993]). Such purification facilitating domains include, but are not limited to, for example, metal chelating peptides such as histidine-tryptophan modules that allow purification on immobilized metals (See. Porath, Protein Expr. Purif. 3:263-281 [11992]), protein A domains that allow purification on immobilized immunoglobulin, and the domain utilized in the FLAGS extension/affinity purification system (e.g., protein A domains available from Immunex Corp., Seattle, Wash.). The inclusion of a cleavable linker sequence such as Factor XA or enterokinase (e.g., sequences available from Invitrogen, San Diego. Calif.) between the purification domain and the heterologous protein also find use to facilitate purification.

Assays for detecting and measuring the enzymatic activity of an enzyme, such as a variant protease of the invention, are well known. Various assays for detecting and measuring activity of proteases (e.g., variant proteases of the invention), are also known to those of ordinary skill in the art. In particular, assays are available for measuring protease activity that are based on the release of acid-soluble peptides from casein or hemoglobin, measured as absorbance at 280 nm or colorimetrically using the Folin method, well known to those skilled in the art. Other exemplary assays involve the solubilization of chromogenic substrates (See e.g., Ward, “Proteinases,” in Fogarty (ed.)., Microbial Enzymes and Biotechnology, Applied Science. London, [1983], pp. 251-317). Other exemplary assays include, but are not limited to succinyl-Ala-Ala-Pro-Phe-para nitroanilide assay (suc-AAPF-pNA) and the 2,4,6-trinitrobenzene sulfonate sodium salt assay (TNBS assay). Numerous additional references known to those in the art provide suitable methods (See e.g., Wells et al., Nucleic Acids Res. 11:7911-7925 [1983]; Christianson et al., Anal. Biochem. 223:119-129 [1994]; and Hsia et al., Anal Biochem. 242:221-227 [1199]).

A variety of methods can be used to determine the level of production of a mature protease (e.g., mature variant proteases of the present invention) in a host cell. Such methods include, but are not limited to, for example, methods that utilize either polyclonal or monoclonal antibodies specific for the protease. Exemplary methods include, but are not limited to enzyme-linked immunosorbent assays (ELISA), radioimmunoassays (RIA), fluorescent immunoassays (FIA), and fluorescent activated cell sorting (FACS). These and other assays are well known in the art (See e.g., Maddox el al., J. Exp. Med. 158:1211 [1983]).

In some other embodiments, the invention provides methods for making or producing a mature variant protease of the invention. A mature variant protease does not include a signal peptide or a propeptide sequence. Some methods comprise making or producing a variant protease of the invention in a recombinant bacterial host cell, such as for example, a Bacillus sp. cell (e.g., a B. subtilis cell). In some embodiments, the invention provides a method of producing a variant protease of the invention, the method comprising cultivating a recombinant host cell comprising a recombinant expression vector comprising a nucleic acid encoding a variant protease of the invention under conditions conducive to the production of the variant protease. Some such methods further comprise recovering the variant protease from the culture.

In some embodiments the invention provides methods of producing a variant protease of the invention, the methods comprising: (a) introducing a recombinant expression vector comprising a nucleic acid encoding a variant protease of the invention into a population of cells (e.g., bacterial cells, such as B. subtilis cells): and (b) culturing the cells in a culture medium under conditions conducive to produce the variant protease encoded by the expression vector. Some such methods further comprise: (c) isolating the variant protease from the cells or from the culture medium.

Unless otherwise noted, all component or composition levels provided herein are made in reference to the active level of that component or composition, and are exclusive of impurities, for example, residual solvents or by-products, which may be present in commercially available sources. Enzyme components weights are based on total active protein. All percentages and ratios are calculated by weight unless otherwise indicated. All percentages and ratios are calculated based on the total composition unless otherwise indicated. In the exemplified detergent compositions, the enzymes levels are expressed by pure enzyme by weight of the total composition and unless otherwise specified, the detergent ingredients are expressed by weight of the total compositions.

As indicated herein, in some embodiments, the cleaning compositions of the present invention further comprise adjunct materials including, but not limited to, surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See e.g., U.S. Pat. Nos. 6,610,642, 6,605,458, 5,705,464, 5,710,115, 5,698,504, 5,695,679, 5,686,014 and 5,646,101, all of which are incorporated herein by reference). Embodiments of specific cleaning composition materials are exemplified in detail below. In embodiments in which the cleaning adjunct materials are not compatible with the variant proteases of the present invention in the cleaning compositions, then suitable methods of keeping the cleaning adjunct materials and the protease(s) separated (i.e., not in contact with each other) until combination of the two components is appropriate are used. Such separation methods include any suitable method known in the art (e.g., gelcaps, encapsulation, tablets, physical separation, etc.).

The cleaning compositions of the present invention are advantageously employed for example, in laundry applications, hard surface cleaning, dishwashing applications, as well as cosmetic applications such as dentures, teeth, hair and skin. In addition, due to the unique advantages of increased effectiveness in lower temperature solutions, the enzymes of the present invention are ideally suited for laundry applications. Furthermore, the enzymes of the present invention find use in granular and liquid compositions.

The variant proteases of the present invention also find use in cleaning additive products. In some embodiments, low temperature solution cleaning applications find use. In some embodiments, the present invention provides cleaning additive products including at least one enzyme of the present invention is ideally suited for inclusion in a wash process when additional bleaching effectiveness is desired. Such instances include, but are not limited to low temperature solution cleaning applications. In some embodiments, the additive product is in its simplest form, one or more proteases. In some embodiments, the additive is packaged in dosage form for addition to a cleaning process. In some embodiments, the additive is packaged in dosage form for addition to a cleaning process where a source of peroxygen is employed and increased bleaching effectiveness is desired. Any suitable single dosage unit form finds use with the present invention, including but not limited to pills, tablets, gelcaps, or other single dosage units such as pre-measured powders or liquids. In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials. Acidic fillers find use to reduce pH. Alternatively, in some embodiments, the cleaning additive includes adjunct ingredients, as more fully described below.

The present cleaning compositions and cleaning additives require an effective amount of at least one of the protease variants provided herein, alone or in combination with other proteases and/or additional enzymes. The required level of enzyme is achieved by the addition of one or more protease variants of the present invention. Typically the present cleaning compositions comprise at least about 0.0001 weight percent, from about 0.0001 to about 10, from about 0.001 to about 1, or even from about 0.01 to about 0.1 weight percent of at least one of the variant proteases of the present invention.

The cleaning compositions herein are typically formulated such that, during use in aqueous cleaning operations, the wash water will have a pH of from about 5.0 to about 11.5 or even from about 7.5 to about 10.5. Liquid product formulations are typically formulated to have a neat pH from about 3.0 to about 9.0 or even from about 3 to about 5. Granular laundry products are typically formulated to have a pH from about 9 to about 11. Techniques for controlling pH at recommended usage levels include the use of buffers, alkalis, acids, etc., and are well known to those skilled in the art.

Suitable “low pH cleaning compositions” typically have a neat pH of from about 3 to about 5, and are typically free of surfactants that hydrolyze in such a pH environment. Such surfactants include sodium alkyl sulfate surfactants that comprise at least one ethylene oxide moiety or even from about 1 to about 16 moles of ethylene oxide. Such cleaning compositions typically comprise a sufficient amount of a pH modifier, such as sodium hydroxide, monoethanolamine or hydrochloric acid, to provide such cleaning composition with a neat pH of from about 3 to about 5. Such compositions typically comprise at least one acid stable enzyme. In some embodiments, the compositions are liquids, while in other embodiments, they are solids. The pH of such liquid compositions is typically measured as a neat pH. The pH of such solid compositions is measured as a 10% solids solution of said composition wherein the solvent is distilled water. In these embodiments, all pH measurements are taken at 20° C., unless otherwise indicated.

In some embodiments, when the variant protease(s) is/are employed in a granular composition or liquid, it is desirable for the variant protease to be in the form of an encapsulated particle to protect the variant protease from other components of the granular composition during storage. In addition, encapsulation is also a means of controlling the availability of the variant protease during the cleaning process. In some embodiments, encapsulation enhances the performance of the variant protease(s) and/or additional enzymes. In this regard, the variant proteases of the present invention are encapsulated with any suitable encapsulating material known in the art. In some embodiments, the encapsulating material typically encapsulates at least part of the catalyst for the variant protease(s) of the present invention. Typically, the encapsulating material is water-soluble and/or water-dispersible. In some embodiments, the encapsulating material has a glass transition temperature (Tg) of 0° C. or higher. Glass transition temperature is described in more detail in WO97/11151. The encapsulating material is typically selected from consisting of carbohydrates, natural or synthetic gums, chitin, chitosan, cellulose and cellulose derivatives, silicates, phosphates, borates, polyvinyl alcohol, polyethylene glycol, paraffin waxes, and combinations thereof. When the encapsulating material is a carbohydrate, it is typically selected from monosaccharides, oligosaccharides, polysaccharides, and combinations thereof. In some typical embodiments, the encapsulating material is a starch (See e.g., EP 0922499 and U.S. Pat. Nos. 4,977,252; 5,354,559 and U.S. Pat. No. 5,935,826). In some embodiments, the encapsulating material is a microsphere made from plastic such as thermoplastics, acrylonitrile, methacrylonitrile, polyacrylonitrile, polymethacrylonitrile and mixtures thereof; commercially available microspheres that find use include, but are not limited to those supplied by EXPANCEL® (Stockviksverken, Sweden), and PM6545, PM 6550. PM7220, PM7228, EXTENDOSPHERES®, LUXSIL®, Q-CEL®, and SPHERICEL® (PQ Corp., Valley Forge, Pa.).

As described herein, the variant proteases of the present invention find particular use in the cleaning industry, including, but not limited to laundry and dish detergents. These applications place enzymes under various environmental stresses. The variant proteases of the present invention provide advantages over many currently used enzymes, due to their stability under various conditions.

Indeed, there are a variety of wash conditions including varying detergent formulations, wash water volumes, wash water temperatures, and lengths of wash time, to which proteases involved in washing are exposed. In addition, detergent formulations used in different geographical areas have different concentrations of their relevant components present in the wash water. For example, European detergents typically have about 4500-5000 ppm of detergent components in the wash water, while Japanese detergents typically have approximately 667 ppm of detergent components in the wash water. In North America, particularly the United States, detergents typically have about 975 ppm of detergent components present in the wash water.

A low detergent concentration system includes detergents where less than about 800 ppm of the detergent components are present in the wash water. Japanese detergents are typically considered low detergent concentration system as they have approximately 667 ppm of detergent components present in the wash water.

A medium detergent concentration includes detergents where between about 800 ppm and about 2000 ppm of the detergent components are present in the wash water. North American detergents are generally considered to be medium detergent concentration systems as they have approximately 975 ppm of detergent components present in the wash water. Brazil typically has approximately 1500 ppm of detergent components present in the wash water.

A high detergent concentration system includes detergents where greater than about 2000 ppm of the detergent components are present in the wash water. European detergents are generally considered to be high detergent concentration systems as they have approximately 4500-5000 ppm of detergent components in the wash water.

Latin American detergents are generally high suds phosphate builder detergents and the range of detergents used in Latin America can fall in both the medium and high detergent concentrations as they range from 1500 ppm to 6000 ppm of detergent components in the wash water. As mentioned above. Brazil typically has approximately 1500 ppm of detergent components present in the wash water. However, other high suds phosphate builder detergent geographies, not limited to other Latin American countries, may have high detergent concentration systems up to about 6000 ppm of detergent components present in the wash water.

In light of the foregoing, it is evident that concentrations of detergent compositions in typical wash solutions throughout the world varies from less than about 800 ppm of detergent composition (“low detergent concentration geographies”), for example about 667 ppm in Japan, to between about 800 ppm to about 2000 ppm (“medium detergent concentration geographies”), for example about 975 ppm in U.S. and about 1500 ppm in Brazil, to greater than about 2000 ppm (“high detergent concentration geographies”), for example about 4500 ppm to about 5000 ppm in Europe and about 6000 ppm in high suds phosphate builder geographies.

The concentrations of the typical wash solutions are determined empirically. For example, in the U.S., a typical washing machine holds a volume of about 64.4 L of wash solution. Accordingly, in order to obtain a concentration of about 975 ppm of detergent within the wash solution about 62.79 g of detergent composition must be added to the 64.4 L of wash solution. This amount is the typical amount measured into the wash water by the consumer using the measuring cup provided with the detergent.

As a further example, different geographies use different wash temperatures. The temperature of the wash water in Japan is typically less than that used in Europe. For example, the temperature of the wash water in North America and Japan is typically between about 10 and about 30° C. (e.g., about 20° C.), whereas the temperature of wash water in Europe is typically between about 30 and about 60° C. (e.g., about 40° C.). However, in the interest of saving energy, many consumers are switching to using cold water washing. In addition, in some further regions, cold water is typically used for laundry, as well as dish washing applications. In some embodiments, the “cold water washing” of the present invention utilizes “cold water detergent” suitable for washing at temperatures from about 10° C. to about 40° C., or from about 20° C. to about 30° C. or from about 15° C. to about 25° C. as well as all other combinations within the range of about 15° C. to about 35° C., and all ranges within 10° C. to 40° C.

As a further example, different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60-120 ppm) to hard (121-181 ppm) water has 60 to 181 parts per million (parts per million converted to grains per U.S. gallon is ppm # divided by 17.1 equals grains per gallon) of hardness minerals.

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (for example about 10.5 to about 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, for example about 3 grains per gallon mixed Ca²⁺/Mg^{2′}.

Accordingly, in some embodiments, the present invention provides variant proteases that show surprising wash performance in at least one set of wash conditions (e.g., water temperature, water hardness, and/or detergent concentration). In some embodiments, the variant proteases of the present invention are comparable in wash performance to other LG12 proteases. In some embodiments of the present invention, the variant proteases provided herein exhibit enhanced oxidative stability, enhanced thermal stability, enhanced cleaning capabilities under various conditions, and/or enhanced chelator stability. In addition, the variant proteases of the present invention find use in cleaning compositions that do not include detergents, again either alone or in combination with builders and stabilizers.

In some embodiments of the present invention, the cleaning compositions comprise at least one variant protease of the present invention at a level from about 0.00001 to about 10% by weight of the composition and the balance (e.g., about 99.999 to about 90.0%) comprising cleaning adjunct materials by weight of composition. In some other embodiments of the present invention, the cleaning compositions of the present invention comprises at least one variant protease at a level of about 0.0001 to about 10%, about 0.001 to about 5%, about 0.001 to about 2%, about 0.005 to about 0.5% by weight of the composition and the balance of the cleaning composition (e.g., about 99.9999 to about 90.0%, about 99.999 to about 98%, about 99.995 to about 99.5% by weight) comprising cleaning adjunct materials.

In some embodiments, the cleaning compositions of the present invention comprise one or more additional detergent enzymes, which provide cleaning performance and/or fabric care and/or dishwashing benefits. Examples of suitable enzymes include, but are not limited to, hemicellulases, cellulases, peroxidases, proteases, xylanases, lipases, phospholipases, esterases, cutinases, pectinases, pectate lyases, mannanases, keratinases, reductases, oxidases, phenoloxidases, lipoxygenases, ligninases, pullulanases, tannases, pentosanases, malanases. ß-glucanases, arabinosidases, hyaluronidases, chondroitinases, laccases, and amylases, or any combinations or mixtures thereof. In some embodiments, a combination of enzymes is used (i.e., a “cocktail”) comprising conventional applicable enzymes like protease, lipase, cutinase and/or cellulase in conjunction with amylase is used.

In addition to the protease variants provided herein, any other suitable protease finds use in the compositions of the present invention. Suitable proteases include those of animal, vegetable or microbial origin. In some embodiments, microbial proteases are used. In some embodiments, chemically or genetically modified mutants are included. In some embodiments, the protease is a serine protease, preferably an alkaline microbial protease or a trypsin-like protease. Examples of alkaline proteases include subtilisins, especially those derived from Bacillus (e.g., subtilisin, lentus, amyloliquefaciens, subtilisin Carlsberg, subtilisin 309, subtilisin 147 and subtilisin 168). Additional examples include those mutant proteases described in U.S. Pat. Nos. RE 34,606, 5,955,340, 5,700,676, 6,312,936, and 6.482,628, all of which are incorporated herein by reference. Additional protease examples include, but are not limited to trypsin (e.g., of porcine or bovine origin), and the Fusariton protease described in WO 89/06270. In some embodiments, commercially available protease enzymes that find use in the present invention include, but are not limited to MAXATASE®, MAXACAL™, MAXAPEM™, OPTICLEAN®, OPTIMASE®, PROPERASE®, PURAFECT®, PURAFECT® OXP, PURAMAX®, EXCELLASE®, and PURAFAST™ (Genencor); ALCALASE®, SAVINASE®, PRIMASE®, DURAZYM™, POLARZYME®, OVOZYME®, KANNASE®, LIQUANASE®, NEUTRASE®, RELASE® and ESPERASE® (Novozymes); BLAP™ and BLAP™ variants (Henkel Kommanditgesellschaft auf Aktien, Duesseldorf, Germany), and KAP (B. alkalophilus subtilisin: Kao Corp., Tokyo. Japan). Various proteases are described in WO95/23221, WO 92/21760. U.S. Pat. Publ. No. 2008/0090747, and U.S. Pat. Nos. 5,801,039, 5,340,735, 5,500,364, 5,855,625, US RE 34,606, 5,955,340, 5,700,676, 6,312,936, and 6,482,628, and various other patents. In some further embodiments, serine proteases find use in the present invention, including but not limited to the neutral serine protease described in WO07/044993.

In addition, any suitable lipase finds use in the present invention. Suitable lipases include, but are not limited to those of bacterial or fungal origin. Chemically or genetically modified mutants are encompassed by the present invention. Examples of useful lipases include H. lanuginosa lipase (See e.g., EP258068 and EP305216), R. miehei lipase (See e.g., EP238023), Candida lipase, such as C. antarctica lipase (e.g., the C. antarctica lipase A or B; See e.g., EP214761), Pseudomonas lipases such as P. alcaligenes lipase and P. pseudoalcaligenes lipase (See e.g., EP218272), P. cepacia lipase (See e.g., EP331376), P. stutzeri lipase (See e.g., GB1372034), P. fluorescens lipase, Bacillus lipase (e.g., B. subtilis lipase [Dartois et al., Biochem. Biophys. Acta 1131:253-260 [1993]); B. stearothermophilus lipase [See e.g., JP 64/744992]; and B. pumilus lipase [See e.g., WO 91/16422]).

Furthermore, a number of cloned lipases find use in some embodiments of the present invention, including but not limited to P. camembertii lipase (See, Yamaguchi et al., Gene 103:61-67 [1991]), G, canddum lipase (See, Schimada et al., J. Biochem., 106:383-388 [1989]), and various Rhizopus lipases such as R. delemar lipase (See. Hass et al., Gene 109:117-113 [1991]), a R. niveus lipase (Kugimiya et al., Biosci. Biotech. Biochem. 56:716-719 [1992]) and R. oryzae lipase.

Other types of lipolytic enzymes such as cutinases also find use in some embodiments of the present invention, including but not limited to the cutinase derived from Pseudomonas mendocina (See, WO88/09367), and the cutinase derived from Fusarium solanipisi (See, WO90/09446).

Additional suitable lipases include commercially available lipases such as M1 LIPASE™, LUMA FAST™, and LIPOMAX™ (Genencor); LIPEX®, LIPOLASE® and LIPOLASE® ULTRA (Novozymes); and LIPASE P™ “Amano” (Amano Pharmaceutical Co. Ltd., Japan).

In some embodiments of the present invention, the cleaning compositions of the present invention further comprise lipases at a level from about 0.00001 to about 10% of additional lipase by weight of the composition and the balance of cleaning adjunct materials by weight of composition. In some other embodiments of the present invention, the cleaning compositions of the present invention also comprise lipases at a level of about 0.0001 to about 10%, about 0.001 to about 5%, about 0.001 to about 2%, about 0.005 to about 0.5% lipase by weight of the composition.

In some embodiments of the present invention, any suitable amylase finds use in the present invention. In some embodiments, any amylase (e.g., alpha and/or beta) suitable for use in alkaline solutions also find use. Suitable amylases include, but are not limited to those of bacterial or fungal origin. Chemically or genetically modified mutants are included in some embodiments. Amylases that find use in the present invention, include, but are not limited to a-amylases obtained from B. licheniformis (See e.g., GB1296839). Commercially available amylases that find use in the present invention include, but are not limited to DURAMYL®, TERMAMYL®, FUNGAMYL®, STAINZYME®, STAINZYME PLUS®, STAINZYME ULTRA®, and BAN™ (Novozymes), as well as POWERASE™, RAPIDASE® and MAXAMYL® P (Genencor).

In some embodiments of the present invention, the cleaning compositions of the present invention further comprise amylases at a level from about 0.00001 to about 10% of additional amylase by weight of the composition and the balance of cleaning adjunct materials by weight of composition. In some other embodiments of the present invention, the cleaning compositions of the present invention also comprise amylases at a level of about 0.0001 to about 10%, about 0.001 to about 5%, about 0.001 to about 2%, about 0.005 to about 0.5% amylase by weight of the composition.

In some further embodiments, any suitable cellulase finds used in the cleaning compositions of the present invention. Suitable cellulases include, but are not limited to those of bacterial or fungal origin. Chemically or genetically modified mutants are included in some embodiments. Suitable cellulases include, but are not limited to H. insolens cellulases (See e.g., U.S. Pat. No. 4,435,307). Especially suitable cellulases are the cellulases having color care benefits (See e.g., EP0495257). Commercially available cellulases that find use in the present include, but are not limited to CELLUZYME®. CAREZYME® (Novozymes), and KAC-500(B)™ (Kao Corporation). In some embodiments, cellulases are incorporated as portions or fragments of mature wild-type or variant cellulases, wherein a portion of the N-terminus is deleted (See e.g., U.S. Pat. No. 5,874,276). In some embodiments, the cleaning compositions of the present invention further comprise cellulases at a level from about 0.00001 to about 10% of additional cellulase by weight of the composition and the balance of cleaning adjunct materials by weight of composition. In some other embodiments of the present invention, the cleaning compositions of the present invention also comprise cellulases at a level of about 0.0001 to about 10%, about 0.001 to about 5%, about 0.001 to about 2%, about 0.005% to about 0.5% cellulase by weight of the composition.

Any mannanase suitable for use in detergent compositions also finds use in the present invention. Suitable mannanases include, for example, those of bacterial or fungal origin. Chemically or genetically modified mutants are included in some embodiments. Various mannanases are known which find use in the present invention (See e.g., U.S. Pat. Nos. 6,566,114, 6,602,842, and 6,440,991. In some embodiments, the cleaning compositions of the present invention further comprise mannanases at a level from about 0.00001 to about 10% of additional mannanase by weight of the composition and the balance of cleaning adjunct materials by weight of composition. In some embodiments of the present invention, the cleaning compositions of the present invention also comprise mannanases at a level of about 0.0001 to about 10%, about 0.001 to about 5%, about 0.001 to about 2%, about 0.005 to about 0.5% mannanase by weight of the composition.

In some embodiments, peroxidases are used in combination with hydrogen peroxide or a source thereof (e.g., a percarbonate, perborate or persulfate) in the compositions of the present invention. In some alternative embodiments, oxidases are used in combination with oxygen. Both types of enzymes are used for “solution bleaching” (i.e., to prevent transfer of a textile dye from a dyed fabric to another fabric when the fabrics are washed together in a wash liquor), preferably together with an enhancing agent (See e.g., WO94/12621 and WO95/01426). Suitable peroxidases/oxidases include, but are not limited to those of plant, bacterial or fungal origin. Chemically or genetically modified mutants are included in some embodiments. In some embodiments, the cleaning compositions of the present invention further comprise peroxidase and/or oxidase enzymes at a level from about 0.00001 to about 10% of additional peroxidase and/or oxidase by weight of the composition and the balance of cleaning adjunct materials by weight of composition. In some other embodiments of the present invention, the cleaning compositions of the present invention also comprise peroxidase and/or oxidase enzymes at a level of about 0.0001 to about 10%, about 0.001 to about 5%, about 0.001 to about 2%, about 0.005 to about 0.5% peroxidase and/or oxidase enzymes by weight of the composition.

In some embodiments, additional enzymes find use, including but not limited to perhydrolases (See e.g., WO05/056782). In addition, in some embodiments, mixtures of the above mentioned enzymes are encompassed herein, in particular one or more additional protease, amylase, lipase, mannanase, and/or at least one cellulase. Indeed, it is contemplated that various mixtures of these enzymes will find use in the present invention. It is also contemplated that the varying levels of the variant protease(s) and one or more additional enzymes may both independently range to about 10%, the balance of the cleaning composition being cleaning adjunct materials. The specific selection of cleaning adjunct materials are readily made by considering the surface, item, or fabric to be cleaned, and the desired form of the composition for the cleaning conditions during use (e.g., through the wash detergent use).

Examples of suitable cleaning adjunct materials include, but are not limited to, surfactants, builders, bleaches, bleach activators, bleach catalysts, other enzymes, enzyme stabilizing systems, chelants, optical brighteners, soil release polymers, dye transfer agents, dye transfer inhibiting agents, catalytic materials, hydrogen peroxide, sources of hydrogen peroxide, preformed peracids, polymeric dispersing agents, clay soil removal agents, structure elasticizing agents, dispersants, suds suppressors, dyes, perfumes, colorants, filler salts, hydrotropes, photoactivators, fluorescers, fabric conditioners, fabric softeners, carriers, hydrotropes, processing aids, solvents, pigments, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See e.g., U.S. Pat. Nos. 6,610,642, 6,605,458, 5,705,464, 5,710,115, 5,698,504, 5,695,679, 5,686,014 and 5,646,101, all of which are incorporated herein by reference). Embodiments of specific cleaning composition materials are exemplified in detail below. In embodiments in which the cleaning adjunct materials are not compatible with the variant proteases of the present invention in the cleaning compositions, then suitable methods of keeping the cleaning adjunct materials and the protease(s) separated (i.e., not in contact with each other) until combination of the two components is appropriate are used. Such separation methods include any suitable method known in the art (e.g., gelcaps, encapsulation, tablets, physical separation, etc.).

In some embodiments, an effective amount of one or more variant protease(s) provided herein is included in compositions useful for cleaning a variety of surfaces in need of proteinaceous stain removal. Such cleaning compositions include cleaning compositions for such applications as cleaning hard surfaces, fabrics, and dishes. Indeed, in some embodiments, the present invention provides fabric cleaning compositions, while in other embodiments the present invention provides non-fabric cleaning compositions. Notably, the present invention also provides cleaning compositions suitable for personal care, including oral care (including dentrifices, toothpastes, mouthwashes, etc., as well as denture cleaning compositions), skin, and hair cleaning compositions. It is intended that the present invention encompass detergent compositions in any form (i.e., liquid, granular, bar, semi-solid, gels, emulsions, tablets, capsules, etc.).

By way of example, several cleaning compositions wherein the variant proteases of the present invention find use are described in greater detail below. In some embodiments in which the cleaning compositions of the present invention are formulated as compositions suitable for use in laundry machine washing method(s), the compositions of the present invention preferably contain at least one surfactant and at least one builder compound, as well as one or more cleaning adjunct materials preferably selected from organic polymeric compounds, bleaching agents, additional enzymes, suds suppressors, dispersants, lime-soap dispersants, soil suspension and anti-redeposition agents and corrosion inhibitors. In some embodiments, laundry compositions also contain softening agents (i.e., as additional cleaning adjunct materials). The compositions of the present invention also find use detergent additive products in solid or liquid form. Such additive products are intended to supplement and/or boost the performance of conventional detergent compositions and can be added at any stage of the cleaning process. In some embodiments, the density of the laundry detergent compositions herein ranges from about 400 to about 1200 g/liter, while in other embodiments, it ranges from about 500 to about 950 g/liter of composition measured at 20° C.

In embodiments formulated as compositions for use in manual dishwashing methods, the compositions of the invention preferably contain at least one surfactant and preferably at least one additional cleaning adjunct material selected from organic polymeric compounds, suds enhancing agents, group 11 metal ions, solvents, hydrotropes and additional enzymes.

In some embodiments, various cleaning compositions such as those provided in U.S. Pat. No. 6,605,458, find use with the variant proteases of the present invention. Thus, in some embodiments, the compositions comprising at least one variant protease of the present invention is a compact granular fabric cleaning composition, while in other embodiments, the composition is a granular fabric cleaning composition useful in the laundering of colored fabrics, in further embodiments, the composition is a granular fabric cleaning composition which provides softening through the wash capacity, in additional embodiments, the composition is a heavy duty liquid fabric cleaning composition. In some embodiments, the compositions comprising at least one variant protease of the present invention are fabric cleaning compositions such as those described in U.S. Pat. Nos. 6,610,642 and 6,376,450. In addition, the variant proteases of the present invention find use in granular laundry detergent compositions of particular utility under European or Japanese washing conditions (See e.g., U.S. Pat. No. 6,610,642).

In some alternative embodiments, the present invention provides hard surface cleaning compositions comprising at least one variant protease provided herein. Thus, in some embodiments, the compositions comprising at least one variant protease of the present invention is a hard surface cleaning composition such as those described in U.S. Pat. Nos. 6,610,642, 6,376,450, and 6,376,450.

In yet further embodiments, the present invention provides dishwashing compositions comprising at least one variant protease provided herein. Thus, in some embodiments, the compositions comprising at least one variant protease of the present invention is a hard surface cleaning composition such as those in U.S. Pat. Nos. 6,610,642 and 6,376,450. In some still further embodiments, the present invention provides dishwashing compositions comprising at least one variant protease provided herein. In some further embodiments, the compositions comprising at least one variant protease of the present invention comprise oral care compositions such as those in U.S. Pat. Nos. 6,376,450 and 6,376,450. The formulations and descriptions of the compounds and cleaning adjunct materials contained in the aforementioned U.S. Pat. Nos. 6,376,450, 6,605,458, 6,605,458, and 6,610,642, find use with the variant proteases provided herein.

The cleaning compositions of the present invention are formulated into any suitable form and prepared by any process chosen by the formulator, non-limiting examples of which are described in U.S. Pat. Nos. 5,879,584, 5,691,297, 5,574,005, 5,569,645, 5,565,422, 5,516,448, 5,489,392, and 5.486,303. When a low pH cleaning composition is desired, the pH of such composition is adjusted via the addition of a material such as monoethanolamine or an acidic material such as HCl.

While not essential for the purposes of the present invention, the non-limiting list of adjuncts illustrated hereinafter are suitable for use in the instant cleaning compositions. In some embodiments, these adjuncts are incorporated for example, to assist or enhance cleaning performance, for treatment of the substrate to be cleaned, or to modify the aesthetics of the cleaning composition as is the case with perfumes, colorants, dyes or the like. It is understood that such adjuncts are in addition to the variant proteases of the present invention. The precise nature of these additional components, and levels of incorporation thereof, will depend on the physical form of the composition and the nature of the cleaning operation for which it is to be used. Suitable adjunct materials include, but are not limited to, surfactants, builders, chelating agents, dye transfer inhibiting agents, deposition aids, dispersants, additional enzymes, and enzyme stabilizers, catalytic materials, bleach activators, bleach boosters, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282, 6,306,812, and 6,326,348. The aforementioned adjunct ingredients may constitute the balance of the cleaning compositions of the present invention.

In some embodiments, the cleaning compositions according to the present invention comprise at least one surfactant and/or a surfactant system wherein the surfactant is selected from nonionic surfactants, anionic surfactants, cationic surfactants, ampholytic surfactants, zwitterionic surfactants, semi-polar nonionic surfactants and mixtures thereof. In some low pH cleaning composition embodiments (e.g., compositions having a neat pH of from about 3 to about 5), the composition typically does not contain alkyl ethoxylated sulfate, as it is believed that such surfactant may be hydrolyzed by such compositions the acidic contents. In some embodiments, the surfactant is present at a level of from about 0.1 to about 60%, while in alternative embodiments the level is from about 1 to about 50%, while in still further embodiments the level is from about 5 to about 40%, by weight of the cleaning composition.

In some embodiments, the cleaning compositions of the present invention comprise one or more detergent builders or builder systems. In some embodiments incorporating at least one builder, the cleaning compositions comprise at least about 1%, from about 3 to about 60% or even from about 5 to about 40% builder by weight of the cleaning composition. Builders include, but are not limited to, the alkali metal, ammonium and alkanolammoniumn salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1, 3, 5-trihydroxy benzene-2, 4, 6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in various embodiments of the present invention.

In some embodiments, the builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present invention, including those known in the art (See e.g., EP2100949).

In some embodiments, the cleaning compositions of the present invention contain at least one chelating agent. Suitable chelating agents include, but are not limited to copper, iron and/or manganese chelating agents and mixtures thereof. In embodiments in which at least one chelating agent is used, the cleaning compositions of the present invention comprise from about 0.1 to about 15% or even from about 3.0 to about 10% chelating agent by weight of the subject cleaning composition.

In some still further embodiments, the cleaning compositions provided herein contain at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

As indicated herein, in some embodiments, anti-redeposition agents find use in some embodiments of the present invention. In some embodiments, non-ionic surfactants find use. For example, in automatic dishwashing embodiments, non-ionic surfactants find use for surface modification purposes, in particular for sheeting, to avoid filming and spotting and to improve shine. These non-ionic surfactants also find use in preventing the re-deposition of soils. In some embodiments, the anti-redeposition agent is a non-ionic surfactant as known in the art (See e.g., EP2100949).

In some embodiments, the cleaning compositions of the present invention include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. In embodiments in which at least one dye transfer inhibiting agent is used, the cleaning compositions of the present invention comprise from about 0.0001 to about 10%, from about 0.01 to about 5%, or even from about 0.1 to about 3% by weight of the cleaning composition.

In some embodiments, silicates are included within the compositions of the present invention. In some such embodiments, sodium silicates (e.g., sodium disilicate, sodium metasilicate, and crystalline phyllosilicates) find use. In some embodiments, silicates are present at a level of from about 1 to about 20%. In some embodiments, silicates are present at a level of from about 5 to about 15% by weight of the composition.

In some still additional embodiments, the cleaning compositions of the present invention also contain dispersants. Suitable water-soluble organic materials include, but are not limited to the homo- or co-polymeric acids or their salts, in which the polycarboxylic acid comprises at least two carboxyl radicals separated from each other by not more than two carbon atoms.

In some further embodiments, the enzymes used in the cleaning compositions are stabilized by any suitable technique. In some embodiments, the enzymes employed herein are stabilized by the presence of water-soluble sources of calcium and/or magnesium ions in the finished compositions that provide such ions to the enzymes. In some embodiments, the enzyme stabilizers include oligosaccharides, polysaccharides, and inorganic divalent metal salts, including alkaline earth metals, such as calcium salts. It is contemplated that various techniques for enzyme stabilization will find use in the present invention. For example, in some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), Tin (II), cobalt (II), copper (II), nickel (II), and oxovanadium (IV). Chlorides and sulfates also find use in some embodiments of the present invention. Examples of suitable oligosaccharides and polysaccharides (e.g., dextrins) are known in the art (See e.g., WO07/145964). In some embodiments, reversible protease inhibitors also find use, such as boron-containing compounds (e.g., borate, 4-formyl phenyl boronic acid) and/or a tripeptide aldehyde find use to further improve stability, as desired.

In some embodiments, bleaches, bleach activators and/or bleach catalysts are present in the compositions of the present invention. In some embodiments, the cleaning compositions of the present invention comprise inorganic and/or organic bleaching compound(s). Inorganic bleaches include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Any suitable salt known in the art finds use in the present invention (See e.g., EP2100949).

In some embodiments, bleach activators are used in the compositions of the present invention. Bleach activators are typically organic peracid precursors that enhance the bleaching action in the course of cleaning at temperatures of 60° C. and below. Bleach activators suitable for use herein include compounds which, under perhydrolysis conditions, give aliphatic peroxoycarboxylic acids having preferably from about 1 to about 10 carbon atoms, in particular from about 2 to about 4 carbon atoms, and/or optionally substituted perbenzoic acid. Additional bleach activators are known in the art and find use in the present invention (See e.g., EP2100949).

In addition, in some embodiments and as further described herein, the cleaning compositions of the present invention further comprise at least one bleach catalyst. In some embodiments, the manganese triazacyclononane and related complexes find use, as well as cobalt, copper, manganese, and iron complexes. Additional bleach catalysts find use in the present invention (See e.g., U.S. Pat. Nos. 4,246,612, 5,227,084, and 4,810,410, and WO 99/06521 and EP2100949).

In some embodiments, the cleaning compositions of the present invention contain one or more catalytic metal complexes. In some embodiments, a metal-containing bleach catalyst finds use. In some embodiments, the metal bleach catalyst comprises a catalyst system comprising a transition metal cation of defined bleach catalytic activity, (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (See e.g., U.S. Pat. No. 4,430,243). In some embodiments, the cleaning compositions of the present invention are catalyzed by means of a manganese compound. Such compounds and levels of use are well known in the art (See e.g., U.S. Pat. No. 5,576,282). In additional embodiments, cobalt bleach catalysts find use in the cleaning compositions of the present invention. Various cobalt bleach catalysts are known in the art (See e.g., U.S. Pat. Nos. 5,597,936 and 5,595,967) and are readily prepared by known procedures.

In some additional embodiments, the cleaning compositions of the present invention include a transition metal complex of a macropolycyclic rigid ligand (MRL). As a practical matter, and not by way of limitation, in some embodiments, the compositions and cleaning processes provided by the present invention are adjusted to provide on the order of at least one part per hundred million of the active MRL species in the aqueous washing medium, and in some embodiments, provide from about 0.005 ppm to about 25 ppm, more preferably from about 0.05 ppm to about 10 ppm, and most preferably from about 0.1 ppm to about 5 ppm, of the MRL in the wash liquor.

In some embodiments, transition-metals in the instant transition-metal bleach catalyst include, but are not limited to manganese, iron and chromium. MRLs also include, but are not limited to special ultra-rigid ligands that are cross-bridged (e.g., 5,12-diethyl-1,5,8,12-tetraazabicyclo[6.6.2]hexadecane). Suitable transition metal MRLs are readily prepared by known procedures (See e.g., WO 2000/32601, and U.S. Pat. No. 6,225,464).

In some embodiments, the cleaning compositions of the present invention comprise metal care agents. Metal care agents find use in preventing and/or reducing the tarnishing, corrosion, and/or oxidation of metals, including aluminum, stainless steel, and non-ferrous metals (e.g., silver and copper). Suitable metal care agents include those described in EP2100949, WO9426860 and WO9426859). In some embodiments, the metal care agent is a zinc salt. In some further embodiments, the cleaning compositions of the present invention comprise from about 0.1% to about 5% by weight of one or more metal care agent.

In some embodiments, the cleaning composition is a high density liquid (HDL) composition having a variant LG12 protease. The HDL liquid laundry detergent can comprise a detersive surfactant (10-40%) comprising anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof): and optionally non-ionic surfactant (selected from a group of linear or branched or random chain, substituted or unsubstituted alkyl alkoxylated alcohol, for example a C₈-C₁₈ alkyl ethoxylated alcohol and/or C₆-C₁₂ alkyl phenol alkoxylates), optionally wherein the weight ratio of anionic detersive surfactant (with a hydrophilic index (HIc) of from 6.0-9) to non-ionic detersive surfactant is greater than 1:1.

The composition can comprise optionally, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05-10 wt %) and/or random graft polymers (typically comprising of hydrophilic backbone comprising monomers selected from the group consisting of: unsaturated C₁-C₆ carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C₄-C₂ alkyl group, polypropylene, polybutylene, vinyl ester of a saturated C₁-C₆ mono-carboxylic acid, C₁-C₆ alkyl ester of acrylic or methacrylic acid, and mixtures thereof.

The composition can comprise additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example Repel-o-tex SF, SF-2 and SRP6, Texcare SRA100, SRA300, SRN100. SRN170, SRN240. SRN300 and SRN325, Marloquest SL), anti-redeposition polymers (0.1 wt % to 10 wt %, include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da): cellulosic polymer (including those selected from alkyl cellulose, alkyl alkoxyalkyl cellulose, carboxyalkyl cellulose, alkyl carboxyalkyl cellulose examples of which include carboxymethyl cellulose, methyl cellulose, methyl hydroxyethyl cellulose, methyl carboxymethyl cellulose, and mixures thereof) and polymeric carboxylate (such as maleate/acrylate random copolymer or polyacrylate homopolymer).

The composition can further comprise saturated or unsaturated fatty acid, preferably saturated or unsaturated C₁₂-C₂₄ fatty acid (0-10 wt %): deposition aids (examples for which include polysaccharides, preferably cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic cellulose such as cationic hydoxyethyl cellulose, cationic starch, cationic polyacylamides, and mixtures thereof.

The composition can further comprise dye transfer inhibiting agents examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA): propylene diamine tetracetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof.

The composition can further comprise enzymes (0.01-0.03 wt % active enzyme) selected from a group of proteases; amylases; lipases; cellulases; choline oxidases; peroxidases/oxidases; pectate lyases; mannanases; cutinases: laccases; phospholipases; lysophospholipases: acyltransferase: perhydrolase; arylesterase and any mixture thereof. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

The composition can further comprise silicone or fatty-acid based suds suppressors: heuing dyes, calcium and magnesium cations, visual signaling ingredients, anti-foam (0.001-4.0 wt %), and/or structurant/thickener (0.01-5 wt %, selected from the group consisting of diglycerides and triglycerides, ethylene glycol distearate, microcrystalline cellulose, cellulose based materials, microfiber cellulose, biopolymers, xanthan gum, gellan gum, and mixtures thereof).

Suitable detersive surfactants also include cationic detersive surfactants (selected from a group of alkyl pyridinium compounds, alkyl quarternary ammonium compounds, alkyl quarternary phosphonium compounds, alkyl ternary sulphonium compounds, and/or mixtures thereof); zwitterionic and/or amphoteric detersive surfactants (selected from a group of alkanolamine sulpho-betaines): ampholytic surfactants; semi-polar non-ionic surfactants and mixtures thereof.

The composition can be any liquid form, for example a liquid or gel form, or any combination thereof. The composition may be in any unit dose form, for example a pouch.

In some embodiments, the cleaning composition is a HDD composition having a variant LG12 protease. The HDD laundry detergent can comprise a detersive surfactant including anionic detersive surfactants (selected from a group of linear or branched or random chain, substituted or unsubstituted alkyl sulphates, alkyl sulphonates, alkyl alkoxylated sulphate, alkyl phosphates, alkyl phosphonates, alkyl carboxylates and/or mixtures thereof), non-ionic detersive surfactant (selected from a group of linear or branched or random chain, substituted or unsubstituted C₈-C₁₈ alkyl ethoxylates, and/or C₆-C₁₂ alkyl phenol alkoxylates), cationic detersive surfactants (selected from a group of alkyl pyridinium compounds, alkyl quaternary ammonium compounds, alkyl quaternary phosphonium compounds, alkyl ternary sulphonium compounds, and mixtures thereof), zwitterionic and/or amphoteric detersive surfactants (selected from a group of alkanolamine sulpho-betaines); ampholytic surfactants; semi-polar non-ionic surfactants and mixtures thereof; builders (phosphate free builders [for example zeolite builders examples of which include zeolite A, zeolite X, zeolite P and zeolite MAP in the range of 0 to less than 10 wt %]; phosphate builders [examples of which include sodium tri-polyphosphate in the range of 0 to less than 10 wt %]; citric acid, citrate salts and nitrilotriacetic acid or salt thereof in the range of less than 15 wt %): silicate salt (sodium or potassium silicate or sodium meta-silicate in the range of 0 to less than 10 wt %, or layered silicate (SKS-6)): carbonate salt (sodium carbonate and/or sodium bicarbonate in the range of 0 to less than 10 wt %); and bleaching agents (photobleaches, examples of which include sulfonated zinc phthalocyanines, sulfonated aluminum phthalocyanines, xanthenes dyes, and mixtures thereof: hydrophobic or hydrophilic bleach activators (examples of which include dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, and nonanoyloxybenzene sulfonate-NOBS, nitrile quats, and mixtures thereof: hydrogen peroxide; sources of hydrogen peroxide (inorganic perhydrate salts examples of which include mono or tetra hydrate sodium salt of perborate, percarbonate, persulfate, perphosphate, or persilicate); preformed hydrophilic and/or hydrophobic peracids (selected from a group consisting of percarboxylic acids and salts, percarbonic acids and salts, perimidic acids and salts, peroxymonosulfuric acids and salts) & mixtures thereof and/or bleach catalyst (such as imine bleach boosters examples of which include iminium cations and polyions; iminium zwitterions; modified amines; modified amine oxides; N-sulphonyl imines; N-phosphonyl imines: N-acyl imines; thiadiazole dioxides: perfluoroimines; cyclic sugar ketones and mixtures thereof; metal-containing bleach catalyst for example copper, iron, titanium, ruthenium, tungsten, molybdenum, or manganese cations along with an auxiliary metal cations such as zinc or aluminum and a sequestrate such as ethylenediaminetetraacetic acid, ethylenediaminetetra(methylenephosphonic acid) and water-soluble salts thereof).

The composition can further comprise enzymes selected from a group of proteases; amylases; lipases: cellulases: choline oxidases; peroxidases/oxidases; pectate lyases; mannanases; cutinases: laccases; phospholipases; lysophospholipases; acyltransferase: perhydrolase: arylesterase and any mixture thereof.

The composition can further comprise additional detergent ingredients including perfume microcapsules, starch encapsulated perfume accord, hueing agents, additional polymers including fabric integrity and cationic polymers, dye lock ingredients, fabric-softening agents, brighteners (for example C.I. Fluorescent brighteners), flocculating agents, chelating agents, alkoxylated polyamines, fabric deposition aids, and/or cyclodextrin.

In some embodiments, the cleaning composition is an automatic dishwashing (ADW) detergent composition having a variant LG12 protease. The ADW detergent can comprise two or more non-ionic surfactants selected from a group of ethoxylated non-ionic surfactants, alcohol alkoxylated surfactants, epoxy-capped poly(oxyalkylated) alcohols, or amine oxide surfactants present in amounts from 0-10% by weight; builders in the range of 5-60% comprising either phosphate (mono-phosphates, di-phosphates, tri-polyphosphates or oligomeric-poylphosphates, preferred sodium tripolyphosphate-STPP or phosphate-free builders [amino acid based compounds, examples of which include MGDA (methyl-glycine-diacetic acid), and salts and derivatives thereof, GLDA (glutamic-N,Ndiacetic acid) and salts and derivatives thereof. IDS (iminodisuccinic acid) and salts and derivatives thereof, carboxy methyl inulin and salts and derivatives thereof and mixtures thereof, nitrilotriacetic acid (NTA), diethylene triamine penta acetic acid (DTPA), B-alaninediacetic acid (B-ADA) and their salts], homopolymers and copolymers of poly-carboxylic acids and their partially or completely neutralized salts, monomeric polycarboxylic acids and hydroxycarboxylic acids and their salts in the range of 0.5-50% by weight: sulfonatedcarboxylated polymers (provide dimensional stability to the product) in the range of about 0.1 to about 50% by weight; drying aids in the range of about 0.1 to about 10% by weight (selected from polyesters, especially anionic polyesters optionally together with further monomers with 3 to 6 functionalities which are conducive to polycondensation, specifically acid, alcohol or ester functionalities, polycarbonate-, polyurethane- and/or polyurea-polyorganosiloxane compounds or precursor compounds thereof of the reactive cyclic carbonate and urea type); silicates in the range from about 1 to about 20% by weight (sodium or potassium silicates for example sodium disilicate, sodium meta-silicate and crystalline phyllosilicates); bleach-inorganic (for example perhydrate salts such as perborate, percarbonate, perphosphate, persulfate and persilicate salts) and organic (for example organic peroxyacids including diacyl and tetraacylperoxides, especially diperoxydodecanedioc acid, diperoxytetradecanedioc acid, and diperoxyhexadecanedioc acid); bleach activators—organic peracid precursors in the range from about 0.1 to about 10% by weight; bleach catalysts (selected from manganese triazacyclononane and related complexes, Co, Cu, Mn and Fe bispyridylamine and related complexes, and pentamine acetate cobalt(III) and related complexes); metal care agents in the range from about 0.1 to 5% by weight (selected from benzatriazoles, metal salts and complexes, and/or silicates); enzymes in the range from about 0.01 to 5.0 mg of active enzyme per gram of automatic dishwashing detergent composition (selected from a group of proteases; amylases; lipases; cellulases: choline oxidases; peroxidases/oxidases: pectate lyases; mannanases; cutinases; laccases: phospholipases; lysophospholipases; acyltransferase; perhydrolase; arylesterase and any mixture thereof); and enzyme stabilizer components (selected from oligosaccharides, polysaccharides and inorganic divalent metal salts).

Representative detergent formulations that beneficially include a serine protease polypeptide of the present invention include the detergent formulations found in WO2013063460, pages 78-152, and in particular the tables of pages 94-152 are hereby incorporated by reference. The serine proteases are normally incorporated into the detergent composition at a level of from 0.00001-10% of enzyme protein by weight of the composition. In some embodiments, the detergent composition comprises more than 0.0001%, 0.001%, 0.01%, or 0.1% of the serine protease by weight of the composition. In some embodiments, the detergent composition comprises less than 1%, 0.1%, 0.01%, or 0.001% of the serine protease by weight of the composition.

The cleaning compositions of the present invention are formulated into any suitable form and prepared by any process chosen by the formulator, non-limiting examples of which are described in U.S. Pat. Nos. 5,879,584, 5,691,297, 5,574,005, 5,569,645, 5,516,448, 5,489,392, and 5,486,303, all of which are incorporated herein by reference. In some embodiments in which a low pH cleaning composition is desired, the pH of such composition is adjusted via the addition of an acidic material such as HCl.

The cleaning compositions disclosed herein of find use in cleaning a situs (e.g., a surface, item, dishware, or fabric). Typically, at least a portion of the situs is contacted with an embodiment of the present cleaning composition, in neat form or diluted in wash liquor, and then the situs is optionally washed and/or rinsed. For purposes of the present invention, “washing” includes but is not limited to, scrubbing, and mechanical agitation. In some embodiments, the cleaning compositions are typically employed at concentrations of from about 500 ppm to about 15.000 ppm in solution. When the wash solvent is water, the water temperature typically ranges from about 5° C. to about 90° C. and, when the situs comprises a fabric, the water to fabric mass ratio is typically from about 1:1 to about 30:1.

The cleaning compositions of the present invention are formulated into any suitable form and prepared by any suitable process chosen by the formulator, (See e.g., U.S. Pat. Nos. 5,879,584, 5,691,297, 5,574,005, 5,569,645, 5,565,422, 5,516,448, 5,489,392, 5,486,303, 4,515,705, 4,537,706, 4,515,707, 4,550,862, 4,561,998, 4,597,898, 4,968,451, 5,565,145, 5,929,022, 6,294,514 and 6,376,445).

In some embodiments, the cleaning compositions of the present invention are provided in unit dose form, including tablets, capsules, sachets, pouches, and multi-compartment pouches. In some embodiments, the unit dose format is designed to provide controlled release of the ingredients within a multi-compartment pouch (or other unit dose format). Suitable unit dose and controlled release formats are known in the art (See e.g., EP 2100949, WO02/102955, U.S. Pat. Nos. 4,765,916 and 4,972,017, and WO 04/111178 for materials suitable for use in unit dose and controlled release formats). In some embodiments, the unit dose form is provided by tablets wrapped with a water-soluble film or water-soluble pouches. Various formats for unit doses are provided in EP2100947, and are known in the art.

In some embodiments, the cleaning compositions of the present invention find use in cleaning surfaces (e.g., dishware), laundry, hard surfaces, contact lenses, etc. In some embodiments, at least a portion of the surface is contacted with at least one embodiment of the cleaning compositions of the present invention, in neat form or diluted in wash liquor, and then the surface is optionally washed and/or rinsed. For purposes of the present invention, “washing” includes, but is not limited to, scrubbing, and mechanical washing. In some embodiments, the cleaning compositions of the present invention are used at concentrations of from about 500 ppm to about 15,000 ppm in solution. In some embodiments in which the wash solvent is water, the water temperature typically ranges from about 5° C. to about 90° C.

The present invention provides methods for cleaning or washing an item or surface (e.g., hard surface) in need of cleaning, including, but not limited to methods for cleaning or washing a dishware item, a tableware item, a fabric item, a laundry item, personal care item, etc., or the like, and methods for cleaning or washing a hard or soft surface (e.g., a hard surface of an item).

In some embodiments, the present invention provides a method for cleaning an item, object, or surface in need of cleaning, the method comprising contacting the item or surface (or a portion of the item or surface desired to be cleaned) with at least one variant LG12 protease of the present invention or a composition of the present invention for a sufficient time and/or under conditions suitable and/or effective to clean the item, object, or surface to a desired degree. Some such methods further comprise rinsing the item, object, or surface with water. For some such methods, the cleaning composition is a dishwashing detergent composition and the item or object to be cleaned is a dishware item or tableware item. As used herein, a “dishware item” is an item generally used in serving or eating food. A dishware item can be, but is not limited to for example, a dish, plate, cup, bowl, etc., and the like. As used herein, “tableware” is a broader term that includes, but is not limited to for example, dishes, cutlery, knives, forks, spoons, chopsticks, glassware, pitchers, sauce boats, drinking vessels, serving items, etc. It is intended that “tableware item” includes any of these or similar items for serving or eating food. For some such methods, the cleaning composition is an automatic dishwashing detergent composition or a hand dishwashing detergent composition and the item or object to be cleaned is a dishware or tableware item. For some such methods, the cleaning composition is a laundry detergent composition (e.g., a power laundry detergent composition or a liquid laundry detergent composition), and the item to be cleaned is a fabric item. In some other embodiments, the cleaning composition is a laundry pre-treatment composition.

In some embodiments, the present invention provides methods for cleaning or washing a fabric item optionally in need of cleaning or washing, respectively. In some embodiments, the methods comprise providing a composition comprising the variant protease, including but not limited to fabric or laundry cleaning composition, and a fabric item or laundry item in need of cleaning, and contacting the fabric item or laundry item (or a portion of the item desired to be cleaned) with the composition under conditions sufficient or effective to clean or wash the fabric or laundry item to a desired degree.

In some embodiments, the present invention provides a method for cleaning or washing an item or surface (e.g., hard surface) optionally in need of cleaning, the method comprising providing an item or surface to be cleaned or washed and contacting the item or surface (or a portion of the item or surface desired to be cleaned or washed) with at least one LG12 variant of the invention or a composition of the invention comprising at least one such LG12 variant for a sufficient time and/or under conditions sufficient or effective to clean or wash the item or surface to a desired degree. Such compositions include, but are not limited to for example, a cleaning composition or detergent composition of the invention (e.g., a hand dishwashing detergent composition, hand dishwashing cleaning composition, laundry detergent or fabric detergent or laundry or fabric cleaning composition, liquid laundry detergent, liquid laundry cleaning composition, powder laundry detergent composition, powder laundry cleaning composition, automatic dishwashing detergent composition, laundry booster cleaning or detergent composition, laundry cleaning additive, and laundry pre-spotter composition, etc.). In some embodiments, the method is repeated one or more times, particularly if additional cleaning or washing is desired. For example, in some instance, the method optionally further comprises allowing the item or surface to remain in contact with the at least one variant protease or composition for a period of time sufficient or effective to clean or wash the item or surface to the desired degree. In some embodiments, the methods further comprise rinsing the item or surface with water and/or another liquid. In some embodiments, the methods further comprise contacting the item or surface with at least one variant protease of the invention or a composition of the invention again and allowing the item or surface to remain in contact with the at least one variant protease or composition for a period of time sufficient to clean or wash the item or surface to the desired degree. In some embodiments, the cleaning composition is a dishwashing detergent composition and the item to be cleaned is a dishware or tableware item. In some embodiments of the present methods, the cleaning composition is an automatic dishwashing detergent composition or a hand dishwashing detergent composition and the item to be cleaned is a dishware or tableware item. In some embodiments of the methods, the cleaning composition is a laundry detergent composition and the item to be cleaned is a fabric item.

The present invention also provides methods of cleaning a tableware or dishware item in an automatic dishwashing machine, the method comprising providing an automatic dishwashing machine, placing an amount of an automatic dishwashing composition comprising at least one LG12 variant of the present invention or a composition of the invention sufficient to clean the tableware or dishware item in the machine (e.g., by placing the composition in an appropriate or provided detergent compartment or dispenser in the machine), putting a dishware or tableware item in the machine, and operating the machine so as to clean the tableware or dishware item (e.g., as per the manufacturer's instructions). In some embodiments, the methods include any automatic dishwashing composition described herein, which comprises, but is not limited to at least one LG12 variant provided herein. The amount of automatic dishwashing composition to be used can be readily determined according to the manufacturer's instructions or suggestions and any form of automatic dishwashing composition comprising at least one variant protease of the invention (e.g., liquid, powder, solid, gel, tablet, etc.), including any described herein, may be employed.

The present invention also provides methods for cleaning a surface, item or object optionally in need of cleaning, the method comprises contacting the item or surface (or a portion of the item or surface desired to be cleaned) with at least one variant LG12 of the present invention or a cleaning composition of the invention in neat form or diluted in a wash liquor for a sufficient time and/or under conditions sufficient or effective to clean or wash the item or surface to a desired degree. The surface, item, or object may then be (optionally) washed and/or rinsed if desired. For purposes of the present invention, “washing” includes, but is not limited to for example, scrubbing and mechanical agitation. In some embodiments, the cleaning compositions are employed at concentrations of from about 500 ppm to about 15,000 ppm in solution (e.g., aqueous solution). When the wash solvent is water, the water temperature typically ranges from about 5° C. to about 90° C.

The present invention also provides methods of cleaning a laundry or fabric item in an washing machine, the method comprising providing an washing machine, placing an amount of a laundry detergent composition comprising at least one variant LG12 of the invention sufficient to clean the laundry or fabric item in the machine (e.g., by placing the composition in an appropriate or provided detergent compartment or dispenser in the machine), placing the laundry or fabric item in the machine, and operating the machine so as to clean the laundry or fabric item (e.g., as per the manufacturer's instructions). The methods of the present invention include any laundry washing detergent composition described herein, comprising but not limited to at least one of any variant LG12 provided herein. The amount of laundry detergent composition to be used can be readily determined according to manufacturer's instructions or suggestions and any form of laundry detergent composition comprising at least one variant protease of the invention (e.g., solid, powder, liquid, tablet, gel, etc.), including any described herein, may be employed.

In a further aspect of the invention, the LG-12 clade protease polypeptides of the present invention can be used as a component of an animal feed composition, animal feed additive and/or pet food comprising a LG-12 clade protease and variants thereof. The present invention further relates to a method for preparing such an animal feed composition, animal feed additive composition and/or pet food comprising mixing the LG-12 clade protease polypeptide with one or more animal feed ingredients and/or animal feed additive ingredients and/or pet food ingredients. Furthermore, the present invention relates to the use of the LG-12 clade protease polypeptide in the preparation of an animal feed composition and/or animal feed additive composition and/or pet food.

The term “animal” includes all non-ruminant and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghorn and nilgai.

In the present context, it is intended that the term “pet food” is understood to mean a food for a household animal such as, but not limited to, dogs, cats, gerbils, hamsters, chinchillas, fancy rats, guinea pigs; avian pets, such as canaries, parakeets, and parrots, reptile pets, such as turtles, lizards and snakes: and aquatic pets, such as tropical fish and frogs.

The terms “animal feed composition.” “feedstuff” and “fodder” are used interchangeably and can comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grain Solubles (DDGS) (particularly corn based Distillers Dried Grain Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

Also contemplated are compositions and methods of treating fabrics (e.g., to desize a textile) using a LG-12 clade protease polypeptide of the present invention. Fabric-treating methods are well known in the art (see, e.g., U.S. Pat. No. 6,077,316). For example, the feel and appearance of a fabric can be improved by a method comprising contacting the fabric with a LG-2 clade protease in a solution. The fabric can be treated with the solution under pressure.

A LG-12 clade protease of the present invention can be applied during or after the weaving of a textile, or during the desizing stage, or one or more additional fabric processing steps. During the weaving of textiles, the threads are exposed to considerable mechanical strain. Prior to weaving on mechanical looms, warp yarns are often coated with sizing starch or starch derivatives to increase their tensile strength and to prevent breaking. A LG-12 clade protease of the present invention can be applied during or after the weaving to remove starch or starch derivatives. After weaving, the LG-12 clade protease can be used to remove the size coating before further processing the fabric to ensure a homogeneous and wash-proof result.

A LG-12 clade protease of the present invention can be used alone or with other desizing chemical reagents and/or desizing enzymes to desize fabrics, including cotton-containing fabrics, as detergent additives, e.g., in aqueous compositions. An amylase also can be used in compositions and methods for producing a stonewashed look on indigo-dyed denim fabric and garments. For the manufacture of clothes, the fabric can be cut and sewn into clothes or garments, which are afterwards finished. In particular, for the manufacture of denim jeans, different enzymatic finishing methods have been developed. The finishing of denim garment normally is initiated with an enzymatic desizing step, during which garments are subjected to the action of proteolytic enzymes to provide softness to the fabric and make the cotton more accessible to the subsequent enzymatic finishing steps. The LG-12 clade protease can be used in methods of finishing denim garments (e.g., a “bio-stoning process”), enzymatic desizing and providing softness to fabrics, and/or finishing process.

The LG-12 clade protease polypeptides described herein find further use in the enzyme aided bleaching of paper pulps such as chemical pulps, semi-chemical pulps, kraft pulps, mechanical pulps or pulps prepared by the sulfite method. In general terms, paper pulps are incubated with a LG-12 clade protease polypeptide of the present invention under conditions suitable for bleaching the paper pulp. In some embodiments, the pulps are chlorine free pulps bleached with oxygen, ozone, peroxide or peroxyacids. In some embodiments, the LG-12 clade protease polypeptides are used in enzyme aided bleaching of pulps produced by modified or continuous pulping methods that exhibit low lignin contents. In some other embodiments, the LG-12 clade protease polypeptides are applied alone or preferably in combination with xylanase and/or endoglucanase and/or alpha-galactosidase and/or cellobiohydrolase enzymes.

The LG-12 clade protease polypeptides described herein find further use in the enzyme aided removal of proteins from animals and their subsequent degradation or disposal, such as feathers, skin, hair, hide, and the like. In some instances, immersion of the animal carcass in a solution comprising a LG-2 clade protease polypeptide of the present invention can act to protect the skin from damage in comparison to the traditional immersion in scalding water or the defeathering process. In one embodiment, feathers can be sprayed with an isolated polypeptide of the present invention under conditions suitable for digesting or initiating degradation of the plumage. In some embodiments, a LG-12 clade protease of the present invention can be used, as above, in combination with an oxidizing agent.

In some embodiments, removal of the oil or fat associated with raw feathers is assisted by using a LG-12 clade protease polypeptide of the present invention. In some embodiments, the LG-12 clade protease polypeptides are used in compositions for cleaning the feathers as well as to sanitize and partially dehydrate the fibers. In some other embodiments, the LG-12 clade protease polypeptides are applied in a wash solution in combination with 95% ethanol or other polar organic solvent with or without a surfactant at about 0.5% (v/v).

In yet other embodiments, the disclosed LG-12 clade protease polypeptides find use in recovering protein from plumage. In some embodiments, the recovered protein can be subsequently used in animal or fish feed.

The LG-12 clade protease polypeptides described herein find further use in the enzyme aided debridement of tissue. This involves the removal of dead or damaged tissue, for example, removal from wounds to aid in healing.

The LG-12 clade protease polypeptides described herein find further use in tissue culture. In particular, LG-12 clade proteases of the present invention can be used to suspend or resuspend cells adherent to a cell culture wall, such as during the process of harvesting cells. LG-12 clade proteases of the present invention can be used to cleave protein bonds between cultured cells and the dish, allowing cells to become suspended in solution.

The LG-12 clade protease polypeptides described herein find further use as a food additive, a digestive aide or a food processing aid.

The LG-12 clade protease polypeptides described herein find further use in leather processing by removing hair from animal hides, soaking, degreasing, or bating, which is a process involving degradation of non-structural proteins during leather making.

### EXAMPLE 1

### Assays

### Performance Index

The performance index (PI) of an enzyme compares the performance of the variant (measured value) with the parent enzyme (theoretical value or measured value) at the same protein concentration. Theoretical concentrations for the parent enzyme can be calculated using the parameters extracted from a Langmuir fit of a standard curve of the parent enzyme. A PI that is greater than 1 (PI>1) indicates improved performance by a variant as compared to a parent or benchmark protease (defined for each analyzed data set), while a PI of 1 (PI=1) identifies a variant that performs the same as the parent, and a PI that is less than 1 (PI<1) identifies a variant that performs worse than the parent.

### Protein Purification

Samples were dialyzed overnight against 25 mM MES, pH 5.4, 1 mM CaCl₂ buffer, and loaded on a 30 mL Source 15S column (GE Healthcare) equilibrated with same buffer. A gradient of 0 to 150 mM NaCl was run, and fractions were collected. Fractions containing target protein were pooled, concentrated, and formulated with 40% Propylene Glycol, and sterile filtered.

### Protease Activity Assays

The protease activity of wild-type LG12 protease and variants thereof was tested by measuring hydrolysis of N-suc-AAPF-pNA, dimethyl casein (DMC), or skim milk substrates.

The reagent solutions used for the AAPF hydrolysis assay were: 100 mM Tris/HCl pH 8.6, containing 0.005% TWEEN®-80 (Tris dilution buffer); 100 mM Tris buffer pH 8.6, containing 10 mM CaCl₂ and 0.005% TWEEN®-80 (Tris/Ca buffer); and 160 mM suc-AAPF-pNA in DMSO (suc-AAPF-pNA stock solution) (Sigma: S-7388). To prepare a substrate working solution, 1 ml suc-AAPF-pNA stock solution was added to 100 ml Tris/Ca buffer and mixed well. An enzyme sample was added to a MTP plate (Greiner 781101) containing 1 mg/suc-AAPF-pNA working solution and assayed for activity at 405 nm over 3 minutes using a SpectraMax plate reader in kinetic mode at RT. The protease activity was expressed as mOD·min⁻¹.

The reagent solutions used for the DMC assay were: 2.5% Dimethylcasein (DMC, Sigma) in 100 mM Sodium Carbonate pH 9.5, 0.075% TNBSA (2,4,6-trinitrobenzene sulfonic acid. Thermo Scientific) in Reagent A. Reagent A: 45.4 g Na₂B₄O₇.10H₂O (Merck) in 15 mL 4N NaOH to reach a final volume of 1000 mL in MQ water. Dilution Solution: 10 mM NaCl, 0.1 mM CaCl₂, 0.005% Tween-80, 0.02% Na-azide. MTPs (Greiner PS-microwell 384) were filled with 47.5 uL DMC substrate following the addition of 2.5 uL of 20 ppm protease supernatant. 50 uL of TNBSA in Reagent A was then added with slow mixing. Activity was measured at 405 nm over 5 min using a SpectraMax plate reader in kinetic mode at RT. Activity was expressed as mOD*min⁻¹.

The reagent solutions used for the skim milk assay were: 40 mg/mL BD Difco skim milk powder in 100 mM Tris, 0.005% Tween-80, pH 8.6 with 10 gpg hardness. The solution was equilibrated for 1 h before use. Activity was measured at 650 nm over 3 min using a SpectraMax plate reader in kinetic mode at RT. Activity was expressed as mOD*min-1.

### Detergents

Proteases were tested for cleaning performance and/or stability in detergents set forth in Table 1. All commercially available HDL laundry detergents except Persil non-Bio and Blue Moon were inactivated to remove enzyme activity by heating to 95° C. for 16 hours in a water bath. Protease activity was assayed following inactivation using the AAPF substrate to ensure complete inactivation. Commercially available HDD laundry detergents were inactivated to remove enzyme activity by preparing a 10× concentrated solution relative to what is used in the final cleaning assay and heating for 16 hours at 95° C. Protease activity was assayed following inactivation using the AAPF substrate. After heating the HDD and HDL detergents for 16 hours, protease activity was non-existent.

TABLE 1
Detergents

| NO. | NAME | TYPE | FORM |
|---|---|---|---|
| 1 | Kirkland Ultra (Sun Products)^{†} | Laundry | HDD |
| 2 | OMO Color (Unilever)^{†} | Laundry | HDD |
| 3 | Surf Excel (Henkel)^{†} | Laundry | HDD |
| 4 | OMO Klein & Krachtig (Unilever)^{†} | Laundry | HDL |
| 5 | Kirkland Ultraclean (Sun Products)^{†} | Laundry | HDL |
| 6 | Blue Moon (Guangzhou Blue Moon)^{†,}* | Laundry | HDL |
| 7 | All 3 (Sun Products)^{†,}* | Laundry | HDL |
| 8 | Purex Dirt Lift (Henkel)^{†,}* | Laundry | HDL |
| 9 | Persil Non-Bio (Unilever)^{†,}* | Laundry | HDL |
| 10 | Test HDL* | Laundry | HDL |
| 11 | GSM-B | Dish | Powder |

^{†}Commercially available detergent;
*Detergent with ≤5 mg/Kg boron

The composition of detergent number 10 is a custom-made in-house HDL detergent (“Test HDL”) having the composition set forth in Table 2A.

TABLE 2A
Test HDL Detergent Composition

| | | | Wt. | | |
|---|---|---|---|---|---|
| Component | Ingredient | Trade name | % | Amount(g) | |
| Solvent | Water (total) | — | 64.89 | 1297.8 | g |
| | C12-C15 Pareth-7 | Empilan KCL 7 | 3 | 60 | g |
| | (Sodium) | NANSA SSA F | 7.5 | 150 | g |
| | Dodecylbenzene- | | | | |
| | sulfonate | | | | |
| Surfactant | K-Cocoate | NANSA PC 38F | 3 | 60 | g |
| | Sodium Laureth | Empicol | 9 | 180 | g |
| | Sulfate | ESB3/M6 | | | |
| Builder | Sodium Citrate | Sodium Citrate | 3 | 60 | g |
| | | Tribasic | | | |
| | | Dihydrate | | | |
| Liquid | Sorbitol | D-Sorbitol | 0.8 | 16 | g |
| properties/ | Propylene glycol | 1,2-Propanediol | 2.5 | 50 | g |
| stability | Glycerin | Glycerol | 0.8 | 16 | g |
| | Triethanolamine | Triethanolamine | 0.5 | 10 | g |
| | Methyliso- | 2-Methyl-4- | 0.01 | 0.2 | g |
| | thiazolinone | isothiazolin-3-one | | | |
| | Ethanol | | 1 | 20 | g |
| Neutralizer | NaOH (4M) | | | 103.3 | mL |

The composition of detergent number 11, GSM-B phosphate-free detergent, was purchased from WFK Testgewebe GmbH, Briiggen, Deutschland (www.testgewebe.de) and has the composition set forth in Table 2B.

TABLE 2B
GSM-B ADW Detergent Composition

| Component | Wt % |
|---|---|
| Sodium citrate dehydrate | 30.0 |
| Maleic acid/acrylic acid copolymer sodium Salt | 12.0 |
| (SOKALAN ® CP5; BASF) | |
| Sodium perborate monohydrate | 5.0 |
| TAED | 2.0 |
| Sodium disilicate: Protil A (Cognis) | 25.0 |
| Linear fatty alcohol ethoxylate | 2.0 |
| Sodium carbonate anhydrous | add to 100 |

### Cleaning Performance in Detergent

Variants were tested for cleaning performance relative to wild-type LG12 or BPN′-Y217L protease on various microswatches: BMI (blood/milk/ink on cotton.—EMPA-116), egg yolk with pigment (aged by heat. CFT CS-38), and POM (pigment/oil/milk, CFT-C10), for laundry-based applications, and egg yolk (egg yolk on polyacryl fabric, aged and colored with carbon black dye. PAS-38) for dish-based applications. Pre-punched (to fit on MPT), rinsed, and filled swatch-containing plates (Corning 3641) were prepared by Center for Testmaterials BV, Vlaardingen. Netherlands. Detergent was added to the microswatch plates prior to enzyme addition. Prior to use, commercial detergents except Persil Non-Bio and Blue Moon were heat-inactivated to remove enzyme activity and then dosed as described in Table 3.

TABLE 3
List of Wash Conditions Used for Performance Assays

| | Final | | | | |
|---|---|---|---|---|---|
| | Wash | Hardness | | | |
| | Conc, | Conc. | | Set | |
| Detergent | (g/L) | (ppm) | Buffer | pH | Type |
| Kirkland | 1.1 | 150 | 2 mM NaCO₃ | 10.6 | Laundry |
| Ultra | | | | | |
| OMO Color | 5.3 | 250 | 2 mM NaCO₃ | 10.6 | Laundry |
| Surf Excel | 4 | 400 | 2 mM NaCO₃ | 10.6 | Laundry |
| Kirkland | 0.17 | 150 | 5 mM sodium | 8.2 | Laundry |
| Ultraclean | | | HEPES | | |
| OMO Klein & | 2.8 | 250 | 5 mM sodium | 8.2 | Laundry |
| Krachtig | | | HEPES | | |
| Persil | 2.7 | 250 | 5 mM sodium | 8 | Laundry |
| Non-Bio | | | HEPES | | |
| Test HDL | 7.5 | 250 | 5 mM sodium | 8 | Laundry |
| | | | HEPES | | |
| Blue Moon | 13 | 250 | 5 mM sodium | 6.5 | Laundry |
| | | | HEPES | | |
| GSM-B | 3 | 374 | Not buffered | 10.5 | Dish |

Laundry cleaning assays with HDL or HDD formulas were carried out at 25° C. for 15-30 min, while dish cleaning assay with GSM-B formula and PAS-38 swatches was carried out at 40° C. for 30 min. Following incubation, 100 uL of supernatant was transferred to a fresh MTP (Costar 9017) and absorbance was measured at 600 nm for EMPA-116 swatches, or at 405 nm for CFT CS-38, CFT CS-10, or PAS-38 swatches using a SpectraMax plate reader. Absorbance results were obtained by subtracting the value for a blank control (no enzyme) from each sample value to determine relative performance.

### Stability Assays

Variants were tested for stability under various stress conditions of buffers or detergents as indicated in Table 4 below by measuring the residual activity following incubation at elevated temperature. The elevated temperature was set to obtain approximately 30% residual activity of the stressed sample compared to the unstressed sample. Diluted enzyme sample was mixed in stressor and unstressed protease activity was measured. Diluted enzyme sample in stressor was incubated at elevated temperature and stressed protease activity was measured either by AAPF. DMC, or skim milk hydrolysis.

The stability assay conditions are described in Table 4 below. The commercial detergents except Persil Non-Bio and Blue Moon were heated to inactivate enzymes prior to use in stability assays:

TABLE 4
Conditions Used for Protease Stability Assays

| | Stress | | |
|---|---|---|---|
| | temp | | |
| Condition | (° C.) | Substrate | Type |
| 0.02% LAS, 2.1 mM EDTA in 50 mM | 53.5 | AAPF/DMC | Laundry |
| HEPES pH 8, 0.005% Tween | | | |
| 10% Kirkland Ultra | 56 | AAPF/DMC | Laundry |
| 10% OMO Klein & Krachtig | 51 | AAPF/DMC | Laundry |
| 10% Persil Non-Bio HDL | 60 | Skim milk | Laundry |
| 10% Test HDL | 60 | DMC | Laundry |
| 50 mM Tris pH 9; 1 mM EDTA; | 51 | AAPF/DMC | Dish |
| 0.005% Tween | | | |
| 50 mM Tris pH 9; 2 mM CaCl₂; | 72.5 | AAPF/DMC | Dish |
| 0.005% Tween | | | |

For the unstressed condition, enzyme was assayed immediately for activity on AAPF, DMC, or skim milk substrates (see above). For the stressed condition, the PCR plate was sealed and incubated at elevated temperature for 5 min using an Eppendorf 385 MasterCycler Pro Thermocycler, then assayed for activity. Stressed and unstressed activity was measured by either the hydrolysis of the synthetic substrate or by the DMC method, as described above. Percent (%) residual activities were calculated by calculating the ratio of the stressed over the unstressed activity and multiplying value by 100. Stability Pls were obtained by dividing the residual activity of a variant by that of the wild type.

### Aged Laundry Cleaning Assay

Each well of a 96 well MTP (Nunc 249946) was filled with a mini stir disc followed by addition of 270 uL 100% Persil Non-Bio detergent. 18.1 uL of enzyme supernatant diluted to −500 ppm was added to the detergent-filled plate and mixed using a magnetic tumbling mixing apparatus (V&P Scientific. VP710 series) for 5 min. For the initial time point (T=0). 25 uL of enzyme containing detergent was transferred to a MTP filled with 125 uL of buffer (5 mM HEPES, 250 ppm 3:1 Ca:Mg water hardness, pH 8). Five uL aliquots of enzyme diluted in detergent was then added to 185 uL buffer (5 mM HEPES, 250 ppm hardness, pH 8) previously added to a MTP containing an EMPA-116 BMI microswatch. At time point 0 (T=0), the cleaning assay was carried out in an analogous manner as described above for fresh cleaning. For the remaining time points, the detergent-enzyme plate was incubated at 55° C. for either: 1.5 h, 5 h, or 24 h; aliquots were removed at the end of each time point; and the cleaning assay carried out in an analogous manner as described above for fresh cleaning. A residual cleaning value was obtained by comparing the cleaning at the various remaining time points to the initial cleaning at T=0.

### Long Term Stability in Boron-free Laundry Detergent

A subset of LG12-clade variants were tested for stability over a span of two weeks in concentrated boron-free HDL detergents: Persil Non-Bio, Blue Moon, All 3X, and Purex Dirt Lift. A boron-free detergent is a detergent to which borate stabilizers have not been added. The amount of boron present in the boron-free detergents of this Example was quantified by elemental analysis, and in all instances, only trace amounts (less than 5 mg) of boron per kg of detergent were detected.

The long term stability experiments were carried out as follows. Purified enzyme was added at 1% w/w to commercially purchased detergent and mixed by gentle pipetting in either a plate or tube-based set up. An initial aliquot of enzyme detergent mixture was removed from each detergent and stored at −20° C. for use as a non-stressed reference. The remaining enzyme detergent samples were placed in a 37° C. incubator to induce stress. Aliquots of the enzyme detergent mixture were removed at various time points from the incubator over the course of the experiment and stored at −20° C. before measuring activity on AAPF substrate. The enzymatic activity of the detergent samples was measured by diluting then with 100 mM Tris pH 8.6, 0.005% Tween-80, 50 mM CaCl₂ buffer 70-150 fold and then assaying on the AAPF substrate as described above in the section on general set up for stability assays. The dilution factor for the detergent samples was adjusted to account for activity loss over the course of the study to remain within the standard curve of BPN′-Y217L subtilisin reference standard.

### EXAMPLE 2

### Generation of LG12-Clade Subtilisins

DNA manipulations to generate LG12-clade subtilisins were carried out via molecular biology techniques known in the art. An E. coli plasmid containing the genes of interest in its expression cassette and the chloramphenicol resistance marker from Staphylococcus aureus (CAT) was constructed. The expression cassette consisted of: B. subtilis aprE promoter, the B. subtilis aprE signal peptide sequence, the B. lentus protease pro-peptide, the region encoding the mature protease sequences for LG12-clade variants (SEQ ID NOs:1 and 3-8 for amino acid sequences) and a B. amyloliquefaciens terminator.

Gibson assembly was performed to assemble the integration cassette containing sequences that target the AprE locus. A suitable B. subtilis strain was transformed with reaction products and the transformed cells were plated on Luria's Agar+5 ppm chloramphenicol containing skim milk. Single colonies from the agar plates were picked into individual wells of a 96-well MTP that contained 150 uL of Luria's broth+5 ppm chloramphenicol in each well. The cultures were grown overnight at 37° C. while being shaken at 250 rpm. 75 uL of 50% glycerol (diluted in water) was added to each well and the MTPs (referred to as library stock plates) frozen at −80° C.

To produce the new proteases, the B. subtilis carrying each gene of interest was cultivated in an enriched semi-defined media for 2 days at 32° C. After centrifugation and filtration, culture supernatants with the novel subtilisins were used for assays and purification.

The mature LG2 wild-type (described as LG-12 SprC in Example 2, page 86, line 16 to page 87, line 14 of PCT Published Application WO2015/038792, filed Sep. 11, 2014, which is hereby incorporated herein by reference) amino acid sequence is set forth in, for example, FIG. 1A-1B as SEQ ID NO:10.

### EXAMPLE 3

### Evaluation of LG12-Clade Protease Variants in Laundry Detergents

### Laundry Cleaning Performance Assay

LG12-clade protease variants listed in Table 5 were tested for cleaning performance relative to BPN′-Y217L protease on BMI (EMPA-116) microswatches. The BPN′-Y217L protease is commercially available and is set forth as SEQ ID NO:48: AQSVPYGVSQIKAPALHSQGYTGSNVKV AVIDSGIDSSHPDLKVAGGASMVPSETNPFQDNNSHGTHVAGTVAALNNSIGVLGVAPSASLYA VKVLGADGSGQYSWIINGIEWAIANNMDVINMSLGGPSGSAALKAAVDKAVASGVVVVAAAG NEGTSGSSSTVGYPGKYPSVIAVGAVDSSNQRASFSSVGPELDVMAPGVSIQSTLPGNKYGALN GTSMASPHVAGAAALILSKHPNWTNTQVRSSLENTTTKLGDSFYYGKGLINVQAAAQKSFPEVV GKTVDQAREYFTLHYPQYDVYFLPEGSPVTLDLRYNRVKVFYNPGTNVVNHVPHVG.

TABLE 5
LG12-clade Protease Variants

| | | No. |
|---|---|---|
| Sample | | mutations |
| ID | Sequence Substitutions Relative to LG12 WT | vs LG12 |
| LG12- | A001T-N076D-T078S-T079I-Y086P-D089E- | 33 |
| CP022 | S097G-T103S-L104V-G106S-I111L-S114A- | |
| | I115G-S116N-N120H-I122A-M124L-G128S- | |
| | S129P-G131P-T133A-A134T-Q136E-C139V- | |
| | N141S-Y143T-N144S-I147V-V148L-I150V- | |
| | S158T-G166D-E248N | |
| LG12- | S052A-N076D-T078S-T079I-Y086P-D089E- | 34 |
| CP180 | S097G-T103S-L104V-G106S-I111L-S114A- | |
| | I115G-S116N-N120H-I122A-M124L-G128S- | |
| | S129P-G131P-T133V-A134T-Q136E-C139V- | |
| | N141S-Y143T-N144S-I147V-V148L-I150V- | |
| | S158T-G166D-F217L-E248N | |
| LG12- | A001T-N076D-T078S-T079I-Y086P-D089E- | 42 |
| CP446 | S097G-T103S-L104V-G106S-I111L-S114A- | |
| | I115G-S116N-N120H-I122A-M124L-G128S- | |
| | S129P-G131P-T133V-A134T-Q136E-C139V- | |
| | N141S-Y143T-N144S-I147V-V148L-I150V- | |
| | S158T-G166D-T209Y-N212S-N213T-S216T- | |
| | F217L-A224T-E248N-K251S-N252S-N256Y | |
| LG12- | N076D-T078S-T079I-Y086P-D089E-S097G- | 38 |
| CP455 | T103S-L104V-G106S-I111L-S114A-I115G- | |
| | S116N-N120H-I122A-M124L-G128S-S129P- | |
| | G131P-T133A-A134T-Q136E-C139V-N141S- | |
| | Y143T-N144S-I147V-V148L-I150V-S158T- | |
| | G166D-T209Y-N212S-N213T-S216T-F217L- | |
| | A224T-E248N | |
| LG12- | N076D-T078S-T079I-Y086P-N087S-A088V- | 35 |
| CP460 | D089S-S097G-T103S-L104V-G106S-I111L- | |
| | S114A-I115G-S116N-N120H-I122A-M124L- | |
| | G128S-S129P-G131P-T133A-A134T-Q136E- | |
| | C139V-N141S-Y143T-N144S-I147V-V148L- | |
| | I150V-S158T-G166D-F217L-E248N | |
| LG12- | A001T-N076D-T078S-T079I-Y086P-D089E- | 39 |
| CP462 | S097G-T103S-L104V-G106S-I111L-S114A- | |
| | I115G-S116N-N120H-I122A-M124L-G128S- | |
| | S129P-G131P-T133A-A134T-Q136E-C139V- | |
| | N141S-Y143T-N144S-I147V-V148L-I150V- | |
| | S158T-C166D-T209Y-N212S-N213T-S216T- | |
| | F217L-A224T-E248N | |
| LG12- | A001T-N076D-T078S-T079I-Y086P-N087S- | 40 |
| CP465 | A088V-D089S-S097G-T103S-L104V-G106S- | |
| | I111L-S114A-I115G-S116N-N120H-I122A- | |
| | M124L-G128S-S129P-G131P-T133A-A134T- | |
| | Q136E-C139V-N141S-Y143T-N144S-I147V- | |
| | V148L-I150V-S158T-G166D-T209Y-N212S- | |
| | N213T-S216T-F217L-E248N | |

The detergents used were commercially available Persil Non-Bio HDL (Unilever) at a final concentration of 2.7 g/L and Test HDL at a final concentration of 7.5 g/L. In both instances, the water hardness concentration was set at 250 ppm, using 5 mM sodium HEPES buffer to achieve pH 8.

Aliquots of purified enzyme were added to the detergent-filled microswatch plates to reach a final volume of 200 uL with a final enzyme concentration of 0.62 ppm. Assays were carried out at 25° C. for 30 min. Following incubation, 100 uL of supernatant was transferred to a fresh MTP (Costar 9017) and absorbance was read at 600 nm using the SpectraMax plate reader. Absorbance results were obtained by subtracting the value for a blank control (no enzyme) from each sample value. The cleaning PI for each assay condition was obtained by dividing the absorbance values for a given variant by that of the control (BPN′-Y217L).

Table 6 shows the results obtained when the cleaning and stability of various LG12-clade protease variants were compared to the benchmark BPN′-Y217L in non-boron containing detergents.

TABLE 6
Comparison of Cleaning and Stability of LG12-clade Protease
Variants to BPN′-Y217L in Non-boron Containing Detergents

| | Microswatch Cleaning | | Stability in 10% HDL, 60° C. | |
|---|---|---|---|---|
| | (PI vs BPN′-Y217L) | | (% Residual protease activity) | |
| | Persil Non- | | Persil Non- | |
| Variant | Bio HDL | Test HDL | Bio HDL | Test HDL |
| LG12-CP022 | 0.8 | 0.7 | 74.6 | 56.8 |
| LG12-CP180 | 0.9 | 0.7 | 69.2 | 44.0 |
| LG12-CP446 | 0.7 | 0.8 | 82.6 | 74.5 |
| LG12-CP455 | 0.9 | 0.8 | 81.2 | 66.4 |
| LG12-CP460 | 0.9 | 0.8 | 88.2 | 70.1 |
| LG12-CP462 | 0.9 | 0.8 | 86.0 | 64.2 |
| LG12-CP465 | 0.9 | 0.9 | 102.4 | 58.0 |
| BPN′-Y217L | 1.0 | 1.0 | 7.6 | 10.4 |

Table 6 data shows that following incubation in non-boron containing HDL detergents and heat stress, the variants: LG12-CP022, LG12-CP180, LG12-CP446, LG12-CP455, LG12-CP460, LG12-CP462, and LG12-CP465 all had significantly improved residual activity over BPN′-Y217L. These variants also retain a significant amount of cleaning efficiency while gaining on average 5-10 fold improvement in detergent stability.

### EXAMPLE 4

### Sequence Comparison of LG12-Clade Protease Variants and LG12 Wild-Type Protease

The sequences of the LG12-clade protease variants of Example 3 (i.e., LG12-CP022 (SEQ ID NO:1), LG12-CP180 (SEQ ID NO:3), LG12-CP446 (SEQ ID NO:4), LG12-CP455 (SEQ ID NO:5), LG12-CP460 (SEQ ID NO:6), LG2-CP462 (SEQ ID NO:7), and LG12-CP465 (SEQ ID NO:8)) were compared to the parent protease sequence of LG12 wild-type (SEQ ID NO:10). Clustal W sequence alignment, shown in FIG. 1, identifies regions of the amino acid sequence where changes occurred.

The LG12-CP022 (SEQ ID NO:1), LG12-CP180 (SEQ ID NO:3), LG12-CP446 (SEQ ID NO:4), LG12-CP455 (SEQ ID NO:5), LG12-CP460 (SEQ ID NO:6), LG12-CP462 (SEQ ID NO:7), and LG12-CP465 (SEQ ID NO:8) protease variants had superior stability and significant cleaning performance in laundry conditions. Most of these LG12-clade protease variants share 31 substitutions at the following positions: 76, 78, 79, 86, 97, 103, 104, 106, 111, 114, 115, 116, 120, 122, 124, 128, 129, 131, 134, 136, 139, 141, 143, 144, 147, 148, 150, 158, 166, 217 and 248, wherein the amino acid positions are numbered by correspondence with the linear LG12 wild-type (SEQ ID NO: 10) amino acid sequence numbering. Furthermore, one or more LG12-clade protease variants share one or more of the following substitutions: N76D, T78S. T79I. Y86P. S97G, T103S. L104V, G106S, I111L, S114A, I115G, S116N, N120H, I122A, M124L, G128S, S129P, G131P, A134T, Q136E, C139V, N141S, Y143T, N144S, I147V, V148L, I150V, S158T, G166D, F217L and E248N.

These sites are distributed primarily in three regions, two of which are structurally closely related. The overall protein fold observed on a crystallographic structure of LG12 wild-type protease is shown in FIG. 2.

FIG. 2 highlights key elements in the LG12 wild-type protease crystallographic model. The overall folding is represented as a schematic in light gray. The characteristic catalytic triad of the subtilisin proteases formed by Aspartic acid (D) 32, Histidine (H) 64 and Serine (S) 221 is shown as black stick figures. These 3 residues are found in a large canyon-like surface formed by segments comprising residues 97-104, 126-130, 150-170, and 207-220. The two calcium sites common to all commercial bacterial subtilisins are represented as dark gray spheres. The key loops forming the substrate binding region are highlighted as black lines and include residues 97-104, 126-130, 150-170 and 207-220. The 97-104 and 126-130 segments are followed by large helices comprising residues 105-117 and 131-145.

FIG. 3 provides a diagram of the 3D structure of the LG12 wild-type protease depicting (i) side chains of substitutions common to the best LG12-Clade protease variants, which substitutions are primarily localized in two segments that form the walls of the substrate binding site and the following helical segments that serve to anchor the segment in structure residues 97-148, (ii) positions 158, 166 and 217, which line the substrate binding cavity, and (iii) positions 76, 78, 79 and 86, which are near one of the critical calcium binding sites.

As can be seen in the image shown in FIG. 3, residues 97, 103, 104, 106, 111, 114, 115, 116, 120, 122, 124, 128, 129, 131, 134, 136, 139, 141, 143, 144, 147, 148, 150, and 158 are found either in the substrate binding region or in the helices following the highlighted segments. As a result, substitutions at these positions may contribute stabilizing inter-helical interactions and position these helices to act in concert with the substitutions in the substrate binding canyon to increase the overall stability and cleaning performance. Other beneficial mutations can be made at positions 76, 78, 79 and 86, which are localized near the tight calcium binding site common to all Bacillus subtilisins, as shown in FIG. 3. In addition, including mutations at position 158, 166 and 217 is shown to impart significant benefits.

### EXAMPLE 5

### Generation of Further LG12-Clade Protease Variants

Design of additional LG12-clade protease variants was performed using information from the site evaluation library screen that is described in PCT Published Application No. WO2015/038792, filed Sep. 11, 2014. From 2 to 23 beneficial mutations were introduced into the LG12 wild-type protease resulting in LG12-Clade protease variants with improved cleaning performance and stability.

A suitable B. subtilis host strain was transformed with expression plasmids containing the variants. The variants were grown in 96-well MTPs in cultivation medium (enriched semi-defined media based on MOPs buffer, with urea as major nitrogen source, glucose as the main carbon source, and supplemented with 1% soytone for robust cell growth) containing 25 ppm tetracycline in square enzyscreen plates for 2 days at 32° C., 300 rpm, with 80% humidity in shaking incubator.

### EXAMPLE 6

### Cleaning Performance and Stability of LG12-Clade Variant Proteases

Additional LG12-clade protease variants generated as described in Example 5 above were screened in MTP assays (as described in Example 1) to compare their cleaning performance and stability to parent molecule LG12 wild-type protease. All variants exhibited expression (HPLC assay) and proteolytic activity on DMC substrate on par with LG12 wild-type protease (SEQ ID NO:10). The variants were grouped according to improved properties. Table 7 sets forth the LG12-clade protease variants constructed and tested.

TABLE 7
LG12-clade Variant Proteases

| | | No. |
|---|---|---|
| Sample | | mutations |
| ID | Sequence Substitutions Relative to LG12 WT | vs. LG12 |
| LG12- | A001G-S024R-I030V-A048Q-S052Q-G053N- | 13 |
| 05502 | S114T-G128N-V149N-F217R-A228S-A236N- | |
| | S272K | |
| LG12- | A001G-S052N-L090P-N117S-V149G-S159E- | 8 |
| 05503 | Y238F-S272N | |
| LG12- | T003V-P009V-A019R-S024T-I030V-A040L- | 20 |
| 05505 | A057P-N076D-S101T-I107V-S116E-I147A- | |
| | S156A-G166Y-T185Q-S194P-A215E-S240F- | |
| | N256K-S272K | |
| LG12- | A001G-A048Q-S052T-T078G-Y091H-S114T- | 17 |
| 05517 | N117S-I147A-S156N-S159N-G166H-F217R- | |
| | M222L-A236N-Y238F-Q245N-V267N | |
| LG12- | A001G-K027T-N038Q-A040L-N043S-S052Q- | 18 |
| 05521 | G053D-G100K-G128N-R145N-V149N-S159D- | |
| | S181A-F217R-K251R-N256R-E271W-S272Q | |
| LG12- | A001G-P009T-A019R-N038A-A057P-V072A- | 20 |
| 05523 | N076D-S099L-T103E-S114T-R145W-V149S- | |
| | S156N-G166H-T185Q-S194P-F217R-Y238F- | |
| | K265S-S272K | |
| LG12- | N123D-S159D-T209F-M222L-V267N | 5 |
| 05524 | | |
| LG12- | A001G-P009I-A019R-S024T-K027T-I030V- | 20 |
| 05525 | N038Q-N043H-A048Q-G053D-S101T-I107T- | |
| | G128N-T133D-V149S-T185Q-A228S-T242S- | |
| | K251R-S272K | |
| LG12- | K045Q-N123D-S156N-T209F-N256P | 5 |
| 05526 | | |
| LG12- | A001G-A048Q-S052T-T078G-Y091H-S114T- | 16 |
| 05527 | N117S-I147A-S156N-S159N-G166H-M222L- | |
| | A236N-Y238F-Q245N-V267N | |
| LG12- | A001G-I030V-S052Q-G053N-S114T-G128N- | 11 |
| 05534 | V149N-S159N-F217R-A228S-A236N | |
| LG12- | A001G-A048Q-S052T-T078G-Y091H-S114T- | 17 |
| 05535 | N117S-I147A-S156N-S159N-G166H-M222L- | |
| | A236N-Y238F-Q245N-N256E-V267N | |
| LG12- | A001G-I030V-A040E-N043D-A048Q-S052Q- | 19 |
| 05536 | G053N-S114T-G128N-V149N-I150V-S156N- | |
| | S159D-G166N-S181A-S216T-M222L-A228S- | |
| | A236N | |
| LG12- | A001G-T003E-A040D-A048Q-S052T-G053E- | 23 |
| 05537 | T078G-Y091H-S114T-N117S-I147A-S156N- | |
| | S159D-G166H-S182E-A215D-F217R-M222L- | |
| | A236N-Y238F-Q245N-N256E-V267N | |
| LG12- | A001G-S024R-I030V-A048Q-S052Q-G053N- | 14 |
| 05540 | S114T-G128N-V149N-S159D-F217R-A228S- | |
| | A236N-S272K | |
| LG12- | A001G-K027S-N038T-G053E-T078D-L090P- | 13 |
| 05541 | V093T-T103E-S159D-G166S-T185Q-F217R- | |
| | A236N | |
| LG12- | A001G-K027T-A040L-N043S-S052Q-G053D- | 15 |
| 05542 | V149N-S159D-G166Y-S181A-F217R-M222L- | |
| | N256R-E271W-S272Q | |
| LG12- | T003V-N043L-G053E-A057P-V072A-N076D- | 19 |
| 05543 | S101T-I107T-S116E-I147A-S159Q-G166A- | |
| | S188Y-S194P-F217R-A236Q-S240F-1246V- | |
| | S272Q | |
| LG12- | A001G-T003E-A040D-A048E-S052T-G053E- | 22 |
| 05546 | T078G-Y091H-S114T-N117S-I147A-S156N- | |
| | S159D-G166H-S182E-A215D-M222L-A236N- | |
| | Y238F-Q245N-N256E-V267N | |
| LG12- | A001G-K027T-N038Q-A040L-N043S-S052Q- | 19 |
| 05552 | G053D-N117E-R145N-V149N-S159D-G166Y- | |
| | S181A-F217R-M222L-K251R-N256R-E271W- | |
| | S272Q | |
| LG12- | A001G-A040D-S052T-T078G-Y091H-S114T- | 17 |
| 05553 | N117S-I147A-S156N-S159N-G166H-S182E- | |
| | M222L-A236N-Y238F-Q245N-V267N | |
| LG12- | A001G-A048Q-S052Q-G053N-S114T-G128N- | 9 |
| 05557 | S159N-S216T-A228S | |
| LG12- | A001G-P009T-S024T-K027S-A057P-V072A- | 20 |
| 05559 | N076D-S101T-S116E-N140Y-V149N-S159N- | |
| | G166N-T185Q-S188Y-S194P-1234V-S240F- | |
| | K251R-E271F | |
| LG12- | A001G-I030V-N043D-A048Q-S052Q-G053N- | 13 |
| 05561 | S114T-G128N-V149N-S159N-S216T-A228S- | |
| | A236N | |
| LG12- | A001G-I030V-A040V-N076D-T078D-T103E- | 9 |
| 05563 | G166S-T185Q-F217R | |
| LG12- | A001G-K027S-A040V-G053N-T078D-L090P- | 11 |
| 05565 | T103E-S156N-S159D-G166S-F217R | |
| LG12- | N038Q-G100A-N117S-I147A-S158T-G166D- | 8 |
| 05566 | N256P-S272K | |
| LG12- | A001G-I030V-A048Q-S0520-G053N-S114T- | 16 |
| 05567 | G128N-V149N-I150V-S156N-S159N-S181A- | |
| | S216T-M222L-A228S-A236N | |
| LG12- | N038Q-G100A-N117S-I147A-S158T-N256P- | 7 |
| 05569 | S272K | |
| LG12- | A001G-I030V-N043D-A048E-S052Q-G053N- | 13 |
| 05571 | S114T-G128N-V149N-S159D-S216T-A228S- | |
| | A236N | |
| LG12- | A001G-K027T-N038Q-A040L-N043S-S052Q- | 18 |
| 05576 | G053D-N117E-R145N-V149N-S159D-G166Y- | |
| | S181A-F217R-M222L-K251R-N256R-E271W | |
| LG12- | S024R-1030V-A040E-N043D-A048E-S052Q- | 21 |
| Q5577 | G053N-S114T-G128N-V149N-I150V-S156N- | |
| | S159D-G166D-S181A-S216T-F217R-A228S- | |
| | A236N-E271W-S272K | |
| LG12- | N076D-G118R-G128A-S129M-S130T-G166D- | 7 |
| 05626 | E248N | |
| LG12- | N076D-G118R-G128D-S129E-S130L-G166D- | 7 |
| 05652 | E248N | |
| LG12- | N076D-G118R-G128D-S129K-S130H-G166D- | 7 |
| 05657 | E248N | |
| LG12- | N076D-G118R-G128D-S129M-S130H-G166D- | 7 |
| 05659 | E248N | |
| LG12- | N076D-G118R-G128D-S129N-S130M-G166D- | 7 |
| 05661 | E248N | |
| LG12- | N076D-G118R-G128D-S129N-S130Y-G166D- | 7 |
| 05662 | E248N | |
| LG12- | N076D-G118R-G128D-S129R-S130I-G166D- | 7 |
| 05668 | E248N | |
| LG12- | N076D-G118R-G128D-S129R-S130T-G166D- | 7 |
| 05670 | E248N | |
| LG12- | N076D-G118R-G128D-S130N-G166D-E248N- | 7 |
| 05671 | G160D | |
| LG12- | N076D-G118R-G128D-S129T-S130F-G166D- | 7 |
| 05672 | E248N | |
| LG12- | N076D-G118R-G128D-S129Y-S130R-G166D- | 7 |
| 05673 | E248N | |
| LG12- | N076D-G118R-G128E-S129G-S130Y-G166D- | 7 |
| 05680 | E248N | |
| LG12- | N076D-G118R-G128E-S129H-S130T-G166D- | 7 |
| 05681 | E248N | |
| LG12- | N076D-G118R-G128E-S129V-S130M-G166D- | 7 |
| 05687 | E248N | |
| LG12- | N076D-G118R-S129D-S130A-G166D-E248N | 6 |
| 05707 | | |
| LG12- | N076D-G118R-S129T-S130A-G166D-E248N | 6 |
| 05710 | | |
| LG12- | N076D-G118R-S129V-S130Q-G166D-E248N- | 7 |
| 05720 | Q275R | |
| LG12- | N076D-G118R-G128H-S129L-S130L-G166D- | 7 |
| 05731 | E248N | |
| LG12- | N076D-G118R-G128K-S129I-S130N-G166D- | 7 |
| 05781 | E248N | |
| LG12- | N076D-G118R-G128N-S129G-S130N-G166D- | 7 |
| 05829 | E248N | |
| LG12- | N076D-G118R-G128N-S129M-S130G-G166D- | 7 |
| 05831 | E248N | |
| LG12- | N076D-G118R-G128N-S129N-S130H-G166D- | 7 |
| 05832 | E248N | |
| LG12- | N076D-G118R-G128N-S129T-S130Q-G166D- | 7 |
| 05837 | E248N | |
| LG12- | N076D-G118R-G128N-S129V-S130V-G166D- | 7 |
| 05838 | E248N | |
| LG12- | N076D-G118R-G128N-S129Y-S130M-G166D- | 7 |
| 05839 | E248N | |
| LG12- | N076D-G118R-G128Q-S129M-S130H-G166D- | 7 |
| 05863 | E248N | |
| LG12- | N076D-G118R-G128S-S129E-S130W-G166D- | 7 |
| 05896 | E248N | |
| LG12- | N076D-G118R-G128S-S129H-S130Q-G166D- | 7 |
| 05899 | E248N | |
| LG12- | N076D-G118R-G128S-S129I-S130R-G166D- | 7 |
| 05901 | E248N | |
| LG12- | N076D-G118R-G128S-S129L-S130E-G166D- | 7 |
| 05902 | E248N | |
| LG12- | N076D-G118R-G128S-S129L-S130M-G166D- | 7 |
| 05904 | E248N | |
| LG12- | N076D-G118R-G128S-S129N-S130V-G166D- | 7 |
| 05908 | E248N | |
| LG12- | N076D-G118R-G128S-S129Q-G166D-E248N | 6 |
| 05911 | | |
| LG12- | N076D-G118R-G128S-S129V-S130L-G166D- | 7 |
| 05912 | E248N | |
| LG12- | N076D-G118R-G128Y-S129G-S130T-G166D- | 7 |
| 06006 | E248N | |
| LG12- | G118R-G128M-S129L-S130Q-G166D-E248N | 6 |
| 06029 | | |
| LG12- | G166H-S159D | 2 |
| 06520 | | |
| LG12- | N038T-N140D-G166Y-T185Q-S159D | 5 |
| 06556 | | |
| LG12- | N038T-S156H-G166Y-T185Q-S272Q-S159D | 6 |
| 06575 | | |
| LG12- | N038T-S156H-G166Y-S272Q-S159D | 5 |
| 06582 | | |
| LG12- | N140D-V149L-S156H-G166N-S272Q | 5 |
| 06593 | | |
| LG12- | N140D-V149L-G166Y-T185Q-S159D | 5 |
| 06596 | | |
| LG12- | N038T-N140D-V149L-S156H-G166Y-T185Q- | 8 |
| 06608 | S272Q-S159D | |
| LG12- | N140D-G166Y-T185Q-S159D | 4 |
| 06612 | | |
| LG12- | A040A-V149V-S156N-G166A-S181S-S182E- | 8 |
| 06801 | S194S-N256N | |
| LG12- | G053D-N076D-G166D-S272K | 4 |
| 06924 | | |
| LG12- | G053D-N076D-G118G-G166D-E248E-N256N- | 7 |
| 06925 | S272S | |
| LG12- | G053D-N076D-G166D-N256R-S272K | 5 |
| 06929 | | |
| LG12- | G053D-G166D-S272K | 3 |
| 06948 | | |
| LG12- | G053D-G166D | 2 |
| 06949 | | |
| LG12- | G053D-G118R-G166D | 3 |
| 06954 | | |
| LG12- | N076D-G166D-S272K | 3 |
| 06960 | | |
| LG12- | N076D-G166D-N256R | 3 |
| 06961 | | |
| LG12- | N076D-G166D-E248K | 3 |
| 06964 | | |
| LG12- | N076D-G118R-G166D | 3 |
| 06969 | | |
| LG12- | G166D-S272K | 2 |
| 06978 | | |
| LG12- | G118R-G166D-N256R-S272K | 4 |
| 06992 | | |
| LG12- | G053D-G118R-G166D-S272K | 4 |
| 07003 | | |
| LG12- | G053D-G118R-G166D-N256R-S272K | 5 |
| 07004 | | |
| LG12- | G053D-N076D-G166D-E248K-S272K | 5 |
| 07016 | | |
| LG12- | N076D-G166D | 2 |
| 07024 | | |
| LG12- | N076D-G118R-G166D-N256R-S272K | 5 |
| 07027 | | |
| LG12- | K027S-G053E-S159D-N256P | 4 |
| 07133 | | |
| LG12- | K045Q-G053E-S159D-N256P | 4 |
| 07143 | | |
| LG12- | P009T-K045R-A048E-T078A-G128N-N140D- | 13 |
| 07158 | S158V-S181N-S194P-T209V-Q245A-N256P- | |
| | S272Q | |

A set of LG2-clade protease variants with improved stability in 10% OMO HDL detergent over LG12 wild-type protease parent molecule had a PI>1.01 for stability in LAS/EDTA and 10% OMO HDL detergent, and a PI>0.75 for stability in 10% Kirkland HDL detergent. These LG12-clade variants are: LG12-07133, LG12-05524, LG12-05526, LG12-05575, LG12-05569, LG12-05503, LG12-05566. LG12-07075, LG12-05557, LG12-05563, LG12-05534, LG12-05565, LG12-05502, LG12-05541, LG12-05561, LG12-05571, LG12-07158, LG12-05540, LG12-05542, LG12-05527, LG12-05567, LG12-05517, LG12-05535, LG12-05553, LG12-05521, LG12-05576, LG12-05536, LG12-05543, LG12-05552, LG12-05505, LG12-05523, LG12-05525, LG2-05559, LG12-05577, LG12-05546, and LG12-05537.

A set of LG12-clade protease variants with improved laundry cleaning in HDD detergent over LG12 wild-type protease parent molecule had a PI>1.01 in Kirkland Ultra HDD detergent. OMO color HDD detergent, and in Surf Excel HDD for those variants marked with an asterisk (*). These LG12-clade variants are: LG12-05526*, LG2-05569*, LG12-05626, LG12-05652, LG12-05657, LG12-05659. LG12-05661, LG12-05662, LG12-05668, LG12-05670, LG12-05672, LG12-05673, LG12-05680, LG12-05681, LG12-05687, LG12-05707, LG12-05710, LG12-05731LG12-05781LG12-05829, LG12-05831, LG12-05832, LG12-05837, LG12-05838, LG12-05839, LG12-05863, LG12-05896, LG12-05899, LG12-05901, LG12-05902, LG12-05904, LG12-05908, LG12-05911, LG12-05912, LG12-06006, LG12-06029, LG12-06924, LG12-06929, LG12-06948, LG12-06949, LG12-06954, LG12-06960, LG12-06961, LG12-06964, LG12-06969, LG12-06978, LG12-06992, LG12-07003, LG12-07004, LG12-07016, LG12-07024, LG12-07027, LG12-05566*, LG12-05671, LG1205720, LG12-06593, LG12-07110*, LG12-05522*, LG12-06520, LG12-06556, LG12-06575, LG12-06582, LG12-06596, LG12-06608, LG12-06612, and LG12-05540*.

A set of LG12-clade protease variants with improved stability and laundry cleaning across all assays over LG12 wild-type protease parent molecule had a PI value >1.01 in all laundry detergents, including Blue Moon HDL. These LG12-clade variants are: LG12-05566, LG12-07065, LG12-07110, LG12-05522, and LG12-05540.

A set of LG12-clade protease variants with improved ADW cleaning over LG12 wild-type protease parent molecule had a PI value >1.01 in GSM-B detergent. These LG2-clade variants are: LG12-05520, LG12-05566, LG12-05569, LG12-05626, LG12-05652, LG12-05657, LG12-05659, LG12-05661, LG12-05662, LG12-05668, LG12-05670, LG12-05671, LG12-05672, LG12-05673, LG12-05680, LG12-05681, LG12-05687, LG12-05707, LG12-05710, LG12-05720, LG12-05731, LG12-05781, LG12-05829 LG12-05831, LG12-05832, LG12-05837, LG12-05838, LG12-05839, LG12-05863, LG12-05896, LG12-05899, LG12-05901, LG12-05902, LG12-05904, LG12-05908, LG12-05911, LG12-05912, LG12-06006, LG12-06029, LG12-06924, LG12-06925, LG12-06929, LG12-06943, LG12-06948, LG12-06949, LG12-06954, LG12-06960, LG12-06961, LG12-06964, LG12-06969, LG12-06978, LG12-06992, LG12-07003, LG12-07004, LG12-07016, LG12-07024, LG12-07027, LG12-07051, LG12-07058, LG12-07098, LG12-07110, LG12-07115, LG12-07118, LG12-07143, LG12-07144, LG12-07158, LG12-07166, and LG12-07194.

A set of LG2-clade protease variants with improved stability and ADW cleaning over LG12-clade wild-type protease parent molecule had a stability PI>1.2 in buffer (50 mM Tris pH9-2 mM CaCl₂ and Tris pH9-1 mM EDTA as described in Table 4) and an ADW cleaning PI>1.0. These LG12-clade variants are: LG12-05566, LG12-05569, LG12-05657, LG12-05668, LG12-05670, LG12-06006, LG12-06029, LG12-06929, LG12-06948, LG12-06949, LG12-06954, LG12-06960, LG12-06961, LG12-06978, LG12-06992, LG12-07003, LG12-07004, LG12-07027, LG12-07143, LG12-07158, LG12-07051, LG12-07058, LG12-07098, LG12-07110, LG12-07115, LG12-07118, and LG12-07166.

Substitutions that occurred frequently in the above improved LG2-clade protease variants include: S052, G053, G118, S129, S130, V149, S156, S159, G166, E248, N256 and S272, which can be observed in further combination with mutations N076D and/or F217R.

### EXAMPLE 7

### Further Evaluation of LG12-Clade Variants

Additional LG12-clade protease variants listed in Table 8 were synthesized and expressed in B. subtilis as described in Example 2.

TABLE 8
LG12-clade Variants Purified for Further Analysis

| | | No. |
|---|---|---|
| Sample | | mutations |
| ID | Sequence Substitutions Relative to LG12 WT | vs LG12 |
| LG12- | G053D-N076D-G166D | 3 |
| 05520 | | |
| LG12- | N038Q-G053E-G100A-N117S-I147A-S158T- | 9 |
| 05522 | G166D-N256P-S272K | |
| LG12- | A001G-1030V-T078D-V093T-T103E-S156N- | 11 |
| 05547 | S159D-G166S-S194P-F217R-A236N | |
| LG12- | S024T-G053E-G166D-M222L-N256P | 5 |
| 05575 | | |
| LG12- | V149L-S156H-T185Q-S272Q-S159D | 5 |
| 06491 | | |
| LG12- | G053D-N076D-G118R-G166D-N256R | 5 |
| 06943 | | |
| LG12- | N038Q-G053E-G100A-N117S-I147A-S158T- | 8 |
| 07051 | G166D-S272K | |
| LG12- | S052T-V149G-S156N-G166H-F217R | 5 |
| 07058 | | |
| LG12- | A001G-S052Q-V149G-S156N-S159N-S182E- | 8 |
| 07065 | Y238F-S272Q | |
| LG12- | A001G-S052Q-S114T-S156N-S159N-S182E- | 8 |
| 07075 | Y238F-S272Q | |
| LG12- | S052T-V149G-S156N-S159D-G166D-F217R | 6 |
| 07098 | | |
| LG12- | N038Q-G053E-N117S-I147A-S158T-G166D- | 8 |
| 07110 | N256P-S272K | |
| LG12- | A040D-S114T-I147A-S159N-G166D-S182E | 6 |
| 07115 | | |
| LG12- | S052T-V149G-S156N-G166D-S182E-F217R | 6 |
| 07118 | | |
| LG12- | S052T-V149G-S156N-G166D-F217R | 5 |
| 07166 | | |

For laundry cleaning assays, aliquots of purified enzyme were added to a detergent-filled microswatch plate to reach a final volume of 200 uL for laundry assays with a final enzyme concentration between 5-0.5 ppm and tested as described in Example 1. Samples of LG112 wild-type parent protease and BPN′-Y217L benchmark protease were also evaluated. The microswatch cleaning for purified LG12-clade protease variants in BMI laundry assays is set forth in Table 9A. PIs were calculated versus LG12 wild-type parent protease.

TABLE 9A
Comparison of Cleaning Performance of Various LG12-
clade Protease Variants in HDD and HDL Laundry Detergents
to LG12 WT and BPN′-Y217L, Reported as PI

| | Kirkland | OMO | Surf | OMO | Kirkland | Blue Moon |
|---|---|---|---|---|---|---|
| Sample ID | HDD | HDD | Excel | HDL | HDL | HDL |
| LG12 WT | 1.0 | 1.0 | 1.0 | 1.0 | 1.0 | 1.0 |
| LG12-05520 | 1.1 | 0.9 | 1.0 | 1.4 | 1.1 | 5.4 |
| LG12-05547 | 0.7 | 0.4 | 0.5 | 1.0 | 0.7 | 3.3 |
| LG12-06491 | 1.6 | 1.4 | 1.1 | 1.9 | 1.7 | 2.4 |
| LG12-06943 | 2.5 | 1.6 | 1.8 | 1.8 | 2.2 | 3.7 |
| LG12-07051 | 2.2 | 0.6 | 0.9 | 2.5 | 2.2 | 4.8 |
| LG12-07058 | 1.2 | 1.1 | 0.9 | 0.9 | 1.0 | 1.0 |
| LG12-07098 | 1.6 | 0.7 | 0.9 | 1.2 | 1.1 | 2.8 |
| LG12-07115 | 1.0 | 0.8 | 0.9 | 1.0 | 1.0 | 4.1 |
| LG12-07118 | 1.3 | 0.8 | 0.8 | 1.1 | 0.9 | 2.5 |
| LG12-07166 | 1.7 | 1.3 | 1.1 | 1.6 | 1.5 | 1.7 |
| BPN′-Y217L | 0.5 | 0.1 | 0.2 | 1.25 | 1.4 | 1.45 |

The stability of several purified LG12-clade protease variants was measured as described in Example 1, Table 4, in LAS/EDTA and 10% HDL (Kirkland and OMO) and compared to LG12 wild-type parent protease and BPN′-Y217L benchmark protease. The results are shown in Table 9B below.

TABLE 9B
Comparison of Stability of Various LG12-clade Protease
Variants in 10% HDL Laundry Detergents and in LAS/EDTA
to LG12 WT and BPN′-Y217L, Reported as PI

| Sample ID | LAS/EDTA | 10% OMO HDL | 10% Kirkland HDL |
|---|---|---|---|
| LG12 WT | 1.0 | 1.0 | 1.0 |
| LG12-05520 | 1.7 | 1.4 | 1.0 |
| LG12-05547 | 2.2 | 1.4 | 1.2 |
| LG12-06491 | 4.1 | 3.0 | 2.3 |
| LG12-06943 | 2.1 | 2.7 | 2.6 |
| LG12-07051 | 4.1 | 3.4 | 3.2 |
| LG12-07058 | 2.1 | 2.7 | 2.6 |
| LG12-07098 | 2.0 | 1.2 | 1.1 |
| LG12-07115 | 1.8 | 1.2 | 1.0 |
| LG12-07118 | 1.9 | 1.2 | 1.0 |
| LG12-07166 | 2.8 | 2.8 | 1.8 |
| BPN′-Y217L | 3.6 | 3.0 | 0.3 |

For ADW cleaning assays, aliquots of clarified culture supernatants were added to GSM-B detergent-filled microswatch (PAS-38) plate and tested as described in Example 1. The stability of these molecules was also tested in 50 mM Tris pH9-2 mM CaCl₂ and Tris pH9-1 mM EDTA, as described in Example 1. Results for ADW cleaning and stability are shown in Table 9C below.

TABLE 9C
Comparison of ADW Cleaning Performance of Various
LG12-clade Protease Variants vs LG12 WT in GSM-B
Detergent and Stability Assays, Reported as PI

| | ADW | | |
|---|---|---|---|
| Sample ID | Cleaning | Stability TRIS-CA | Stability TRIS-EDTA |
| LG12 WT | 1.0 | 1.0 | 1.0 |
| LG12-05668 | 2.2 | 2.2 | 1.9 |
| LG12-07051 | 1.1 | 2.1 | 2.1 |
| LG12-07058 | 1.3 | 1.6 | 2.3 |
| LG12-07098 | 1.5 | 1.7 | 2.2 |
| LG12-07110 | 1.6 | 2.0 | 2.1 |
| LG12-07115 | 1.3 | 1.7 | 2.0 |
| LG12-07118 | 1.5 | 1.8 | 2.6 |
| LG12-07166 | 1.6 | 2.0 | 2.6 |

The stability of several LG12-clade protease variants was also measured under long-term accelerated stability conditions in various boron-free detergents, and compared to LG12 wild-type parent protease and BPN′-Y217L benchmark protease. The results are shown in Table 10. These detergents represent a cross-section of geographies and formulations, and results indicate that stability improvements can be made for all the formulations examined.

The long term stability experiments were carried out as follows. Purified enzyme was added at 1% w/w to commercially purchased detergent and mixed by gentle pipetting in either a plate or tube-based set up. An initial aliquot of enzyme detergent mixture was removed from each detergent and stored at −20° C. for use as a non-stressed reference. The remaining enzyme detergent samples were placed in a 37° C. incubator to induce stress. Aliquots of the enzyme detergent mixture were removed at various time points from the incubator over the course of the experiment and stored at −20° C. before measuring activity on AAPF. The enzymatic activity of the detergent samples was measured by diluting with 100 mM Tris pH 8.6, 0.005% Tween-80, 50 mM CaCl₂ buffer 70-150 fold before assaying on the AAPF substrate as described above in Example 1. The dilution factor for the detergent samples was adjusted to account for activity loss over the course of the study to remain within the standard curve of BPN′-Y217L.

The activity at each time point for a particular variant was measured and the residual activity was calculated by comparison to the initial unstressed activity for that variant (T=0). The loss in activity was fit to a one phase decay curve and the T₅₀ (half-life) parameter was extracted. The T₅₀ of each variant was compared to that of BPN′-Y217L to calculate the long term stability PI set forth in Table 10.

TABLE 10
Long term Stability of LG12-clade Protease Variants
in Various Boron-free Detergents vs BPN′-
Y217L Protease Benchmark, Reported as PI

| Sample ID | Persil Non-Bio | All 3X | Purex Dirt Lift | Blue Moon |
|---|---|---|---|---|
| LG12 WT | 0.7 | 0.8 | 1.7 | 0.7 |
| LG12-05520 | 0.5 | 1.2 | 1.7 | NA* |
| LG12-06491 | 0.5 | 0.4 | 0.9 | 1.4 |
| LG12-06943 | 0.6 | 1 | 8.7 | 16 |
| LG12-07051 | 0.6 | 1.3 | 6.7 | NA* |
| LG12-07058 | 0.7 | 1.8 | 12.7 | 0.70 |
| LG12-07166 | 1.03 | 1.5 | 2.0 | 5.3 |
| BPN′-Y217L | 1.0 | 1.0 | 1.0 | 1.0 |

*NA denotes Not Analyzed

### EXAMPLE 8

### Laundry Cleaning in Boron-Free Detergents

Several highly stable variants from the long term stability experiment were tested for cleaning in boron-free formulations as shown in Table 11, where PI values are reported versus LG12 wild-type protease parent.

The detergents used were the same as those used in the long term stability experiment (see above) and included Persil Non-Bio (Unilever), Blue Moon (Blue Moon). All 3X (Sun), and Purex Dirt Lift (Henkel). Superior cleaning was seen for LG12-06943 relative to LG12 wild-type in the two North American formulations (Purex and All 3X), the European formulation (Persil Non-Bio), and the Chinese formulation (Blue Moon).

TABLE 11
Cleaning of Select LG12-clade Protease Variants with Improved
Performance vs LG12 WT in Boron-free Detergents, Reported as PI

| Sample ID | All 3X | Purex Dirt Lift | Persil Non-Bio | Blue Moon |
|---|---|---|---|---|
| LG12 WT | 1.0 | 1.0 | 1.0 | 1.0 |
| LG12-06943 | 1.8 | 1.6 | 1.5 | 1.3 |
| LG12-07051 | 1.5 | 2.0 | 2.0 | 1.4 |
| LG12-07166 | 0.7 | 1.2 | 1.2 | 1.0 |

### EXAMPLE 9

### Evaluation of Amino Acid Deletions in the Region of Residues 53 to 58 of LG12 Wild-Type Protease

One or more deletions at positions 53, 55, 56, 57, and/or 58 were made in the LG12 wild-type backbone. These variants were tested using the methods described in Example 1. Results for cleaning performance across multiple assays are reported in Table 12 as compared to the LG12 wild-type parent protease.

TABLE 12
Relative Performance of various LG12 variants

| | Sample ID | | | | | |
|---|---|---|---|---|---|---|
| | LG12 | LG12- | LG12- | LG12- | LG12- | LG12- |
| | WT | 07164 | 07174 | 07184 | 07194 | 07144 |
| Description | parent | del053 | del055 | del057 | del058 | del056- |
| | | | | | | 058 |
| Expression | ++++ | +++ | ++++ | ++++ | ++++ | ++++ |
| AAPF Activity | ++++ | +++++ | ++++ | +++ | +++ | ++++ |
| DMC Activity | ++++ | ++++ | ++++ | +++ | +++ | ++++ |
| Kirkland | ++++ | +++++ | +++++ | ++++ | ++++ | ++++ |
| HDD BMI | | | | | | |
| OMO HDD | ++++ | +++++ | +++++ | +++ | +++ | +++ |
| BMI | | | | | | |
| Kirkland | ++++ | +++++ | +++++ | +++ | +++ | ++++ |
| HDL BMI | | | | | | |
| OMO HDL | ++++ | NA* | +++ | +++ | ++ | ++ |
| BMI | | | | | | |
| Blue Moon | ++++ | +++++ | +++++ | ++++ | +++++ | ++++ |
| HDL BMI | | | | | | |
| ADW PAS-38 | ++++ | ++ | +++ | +++ | ++++ | ++++ |

*Not analyzed

This data suggests that modifications to decrease the size of the 55 to 59 loop formed by these residues in the LG12 structure can be accommodated and may in some cases impart improved cleaning performance.

### EXAMPLE 10

### Sequence Comparison of LG12-Clade Protease Variants

Alignment of Select LG12 Variants with Other Subtilisins

The amino acid sequences of LG12 wild-type (SEQ ID NO: 10), LG12-05520 (SEQ ID NO:37), LG12-05522 (SEQ ID NO:38), LG12-05547 (SEQ ID NO:29), LG12-05575 (SEQ ID NO:41), LG12-06491 (SEQ ID NO:34), LG12-06943 (SEQ ID NO:42), LG12-07051 (SEQ ID NO:39), LG12-07058 (SEQ ID NO:30), LG12-07065 (SEQ ID NO:35), LG12-07075 (SEQ ID NO:36), LG12-07098 (SEQ ID NO:31), LG12-07110 (SEQ ID NO:40), LG12-07115 (SEQ ID NO:43), LG12-07118 (SEQ ID NO:33), and LG12-07166 (SEQ ID NO:32) were aligned with sequences of previously identified LG12-clade members: Bhon03321 (SEQ ID NO:18), DSM_9712 (SEQ ID NO:19), DSM_9711 (SEQ ID NO:45), and DSM_6951 (SEQ ID NO:46). The alignment also included sequences of the following additional subtilisin proteases: B. amyloliquefaciens CAA24990 (SEQ ID NO:21), B_lentus_P29600 (SEQ ID NO:22). AprL (SEQ ID NO:23), Bag00927 (SEQ ID NO:25). Bgi02446 (SEQ ID NO:24), BspAL03279 (SEQ ID NO:27), BspMO4033 (SEQ ID NO:26), and BspM04284 (SEQ ID NO:28) using CLUSTALW software (Thompson et al., Nucleic Acids Research. 22:4673-4680, 1994) with the default parameters. The alignment was performed using the following parameters: Kimura's correction for sequence distance and ignoring positions with gaps. The NJ method works on a matrix of distances between all pairs of sequences to be analyzed. These distances are related to the degree of divergence between the sequences. FIG. 4 shows the alignment of these protease sequences. The position of the 4 amino acid delete that is observed in LG12 SprC when compared to B. lentus (P29600) sequence, located at positions encompassing G160DEL-N161DEL-R162DEL-N163DEL, was manually adjusted on this alignment to match details from the crystal structures.

### EXAMPLE 11

### Phylogenetic Tree for LG12-Clade Protease Variants and Other Subtilisins

A phylogenetic tree for amino acid sequences of LG12 (SEQ ID NO:10), LG12-05520 (SEQ ID NO:37), LG12-05522 (SEQ ID NO:38), LG12-05547 (SEQ ID NO:29), LG12-05575 (SEQ ID NO:41), LG12-06491 (SEQ ID NO:34), LG12-06943 (SEQ ID NO:42), LG12-07051 (SEQ ID NO:39). LG12-07058 (SEQ ID NO:30), LG12-07065 (SEQ ID NO:35), LG12-07075 (SEQ ID NO:36), LG12-07098 (SEQ ID NO:31), LG12-07110 (SEQ ID NO:40), LG12-07115 (SEQ ID NO:43), LG12-07118 (SEQ ID NO:33), LG12-07166 (SEQ ID NO:32), LG12-CP022 (SEQ ID NO:1), LG12-CP180 (SEQ ID NO:3), LG12-CP446 (SEQ ID NO:4), LG12-CP455 (SEQ ID NO:5), LG12-CP460 (SEQ ID NO:6). LG12-CP462 (SEQ ID NO:7), and LG12-CP465 (SEQ ID NO:8) was built using sequences of previously identified LG12-clade members: Bhon03321 (SEQ ID NO: 18), DSM_9712 (SEQ ID NO: 19, DSM_9711 (SEQ ID NO:45), DSM_6951 (SEQ ID NO:46), and sequences of additional subtilisin proteases: B. amyloliquefaciens_CAA24990 (SEQ ID NO:21), B_lentus_P29600 (SEQ ID NO:22), AprL (SEQ ID NO:23), Bag00927 (SEQ ID NO:25), Bgi02446 (SEQ ID NO:24). BspAL03279 (SEQ ID NO:27), BspMO4033 (SEQ ID NO:26), and BspM04284 (SEQ ID NO:28). The sequences were entered in the Vector NTI Advance suite and a Guide Tree was created using the Neighbor Joining (NJ) method (Saitou and Nei, Mol Biol Evol. 4:406-425, 1987). The tree construction was calculated using the following parameters: Kimura's correction for sequence distance and ignoring positions with gaps. The phylogenetic tree shown in FIG. 5 was visualized using Geneious 8.1 (Biomatters Ltd.). The branch length is a measure of the amount of divergence between two nodes in the tree and is expressed in units of substitutions per site of the sequence alignment.

### EXAMPLE 12

### Sequence Motif and Structural Relationship of the LG12-clade Proteases

The LG12-clade motif EPNALQDGNGHGTH includes the penta residue segment proline, asparagine, alanine, leucine, and glutamine at positions 54-59, which is a segment leading into the Histidine 64 of the catalytic triad. The relative juxtaposition of this segment with respect to the catalytic triad in the crystallographic model of LG12 is shown in FIG. 6. FIG. 6 shows a schematic of the overall fold of the main chain from the crystallographic model of LG12 wild-type protease in light gray. The side chains of the catalytic triad formed by Aspartic acid 32, Histidine 64 and Serine 221 are shown as black stick figures. Histidine 64 is indicated by the arrow. The region including residues 54-59 leading into His 64 of the triad are also shown as black stick figures.

This residue 54-59 segment of the molecule is seen to be in a loop leading into the helix that begins with Histidine 64. This residue 54-59 loop is critical in that it leads into and affects the juxtaposition of the following His64 and also that it forms part of the substrate binding surface.

Several subtilisin clades can be characterized by the sequences found between the Asp and Histidine that form the characteristic triad found in all classic serine hydrolases, both of the subtilisin and chymotrypsin type families. The conformation of the LG12-clade loop for the segment corresponding to residues 54-59 is distinctly different from that same loops found in the crystallographic models of sustilisin BPN′, subtilisin Carlsberg and Bacillus lentus proteases, as shown in FIGS. 7, 8, and 9, respectively.

In FIG. 7, the residue 54-59 sequence EPNALQ of the crystallographic structure of LG12 wild-type protease (shown as black sticks) is compared with the equivalent loop segment in subtilisin BPN′ (pdb entry 2ST1) (shown as gray lines). The proline 55 of LG12 is found at the top. The main chain conformation of the loops in subtilisin LG12 and BPN′ share the same overall fold while the sequences are quite different.

In FIG. 8, the residue 54-59 sequence EPNALQ of the crystallographic structure of LG12 wild-type protease (shown as black sticks) is compared with the equivalent loop segment in subtilisin Carlsberg (pdb entry 3UNX) (shown as gray lines). In subtilisin Carlberg, there is a radical departure in the main chain conformation in this loop relative to LG12 in the vicinity of Pro55 of LG12. In the crystallographic model, this segment shows some disorder as modelled for the tyrosine side chain in the coordinates of pdb entry 3UNX.

In FIG. 9, the residue 54-59 sequence EPNALQ of the crystallographic structure of LG12 wild-type protease (shown as black sticks) is compared with the equivalent loop segment in subtilisin from B. lentus (shown as gray lines). In the B. lentus subtilisin the main chain diverges (including a deletion of a residue in this loop in B. lentus) from that seen for LG12.

The LG12-clade sequence motif, extending from residue E54 to H67 in single letter code is EPNALQDGNGHGTH (SEQ ID NO:44) wherein the amino acid positions of the motif are numbered by correspondence with the amino acid sequence of LG12 wild-type set forth in SEQ ID NO:10.

### EXAMPLE 13

### Generation of Additional LG12-Clade Subtilisins

Additional LG12-clade variant proteases were generated using methods described in Example 2, resulting in from 2 to 13 beneficial mutations being introduced into the LG2 wild-type protease as shown in Table 13A. These additional LG12-clade protease variants demonstrated improved ADW cleaning performance and/or stability over LG2 wild-type, which data is set forth in Table 13B.

TABLE 13A
LG12-clade Variant Proteases

| | | No. |
|---|---|---|
| Sample | | Mutations |
| ID | Sequence Substitutions Relative to LG12 WT | vs LG12 |
| LG12- | N076D-G118R-G128D-S129F-S130Y-G166D- | 7 |
| 05653 | E248N | |
| LG12- | N076D-G118R-G128D-S129P-S130F-G166D- | 7 |
| 05663 | E248N | |
| LG12- | N076D-G118R-G128D-S129P-S130Y-G166D- | 7 |
| 05664 | E248N | |
| LG12- | N076D-G118R-G128E-S130R-G166D-E248N | 6 |
| 05685 | | |
| LG12- | N076D-G118R-G128H-S129K-S130W-G166D- | 7 |
| 05729 | E248N | |
| LG12- | N076D-G118R-G128H-S129M-S130Y-G166D- | 7 |
| 05734 | E248N | |
| LG12- | N076D-G118R-G128H-S129N-S130V-G166D- | 7 |
| 05735 | E248N | |
| LG12- | N076D-G118R-G128H-S129P-S130L-G166D- | 7 |
| 05736 | E248N | |
| LG12- | N076D-G118R-G128H-S129P-S130N-G166D- | 7 |
| 05737 | E248N | |
| LG12- | N076D-G118R-G128H-S129P-G166D-E248N | 6 |
| 05740 | | |
| LG12- | N076D-G118R-G128H-S129Q-S130I-G166D- | 7 |
| 05741 | E248N | |
| LG12- | N076D-G118R-G128H-S129Q-S130W-G166D- | 7 |
| 05743 | E248N | |
| LG12- | N076D-G118R-G128H-S129R-S130H-G166D- | 7 |
| 05744 | E248N | |
| LG12- | N076D-G118R-G128H-S130I-G166D-E248N | 6 |
| 05746 | | |
| LG12- | N076D-G118R-G128H-S129T-S130H-G166D- | 7 |
| 05747 | E248N | |
| LG12- | N076D-G118R-G128H-S129T-S130T-G166D- | 7 |
| 05748 | E248N | |
| LG12- | N076D-G118R-G128H-S129V-S130H-G166D- | 7 |
| 05749 | E248N | |
| LG12- | N076D-G118R-G128Q-S129R-S130N-G166D- | 7 |
| 05865 | E248N | |
| LG12- | N076D-G118R-G128R-S129V-S130E-G166D- | 7 |
| 05890 | E248N | |
| LG12- | N076D-G118R-G128S-S129P-S130A-G166D- | 7 |
| 05909 | E248N | |
| LG12- | N076D-G118R-G128S-S129V-S130Q-G166D- | 7 |
| 05913 | E248N | |
| LG12- | N076D-G118R-G128T-S129K-S130L-G166D- | 7 |
| 05924 | E248N | |
| LG12- | N076D-G118R-G128V-S129H-S130E-G166D- | 7 |
| 05958 | E248N | |
| LG12- | N076D-G118R-G128V-S129M-S130H-G166D- | 7 |
| 05961 | E248N | |
| LG12- | N076D-G118R-G128W-S129H-S130D-G166D- | 7 |
| 05980 | E248N | |
| LG12- | N076D-G118R-G128W-S129L-G166D-E248N | 6 |
| 05984 | | |
| LG12- | N076D-G118R-G128W-S129Q-S130F-G166D- | 7 |
| 05986 | E248N | |
| LG12- | N076D-G118R-G128W-S129Y-S130D-G166D- | 7 |
| 05993 | E248N | |
| LG12- | N076D-G118R-G128Y-S129P-S130D-G166D- | 7 |
| 06010 | E248N | |
| LG12- | N076D-G118R-G128Y-S129V-S130M-G166D- | 7 |
| 06017 | E248N | |
| LG12- | G128T-S129H-S130D-G166D-E248N | 5 |
| 06024 | | |
| LG12- | G118R-G128L-S129M-S130N-G166D-E248N | 6 |
| 06025 | | |
| LG12- | N038T-S156H-G166Y-M222L | 4 |
| 06581 | | |
| LG12- | G053D-N076D-G118R-N256R-S272K | 5 |
| 06945 | | |
| LG12- | N076D-G118R-G166D-N256R | 4 |
| 06971 | | |
| LG12- | A001G-A040D-A048Q-S052T-G053E-Y091H- | 13 |
| 07057 | S114T-I147A-S156N-S159N-G166H-S182E- | |
| | Y238F | |
| LG12- | A048E-S052T-V149G-S156N-G166D-F217R | 6 |
| 07078 | | |
| LG12- | S052T-V149G-S156N-S159D-G166H-F217R | 6 |
| 07088 | | |
| LG12- | S159D-G166D-S182E-F217R | 4 |
| 07189 | | |
| LG12- | G100A-G128H-S129F-S272K | 4 |
| 07525 | | |
| LG12- | N038Q-S052T-G100A-N117S-G118R-G128H | 6 |
| 07638 | | |
| LG12- | A040D-S052Q-G118R-G128M-S130Y-G166H- | 13 |
| 07890 | Y238F-N269K-V270C-E271G-S272K-A273V- | |
| | Q275Y | |
| LG12- | S052T-N076D-G118R-G128M-S129V-S130F- | 8 |
| 07990 | G166H-Y238F | |
| LG12- | A040D-S052T-N076D-G128M-S129F-S130Y- | 7 |
| 07997 | G166H | |
| LG12- | S052T-N076D-G128S-S129F-S130F | 5 |
| 08062 | | |
| LG12- | S052T-S129F | 2 |
| 08079 | | |
| LG12- | N076D-G118R-G128M-S129F-S130F-Y238F | 6 |
| 08114 | | |
| LG12- | A040D-S052Q-N076D-G128S-S129F-S130Y- | 7 |
| 08137 | Y238F | |
| LG12- | K027M-G053Y-G128D-S129R-S130I-G166D- | 10 |
| 08219 | F217R-E248K-N256E-S272K | |
| LG12- | G007C-S052T-N076D-G128D-S129R-S130I- | 10 |
| 08225 | S156N-G166D-F217R-S272K | |
| LG12- | G128D-S129R-S130I-S156T-G166D-F217R- | 7 |
| 08281 | S272K | |
| LG12- | S052T-N076D-G118R-G128D-S129R-S130I- | 9 |
| 08308 | G166D-F217R-N256R | |
| LG12- | G128D-S129R-S130I-G166H-F217R-S272K | 6 |
| 08355 | | |
| LG12- | G053D-G118R-G128D-S129R-S130I-S156N- | 9 |
| 08419 | G166D-F217R-E248K | |
| LG12- | S052T-G053Y-N076D-G118R-G128D-S129F- | 12 |
| 08478 | S130Y-S156N-G166D-F217R-N256E-S272K | |
| LG12- | S052T-G053D-N076D-G128D-S129F-S130Y- | 10 |
| 08598 | S156N-F217R-N256E-S272K | |
| LG12- | S052F-N076D-G118R-G128D-S129F-S130Y- | 11 |
| 08634 | S156N-G166D-F217R-E248N-S272K | |
| LG12- | S052F-G053Y-N076D-G118R-G128D-S129F- | 13 |
| 08636 | S130Y-S156N-G166D-F217R-E248N-N256E- | |
| | S272K | |
| LG12- | S052F-N076D-G118R-G128D-S129F-S130Y- | 9 |
| 08653 | G166D-F217R-E248N | |
| LG12- | N076D-G118R-G128D-S129F-S130Y-G166D- | 10 |
| 08677 | F217R-E248N-N256E-S272K | |
| LG12- | N076D-G118R-G128D-S129F-S130Y-G166D- | 9 |
| 08680 | F217R-E248N-N256E | |
| LG12- | N076D-G118R-G128D-S129F-S130Y-S156N- | 9 |
| 08684 | G166D-F217R-E248N | |
| LG12- | S052T-N076D-G118R-G128D-S129F-S130Y- | 10 |
| 08705 | G166D-F217R-E248N-S272K | |
| LG12- | N076D-G118R-G128D-S129F-S130Y-S156N- | 10 |
| 08706 | G166D-F217R-E248N-S272K | |
| LG12- | S052F-G053Y-N076D-G118R-G128D-S129F- | 12 |
| 08721 | S130Y-G166D-F217R-E248N-N256R-S272K | |
| LG12- | S052T-G053Y-N076D-G118R-G128D-S129F- | 10 |
| 08727 | S130Y-S156N-G166D-E248N | |
| LG12- | S052F-N076D-G118R-G128D-S129F-S130Y- | 11 |
| 08731 | G166D-F217R-E248N-N256E-S272K | |
| LG12- | S052F-N076D-G118R-G128D-S129F-S130Y- | 10 |
| 08740 | G166D-F217R-E248N-N256E | |
| LG12- | G007C-S052T-G053D-N076D-G118R-G128D- | 12 |
| 08755 | S129F-S130Y-G166D-F217R-E248N-N256E | |
| LG12- | S052F-G053Y-N076D-G118R-G128D-S129F- | 11 |
| 08756 | S130Y-G166D-E248N-N256E-S272K | |
| LG12- | N076D-G118R-G128D-S129F-S130Y-S156N- | 9 |
| 08759 | G166D-E248N-S272K | |
| LG12- | S052F-G053Y-G118R-G128D-S129F-S130Y- | 11 |
| 08764 | S156N-G166D-E248N-N256E-S272K | |
| LG12- | G053D-N076D-G118R-G128D-S129F-S130Y- | 9 |
| 08780 | G166D-E248N-S272K | |
| LG12- | S052F-N076D-G118R-G128D-S129F-S130Y- | 10 |
| 08781 | G166D-F217R-E248N-Q275R | |
| LG12- | S052F-N076D-G118R-G128D-S129F-S130Y- | 10 |
| 08830 | G166D-F217R-E248N-N256R | |
| LG12- | S052F-N076D-G118R-G128D-S129F-S130Y- | 11 |
| 08841 | G166D-F217R-E248N-N256R-S272K | |
| LG12- | S052F-N076D-G118R-G128D-S129F-S130Y- | 10 |
| 08843 | G166D-F217R-E248N-S272K | |
| LG12- | S052F-G053Y-N076D-G118R-G128D-S129F- | 11 |
| 08863 | S130Y-G166D-F217R-E248N-S272K | |
| LG12- | S052F-N076D-G118R-G128D-S129F-S130Y- | 11 |
| 08867 | G166H-F217R-E248N-N256E-S272K | |
| LG12- | S052T-G053Y-N076D-G118R-G128D-S129F- | 11 |
| 08870 | S130Y-S156N-G166D-E248N-S272K | |
| LG12- | S052T-N076D-G118R-G128D-S129F-S130Y- | 11 |
| 08875 | S156N-G166D-E248N-N256E-S272K | |
| LG12- | N076D-G118R-G128D-S129F-S130Y-S156N- | 9 |
| 08883 | G166D-E248N-N256R | |
| LG12- | S052T-N076D-G118R-G128D-S129F-S130Y- | 11 |
| 08888 | S156N-G166H-F217R-E248N-N256R | |
| LG12- | S052F-N076D-G118R-G128D-S129F-S130Y- | 10 |
| 08904 | S156N-G166D-E248N-S272K | |
| LG12- | N076D-G118R-I122V-G128D-S129F-S130Y- | 9 |
| 08913 | G166D-F217R-E248N | |
| LG12- | S052T-N076D-G118R-G128D-S129F-S130Y- | 10 |
| 08921 | G166D-F217R-E248N-N256R | |
| LG12- | S052F-G053Y-N076D-G118R-G128D-S129F- | 11 |
| 08933 | S130Y-G166D-F217R-E248N-N256E | |
| LG12- | S052F-N076D-G118R-G128D-S129F-S130Y- | 9 |
| 08940 | S156N-G166D-E248N | |
| LG12- | S052T-G128V-S129M-S130H-S156D-G166D- | 9 |
| 08957 | F217R-E248N-S272K | |
| LG12- | G128V-S129M-S130H-G166D-F217R-E248N- | 7 |
| 08960 | S272K | |
| LG12- | S052T-G053Y-G128V-S129M-S130H-S156D- | 10 |
| 08968 | G166H-F217R-E248N-N256E | |
| LG12- | G128V-S129M-S130H-G166D-E248N-S272K | 6 |
| 08981 | | |
| LG12- | S052T-N076D-G128V-S129M-S130H-G166D- | 10 |
| 08982 | F217R-E248N-N256E-S272K | |
| LG12- | S052T-N076D-G118R-G128V-S129M-S130H- | 10 |
| 08996 | G166D-F217R-N256E-S272K | |
| LG12- | N076D-G128V-S129M-S130H-S156D-G166D- | 9 |
| 09018 | F217R-E248N-S272K | |
| LG12- | S052T-G128V-S129M-S130H-G166D-F217R- | 9 |
| 09029 | E248N-N256E-S272K | |
| LG12- | S052T-N076D-G118R-G128V-S129M-S130H- | 9 |
| 09062 | G166D-217R-S272K | |
| LG12- | S052T-G118R-G128V-S129M-S130H-G166D- | 8 |
| 09071 | F217R-S272K | |
| LG12- | G118R-G128V-S129M-S130H-S156D-G166D- | 8 |
| 09079 | F217R-E248N | |
| LG12- | S052T-N076D-G118R-G128V-S129M-S130H- | 11 |
| 09092 | G166D-F217R-E248N-N256E-S272K | |
| LG12- | N076D-G118R-G128V-S129M-S130H-S156D- | 9 |
| 09107 | G166D-F217R-E248N | |
| LG12- | S052T-G053Y-G128V-S129M-S130H-S156D- | 10 |
| 09111 | G166H-F217R-N256E-S272K | |
| LG12- | G128V-S129M-S130H-G166D-F217R-E248N | 6 |
| 09112 | | |
| LG12- | S052T-G053Y-G128V-S129M-S130H-G166D- | 8 |
| 09119 | E248N-S272K | |
| LG12- | S052T-G053Y-N076D-G118R-G128V-S129M- | 10 |
| 09124 | S130H-G166D-F217R-E248N | |
| LG12- | G053Y-G118R-G128V-S129M-S130H-G166D- | 9 |
| 09142 | E248N-N256E-S272K | |
| LG12- | G128V-S129M-S130H-G166D-F217R-E248D- | 7 |
| 09147 | S272K | |
| LG12- | G128V-S129M-S130H-G166D-E248N | 5 |
| 09156 | | |
| LG12- | N076D-G118R-G128V-S129M-S130H-G166D- | 9 |
| 09158 | E248N-N256E-S272K | |
| LG12- | S052T-G053Y-N076D-G118R-G128V-S129M- | 12 |
| 09167 | S130H-S156D-G166D-F217R-E248N-S272K | |
| LG12- | S052T-N076D-G118R-G128V-S129M-S130H- | 10 |
| 09176 | G166D-E248N-N256E-S272K | |
| LG12- | N076D-G118R-GI28V-S129M-S130H-G166D- | 7 |
| 09210 | F217R | |
| LG12- | S052T-N076D-G118R-G128V-S129M-S130H- | 12 |
| 09245 | S156D-G166D-F217R-E248N-N256E-S272K | |
| LG12- | G128V-S129M-S130H-F217R-E248N | 5 |
| 09260 | | |

TABLE 13B
Cleaning performance of Additional
LG12-clade Variants, Reported as PI

| | PAS-38 in | | |
|---|---|---|---|
| Sample ID | GSM-B | Stability TRIS-CA | Stability TRIS-EDTA |
| LG12 WT | 1.0 | 1.0 | 1.0 |
| LG12-05653 | 2.6 | 0.4 | 0.5 |
| LG12-05663 | 2.6 | 2.2 | 1.8 |
| LG12-05664 | 2.8 | 2.5 | 2.0 |
| LG12-05685 | 2.6 | 1.6 | 1.2 |
| LG12-05729 | 4.0 | 2.5 | 1.8 |
| LG12-05734 | 2.9 | 1.9 | 1.4 |
| LG12-05735 | 2.3 | 1.9 | 1.6 |
| LG12-05736 | 3.4 | 2.6 | 1.7 |
| LG12-05737 | 3.1 | 2.7 | 1.8 |
| LG12-05740 | 4.1 | 2.5 | 1.7 |
| LG12-05741 | 2.5 | 2.1 | 1.3 |
| LG12-05743 | 3.7 | 1.5 | 1.3 |
| LG12-05744 | 2.7 | 2.4 | 1.4 |
| LG12-05746 | 2.4 | 2.3 | 1.7 |
| LG12-05747 | 2.8 | 2.2 | 1.4 |
| LG12-05748 | 3.3 | 2.3 | 1.7 |
| LG12-05749 | 2.9 | 1.7 | 1.3 |
| LG12-05865 | 2.4 | 2.0 | 1.3 |
| LG12-05890 | 2.1 | 1.5 | 1.5 |
| LG12-05909 | 2.2 | 2.2 | 1.2 |
| LG12-05913 | 5.1 | 1.8 | 1.4 |
| LG12-05924 | 2.1 | 1.8 | 1.4 |
| LG12-05958 | 1.6 | 2.0 | 1.3 |
| LG12-05961 | 6.9 | 1.1 | 1.1 |
| LG12-05980 | 1.5 | 1.7 | 1.5 |
| LG12-05984 | 1.8 | 1.6 | 1.8 |
| LG12-05986 | 2.2 | 2.4 | 2.2 |
| LG12-05993 | 2.2 | 1.9 | 1.4 |
| LG12-06010 | 1.3 | 2.1 | 1.6 |
| LG12-06017 | 2.0 | 1.5 | 1.3 |
| LG12-06024 | 1.5 | 1.6 | 1.4 |
| LG12-06025 | 1.9 | 1.6 | 1.4 |
| LG12-06581 | 2.7 | 1.3 | 1.4 |
| LG12-06945 | 1.7 | 2.3 | 1.2 |
| LG12-06971 | 1.8 | 2.0 | 1.4 |
| LG12-07057 | 1.4 | 1.8 | 2.2 |
| LG12-07078 | 1.5 | 2.0 | 2.3 |
| LG12-07088 | 1.6 | 1.7 | 2.1 |
| LG12-07189 | 1.3 | 2.1 | 2.7 |
| LG12-07525 | 1.6 | 1.3 | 1.3 |
| LG12-07638 | 2.0 | 1.4 | 1.3 |
| LG12-07890 | 1.3 | 2.4 | 1.2 |
| LG12-07990 | 4.2 | 1.8 | 1.4 |
| LG12-07997 | 1.3 | 1.7 | 1.3 |
| LG12-08062 | 1.6 | 1.2 | 1.4 |
| LG12-08079 | 1.4 | 1.7 | 1.2 |
| LG12-08114 | 2.9 | 1.3 | 1.7 |
| LG12-08137 | 2.2 | 1.3 | 1.7 |
| LG12-08219 | 2.3 | 4.1 | 2.0 |
| LG12-08225 | 1.9 | 1.4 | 1.3 |
| LG12-08281 | 1.8 | 2.9 | 2.9 |
| LG12-08308 | 1.2 | 3.6 | 3.0 |
| LG12-08355 | 1.4 | 2.5 | 2.4 |
| LG12-08419 | 3.7 | 2.3 | 3.0 |
| LG12-08478 | 1.4 | 9.8 | 3.9 |
| LG12-08598 | 1.7 | 3.5 | 2.6 |
| LG12-08634 | 1.2 | 2.2 | 2.2 |
| LG12-08636 | 2.0 | 3.0 | 3.7 |
| LG12-08653 | 1.5 | 2.2 | 2.1 |
| LG12-08677 | 1.4 | 2.3 | 2.3 |
| LG12-08680 | 1.3 | 2.4 | 2.1 |
| LG12-08684 | 1.4 | 3.3 | 2.6 |
| LG12-08705 | 1.3 | 1.4 | 1.9 |
| LG12-08706 | 1.3 | 2.7 | 2.7 |
| LG12-08721 | 5.7 | 1.6 | 3.2 |
| LG12-08727 | 1.3 | 2.0 | 1.8 |
| LG12-08731 | 1.4 | 1.7 | 2.3 |
| LG12-08740 | 1.4 | 2.1 | 2.2 |
| LG12-08755 | 1.4 | 1.5 | 1.5 |
| LG12-08756 | 3.5 | 1.4 | 2.0 |
| LG12-08759 | 1.3 | 3.2 | 2.2 |
| LG12-08764 | 2.3 | 2.6 | 2.2 |
| LG12-08780 | 1.2 | 1.3 | 1.6 |
| LG12-08781 | 2.0 | 2.2 | 2.3 |
| LG12-08830 | 1.4 | 2.0 | 1.8 |
| LG12-08841 | 1.3 | 1.4 | 2.4 |
| LG12-08843 | 1.3 | 1.5 | 2.2 |
| LG12-08863 | 2.2 | 2.2 | 2.6 |
| LG12-08867 | 1.3 | 1.3 | 2.0 |
| LG12-08870 | 1.3 | 2.4 | 2.1 |
| LG12-08875 | 1.3 | 2.0 | 2.2 |
| LG12-08883 | 1.2 | 1.8 | 2.3 |
| LG12-08888 | 1.4 | 2.0 | 2.4 |
| LG12-08904 | 1.4 | 2.4 | 2.1 |
| LG12-08913 | 1.6 | 1.5 | 2.4 |
| LG12-08921 | 1.4 | 1.5 | 2.3 |
| LG12-08933 | 2.9 | 1.7 | 3.0 |
| LG12-08940 | 1.5 | 3.0 | 2.4 |
| LG12-08957 | 1.6 | 1.2 | 1.6 |
| LG12-08960 | 1.4 | 2.7 | 2.6 |
| LG12-08968 | 1.6 | 2.3 | 1.9 |
| LG12-08981 | 2.9 | 2.3 | 2.1 |
| LG12-08982 | 1.2 | 2.5 | 2.3 |
| LG12-08996 | 1.3 | 3.2 | 3.2 |
| LG12-09018 | 1.2 | 2.3 | 1.4 |
| LG12-09029 | 1.4 | 3.0 | 2.1 |
| LG12-09062 | 1.5 | 3.1 | 2.3 |
| LG12-09071 | 1.3 | 2.7 | 2.5 |
| LG12-09079 | 1.3 | 1.3 | 1.5 |
| LG12-09092 | 1.3 | 3.1 | 2.6 |
| LG12-09107 | 1.3 | 1.5 | 1.3 |
| LG12-09111 | 1.4 | 2.3 | 2.8 |
| LG12-09112 | 1.3 | 2.0 | 2.4 |
| LG12-09119 | 2.3 | 2.5 | 2.0 |
| LG12-09124 | 1.5 | 1.8 | 2.0 |
| LG12-09142 | 1.8 | 1.9 | 2.3 |
| LG12-09147 | 2.2 | 2.1 | 1.9 |
| LG12-09156 | 3.6 | 1.8 | 2.7 |
| LG12-09158 | 2.0 | 2.6 | 2.4 |
| LG12-09167 | 1.2 | 1.5 | 1.7 |
| LG12-09176 | 1.9 | 2.5 | 2.4 |
| LG12-09210 | 1.2 | 2.7 | 2.4 |
| LG12-09245 | 1.3 | 1.8 | 1.7 |
| LG12-09260 | 1.3 | 3.2 | 2.4 |

### EXAMPLE 14

### Generation of Additional LG12-Clade Subtilisins

Additional LG12-clade variant proteases were generated using methods described in Example 2, resulting in from 36 to 41 beneficial mutations being introduced into the LG12 wild-type protease as set forth in Table 14.

TABLE 14
LG12-clade Variant Proteases

| | | No. |
|---|---|---|
| Sample | | mutations |
| ID | Sequence Substitutions Relative to LG12 WT | vs LG12 |
| LG12- | S052A-N076D-T078S-T079I-Y086P-A088V- | 36 |
| CP00172 | D089S-S097G-T103S-L104V-G106S-I111L- | |
| | S114A-I115G-S116N-N120H-I122A-M124L- | |
| | G128S-S129P-G131P-T133A-A134T-Q136E- | |
| | C139V-N141S-Y143T-N144S-I147V-V148L- | |
| | I150V-S158T-G166D-F217L-A224T-E248N | |
| LG12- | S052A-N076D-Y086P-N087S-A088V-D089S- | 39 |
| CP00195 | S097G-T103S-L104V-G106S-I111L-S114A- | |
| | I115G-S116N-N120H-I122A-M124L-G128S- | |
| | S129P-G131P-T133A-A134T-Q136E-C139V- | |
| | N141S-Y143T-N144S-I147V-V148L-I150V- | |
| | S158T-G166D-T209Y-N212S-N213T-S216T- | |
| | F217L-A224T-E248N | |
| LG12- | A001T-N076D-T078S-T079I-Y086P-D089E- | 38 |
| CP00227 | S097G-T103S-L104V-G106S-I111L-S114A- | |
| | I115G-S116N-N120H-I122A-M124L-G128S- | |
| | S129P-G131P-T133A-A134T-Q136E-C139V- | |
| | N141S-Y143T-N144S-I147V-V148L-I150V- | |
| | S158T-G166D-T209Y-N212S-N213T-S216T- | |
| | F217L-E248N | |
| LG12- | A001T-S052A-N076D-Y086P-N087S-A088V- | 41 |
| CP00234 | D089S-S097G-T103S-L104V-G106S-I111L- | |
| | S114A-I115G-S116N-N120H-I122A-M124L- | |
| | G128S-S129P-G131P-T133A-A134T-Q136E- | |
| | C139V-N141S-Y143T-N144S-I147V-V148L- | |
| | I150V-A153S-S158T-G166D-T209Y-N212S- | |
| | N213T-S216T-F217L-A224T-E248N | |
| LG12- | A001T-N076D-Y086P-N087S-A088V-D089S- | 40 |
| CP00376 | S097G-T103S-L104V-G106S-I111L-S114A- | |
| | I115G-S116N-N120H-I122A-M124L-G128S- | |
| | S129P-G131P-T133A-A134T-Q136E-C139V- | |
| | N141S-Y143T-N144S-I147V-V148L-I150V- | |
| | A153S-S158T-G166D-T209Y-N212S-N213T- | |
| | S216T-F217L-A224T-E248N | |
| LG12- | A001T-N076D-Y086P-N087S-A088V-D089S- | 40 |
| CP00415 | S097G-T103S-L104V-G106S-I111L-S114A- | |
| | I115G-S116N-N120H-I122A-M124L-G128S- | |
| | S129P-G131P-T133A-A134T-Q136E-C139V- | |
| | N141S-Y143T-N144S-I147V-V148L-I150V- | |
| | S158T-G166D-T209Y-N212S-N213T-S216T- | |
| | F217L-A224T-E248N-Q275H | |
| LG12- | A001T-N076D-T078S-T079I-Y086P-N087S- | 41 |
| CP00466 | A088V-D089S-S097G-T103S-L104V-G106S- | |
| | I111L-S114A-I115G-S116N-N120H-I122A- | |
| | M124L-G128S-S129P-G131P-T133A-A134T- | |
| | Q136E-C139V-N141S-Y143T-N144S-I147V- | |
| | V148L-I150V-S158T-G166D-T209Y-N212S- | |
| | N213T-S216T-F217L-A224T-E248N | |

### EXAMPLE 15

### Cleaning Performance and Stability of LG12-Clade Variant Proteases

LG12-clade protease variants generated as described in Example 14 above and LG12 wild-type protease (SEQ ID NO:10) were screened in MTP assays, as described in Example 1 to compare their cleaning performance and/or stability to LG12-CP462 protease (SEQ ID NO:7). The results of these comparisons are set forth in Table 15.

TABLE 15
Cleaning and Stability of LG12-clade Protease
Variants Compared to LG12-CP462. reported as PI

| | EMPA-116 | | CFT CS-38 | | CFT C-10 | | Stability |
|---|---|---|---|---|---|---|---|
| | Persil | | Persil | | Persil | | Persil |
| | Non- | Test | Non- | Test | Non- | Test | Non- |
| Sample ID | Bio | HDL | Bio | HDL | Bio | HDL | Bio |
| LG12-CP462 | 1.0 | 1.0 | 1.0 | 1.0 | 1.0 | 1.0 | 1.0 |
| LG12-CP00466 | 1.0 | 0.9 | 1.1 | 1.0 | 1.2 | 1.1 | 0.9 |
| LG12-CP00195 | 0.9 | 0.9 | 1.2 | 1.3 | 1.2 | 1.2 | 1.0 |
| LG12-CP00234 | 0.9 | 0.9 | 1.2 | 1.1 | 1.2 | 1.1 | 1.0 |
| LG12-CP00172 | 0.9 | 0.9 | 1.7 | 2.1 | 1.4 | 1.2 | 1.6 |
| LG12-CP00376 | 0.9 | 0.9 | 1.2 | 1.3 | 0.7 | 1.1 | 1.0 |
| LG12-CP00227 | 0.9 | 0.9 | 1.0 | 0.8 | 0.6 | 1.0 | 1.0 |
| LG12-CP00415 | 0.9 | 0.9 | 1.6 | 1.5 | 1.7 | 1.3 | 0.9 |
| LG12 WT | 0.7 | ND | 0.6 | ND | 1.2 | ND | ND |

### EXAMPLE 16

### Comparison of LG12-Clade Protease Variants to Related Molecules Identification of Homologous Proteases

Homologs were identified by a BLAST search (Altschul et al., Nucleic Acids Res, 25:3389-402, 1997) against the NCBI non-redundant protein database and the Genome Quest (GQ) Patent database with search parameters set to default values using the mature protein amino acid sequence for LG12-CP462 protease (SEQ ID NO:7) as the query sequence. Percent identity (PID) is defined as the number of identical residues divided by the number of aligned residues in the pairwise alignment. Value labeled “sequence length” on tables corresponds to the length (in amino acids) for the proteins referenced with the listed Accession numbers, while “aligned length” refers to sequence used for alignment and PID calculation. Table 16A provides a list of sequences with the PID to LG12-CP462 from the NCBI non-redundant protein database and Table 16B provides a list of sequences from the GQ patent database.

TABLE 16A
NCBI Non-redundant Protein Database Results for LG12-CP462

| | | | Sequence | Alignment |
|---|---|---|---|---|
| Accession # | PID | Organism | Length | Length |
| AAC43580 | 86.1 | Bacillus sp. | 378 | 274 |
| WP_047970940 | 82.8 | Bacillus sp. LL01 | 377 | 274 |
| KPB03414 | 80.3 | Bacillus sp. CHD6a | 377 | 274 |
| WP_010192403 | 80.3 | Bacillus sp. m3-13 | 381 | 274 |
| AAA22212.1 | 75.4 | B. alcalophilus | 380 | 272 |
| BAA02442.1 | 75.0 | B. alcalophilus | 380 | 272 |
| AFR78140 | 74.3 | synthetic construct | 269 | 272 |
| BAD11988 | 71.9 | Bacillus sp. | 376 | 274 |
| | | KSM-LD1 SA | | |
| BAD21128 | 71.0 | Bacillus sp. | 377 | 276 |
| | | KSM-LD1 SB | | |
| KJD53753 | 70.7 | B. amyloliquefaciens | 333 | 229 |
| WP_026080796 | 70.4 | B. licheniformis | 378 | 274 |
| AAG00492 | 70.4 | B. licheniformis | 379 | 274 |
| ADK11044 | 70.4 | B. licheniformis | 379 | 274 |
| WP_031314535 | 70.1 | B. licheniformis | 378 | 274 |
| EQM28932 | 70.1 | B. licheniformis | 379 | 274 |
| | | CG-B52 | | |
| AAG00493 | 70.1 | B. licheniformis | 310 | 274 |

TABLE 16B
GQ Patent Database Results for LG12-CP462

| | | | Sequence | Alignment |
|---|---|---|---|---|
| Patent ID # | PID | Organism | Length | Length |
| WO2015038792- | 86.5-85.8 | Bacillus sp.; | 275 | 274-273 |
| multiple | | LG12 Synthetic | | |
| sequences | | | | |
| WO2015038792- | 86.1 | Bacillus sp.; | 381 | 274 |
| 0004 | | LG12 | | |
| WO2015038792- | 83.6 | B. horikoshii; | 275 | 274 |
| 0019 | | DSM 69511 | | |
| WO2015038792- | 83.2 | B. horikoshii | 275 | 274 |
| 0016 | | DSM 97121 | | |
| WO2015038792- | 83.2 | B. horikoshii | 275 | 274 |
| 0010 | | DSM 87192 | | |
| WO2015038792- | 82.1 | Synthetic | 268 | 274 |
| 0027 | | Construct | | |
| WO2015038792- | 80.3 | Bacillus sp. | 275 | 274 |
| 0007 | | m3-134 | | |
| WO2015038792- | 80.3 | Bacillus sp. | 383 | 274 |
| 0006 | | m3-132 | | |
| EP1160327- | 76.1-75.7 | Bacillus sp. | 269 | 272 |
| multiple | | Synthetic | | |
| sequences | | | | |
| WO9402618 | 75.7 | Bacillus novalis | 269 | 272 |
| US20130123162- | 75.74 | B. lentus | 269 | 272 |
| multiple | | Synthetic | | |
| sequences | | | | |
| WO2011140364- | 75.7-75.4 | B. lentus | 380-377 | 272 |
| multiple | | Synthetic | | |
| sequences | | | | |
| US20140045268- | 75.4 | Synthetic | 353 | 272 |
| 0040 | | Construct | | |
| WO2015144932- | 75.4 | Artificial | 269 | 272 |
| 0006 | | Sequence, | | |
| | | B. lentus | | |

### EXAMPLE 17

### Sequence Comparison of LG12-Clade Protease Variants

Alignment of LG12-Clade Protease Variants with Other Subtilisins

The amino acid sequences of LG12 wild-type (SEQ ID NO:10): variants: LG12-CP022 (SEQ ID NO:1), LG12-CP180 (SEQ ID NO:3), LG12-CP446 (SEQ ID NO:4), LG12-CP455 (SEQ ID NO:5), LG12-CP460 (SEQ ID NO:6), LG12-CP462 (SEQ ID NO:7), LG12-CP465 (SEQ ID NO:8), LG12-05520 (SEQ ID NO:37), LG12-05522 (SEQ ID NO:38), LG12-05547 (SEQ ID NO:29), LG12-05575 (SEQ ID NO:41), LG12-06491 (SEQ ID NO:34), LG12-06943 (SEQ ID NO:42), LG12-07051 (SEQ ID NO:39), LG12-07058 (SEQ ID NO:30), LG12-07065 (SEQ ID NO:35), LG12-07075 (SEQ ID NO:36), LG12-07098 (SEQ ID NO:31), LG12-07110 (SEQ ID NO:40), LG12-07115 (SEQ ID NO:43), LG12-07118 (SEQ ID NO:33), LG12-07166 (SEQ ID NO:32), LG12-CP00172 (SEQ ID NO:59), LG12-CP00195 (SEQ ID NO:61), LG12-CP00227 (SEQ ID NO:60), LG12-CP00234 (SEQ ID NO:64), LG12-CP00376 (SEQ ID NO:62), LG12-CP00415 (SEQ ID NO:63), LG12-CP00466 (SEQ ID NO:65), LG12-05653 (SEQ ID NO:49), LG12-05668 (SEQ ID NO:50), LG12-05961 (SEQ ID NO:51), LG12-08308 (SEQ ID NO:53), LG12-08721 (SEQ ID NO:55), LG12-09176 (SEQ ID NO:54), LG12-07990 (SEQ ID NO:52), and LG12-07057 (SEQ ID NO:56); previously identified LG2-clade members: Bhon03321 (SEQ ID NO:18), DSM_9712 (SEQ ID NO:19), DSM_9711 (SEQ ID NO:45), DSM_6951 (SEQ ID NO:46), Bac.sp.WP_010192403 (SEQ ID NO:66); and more recently identified LG12-clade members: Bac.sp.LL01_WP_04797094) (SEQ ID NO:57) and Bac.sp.CHD6a_KPB03414 (SEQ ID NO:58), were aligned using CLUSTALW software (Thompson et al., Nucleic Acids Research, 22:4673-4680, 1994) with the default parameters. The alignment was performed using the following parameters: Kimura's correction for sequence distance and ignoring positions with gaps. The NJ method works on a matrix of distances between all pairs of sequences to be analyzed. These distances are related to the degree of divergence between the sequences, FIG. 10A-F shows the alignment of these protease sequences. The sequences were entered in the Vector NTI Advance suite and a Guide Tree was created using the Neighbor Joining (NJ) method (Saitou and Nei, Mol Biol Evol. 4:406-425, 1987). The tree construction was calculated using the following parameters: Kimura's correction for sequence distance and ignoring positions with gaps. AlignX displays the calculated distance values in parenthesis following the molecule name displayed on the phylogenetic tree shown in FIG. 11.

We claim:

1. A protease variant, or a recombinant polypeptide or an active fragment thereof comprising an amino acid sequence comprising two or more substitutions at one or more positions selected from:
(i) 1, 3, 7, 9, 19, 24, 27, 30, 38, 40, 43, 45, 48, 52, 53, 57, 72, 76, 78, 79, 86, 87, 88, 89, 90, 91, 93, 97, 99, 100, 101, 103, 104, 106, 107, 111, 114, 115, 116, 117, 118, 120, 122, 123, 124, 128, 129, 130, 131, 133, 134, 136, 139, 140, 141, 143, 144, 145, 147, 148, 149, 150, 153, 156, 158, 159, 160, 166, 181, 182, 185, 188, 194, 209, 212, 213, 215, 216, 217, 222, 224, 228, 234, 236, 238, 240, 242, 245, 246, 248, 251, 252, 256, 265, 267, 269, 270, 271, 272, 273, and 275;
(ii) 1, 3, 9, 19, 24, 27, 30, 38, 40, 43, 45, 48, 52, 53, 57, 72, 76, 78, 79, 86, 87, 88, 89, 90, 91, 93, 97, 99, 100, 101, 103, 104, 106, 107, 111, 114, 115, 116, 117, 118, 120, 122, 123, 124, 128, 129, 130, 131, 133, 134, 136, 139, 140, 141, 143, 144, 145, 147, 148, 149, 150, 153, 156, 158, 159, 160, 166, 181, 182, 185, 188, 194, 209, 212, 213, 215, 216, 217, 222, 224, 228, 234, 236, 238, 240, 242, 245, 246, 248, 251, 252, 256, 265, 267, 271, 272, and 275;
(iii) 1, 7, 9, 27, 38, 40, 45, 48, 52, 53.76, 78, 91, 100, 114, 117, 118, 122, 128, 129, 130, 140, 147, 149, 156, 158, 159, 160, 166, 181, 182, 194, 209, 217, 222, 228, 234, 236, 238, 245, 248, 256, 269, 270, 271, 272, 273, and 275;
(iv) 1, 24, 30, 38, 40, 48, 52, 53, 76, 78, 79, 86, 87, 88, 89, 91, 93, 97, 100, 103, 104, 106, 111, 114, 115, 116, 117, 118, 120, 122, 124, 128, 129, 130, 131, 133, 134, 136, 139, 141, 143, 144, 147, 148, 149, 150, 153, 156, 158, 159, 166, 182, 185, 194, 209, 212, 213, 216, 217, 222, 224, 236, 238, 248, 251, 252, 256, 272, and 275;
(v) 1, 24, 30, 38, 40, 52, 53, 76, 78, 79, 86, 87, 88, 89, 93, 97, 100, 103, 104, 106, 111, 114, 115, 116, 117, 118, 120, 122, 124, 128, 129, 131, 133, 134, 136, 139, 141, 143, 144, 147, 148, 149, 150, 153, 156, 158, 159, 166, 182, 185, 194, 209, 212, 213, 216, 217, 222, 224, 236, 238, 248, 251, 252, 256, 272, and 275; or
(vi) 1.38, 40, 48, 52, 53, 76, 91, 114, 117, 118, 128, 129, 130, 147, 149, 156, 158, 159, 166, 182, 217, 238, 248, 256, and 272;
wherein the amino acid positions of the variant or recombinant polypeptide or active fragment thereof are numbered by correspondence with the amino acid sequence of SEQ ID NO: 10.

2. The protease variant, or recombinant polypeptide or active fragment thereof of any preceding claim, wherein said variant or recombinant polypeptide or active fragment thereof comprises an amino acid sequence comprising two or more substitutions selected from:
(i) A1X, T3X, G7X, P9X, A19X, S24X, K27X, I30X, N38X, A40X, N43X, K45X, A48X, S52X, G53X, A57X, V72X, N76X, T78X, T79X, Y86X, N87X, A88X, D89X, L90X, Y91X, V93X, S97X, S99X, G100X, S101X, T103X, L104X, G106X, I107X, I11X, S114X, I115X, S116X, N117X, G118X, N120X, I122X, N123X, M124X, G128X, S129X, S130X, G131X, T133X, A134X, Q136X, C139X, N140X, N141X, Y143X, N144X, R145X, I147X, V148X, V149X, I150X, A153X, S156X, S158X, S159X, G160X, G166X, S181X, S182X, T185X, S188X, S194X, T209X, N212X, N213X, A215X, S216X, F217X, M222X, A224X, A228X, I234X, A236X, Y238X, S240X, T242X, Q245X, I246X, E248X, K251X, N252X, N256X, K265X, V267X, N269X, V270X, E271X, S272X, A273X, and Q275X;
(ii) A1X, T3X, P9X, A19X, S24X, K27X, 130X, N38X, A40X, N43X, K45X, A48X, S52X, G53X, A57X, V72X, N76X, T78X, T79X, Y86X, N87X, A88X, D89X, L90X, Y91X, V93X, S97X, S99X, G100X, S101X, T103X, L104X, G106X, I107X, I111X, S114X, I115X, S116X, N117X, G118X, N120X, I122X, N123X, M124X, G128X, S129X, S130X, G131X, T133X, A134X, Q136X, C139X, N140X, N141X, Y143X, N144X, R145X, I147X, V148X, V149X, I150X, A153X, S156X, S158X, S159X, G160X, G166X, S181X, S182X, T185X, S188X, S194X, T209X, N212X, N213X, A215X, S216X, F217X, M222X, A224X, A228X, I234X, A236X, Y238X, S240X, T242X, Q245X, I246X, E248X, K251X, N252X, N256X, K265X, V267X, E271X, S272X, and Q275X;
(iii) A1X, G7X, P9X, K27X, N38X, A40X, K45X, A48X, S52X, G53X, N76X, T78X, Y91X, G100X, S114X, N117X, G118X, I122X, G128X, S129X, S130X, N140X, I147X, V149X, S156X, S158X, S159X, G160X, G166X, S181X, S182X, S194X, T209X, F217X, M222X, A228X, I234X, A236X, Y238X, Q245X, E248X, N256X, N269X, V270X, E271X, S272X, A273X, and Q275X;
(iv) A1X, S24X, I30X, N38X, A40X, A48X, S52X, G53X, N76X, T78X, T79X, Y86X, N87X, A88X, D89X, Y91X, V93X, S97X, G100X, T103X, L104X, G106X, I111X, S114X, I115X, S116X, N117X, G118X, N120X, I122X, M124X, G128X, S129X, S130X, G131X, T133X, A134X, Q136X, C139X, N141X, Y143X, N144X, I147X, V148X, V149X, I150X, A153X, S156X, S158X, S159X, G166X, S182X, T185X, S194X, T209X, N212X, N213X, S216X, F217X, M222X, A224X, A236X, Y238X, E248X, K251X, N252X, N256X, S272X, and Q275X;
(v) A1X, S24X, I30X, N38X, A40X, S52X, G53X, N76X, T78X, T79X, Y86X, N87X, A88X, D89X, V93X, S97X, G100X, T103X, L104X, G106X, I111X, S114X, I115X, S116X, N117X, G118X, N120X, I122X, M124X, G128X, S129X, G131X, T133X, A134X, Q136X, C139X, N141X, Y143X, N144X, I147X, V148X, V149X, I150X, A153X, S156X, S158X, S159X, G166X, S182X, T185X, S194X, T209X, N212X, N213X, S216X, F217X, M222X, A224X, A236X, Y238X, E248X, K251X, N252X, N256X, S272X, and Q275X; or
(vi) A1X, N38X, A40X, A48X, S52X, G53X, N76X, Y91X, S114X, N117X, G118X, G128X, S129X, S130X, I147X, V149X, S156X, S158X, S159X, G166X, S182X, F217X, Y238X, E248X, N256X, and S272X;
wherein X is any amino acid and the amino acid positions of the variant or recombinant polypeptide or active fragment thereof are numbered by correspondence with the amino acid sequence of SEQ ID NO:10.

3. The protease variant, or recombinant polypeptide or active fragment thereof of any preceding claim, wherein said variant or recombinant polypeptide or active fragment thereof comprises an amino acid sequence comprising two or more substitutions selected from:
(i) X1G, X1T, X3E, X3V, X7C, X9I, X9T, X9V, X19R, X24R, X24T, X27S, X27T, X30V, X38A, X38Q, X38T, X40D, X40E, X40L, X40V, X43D, X43H, X43L, X43S, X45Q, X45R, X48E, X48Q, X52A, X52N, X52Q, X52T, X53D, X53E, X53N, X57P, X72A, X76D, X78A, X78D, X78G, X78S, X79I, X86P, X87S, X88V, X89E, X89S, X90P, X91H, X93T, X97G, X99L, X100A, X100K, X101T, X103E, X103S, X104V, X106S, X107T, X107V, X111L, X114A, X114T, X115G, X116E, X116N, X117E, X117S, X118R, X120H, X122A, X123D, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130A, X130E, X130F, X130G, X130H, X130I, X130L, X130M, X130N, X130Q, X130R, X130T, X130V, X130W, X130Y, X131P, X133A, X133D, X133V, X134T, X136E, X139V, X140D, X140Y, X141S, X143T, X144S, X145N, X145W, X147A, X147V, X148L, X149G, X149L, X149N, X149S, X150V, X153S, X156A, X156H, X156N, X158T, X158V, X159D, X159E, X159N, X159Q, X160D, X166A, X166D, X166H, X166N, X166S, X166Y, X181A, X181N, X182E, X185Q, X188Y, X194P, X209F, X209V, X209Y, X212S, X213T, X215D, X215E, X216T, X217L, X217R, X222L, X224T, X228S, X234V, X236N, X236Q, X238F, X240F, X242S, X245A, X245N, X246V, X248K, X248N, X251R, X251S, X252S, X256E, X256K, X256P, X256R, X256Y, X265S, X267N, X269K, X270C, X271F, X271W, X272K, X272N, X272Q, X273V, X275H, and X275R;
(ii) X1G, X1T, X3E, X3V, X9I, X9T, X9V, X19R, X24R, X24T, X27S, X27T, X30V, X38A, X38Q, X38T, X40D, X40E, X40L, X40V, X43D, X43H, X43L, X43S, X45Q, X45R, X48E, X48Q, X52A, X52N, X52Q, X52T, X53D, X53E, X53N, X57P, X72A, X76D, X78A, X78D, X78G, X78S, X791, X86P, X87S, X88V, X89E, X89S, X90P, X91H, X93T, X97G, X99L, X100A, X100K, X101T, X103E, X103S, X104V, X106S, X107T, X107V, X111L, X114A, X114T, X115G, X116E, X116N, X117E, X117S, X118R, X120H, X122A, X123D, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130A, X130E, X130F, X130G, X130H, X130O, X130L, X130M, X130N, X130Q, X130R, X130T, X130V, X130W, X130Y, X131P, X133A, X133D, X133V, X134T, X136E, X139V, X140D, X140Y, X141S, X143T, X144S, X145N, X145W, X147A, X147V, X148L, X149G, X149L, X149N, X149S, X150V, X153S, X156A, X156H, X156N, X158T, X158V, X159D, X159E, X159N, X159Q, X160D, X166A, X166D, X166H, X166N, X166S, X166Y, X181A, X181N, X182E, X185Q, X188Y, X194P, X209F, X209V, X209Y, X212S, X213T, X215D, X215E, X216T, X217L, X217R, X222L, X224T, X228S, X234V, X236N, X236Q, X238F, X240F, X242S, X245A, X245N, X246V, X248K, X248N, X251R, X251S, X252S, X256E, X256K, X256P, X256R, X256Y, X265S, X267N, X271F, X271W, X272K, X272N, X272Q, X275H, and X275R;
(iii) X1G, X7C, X9T, X27M, X38Q, X38T, X40D, X45Q, X45R, X48E, X48Q, X52F, X52Q, X52T, X53D, X53E, X53Y, X76D, X78A, X91H, X100A, X114T, X117S, X118R, X122V, X128A, X128D, X128E, X128H, X128K, X128L, X128M, X128N, X128Q, X128R, X128S, X128T, X128V, X128W, X128Y, X129D, X129E, X129F, X129G, X129H, X129I, X129K, X129L, X129M, X129N, X129P, X129Q, X129R, X129T, X129V, X129Y, X130A, X130D, X130E, X130F, X130G, X130H, X130I, X130L, X130M, X130N, X130Q, X130R, X130T, X130V, X130W, X130Y, X140D, X147A, X149G, X156D, X156H, X156N, X156T, X158T, X158V, X159D, X159N, X160D, X166D, X166H, X166Y, X181N, X182E, X194P, X209V, X217R, X222L, X228S, X234V, X236N, X236Q, X238F, X245A, X248D, X248K, X248N, X256E, X256P, X256R, X269K, X270C, X271G, X272K, X272Q, X273V, X275R, and X275Y;
(iv) X1G, X1T, X24R, X24T, X30V, X38A, X38Q, X38T, X40D, X40E, X40L, X40V, X48Q, X52A, X52N, X52Q, X52T, X53D, X53E, X53N, X76D, X78A, X78D, X78G, X78S, X79I, X86P, X87S, X88V, X89E, X91H, X93T, X97G, X100A, X100K, X103E, X103S, X104V, X106S, X111L, X114A, X114T, X115G, X116E, X116N, X117E, X117S, X118R, X120H, X122A, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X130F, X130H, X130I, X130Y, X131P, X133A, X133D, X133V, X134T, X136E, X139V, X141S, X143T, X144S, X147A, X147V, X148L, X149G, X149L, X149N, X149S, X150V, X153S, X156A, X156H, X156N, X158T, X158V, X159D, X159E, X159N, X159Q, X166A, X166D, X166H, X166N, X166S, X166Y, X182E, X185Q, X194P, X209F, X209V, X209Y, X212S, X213T, X216T, X217L, X217R, X222L, X224T, X236N, X236Q, X238F, X248K, X248N, X251R, X251S, X252S, X256E, X256K, X256P, X256R, X256Y, X272K, X272N, X272Q, X275H, and X275R;
(v) X1G, X1T, X24R, X24T, X30V, X38A, X38Q, X38T, X40D, X40E, X40L, X40V, X52A, X52N, X52Q, X52T, X53D, X53E, X53N, X76D, X78A, X78D, X78G, X78S, X791, X86P, X87S, X88V, X89E, X93T, X97G, X100A, X100K, X103E, X103S, X104V, X106S, X111L, X114A, X114T, X115G, X16E, X16N, X117E, X117S, X18R, X12H, X122A, X124L, X128A, X128D, X128E, X128H, X128K, X128M, X128N, X128Q, X128S, X128Y, X129D, X129E, X129G, X129H, X129I, X129K, X129L, X129M, X129N, X129P, X129Q, X129R, X129T, X129V, X129Y, X131P, X133A, X133D, X133V, X134T, X136E, X139V, X141S, X143T, X144S, X147A, X147V, X148L, X149G, X149L, X149N, X149S, X150V, X153S, X156A, X156H, X156N, X158T, X158V, X591D, X159E, X159N, X159Q, X166A, X166D, X160H, X166N, X116S, X160Y, X182E, X185Q, X194P, X209F, X209V, X209Y, X212S, X213T, X216T, X217L, X217R, X222L, X224T, X236N, X236Q, X238F, X248K, X248N, X251R, X251S, X252S, X256E, X256K, X256P, X256R, X256Y, X272K, X272N, X272Q, X275H, and X275R; or
(vi) X1G, X38Q, X40D, X48Q, X52F, X52T, X53E, X53Y, X76D, X91H, X114T, X117S, X118R, X128D, X128M, X128V, X129F, X129M, X129R, X129V, X130F, X130H, X130I, X130Y, X147A, X149G, X156N, X158T, X159N, X166D, X166H, X182E, X217R, X238F, X248N, X256E, X256P, X256R, and X272K;
wherein X is any amino acid and the amino acid positions of the variant or recombinant polypeptide or active fragment thereof are numbered by correspondence with the amino acid sequence of SEQ ID NO:10.

4. The protease variant, or recombinant polypeptide or active fragment thereof of any preceding claim, wherein said variant or recombinant polypeptide or active fragment thereof comprises an amino acid sequence comprising two or more substitutions selected from:
(i) A001G, A001T, T003E, T003V, G007C, P009I, P009T, P009V, A019R, S024R, S024T, K027S, K027T, I030V, N038A, N038Q, N038T, A040D, A040E, A040L, A040V, N043D, N043H, N043L, N043S, K045Q, K045R, A048E, A048Q, S052A, S052N, S052Q, S052T, G531D, G053E, G053N, A057P, V072A, N076D, T078A, T078D, T078G, T078S, T079I, Y086P, N087S, A088V, D089E, D089S, L090P, Y091H, V093T, S097G, S099L, G100A, G100K, S101T, T103E, T103S, L104V, G106S, I107T, I107V, I111L, S114A, S114T, I115G, S116E, S116N, N117E, N117S, G118R, N120H, I122A, N123D, M124L, G128A, G128D, G128E, G128H, G128K, G128M, G128N, G128Q, G128S, G128Y, S129D, S129E, S129G, S129H, S129I, S129K, S129L, S129M, S129N, S129P, S129Q, S129R, S129T, S129V, S129Y, S130A, S130E, S130F, S130G, S130H, S130I, S130L, S130M, S130N, S130Q, S130R, S130T, S130V, S130W, S130Y, G131P, T133A, T133D, T133V, A134T, Q136E, C139V, N140D, N140Y, N141S, Y143T, N144S, R145N, R145W, I147A, I147V, V148L, V149G, V149L, V149N, V149S, I150V, A153S, S156A, S156H, S156N, S158T, S158V, S159D, S159E, S159N, S159Q, G160D, G166A, G166D, G166H, G166N, G166S, G166Y, S181A, S181N, S182E, T185Q, S188Y, S194P, T209F, T209V, T209Y, N212S, N213T, A215D, A215E, S216T, F217L, F217R, M222L, A224T, A228S, I234V, A236N, A236Q, Y238F, S240F, T242S, Q245A, Q245N, I246V, E248K, E248N, K251R, K251S, N252S, N256E, N256K, N256P, N256R, N256Y, K265S, V267N, N269K, V270C, E271F, E271W, S272K, S272N, S272Q, A273V, Q275H, and Q275R;
(ii) A001G, A001T, T003E, T003V, P009I, P009T, P009V, A019R, S024R, S024T, K027S, K027T, I030V, N038A, N038Q, N038T, A040D, A040E, A040L, A040V, N043D, N043H, N043L, N043S, K045Q, K045R, A048E, A048Q, S052A, S052N, S052Q, S052T, G053D, G053E, G053N, A057P, V072A, N076D, T078A, T078D, T078G, T078S, T079I, Y086P, N087S, A088V, D089E, D089S, L090P, Y091H, V093T, S097G, S099L, G100A, G100K, S101T, T103E, T103S, L104V, G106S, I107T, I107V, I111L, S114A, S114T, I115G, S116E, S116N, N117E, N117S, G118R, N120H, I122A, N123D, M124L, G128A, G128D, G128E, G128H, G128K, G128M, G128N, G128Q, G128S, G128Y, S291D, S129E, S129G, S129H, S129I, S129K, S129L, S129M, S129N, S129P, S129Q, S129R, S129T, S129V, S129Y, S130A, S130E, S130F, S130G, S130H, S130I, S130L, S130M, S130N, S130Q, S130R, S130T, S130V, S130W, S130Y, G131P, T133A, T133D, T133V, A134T, Q136E, C139V, N140D, N140Y, N141S, Y143T, N144S, R145N, R145W, I147A, I147V, V148L, V149G, V149L, V149N, V149S, I150V, A153S, S156A, S156H, S156N, S158T, S158V, S159D, S159E, S159N, S159Q, G160D, G166A, G166D, G166H, G166N, G166S, G166Y, S181A, S181N, S182E, T185Q, S188Y, S194P, T209F, T209V, T209Y, N212S, N213T, A215D, A215E, S216T, F217L, F217R, M222L, A224T, A228S, I234V, A236N, A236Q, Y238F, S240F, T242S, Q245A, Q245N, I246V, E248K, E248N, K251R, K251S, N252S, N256E, N256K, N256P, N256R, N256Y, K265S, V267N, E271F, E271W, S272K, S272N, S272Q, Q275H, and Q275R;
(iii) A001G, G007C, P009T, K027M, N038Q, N038T, A040D, K045Q, K045R, A048E, A048Q, S052F, S052Q, S052T, G053D, G053E, G053Y, N076D, T078A, Y091H, G100A, S114T, N117S, G118R, I122V, G128A, G128D, G128E, G128H, G128K, G128L, G128M, G128N, G128Q, G128R, G128S, G128T, G128V, G128W, G128Y, S129D, S129E, S129F, S129G, S129H, S129I, S129K, S129L, S129M, S129N, S129P, S129Q, S129R, S129T, S129V, S129Y, S130A, S130D, S130E, S130F, S130G, S130H, S130I, S130L, S130M, S130N, S130Q, S130R, S130T, S130V, S130W, S130Y, N140D, I147A, V149G, S156D, S156H, S156N, S156T, S158T, S158V, S159D, S159N, G160D, G166D, G166H, G166Y, S181N, S182E, S194P, T209V, F217R, M222L, Y238F, Q245A, E248D, E248K, E248N, N256E, N256P, N256R, N269K, V270C, E271G, S272K, S272Q, A273V, Q275R, and Q275Y;
(iv) A001G, A001T, S024T, I030V, N038Q, A040D, A048Q, S052A, S052Q, S052T, G053D, G053E, N076D, T078D, T078S, T079I, Y086P, N087S, A088V, D089E, D089S, Y091H, V093T, S097G, G100A, T103E, T103S, L104V, G106S, I111L, S114A, S114T, I115G, S116N, N117S, G118R, N120H, I122A, M124L, G128S, S129P, S130F, S130H, S130I, S130Y, G131P, T133A, T133V, A134T, Q136E, C139V, N141S, Y143T, N144S, I147A, I147V, V148L, V149G, V149L, I150V, A153S, S156H, S156N, S158T, S159D, S159N, G166D, G166H, G166S, S182E, T185Q, S194P, T209Y, N212S, N213T, S216T, F217L, F217R, M222L, A224T, A236N, Y238F, E248N, K251S, N252S, N256P, N256R, N256Y, S272K, S272Q, and Q275H;
(v) A001G, A001T, S024T, I030V, N038Q, A040D, S052A, S052Q, S052T, G053D, G053E, N076D, T078D, T078S, T079I, Y086P, N087S, A088V, D089E, D089S, V093T, S097G, G100A, T103E, T103S, L104V, G16S, I111L, S114A, S114T, I115G, S116N, N117S, G118R, N120H, I122A, M124L, G128S, S129P, G131P, T133A, T133V, A134T, Q136E, C139V, N141S, Y143T, N144S, I147A, I147V, V148L, V149G, V149L, I150V, A153S, S156H, S156N, S158T, S159D, S159N, G166D, G166H, G166S, S182E, T185Q, S194P, T209Y, N212S, N213T, S216T, F217L, F217R, M222L, A224T, A236N, Y238F, E248N, K251S, N252S, N256P, N256R, N256Y, S272K, S272Q, and Q275H; or
(vi) A001G, N038Q, A040D, A048Q, S052F, S052T, G053E, G053Y, N076D, Y091H, S114T, N117S, G118R, G128D, G128M, G128V, S129F, S129M, S129R, S129V, S130F, S130H, S130I, S130Y, I147A, V149G, S156N, S158T, S159N, G166D, G166H, S182E, F217R, Y238F, E248N, N256E, N256P, N256R, and S272K;
wherein the amino acid positions of the variant or recombinant polypeptide or active fragment thereof are numbered by correspondence with the amino acid sequence of SEQ ID NO: 10.

5. The protease variant, or recombinant polypeptide or active fragment thereof of any preceding claim, wherein said variant or recombinant polypeptide or active fragment thereof comprises an amino acid sequence comprising two or more substitutions at one or more position selected from:
(i) 52, 53, 76, 86, 97, 103, 104, 106, 111, 114, 115, 116, 118, 120, 122, 124, 128, 129, 130, 131, 134, 136, 139, 141, 143, 144, 147, 148, 150, 156, 158, 159, 166, 217, 248, and 272;
(ii) 53, 76, 106, 128, 131, 134, 139, 158, 159, 166, 217, 248, and 272;
(iii) 52, 53, 76, 106, 118, 128, 129, 130, 131, 134, 139, 156, 158, 166, 217, 248, and 272;
(iv) 52, 53, 76, 128, 118, 156, 158, 166, 217, and 248;
(v) 53, 76, 128, 158, 166, and 248;
(vi) 52, 53, 76, 118, 128, 156, 158, 166, 217, and 248; or
(vii) 52-129, 53-166, 76-166, 166-159, 166-27, 53-76-166, 53-118-166, 53-166-272, 76-118-166, 76-166-248, 76-166-256, and 76-166-272;
wherein the amino acid positions of the variant or recombinant polypeptide or active fragment thereof are numbered by correspondence with the amino acid sequence of SEQ ID NO:10.

6. The protease variant, or recombinant polypeptide or active fragment thereof of any preceding claim, wherein said variant or recombinant polypeptide or active fragment thereof comprises an amino acid sequence comprising two or more substitutions selected from:
(i) S52X, G53X, N76X, Y86X, S97X, T103X, L104X, G106X, I111X, S114X, I115X, S116X, G118X, N120X, I122X, M124X, G128X, S129X, S130X, G131X, A134X, Q136X, C139X, N141X, Y143X, N144X, I147X, V148X, I150X, S156X, S158X, S159X, G166X, F217X, E248X, and S272X;
(ii) G53X, N76X, G106X, G128X, G131X, A134X, C139X, S158X, S159X, G166X, F217X, E248X, and S272X;
(iii) S52X, G53X, N76X, G106X, G118X, G128X, S129X, S130X, G131X, A134X, C139X, S156X, G166X, F217X, E248X, and S272X,
(iv) S52X, G53X, N76X, G118X, G128X, S156X, S158X, G166X, F217X, and E248X;
(v) G53X, N76X, G128X, S158X, G166X, and E248X;
(vi) S52X, G53X, N76X, G118X, G128X, S156X, S158X, G166X, F217X, and E248X; or
(vii) S52X-S129X, G53X-G166X, N76X-G166X, G166X-S159X, G166X-S272X, G53X-N76X-G166X, G3X-G118X-G166X, G53X-G166X-S272X, N76X-G118X-G166X, N76X-G166X-E248X, N76X-G166X-N256X, and N76X-G166X-S272X;
wherein X is any amino acid and the amino acid positions of the variant or recombinant polypeptide or active fragment thereof are numbered by correspondence with the amino acid sequence of SEQ ID NO:10.

7. The protease variant, or recombinant polypeptide or active fragment thereof of any preceding claim, wherein said variant or recombinant polypeptide or active fragment thereof comprises an amino acid sequence comprising two or more substitutions selected from:
(i) X52T, X53D, X76D, X86P, X97G, X103S, X104V, X006S, X111L, X114A, X115G, X116N, X118R, X120H, X122A, X124L, X128D, X128S, X129F, X130Y, X131P, X134T, X136E, X139V, X141S, X143T, X144S, X147V, X148L, X150V, X156N, X158T, X159D, X166D, X217R, X248N, and X272K;
(ii) X53D, X76D, X106S, X128S, X131P, X134T, X139V, X158T, X159D, X166D, X217R, X248N, and X272K;
(iii) X52T, X53D, X76D, X106S, X118R, X128D, X129F, X130Y, X131P, X134T, X139V, X156N, X166D, X217R, X248N, and X272K;
(iv) X52T, X53D, X76D, X118R, X128S, X156N, X158T, X166D, X217R, and X248N;
(v) X53D, X76D, X128S, X158T, X166D, and X248N;
(vi) X52T, X53D, X76D, X118R, X128S, X156N, X158T, X166D, X217R, and X248N; or
(vii) X52T-X129F, X53D-X166D, X76D-X166D, X166H-X159D, X166D-X272K, X53D-X76D-X166D, X53D-X118R-X166D, X53D-X166D-X272K, X76D-X118R-X166D, X76D-X166D-X248K, X76D-X166D-X256R, and X76D-X166D-X272K;
wherein X is any amino acid and the amino acid positions of the variant or recombinant polypeptide or active fragment thereof are numbered by correspondence with the amino acid sequence of SEQ ID NO:10.

8. The protease variant, or recombinant polypeptide or active fragment thereof of any preceding claim, wherein said variant or recombinant polypeptide or active fragment thereof comprises an amino acid sequence comprising two or more substitutions selected from:
(i) S52T, G53D, N76D, Y086P, S097G, T103S, L104V, G106S, I111L, S114A, I115G, S116N, G118R, N120H, I122A, M124L, G128D, G128S, S129F, S129P, S130Y, G131P, A134T, Q136E, C139V, N141S, Y143T, N144S, I147V, V148L, I150V, S156N, S158T, S159D, G166D, F217R, E248N, and S272K;
(ii) G53D, N76D, G106S, G128S, G131P, A134T, C139V, S158T, S159D, G166D, F217R, E248N, and S272K;
(iii) S52T, G53D, N76D, G106S, G118R, G128D, S129F, S130Y, G131P, A134T, C139V, S156N, G166D, F217R, E248N, and S272K;
(iv) S52T, G53D, N76D, G118R, G128S, S156N, S158T, G166D, F217R, and E248N;
(v) G53D, N76D, G128S, S158T, G166D, and E248N;
(vi) S52T, G53D, N76D, G118R, S156N, S158T, G166D, F217R, and E248N; or
(vii) S52T-S129F, G53D-G166D, N76D-G166D, G166H-S159D, G166D-S272K, G53D-N76D-G166D, G53D-G118R-G166D, G53D-G166D-S272K, N76D-G118R-G166D, N76D-G166D-E248K, N76D-G166D-N256R, and N76D-G166D-S272K;
wherein the amino acid positions of the variant or recombinant polypeptide or active fragment thereof are numbered by correspondence with the amino acid sequence of SEQ ID NO: 10.

9. The protease variant, or recombinant polypeptide or active fragment thereof of any preceding claim, wherein said two or more substitutions is selected from S052T-S129F; G053D-G166D; N076D-G166D; G166H-S159D; G166D-S272K; G053D-N076D-G166D; G053D-G118R-G166D; G053D-G166D-S272K; N076D-G118R-G166D; N076D-G166D-E248K; N076D-G166D-N256R; N076D-G166D-S272K; S024T-G053E-G166D-M222L-N256P; V149L-S156H-T185Q-S272Q-S159D; G053D-N076D-G118R-G166D-N256R; S052T-V149G-S156N-G166H-F217R; S052T-V149G-S156N-G166D-F217R; S052T-V149G-S156N-S159D-G166D-F217R; S052T-V149G-S156N-G166D-S182E-F217R; A040D-S114T-I147A-S159N-G166D-S182E; N038Q-G053E-G100A-N117S-I147A-S158T-G166D-S272K; N038Q-G053E-N117S-I147A-S158T-G166D-N256P-S272K; A001G-S052Q-V149G-S156N-S159N-S182E-Y238F-S272Q; A001G-S052Q-S114T-S156N-S159N-S182E-Y238F-S272Q; N038Q-G053E-G100A-N117S-I147A-S158T-G166D-N256P-S272K; A001G-I030V-T078D-V093T-T003E-S156N-S159D-G166S-S194P-F217R-A236N; A001T-N076D-T078S-T079I-Y086P-D089E-S097G-T103S-L104V-G106S-I111L-S114A-I115G-S116N-N I120H-I122A-M124L-G128S-S129P-G131P-T133A-A134T-Q136E-C139V-N141S-Y143T-N144S-I147V-V148L-I150V-S158T-G166D-E248N; S052A-N076D-T078S-T079I-Y086P-D089E-S097G-T103S-L104V-G106-I111L-S114A-I115G-S116N-N120H-I122A-M124L-G128S-S129P-G131P-T133V-A134T-Q136E-C139V-N141S-Y143T-N144S-I147V-V148L-I150Y-S158T-G166D-F217L-E248N; A001T-N076D-T078S-T079I-Y086P-D089E-S097G-T103S-L104V-G106S-I111L-S114A-I115G-S116N-N120H-I122A-M124L-G128S-S129P-G131P-T133V-A134T-Q136E-C139V-N141S-Y143T-N144S-I147V-V148L-I150V-S158T-G166D-T209Y-N212S-N213T-S216T-F217L-A224T-E248N-K251S-N252S-N256Y; N076D-T078S-T079I-Y086P-D089E-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150Y-S158T-G166D-T209Y-N212S-N213T-S216T-F217L-A224T-E248N; N076D-T078S-T079I-Y086P-N087S-A088V-D089S-S097G-T103S-L104V-G106S-I111L-S114A-I150G-S116N-N120H-I122A-M124L-G128S-S129P-G131P-T133A-A134T-Q136E-C139V-N141S-Y143T-N144S-I147V-V148L-I104V-S158T-G166D-F217L-E248N; A001T-N076D-T078S-T079I-Y086P-D089E-S097G-T103S-L 104V-G106S-I111L-S114A-I115G-S116N-N120H-I122A-M124L-G128S-S129P-G131P-T133A-A134T-Q136E-C139V-N141S-Y143T-N144S-I147V-V148L-I150Y-S158T-G166D-T209Y-N212S-N213T-S216T-F217L-A224T-E248N; A001T-N076D-T078S-T079I-Y086P-N087S-A08V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I110S-S158T-G166D-T209Y-N212S-N213T-S216T-F217L-E248N; S052A-N076D-T078S-T079I-Y086P-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-Y148L-I150V-S158T-G166D-F217L-A224T-E248N; S052A-N076D-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I110Y-S158T-G166D-T209Y-N212S-N213T-S216T-F217L-A224T-E248N; A001T-N076D-T078S-T079I-Y086P-D089E-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-E248N; A001T-S052A-N076D-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-A153S-S158T-G166D-T209Y-N212S-N213T-S216T-F217L-A224T-E248N; A001T-N076D-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-A153S-S158T-G166D-T209Y-N212S-N213T-S216T-F217L-A224T-E248N; A001T-N076D-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-A224T-E248N-Q275H; A001T-N076D-T078S-T079I-Y086P-N087S-A088V-D089S-S097G-T103S-L104V-G106S-I111L-S114A-I115G-S116N-N120H-I122A-M124L-G128S-S129P-G131P-T133A-A134T-Q136E-C139V-N141S-Y143T-N144S-I147V-V148L-I150V-S158T-G166D-T209Y-N212S-N213T-S216T-F217L-A224T-E248N; N076D-G118R-G128D-S129R-S130I-G166D-E248N; N076D-G118R-G128D-S129F-S130Y-G166D-E248N; N076D-G118R-G128V-S129M-S130H-G166D-E248N; S052T-N076D-G118R-G128M-S129V-S130F-G166H-Y238F; S052T-N076D-G118R-G128D-S129R-S130I-G166D-F217R-N256R; S052T-N076D-G118R-G128V-S129M-S130H-G166D-E248N-N256E-S272K; S052F-G053Y-N076D-G118R-G128D-S129F-S130Y-G166D-F217R-E248N-N256R-S272K; and A001G-A040D-A048Q-S052T-G053E-Y091H-S114T-I147A-S156N-S159N-G166H-S182E-Y238F; wherein the amino acid positions of the variant or recombinant polypeptide or active fragment thereof are numbered by correspondence with the amino acid sequence of SEQ ID NO: 10.

10. A protease variant, or a recombinant polypeptide or an active fragment thereof comprising an amino acid sequence comprising one or more deletions at one or more positions selected from 53, 55, 56, 57, and 58 or G053, P055, N056, A057 and L058; wherein the amino acid positions of the variant or recombinant polypeptide or active fragment thereof are numbered by correspondence with the amino acid sequence of SEQ ID NO:10.

11. The protease variant, or a recombinant polypeptide or an active fragment thereof of any one of claims 1-9, wherein said variant or recombinant polypeptide or active fragment thereof further comprises an EPNALQDGNGHGTH (SEQ ID NO:44) motif.

12. The protease variant, or recombinant polypeptide or active fragment thereof of claim 11, wherein said motif extends from residue E54 to H67, wherein the amino acid positions are numbered by correspondence with the amino acid sequence of SEQ ID NO:10.

13. The protease variant, or recombinant polypeptide or active fragment thereof of claim 11 or 12, with the proviso that the variant or recombinant polypeptide or active fragment thereof does not comprise WP_047970940, WP_010192403, KPB_03414, BAD11988, BAD21128, Bhon03321, DSM9712_SprC, DSM6951_SprC, or DSM9711_SprC.

14. The protease variant, or recombinant polypeptide or active fragment thereof of any one of claims 1-12, wherein said variant or recombinant polypeptide or active fragment thereof further comprises an amino acid sequence having at least 75% amino acid sequence identity to SEQ ID NO: 10.

15. The protease variant, or recombinant polypeptide or active fragment thereof of claim 14, with the proviso that the variant or recombinant polypeptide or active fragment thereof does not comprise WP_047970940, WP_010192403, KPB_03414, BAD11988, BAD21128, Bhon03321, DSM9712_SprC, DSM6951_SprC, or DSM9711_SprC.

16. The protease variant, or recombinant polypeptide or active fragment thereof of any preceding claim, wherein said protease variant, or recombinant polypeptide or active fragment thereof has protease activity in the presence of a surfactant, cleaning activity in a detergent composition, or a combination thereof.

17. The protease variant, or recombinant polypeptide or active fragment thereof of claim 16, wherein the detergent composition is an automatic dish washing detergent or a laundry detergent and/or the cleaning activity comprises hydrolysis of a substrate on a protein stain.

18. The protease variant, or recombinant polypeptide or active fragment thereof of any preceding claim, wherein said protease variant, or recombinant polypeptide or active fragment thereof has one or more improved property selected from improved cleaning performance, improved stability, and improved residual protease activity when compared to SEQ ID NO: 10 and/or 48.

19. The protease variant, or recombinant polypeptide or active fragment thereof of claim 18, wherein said improved property is
(i) improved residual protease activity and said protease variant, or recombinant polypeptide or active fragment thereof has 15-100%, 20-100%, 25-100%, 30-100%, 35-100%, or 40-100% residual protease activity when measured in accordance with the Stability Assay of Example 1;
(ii) improved cleaning performance and said protease variant, or recombinant polypeptide or active fragment thereof has a BMI cleaning PI of ≥0.7 in comparison to SEQ ID NO:48 when measured in accordance with the Cleaning Performance in Detergent assay of Example 1;
(iii) improved stability and said protease variant, or recombinant polypeptide or active fragment thereof has a stability PI≥1.01 or 1.1 or 1.2 in comparison to SEQ ID NO:10 when measured in accordance with the Stability Assay of Example 1; and/or
(iv) improved cleaning performance and said protease variant, or recombinant polypeptide or active fragment thereof has a cleaning PI≥1.01 or 1.1 or 1.2 in comparison to SEQ ID NO:10 when measured in accordance with the Cleaning Performance in Detergent Assay of Example 1.

20. The protease variant, or recombinant polypeptide or active fragment thereof of any preceding claim, wherein said protease variant, or recombinant polypeptide or active fragment thereof is derived from a reference polypeptide selected from SEQ ID NOs:10, 18, 19, 45, 46, 57, 58, and 66.

21. The protease variant, or recombinant polypeptide or active fragment thereof of claim 20, wherein the reference polypeptide is SEQ ID NO: 10 and said protease variant, or recombinant polypeptide or active fragment thereof has one or more substitutions in one or more molecular segments selected from Segment 1 comprising positions 76-79 and 86, Segment 2 comprising positions 97-148, Segment 3 comprising positions 150-170, and Segment 4 comprising positions 207-220.

22. An LG12-Clade of proteases comprising a protease variant or a recombinant polypeptide or an active fragment thereof of any one of claims 1-9 and 11-21.

23. A cleaning composition comprising the protease variant, or recombinant polypeptide or active fragment thereof of any one of claims 1-21.

24. The cleaning composition of claim 23, wherein said composition further comprises a surfactant; at least one calcium ion and/or zinc ion; at least one stabilizer; from about 0.001% to about 1.0 weight % of said recombinant polypeptide; at least one bleaching agent; at least one adjunct ingredient; one or more additional enzymes or enzyme derivatives selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, galactanases, glucoamylases, hemicellulases, hyaluronidases, keratinases, laccases, lactases, ligninases, lipases, lipoxygenases, mannanases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, metalloproteases, additional serine proteases, or a combination thereof.

25. The cleaning composition of claim 23 or 24, wherein said composition contains phosphate, is phosphate-free, contains borate, is boron-free, or combinations thereof.

26. A method of cleaning comprising contacting a surface or an item in need of cleaning with the protease variant, or recombinant polypeptide or active fragment thereof of any one of claims 1-21 or the cleaning composition of any one of claims 23-25; and optionally further comprising the step of rinsing said surface or item after contacting said surface or item with said variant or composition.

27. A polynucleotide comprising a nucleic acid sequence that encodes the protease variant, or recombinant polypeptide or active fragment thereof of any one of claims 1-21.
